(19) United States
(12) Patent Application Publication
(10) Pub. No.: US 2024/0107877 A1
(43) Pub. Date: Mar. 28, 2024

(54) LIGHT EMITTING DEVICE AND AMINE COMPOUND FOR LIGHT EMITTING DEVICE

(71) Applicant: Samsung Display Co., Ltd., Yongin-si (KR)

(72) Inventors: Jeong Min LEE, Yongin-si (KR); MINJI KIM, Yongin-si (KR); HANKYU PAK, Yongin-si (KR); BYEONGWOOK YOO, Yongin-si (KR); SOHEE JO, Yongin-si (KR)

(73) Assignee: Samsung Display Co., Ltd., Yongin-si (KR)

(21) Appl. No.: 18/322,760

(22) Filed: May 24, 2023

(30) Foreign Application Priority Data
Aug. 4, 2022 (KR) 10-2022-0097374

(51) Int. Cl. H10K 85/60; C07C 211/61; C09K 11/06

(52) U.S. Cl. CPC H10K 85/633; C07C 211/61; C09K 11/06; H10K 50/156

(57) ABSTRACT

Embodiments provide a light emitting device that includes a first electrode, a second electrode facing the first electrode, and a plurality of functional layers disposed between the first electrode and the second electrode. At least one of the functional layers includes an amine compound represented by Formula 1, which is explained in the specification:

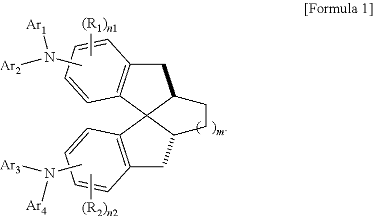

### CROSS-REFERENCE TO RELATED APPLICATION(S)

This application claims priority to and benefits of Korean Patent Application No. 10-2022-0097374 under 35 U.S.C. § 119, filed on Aug. 4, 2022, in the Korean Intellectual Property Office, the entire contents of which are incorporated herein by reference.

### BACKGROUND

### 1. Technical Field

The disclosure relates to an amine compound used as a hole transport material and a light emitting device including the same.

### 2. Description of the Related Art

Active development continues for an organic electroluminescence display apparatus as an image display apparatus. Unlike liquid crystal display apparatuses and the like, the organic electroluminescence display apparatus is a so-called self-luminescent display apparatus in which holes and electrons respectively injected from a first electrode and a second electrode recombine in an emission layer, so that a luminescent material including an organic compound in the emission layer emits light to achieve display.

In the application of an organic electroluminescence device to a display apparatus, there is a demand for an organic electroluminescence device having a low driving voltage, high luminous efficiency, and a long service life, and continuous development is required on materials for an organic electroluminescence device that are capable of stably achieving such characteristics.

In order to achieve such characteristics, materials for a hole transport layer are being developed in order to achieve a highly efficient organic electroluminescence device.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light emitting device in which luminous efficiency and device service life are improved.

The disclosure also provides an amine compound capable of improving luminous efficiency and device service life of a light emitting device.

An embodiment provides a light emitting device which may include a first electrode, a second electrode facing the first electrode, and functional layers disposed between the first electrode and the second electrode, wherein at least one of the functional layers may include an amine compound represented by Formula 1:

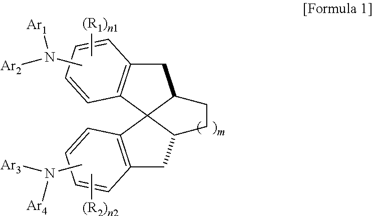

In Formula 1, Ar₁ to Ar₄ may each independently be a substituted or unsubstituted aryl group having 6 to 30 ring-forming carbon atoms or a substituted or unsubstituted heteroaryl group having 2 to 30 ring-forming carbon atoms; R₁ and R₂ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; n1 and n2 may each independently be an integer from 0 to 3, and m may be an integer from 1 to 4.

In an embodiment, the functional layers may include a hole transport region disposed on the first electrode, an emission layer disposed on the hole transport region, and an electron transport region disposed on the emission layer; and the hole transport region may include the amine compound.

In an embodiment, the hole transport region may include a hole injection layer disposed on the first electrode, and a hole transport layer disposed on the hole injection layer; and the hole transport layer may include the amine compound.

In an embodiment, the amine compound represented by Formula 1 may be represented by Formula 2:

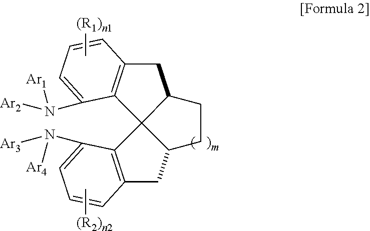

In Formula 2, Ar₁ to Ar₄, R₁, R₂, n1, n2, and m are the same as defined in Formula 1.

In an embodiment, the amine compound represented by Formula 1 may be represented by any one of Formula 3-1 to Formula 3-4:

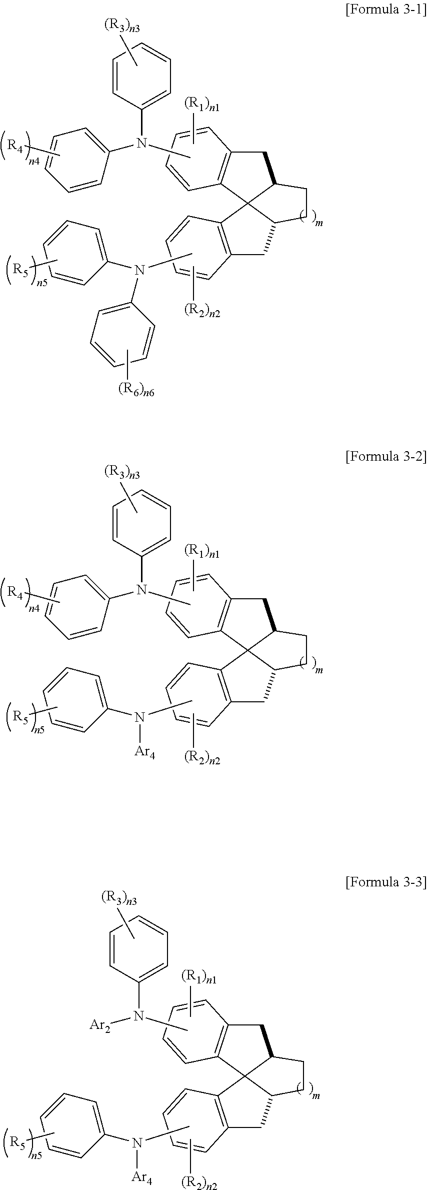

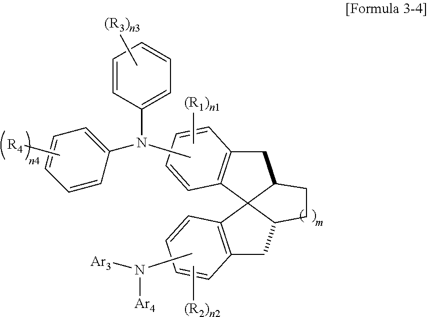

In Formula 3-1 to Formula 3-4, R₃ to R₆ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; and n3 to n6 may each independently be an integer from 0 to 5.

In Formula 3-1 to Formula 3-4, Ar₂ to Ar₄, R₁, R₂, n1, n2, and m are the same as defined in Formula 1.

In an embodiment, the amine compound represented by Formula 1 may be represented by any one of Formula 4-1 to Formula 4-4:

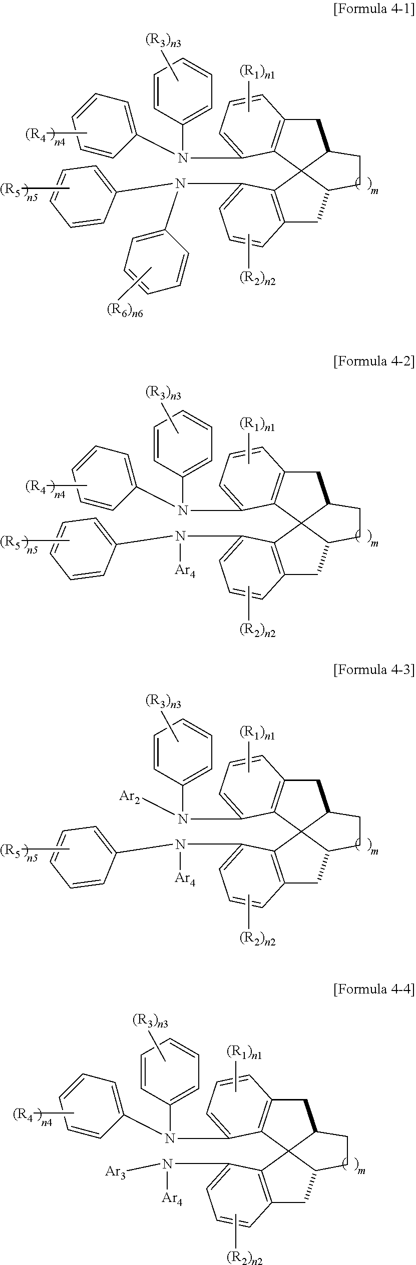

In Formula 4-1 to Formula 4-4, Ar₁ to Ar₄, R₁, R₂, n1, n2, and m are the same as defined in Formula 1; and R₃ to R₆ and n3 to n6 are the same as defined in Formula 3-1 to Formula 3-4.

In an embodiment, the amine compound represented by Formula 1 may be represented by any one of Formula 5-1 to Formula 5-4:

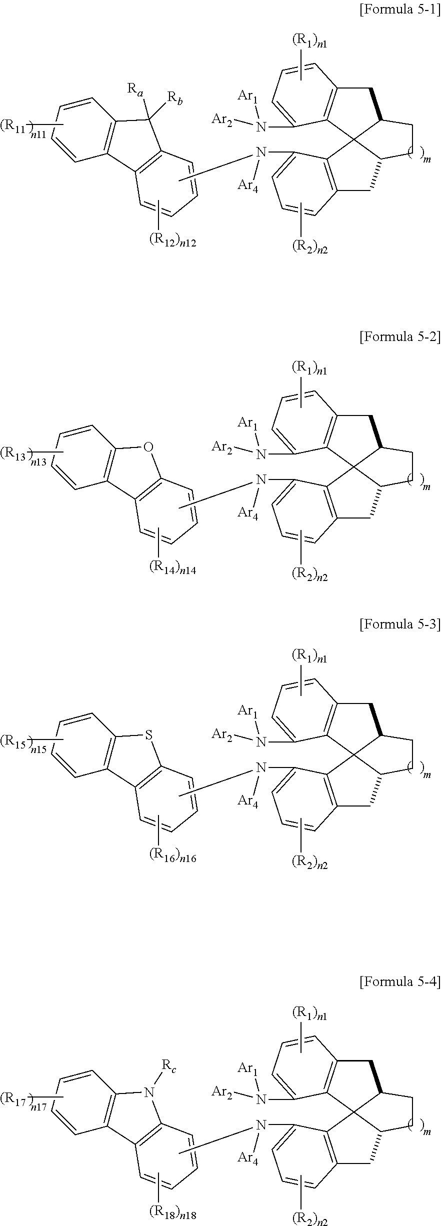

In Formula 5-1 to Formula 5-4, R₁₁ to R₁₈ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; Rₐ to R_{c} may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring; n11, n13, n15, and n17 may each independently be an integer from 0 to 4; and n12, n14, n16, and n18 may each independently be an integer from 0 to 3.

In Formula 5-1 to Formula 5-4, Ar₁, Ar₂, Ar₄, R₁, R₂, n1, n2, and m are the same as defined in Formula 1.

In an embodiment, the amine compound represented by Formula 1 may be represented by any one of Formula 6-1 to Formula 6-6:

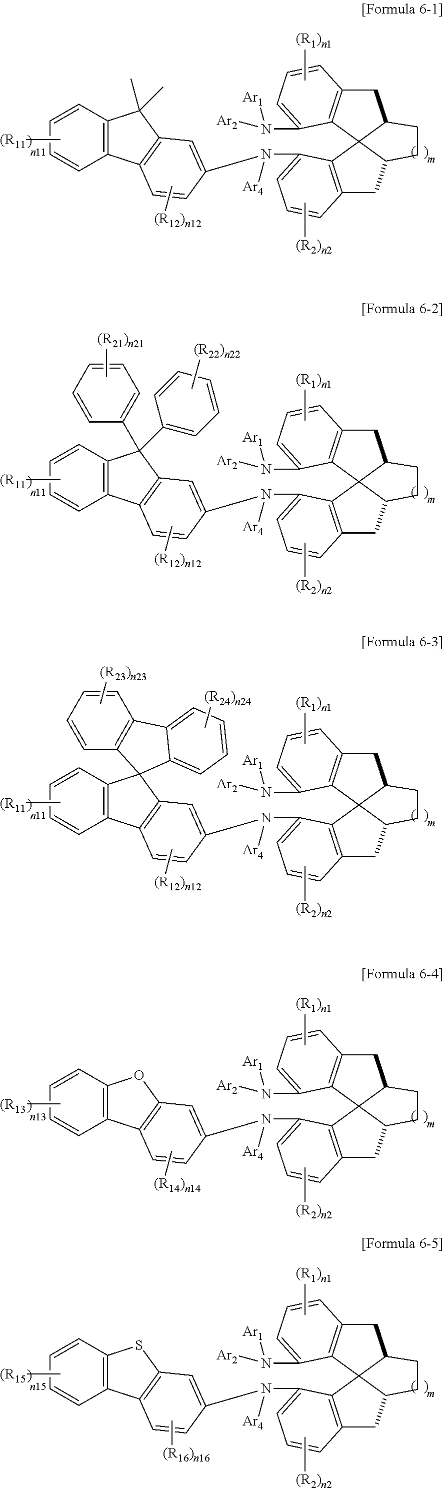

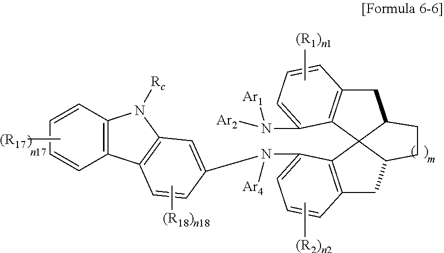

In Formula 6-1 to Formula 6-6, R₂₁ to R₂₄ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms; n21 and n22 may each independently be an integer from 0 to 5; and n23 and n24 may each independently be an integer from 0 to 4.

In Formula 6-1 to Formula 6-6, Ar₁, Ar₂, Ar₄, R₁, R₂, n1, n2, and m are the same as defined in Formula 1; and R₁₁ to R₁₈, R_{c}, and n11 to n18 are the same as defined in Formula 5-1 to Formula 5-4.

In an embodiment, at least one of Ar₁ to Ar₄ may each independently be a group represented by any one of Formula 7-1 to Formula 7-7:

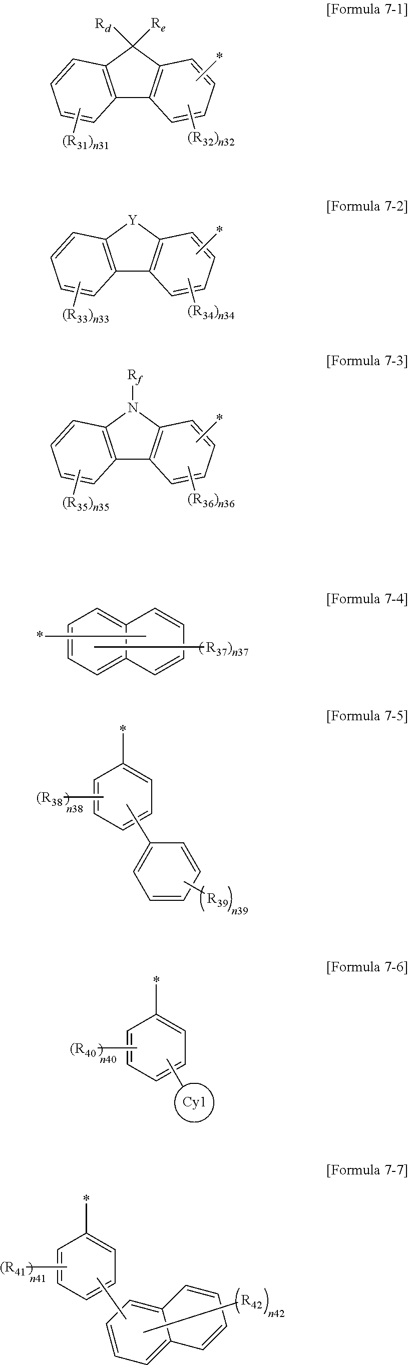

In Formula 7-1 to Formula 7-7, Y may be O or S; R₃₁ to R₄₂ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms;

R_{d} to R_{f} may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring; ring Cy1 may be a substituted or unsubstituted cycloalkyl group having 3 to 20 ring-forming carbon atoms; n31, n33, n35, n38, n40, and n41 may each independently be an integer from 0 to 4; n32, n34, and n36 may each independently be an integer from 0 to 3; n37 and n42 may each independently be an integer from 0 to 7; n39 may be an integer from 0 to 5, and -* represents a bonding site to a nitrogen atom in Formula 1.

In an embodiment, the amine compound represented by Formula 1 may be represented by Formula 8-1 or Formula 8-2:

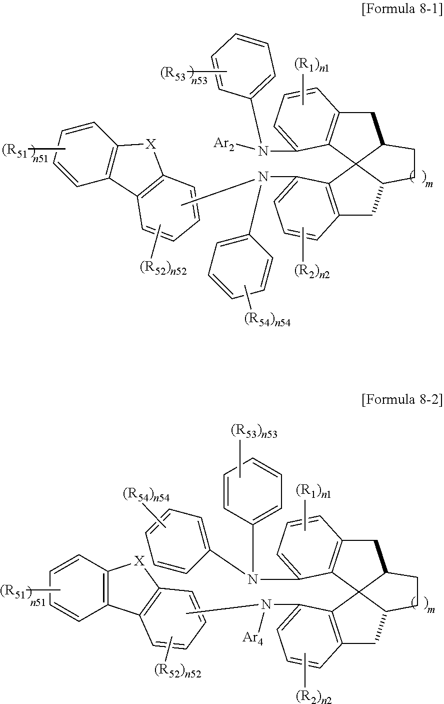

In Formula 8-1 and Formula 8-2, X may be N(R₅₅), C(R₅₆)(R₅₇), O, or S; and R₅₁ to R₅₇ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula 8-1 and Formula 8-2, Ar₂, Ar₄, R₁, R₂, n1, n2, and m are the same as defined in Formula 1.

In an embodiment, the amine compound represented by Formula 1 may be represented by any one of Formula 9-1 to Formula 9-4:

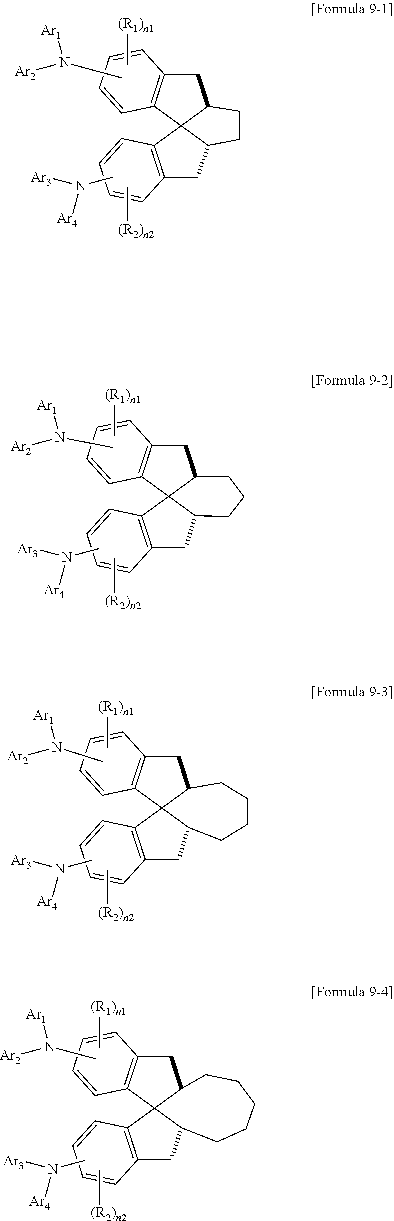

In Formula 9-1 to Formula 9-4, Ar₁ to Ar₄, R₁, R₂, n1, and n2 are the same as defined in Formula 1.

In an embodiment, the amine compound may include at least one compound selected from Compound Group 1, which is explained below.

Another embodiment provides an amine compound which may be represented by Formula 1, which is described herein.

In an embodiment, the amine compound represented by Formula 1 may be represented by Formula 2, which is explained herein.

In an embodiment, the amine compound represented by Formula 1 may be represented by any one of Formula 4-1 to Formula 4-4, which are explained herein.

In an embodiment, the amine compound represented by Formula 1 may be represented by any one of Formula 5-1 to Formula 5-4, which are explained herein.

In an embodiment, at least one of Ar₁ to Ar₄ may each independently be a group represented by any one of Formula 7-1 to Formula 7-7, which are explained herein.

In an embodiment, the amine compound represented by Formula 1 may be represented by Formula 8-1 or Formula 8-2, which are explained herein.

In an embodiment, the amine compound represented by Formula 1 may be represented by any one of Formula 9-1 to Formula 9-4, which are explained herein.

In an embodiment, the amine compound may be selected from Compound Group 1, which is explained below.

It is to be understood that the embodiments above are described in a generic and explanatory sense only and not for the purpose of limitation, and the disclosure is not limited to the embodiments described above.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a schematic plan view of a display apparatus according to an embodiment;

FIG. 2 is a schematic cross-sectional view of a display apparatus according to an embodiment;

FIG. 3 is a schematic cross-sectional view of a light emitting device according to an embodiment;

FIG. 4 is a schematic cross-sectional view of a light emitting device according to an embodiment;

FIG. 5 is a schematic cross-sectional view of a light emitting device according to an embodiment;

FIG. 6 is a schematic cross-sectional view of a light emitting device according to an embodiment;

FIG. 7 is a schematic cross-sectional view of a display apparatus according to an embodiment;

FIG. 8 is a schematic cross-sectional view of a display apparatus according to an embodiment;

FIG. 9 is a schematic cross-sectional view of a display apparatus according to an embodiment; and

FIG. 10 is a schematic cross-sectional view of a display apparatus according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may describe a group that is substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the phrase “bonded to an adjacent group to form a ring” may be interpreted as a group that is bonded to an adjacent group to form a substituted or unsubstituted hydrocarbon ring, or a substituted or unsubstituted heterocycle. The hydrocarbon ring may be an aliphatic hydrocarbon ring or an aromatic hydrocarbon ring. The heterocycle may be an aliphatic heterocycle or an aromatic heterocycle. The hydrocarbon ring and the heterocycle may each independently be monocyclic or polycyclic. A ring that is formed by adjacent groups being bonded to each other may itself be connected to another ring to form a spiro structure.

In the specification, the term “adjacent group” may be interpreted as a substituent that is substituted for an atom which is directly linked to an atom substituted with a corresponding substituent, as another substituent substituted for an atom which is substituted with a corresponding substituent, or as a substituent sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. For example, two methyl groups in 4,5-dimethylphenanthrene may be interpreted as “adjacent groups” to each other.

In the specification, examples of a halogen atom may include a fluorine atom, a chlorine atom, a bromine atom, or an iodine atom.

In the specification, an alkyl group may be linear or branched. The number of carbon atoms in an alkyl group may be 1 to 50, 1 to 30, 1 to 20, 1 to 10, or 1 to 6. Examples of an alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments are not limited thereto.

In the specification, a cycloalkyl group may be a cyclic alkyl group. The number of carbon atoms in a cycloalkyl group may be 3 to 50, 3 to 30, 3 to 20, or 3 to 10. Examples of a cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments are not limited thereto.

In the specification, an alkenyl group may be a hydrocarbon group that includes at least one carbon-carbon double bond in the middle or at a terminus of an alkyl group having 2 or more carbon atoms. An alkenyl group may be linear or branched. The number of carbon atoms in an alkenyl group is not specifically limited, but may be 2 to 30, 2 to 20, or 2 to 10. Examples of an alkenyl group may include a vinyl group, a 1-butenyl group, a 1-pentenyl group, a 1,3-butadienyl aryl group, a styrenyl group, a styryl vinyl group, etc., but embodiments are not limited thereto.

In the specification, an alkynyl group may be a hydrocarbon group that includes at least one carbon-carbon triple bond in the middle or at a terminus of an alkyl group having 2 or more carbon atoms. An alkynyl group may be linear or branched. The number of carbon atoms is not specifically limited, but may be 2 to 30, 2 to 20, or 2 to 10. Examples of an alkynyl group may include an ethynyl group, a propynyl group, etc., without limitation.

In the specification, a hydrocarbon ring group may be any functional group or substituent derived from an aliphatic hydrocarbon ring. For example, a hydrocarbon ring group may be a saturated hydrocarbon ring group having 5 to 20 ring-forming carbon atoms.

In the specification, an aryl group may be any functional group or substituent derived from an aromatic hydrocarbon ring. An aryl group may be monocyclic or polycyclic. The number of ring-forming carbon atoms in an aryl group may be 6 to 30, 6 to 20, or 6 to 15. Examples of an aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments are not limited thereto.

In the specification, a fluorenyl group may be substituted, and two substituents may be bonded to each other to form a spiro structure. Examples of substituted fluorenyl groups are as follows. However, embodiments are not limited thereto.

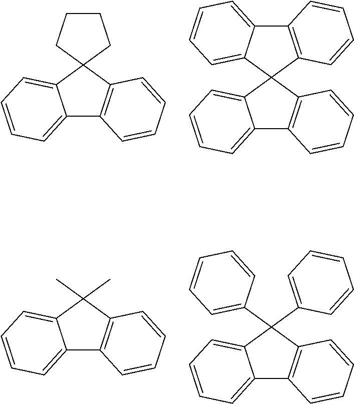

In the specification, a heterocyclic group may be any functional group or substituent derived from a ring that includes at least one of B, O, N, P, S, Si, or Se as a heteroatom. A heterocyclic group may be an aliphatic heterocyclic group or an aromatic heterocyclic group. An aromatic heterocyclic group may be a heteroaryl group. The aliphatic heterocycle and the aromatic heterocycle may each independently be monocyclic or polycyclic.

In the specification, a heterocyclic group may include at least one of B, O, N, P, S, Si, or Se as a heteroatom. If a heterocyclic group contains two or more heteroatoms, the two or more heteroatoms may be the same as or different from each other. A heterocyclic group may be monocyclic or polycyclic, and a heterocyclic group may be a heteroaryl group. The number of ring-forming carbon atoms in a heterocyclic group may be 2 to 30, 2 to 20, or 2 to 10.

In the specification, an aliphatic heterocyclic group may include at least one of B, O, N, P, Si, or S as a heteroatom. The number of ring-forming carbon atoms in an aliphatic heterocyclic group may be 2 to 30, 2 to 20, or 2 to 10. Examples of an aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group etc., but embodiments are not limited thereto.

In the specification, a heteroaryl group may include at least one of B, O, N, P, Si, or S as a heteroatom. When a heteroaryl group contains two or more heteroatoms, the two or more heteroatoms may be the same as or different from each other. A heteroaryl group may be monocyclic or polycyclic. The number of ring-forming carbon atoms in a heteroaryl group may be 2 to 30, 2 to 20, or 2 to 10. Examples of a heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments are not limited thereto.

In the specification, the above description of the aryl group may be applied to an arylene group, except that the arylene group is a divalent group. The above description of the heteroaryl group may be applied to a heteroarylene group, except that the heteroarylene group is a divalent group.

In the specification, a silyl group may be an alkylsilyl group or an arylsilyl group. Examples of a silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments are not limited thereto.

In the specification, the number of carbon atoms in an amino group is not specifically limited, but may be 1 to 30. An amino group may be an alkyl amino group, an aryl amino group, or a heteroaryl amino group. Examples of an amino group may include a methylamino group, a dimethylamino group, a phenylamino group, a diphenylamino group, a naphthylamino group, a 9-methyl-anthracenylamino group, a triphenylamino group, etc., but are not limited thereto.

In the specification, the number of ring-forming carbon atoms in a carbonyl group is not specifically limited, but may be 1 to 40, 1 to 30, or 1 to 20. For example, a carbonyl group may have one of the following structures, but embodiments are not limited thereto.

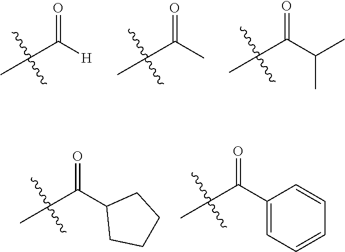

In the specification, the number of carbon atoms in a sulfinyl group or a sulfonyl group is not particularly limited, but may be 1 to 30. A sulfinyl group may be an alkyl sulfinyl group or an aryl sulfinyl group. A sulfonyl group may be an alkyl sulfonyl group or an aryl sulfonyl group.

In the specification, a thio group may be an alkylthio group or an arylthio group. A thio group may be a sulfur atom that is bonded to an alkyl group or an aryl group as defined above. Examples of a thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments are not limited thereto.

In the specification, an oxy group may be an oxygen atom that is bonded to an alkyl group or an aryl group as defined above. An oxy group may be an alkoxy group or an aryl oxy group. An alkoxy group may be linear chain, branched, or cyclic. The number of carbon atoms in an alkoxy group or an aryl oxy is not specifically limited, but may be, for example, 1 to 20 or 1 to 10. Examples of an oxy group may include methoxy, ethoxy, n-propoxy, isopropoxy, butoxy, pentyloxy, hexyloxy, octyloxy, nonyloxy, decyloxy, benzyloxy, etc., but embodiments are not limited thereto.

In the specification, a boron group may be a boron atom that is bonded to an alkyl group or an aryl group as defined above. A boron group may be an alkyl boron group or an aryl boron group. Examples of a boron group may include a dimethylboron group, a trimethylboron group, a t-butyldimethylboron group, a diphenylboron group, a phenylboron group, etc., but embodiments are not limited thereto.

In the specification, the number of carbon atoms in an amine group is not specifically limited, but may be 1 to 30. An amine group may be an alkyl amine group or an aryl amine group. Examples of an amine group may include a methylamine group, a dimethylamine group, a phenylamine group, a diphenylamine group, a naphthylamine group, a 9-methyl-anthracenylamine group, a triphenylamine group, etc., but embodiments are not limited thereto.

In the specification, the alkyl group in an alkylthio group, an alkylsulfoxy group, an alkylaryl group, an alkylamino group, an alkyl boron group, an alkyl silyl group, or an alkyl amine group may be the same as an example of an alkyl group as described above.

In the specification, the aryl group in an aryloxy group, an arylthio group, an arylsulfoxy group, an arylamino group, an arylboron group, an arylsilyl group, or an arylamine group may be the same as an example of an aryl group as described above.

In the specification, a direct linkage may be a single bond.

In the specification, the symbol -* represents a bonding site to a neighboring atom.

Hereinafter, embodiments will be described with reference to the accompanying drawings.

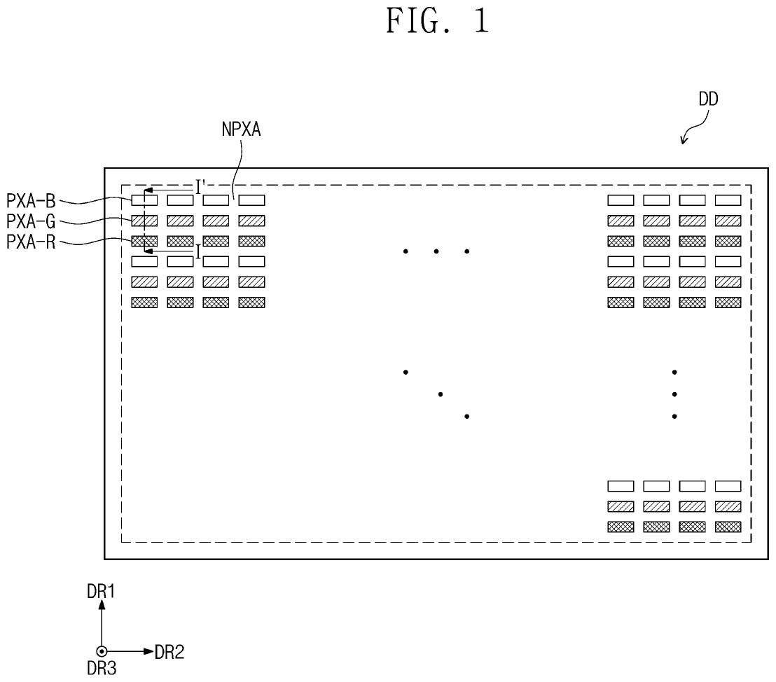

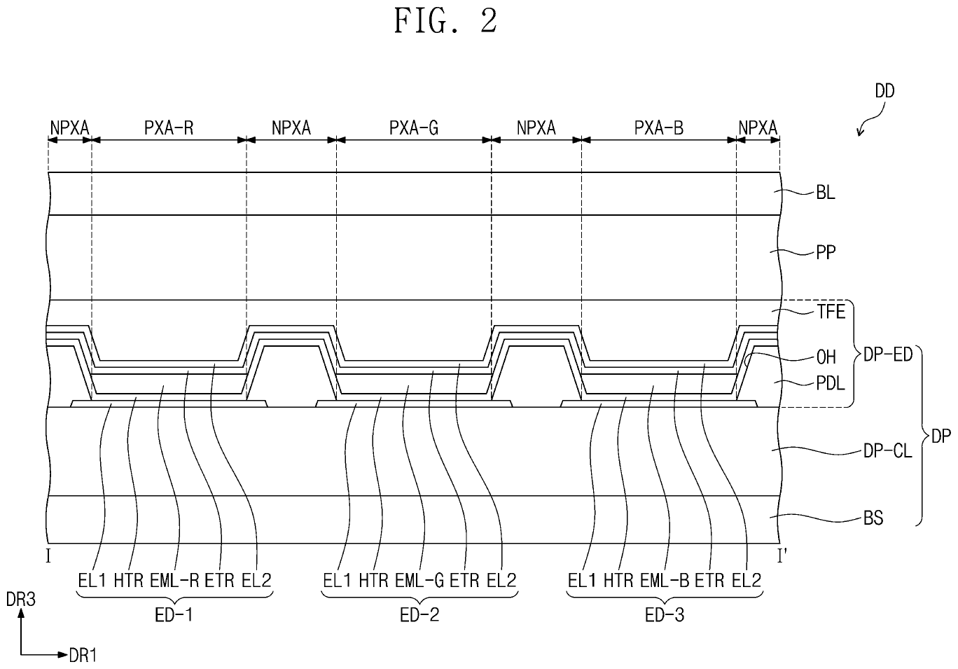

FIG. 1 is a plan view illustrating an embodiment of a display apparatus DD. FIG. 2 is a schematic cross-sectional view of the display apparatus DD of the embodiment. FIG. 2 is a schematic cross-sectional view illustrating a part taken along line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include multiples of each of the light emitting devices ED-1, ED-2, and ED-3. The optical layer PP may be disposed on the display panel DP and may control light that is reflected at the display panel DP from an external light. The optical layer PP may include, for example, a polarization layer or a color filter layer. Although not shown in the drawing, in an embodiment, the optical layer PP may be omitted from the display apparatus DD.

A base substrate BL may be disposed on the optical layer PP. The base substrate BL may provide a base surface on which the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

The display apparatus DD according to an embodiment may further include a filling layer (not shown). The filling layer (not shown) may be disposed between a display device layer DP-ED and the base substrate BL. The filling layer (not shown) may be an organic material layer. The filling layer (not shown) may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, light emitting devices ED-1, ED-2, and ED-3 disposed between portions of the pixel defining film PDL, and an encapsulation layer TFE disposed on the light emitting devices ED-1, ED-2, and ED-3.

The base layer BS may provide a base surface on which the display device layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). The transistors (not shown) may each include a control electrode, an input electrode, and an output electrode. For example, the circuit layer DP-CL may include a switching transistor and a driving transistor for driving the light emitting devices ED-1, ED-2, and ED-3 of the display device layer DP-ED.

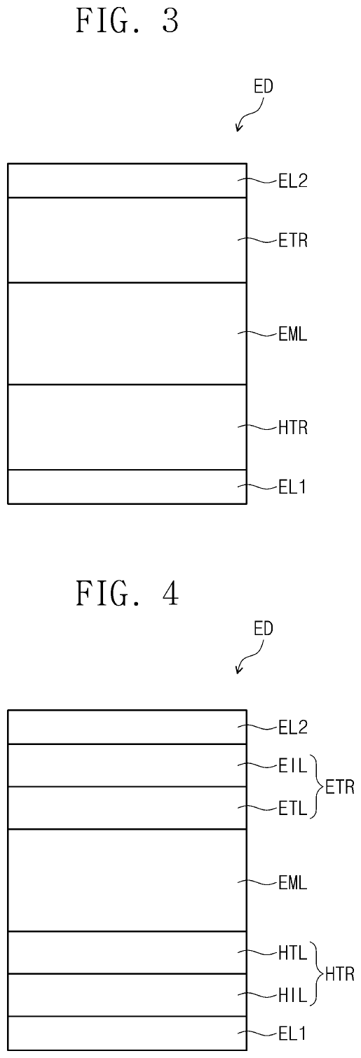

The light emitting devices ED-1, ED-2, and ED-3 may each have a structure of a light emitting device ED of an embodiment according to any of FIGS. 3 to 6, which will be described later. The light emitting devices ED-1, ED-2, and ED-3 may each include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting devices ED-1, ED-2, and ED-3 are disposed in openings OH defined in the pixel defining film PDL, and a hole transport region HTR, an electron transport region ETR, and a second electrode EL2 are each provided as a common layer for all of the light emitting devices ED-1, ED-2, and ED-3. However, embodiments are not limited thereto. Although not shown in FIG. 2, in an embodiment, the hole transport region HTR and the electron transport region ETR may each be provided by being patterned inside the openings OH defined in the pixel defining film PDL. For example, the hole transport region HTR, the emission layers EML-R, EML-G, and EML-B, and the electron transport region ETR of the light emitting devices ED-1, ED-2, and ED-3 may each be provided by being patterned through an inkjet printing method.

The encapsulation layer TFE may cover the light emitting devices ED-1, ED-2, and ED-3. The encapsulation layer TFE may seal the display device layer DP-ED. The encapsulation layer TFE may be a thin film encapsulation layer. The encapsulation layer TFE may be formed of a single layer or of multiple layers. The encapsulation layer TFE may include at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film may protect the display device layer DP-ED from moisture and/or oxygen, and the encapsulation-organic film may protect the display device layer DP-ED from foreign substances such as dust particles. The encapsulation-inorganic film may include silicon nitride, silicon oxynitride, silicon oxide, titanium oxide, aluminum oxide, or the like, but embodiments are not limited thereto. The encapsulation-organic film may include an acrylic-based compound, an epoxy-based compound, or the like. The encapsulation-organic film may include a photopolymerizable organic material, but embodiments are not limited thereto.

The encapsulation layer TFE may be disposed on the second electrode EL2 and may be disposed to fill the openings OH.

Referring to FIGS. 1 and 2, the display apparatus DD may include non-light emitting regions NPXA and light emitting regions PXA-R, PXA-G, and PXA-B. The light emitting regions PXA-R, PXA-G, and PXA-B may each be a region which emits light generated by the respective light emitting devices ED-1, ED-2, and ED-3. The light emitting regions PXA-R, PXA-G, and PXA-B may be spaced apart from each other in a plan view.

The light emitting regions PXA-R, PXA-G, and PXA-B may each be a region separated by the pixel defining film PDL. The non-light emitting regions NPXA may be areas between neighboring light emitting areas PXA-R, PXA-G, and PXA-B, and may correspond to the pixel defining film PDL. For example, in an embodiment, the light emitting regions PXA-R, PXA-G, and PXA-B may each respectively correspond to a pixel. The pixel defining film PDL may separate the light emitting devices ED-1, ED-2, and ED-3. The emission layers EML-R, EML-G, and EML-B of the light emitting devices ED-1, ED-2, and ED-3 may be disposed in the openings OH defined in the pixel defining film PDL and separated from each other.

The light emitting regions PXA-R, PXA-G, and PXA-B may be arranged into groups according to the color of light generated from the light emitting devices ED-1, ED-2, and ED-3. In the display apparatus DD according to an embodiment illustrated in FIGS. 1 and 2, three light emitting regions PXA-R, PXA-G, and PXA-B, which respectively emit red light, green light, and blue light are illustrated. For example, the display apparatus DD may include the red light emitting region PXA-R, the green light emitting region PXA-G, and the blue light emitting region PXA-B, which are separated from each other.

In the display apparatus DD according to an embodiment, the light emitting devices ED-1, ED-2 and ED-3 may emit light having wavelengths that are different from each other.

For example, in an embodiment, the display apparatus DD may include a first light emitting device ED-1 that emits red light, a second light emitting device ED-2 that emits green light, and a third light emitting device ED-3 that emits blue light. For example, the red light emitting region PXA-R, the green light emitting region PXA-G, and the blue light emitting region PXA-B of the display apparatus DD may respectively correspond to the first light emitting device ED-1, the second light emitting device ED-2, and the third light emitting device ED-3.

However, embodiments are not limited thereto, and the first to third light emitting devices ED-1, ED-2, and ED-3 may each emit light in a same wavelength range, or at least one light emitting device may emit light in a wavelength range that is different from the others. For example, the first to third light emitting devices ED-1, ED-2, and ED-3 may all emit blue light.

The light emitting regions PXA-R, PXA-G, and PXA-B in the display apparatus DD according to an embodiment may be arranged in a stripe configuration. Referring to FIG. 1, the red light emitting regions PXA-R, the green light emitting regions PXA-G, and the blue light emitting regions PXA-B may each be arranged along a second directional axis DR2. In another embodiment, the red light emitting region PXA-R, the green light emitting region PXA-G, and the blue light emitting region PXA-B may be alternately arranged in this order along a first directional axis DR1.

FIGS. 1 and 2 illustrate that the light emitting regions PXA-R, PXA-G, and PXA-B all have a similar area, but embodiments are not limited thereto. The light emitting regions PXA-R, PXA-G, and PXA-B may have different areas from each other according to a wavelength range of emitted light. For example, the areas of the light emitting regions PXA-R, PXA-G, and PXA-B may be areas in a plan view that are defined by the first directional axis DR1 and the second directional axis DR2.

An arrangement of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in various combinations according to the display quality characteristics that are required for the display apparatus DD. For example, the light emitting regions PXA-R, PXA-G, and PXA-B may be arranged in a pentile configuration (such as a PENTILE™ configuration) or in a diamond configuration (such as a Diamond Pixel™ configuration).

The areas of the light emitting regions PXA-R, PXA-G, and PXA-B may be different in size from each other. For example, in an embodiment, an area of the green light emitting region PXA-G may be smaller than an area of the blue light emitting region PXA-B, but embodiments are not limited thereto.

FIGS. 3 to 6 are each a schematic cross-sectional view illustrating a light emitting device ED according to an embodiment. The light emitting devices ED according to embodiments may each include a first electrode EL1, a hole transport region HTR, an emission layer EML, an electron transport region ETR, and a second electrode EL2 that are stacked in that order.

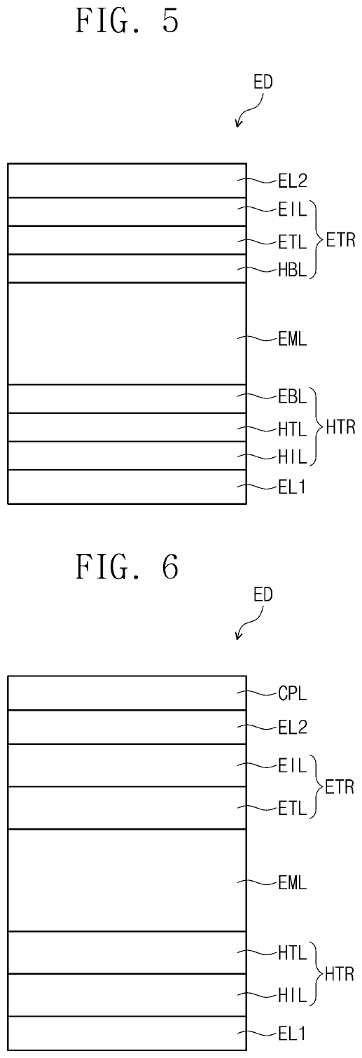

In comparison to FIG. 3, FIG. 4 illustrates a schematic cross-sectional view of a light emitting device ED according to an embodiment, in which a hole transport region HTR includes a hole injection layer HIL and a hole transport layer HTL, and an electron transport region ETR includes an electron injection layer EIL and an electron transport layer ETL. In comparison to FIG. 3, FIG. 5 illustrates a schematic cross-sectional view of a light emitting device ED according to an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 illustrates a schematic cross-sectional view of a light emitting device ED according to an embodiment that includes a capping layer CPL disposed on a second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. For example, the first electrode EL1 may be a pixel electrode. The first electrode EL1 may be a transmissive electrode, a transflective electrode, or a reflective electrode. The first electrode EL1 may be a transmissive electrode, a transflective electrode, or a reflective electrode. The first electrode EL1 may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, or a mixture thereof.

If the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In another embodiment, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but embodiments are not limited thereto. The first electrode EL1 may include the above-described metal materials, combinations of at least two metal materials of the above-described metal materials, oxides of the above-described metal materials, or the like. A thickness of the first electrode EL1 may be in a range of about 700 Å to about 10,000 Å. For example, the thickness of the first electrode EL1 may be in a range of about 1,000 Å to about 3,000 Å.

The hole transport region HTR is provided on the first electrode EL1. The hole transport region HTR may include at least one of a hole injection layer HIL, a hole transport layer HTL, a buffer layer (not shown), an emission-auxiliary layer (not shown), or an electron blocking layer EBL. A thickness of the hole transport region HTR may be, for example, in a range of about 50 Å to about 15,000 Å.

The hole transport region HTR may be a layer consisting of a single material, a layer including different materials, or a structure including multiple layers including different materials.

For example, the hole transport region HTR may have a single layer structure of a hole injection layer HIL or a hole transport layer HTL, or may have a single layer structure formed of a hole injection material and a hole transport material. In embodiment, the hole transport region HTR may have a single layer structure formed of different materials, or may have a structure in which a hole injection layer HIL/hole transport layer HTL, a hole injection layer HIL/hole transport layer HTL/buffer layer (not shown), a hole injection layer HIL/buffer layer (not shown), a hole transport layer HTL/buffer layer (not shown), or a hole injection layer HIL/hole transport layer HTL/electron blocking layer EBL are stacked in its respective stated order from the first electrode EL1, but embodiments are not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR of the light emitting device ED may include an amine compound according to an embodiment. The hole transport region HTR of the light emitting device ED according to an embodiment may include an amine compound represented by Formula 1, which is explained below. In embodiments, the hole transport region HTR of the light emitting device ED may include at least one of a hole injection layer HIL, a hole transport layer HTL, or an electron blocking layer EBL, and at least one of the hole injection layer HIL, the hole transport layer HTL, or the electron blocking layer EBL may include the amine compound represented by Formula 1. For example, the hole transport layer HTL of the light emitting device ED may include an amine compound represented by Formula 1.

The amine compound according to an embodiment may include a spirobiindane moiety and two amine groups linked to the spirobiindane moiety, and the amine compound may include an alkyl substituent which is linked to two indane rings to form a ring in the spirobiindane moiety. The spirobiindane moiety may have a structure in which a first indane ring and a second indane ring are spiro-bonded at the position of carbon 1 that is an sp³ carbon. The carbon numbers of the spirobiindane moiety is shown in Formula S:

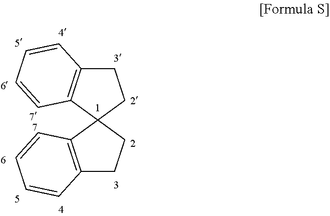 [SMILES: c1ccc2c(c1)CCC21CCc2ccccc21]

With respect to the carbon numbering of the spirobiindane moiety, the numbers are assigned in order in a clockwise direction from the spiro atom among the carbon atoms constituting the second indane ring disposed in a lower part like Formula S above, and the numbers of carbon atoms constituting the first indane ring disposed in an upper part are assigned in order in a counterclockwise direction from the spiro atom. The numbers of the first indane ring are indicated by marking a prime mark (′) to the carbon number except for the spiro atom. In the spirobiindane moiety, the carbon numbers at the ring condensation portions are excluded.

The amine compound according to an embodiment includes a spirobiindane moiety and includes a first amine group and a second amine group linked to the spirobiindane moiety. The first amine group and the second amine group may be respectively linked to a first benzene ring of the first indane ring and a second benzene ring of the second indane ring. An alkyl substituent may link the first indane ring and the second indane ring. The alkyl substituent may be linked to carbon 2 and carbon 2′ of the spirobiindane moiety. In an embodiment, the alkyl substituent may be linked to carbon 2 and carbon 2′ of the spirobiindane moiety to form a cyclopentyl ring, a cyclohexyl ring, or a cyclooctyl ring. In an embodiment, the alkyl substituent may be an ethyl group, a propyl group, a butyl group, or a pentyl group.

The amine compound according to an embodiment is represented by Formula 1:

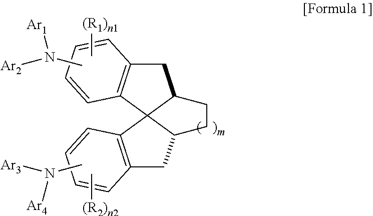

In Formula 1, Ar₁ to Ar₄ may each independently be a substituted or unsubstituted aryl group having 6 to 30 ring-forming carbon atoms or a substituted or unsubstituted heteroaryl group having 2 to 30 ring-forming carbon atoms. For example, Ar₁ to Ar₄ may each independently be a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted spirobifluorene group, a substituted or unsubstituted dibenzofuran group, a substituted or unsubstituted dibenzothiophene group, a substituted or unsubstituted carbazole group, a substituted or unsubstituted phenylnaphthyl group, or a substituted or unsubstituted naphthyl phenyl group. In Formula 1, the amine group to which Ar₁ and Ar₂ are linked corresponds to the first amine group, and the amine group to which Ar₃ and Ar₄ are linked corresponds to the second amine group.

In Formula 1, R₁ and R₂ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₁ and R₂ may each be a hydrogen atom.

In Formula 1, n1 and n2 may each independently be an integer from 0 to 3. If n1 and n2 are each 0, the amine compound may not be substituted with R₁ and R₂. A case where n1 and n2 are each 3 and R₁ groups and R₂ groups are each hydrogen atoms may be the same as a case where n1 and n2 are each 0 in Formula 1. When n1 and n2 are each 2 or more, multiple R₁ groups and multiple R₂ groups may be the same as each other, or at least one thereof may be different.

In Formula 1, m may be an integer from 1 to 4. If m is 1, the alkyl substituent is an ethyl group, and this is a case where in the amine compound represented by Formula 1, the alkyl substituent is linked to carbon 2 and carbon 2′ of the spirobiindane moiety to form a cyclopentyl ring. If m is 2, the alkyl substituent is a propyl group, and this is a case where in the amine compound represented by Formula 1, the alkyl substituent is linked to carbon 2 and carbon 2′ of the spirobiindane moiety to form a cyclohexyl ring. If m is 3, the alkyl substituent is a butyl group, and this is a case where in the amine compound represented by Formula 1, the alkyl substituent is linked to carbon 2 and carbon 2′ of the spirobiindane moiety to form a cycloheptyl ring. If m is 4, the alkyl substituent is a pentyl group, and this is a case where in the amine compound represented by Formula 1, the alkyl substituent is linked to carbon 2 and carbon 2′ of the spirobiindane moiety to form a cyclooctyl ring.

The amine compound represented by Formula 1 may be a chiral compound. In the specification, a chiral compound may be a compound in which a real image and a mirror image are not superimposed. The amine compound represented by Formula 1 may be represented by Formula 1-1 or Formula 1-2, which are explained below. The amine compound represented by Formula 1 may include at least one of an R configuration enantiomer represented by Formula 1-1, an S configuration enantiomer represented by Formula 1-2, or a racemic mixture thereof. This description may be equally applied in Formula 2, Formula 3-1 to Formula 3-4, Formula 4-1 to Formula 4-4, Formula 5-1 to Formula 5-4, Formula 6-1 to Formula 6-6, Formula 8-1, Formula 8-2, and Formula 9-1 to Formula 9-4, which will be described later.

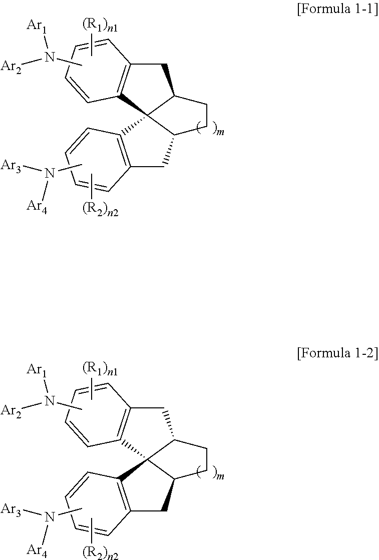

In Formula 1-1 and Formula 1-2, Ar₁ to Ar₄, R₁, R₂, n1, n2, and m are the same as described in Formula 1.

In an embodiment, the amine compound represented by Formula 1 may be represented by Formula 2:

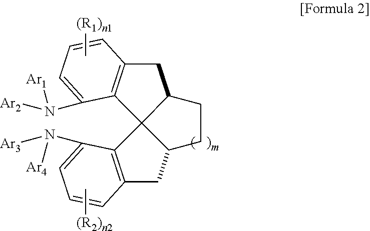

Formula 2 represents a case where a position at which a first nitrogen atom of the first amine group is linked to the spirobiindane moiety and a position at which a second nitrogen atom of the second amine group is linked to the spirobiindane moiety are each specified in Formula 1. Formula 2 corresponds to a case where the first nitrogen atom of the first amine group is linked to carbon 7′ of the spirobiindane moiety and the second nitrogen atom of the second amine group is linked to carbon 7 of the spirobiindane moiety in Formula 1.

In Formula 2, Ar₁ to Ar₄, R₁, R₂, n1, n2, and m are the same as described in Formula 1.

In an embodiment, the amine compound represented by Formula 1 may be represented by any one of Formula 3-1 to Formula 3-4:

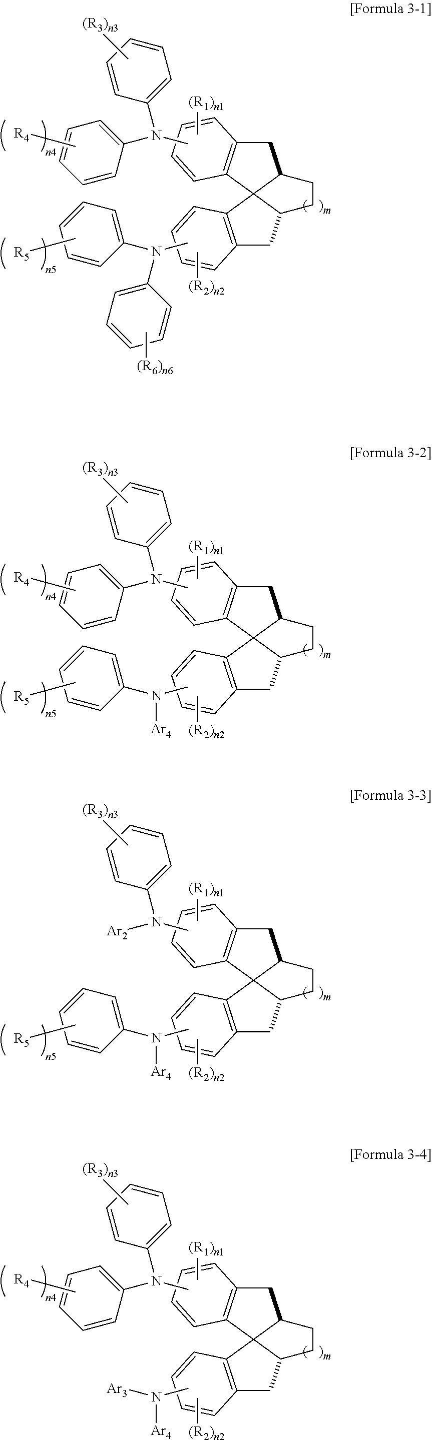

Formula 3-1 to Formula 3-4 each represent a case where the types of substituents linked to the first amine group and/or the second amine group are specified in the structure of Formula 1. Formula 3-1 to Formula 3-4 each represent a case where at least two of Ar₁ to Ar₄ are specified in the structure of Formula 1. Formula 3-1 represents a case where Ar₁ to Ar₄ in Formula 1 are all substituted or unsubstituted phenyl groups. Formula 3-2 represents a case where Ar₁ to Ar₃ in Formula 1 are all substituted or unsubstituted phenyl groups. Formula 3-3 represents a case where Ar₁ and Ar₃ in Formula 1 are each a substituted or unsubstituted phenyl group. Formula 3-4 represents a case where Ar₁ and Ar₂ in Formula 1 are each a substituted or unsubstituted phenyl group.

In Formula 3-1 to Formula 3-4, R₃ to R₆ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₃ to R₆ may each be a hydrogen atom.

In Formula 3-1 to Formula 3-4, n3 to n6 may each independently be an integer from 0 to 5. If n3 to n6 are each 0, the amine compound may not be substituted with each of R₃ to R₆. A case where n3 to n6 are each 5 and R₃ groups to R₆ groups are each hydrogen atoms may be the same as a case where n3 to n6 are each 0 in Formula 3-1 to Formula 3-4. When n3 to n6 are each 2 or more, multiple groups of each of R₃ to R₆ may be the same as each other, or at least one thereof may be different from the others.

In Formula 3-1 to Formula 3-4, Ar₂ to Ar₄, R₁, R₂, n1, n2, and m are the same as described in Formula 1.

In an embodiment, the amine compound represented by Formula 1 may be represented by any one of Formula 4-1 to Formula 4-4:

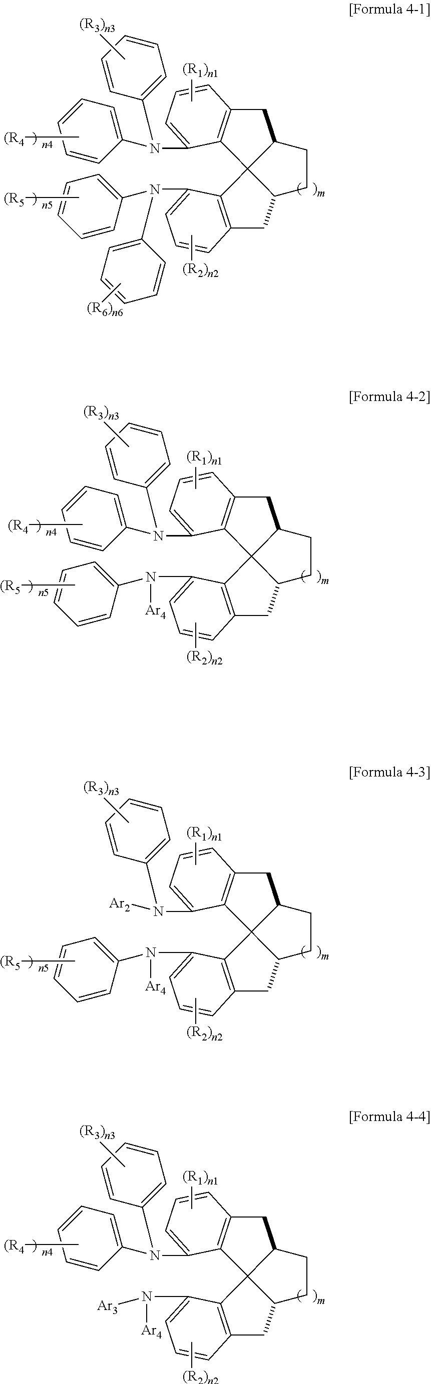

Formula 4-1 to Formula 4-4 each represent a case where in Formula 1, carbon positions at which the first amine group and the second amine group are linked to the spirobiindane moiety are specified and the types of substituents linked to the first amine group and/or the second amine group are specified.

In Formula 4-1 and Formula 4-4, Ar₂ to Ar₄, R₁, R₂, n1, n2, and m are the same as described in Formula 1, and R₃ to R₆ and n3 to n6 are the same as defined in Formula 3-1 to Formula 3-4.

In an embodiment, the amine compound represented by Formula 1 may be represented by any one of Formula 5-1 to Formula 5-4:

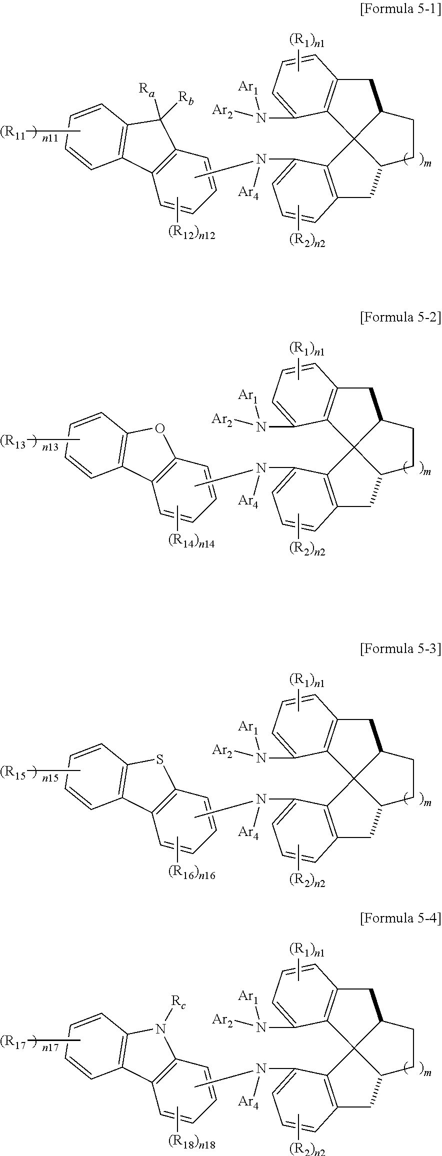

Formula 5-1 to Formula 5-4 each represent a case where Ar₃ is specified in the structure of Formula 1. Formula 5-1 represents a case where Ar₃ in Formula 1 is a substituted or unsubstituted fluorenyl group, or a substituted or unsubstituted spirobifluorenyl group. Formula 5-2 represents a case where Ar₃ in Formula 1 is a substituted or unsubstituted dibenzofuran group. Formula 5-3 represents a case where Ar₃ in Formula 1 is a substituted or unsubstituted dibenzothiophene group. Formula 5-4 represents a case where Ar₃ in Formula 1 is a substituted or unsubstituted carbazole group.

In Formula 5-1 to Formula 5-4, R₁₁ to R₁₈ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₁₁ to R₁₈ may each independently be a hydrogen atom or a substituted or unsubstituted phenyl group.

In Formula 5-1 and Formula 5-4, Rₐ to R_{c} may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, Rₐ to R_{c} may each independently be a substituted or unsubstituted phenyl group. As another example, Rₐ and R_{b} may be bonded to each other to form a ring. When Rₐ and R_{b} are bonded to each other to form a ring, the fluorenyl group linked to the amine compound represented by Formula 5-1 may have a spiro structure.

In Formula 5-1 to Formula 5-4, n1l, n13, n15, and n17 may each independently be an integer from 0 to 4. If n11, n13, n15, and n17 are each 0, the amine compound may not be substituted with R₁, R₁₃, R₁₅, and R₁₇. A case where n11, n13, n15, and n17 are each 4 and R₁ groups, R₁₃ groups, R₁₅ groups, and R₁₇ groups are each hydrogen atoms may be the same as a case where n11, n13, n15, and n17 are each 0. If n11, n13, n15, and n17 are each 2 or more, multiple groups of each of R₁, R₁₃, R₁₈, and R₁₇ may be the same as each other, or at least one thereof may be different from the others.

In Formula 5-1 to Formula 5-4, n12, n14, n16, and n18 may each independently be an integer from 0 to 3. If n12, n14, n16, and n18 are each 0, the amine compound may not be substituted with R₁₂, R₁₄, R₁₆, and R₁₈. A case where n12, n14, n16, and n18 are each 3 and R₁₂ groups, R₁₄ groups, R₁₆ groups, and R₁₈ groups are each hydrogen atoms may be the same as a case where n12, n14, n16, and n18 are each 0. If n12, n14, n16, and n18 are each 2 or more, multiple groups of each of R₁₂, R₁₄, R₁₆, and R₁₈ may be the same as each other, or at least one thereof may be different from the others.

In Formula 5-1 to Formula 5-4, Ar₁, Ar₂, Ar₄, R₁, R₂, n1, n2, and m are the same as described in Formula 1.

In an embodiment, the amine compound represented by Formula 1 may be represented by any one of Formula 6-1 to Formula 6-6:

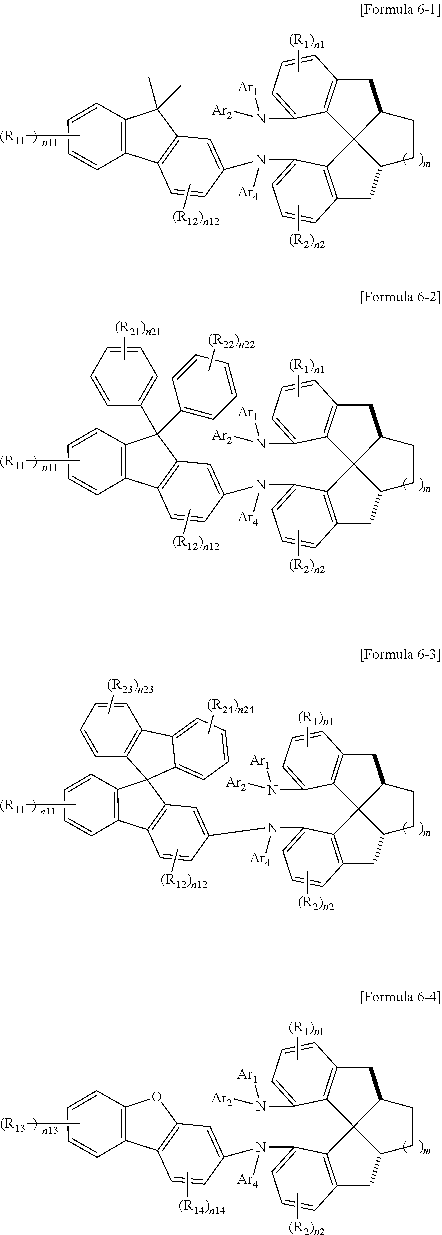

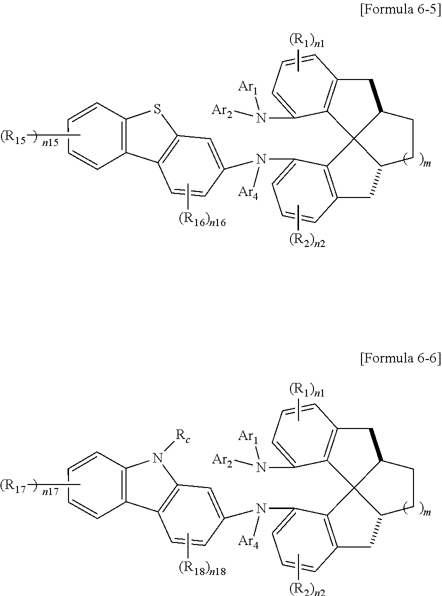

Formula 6-1 to Formula 6-6 each represent a case where Ar₃ and its bonding positions are specified in the structure of Formula 1.

In Formula 6-2 and Formula 6-3, R₂₁ to R₂₄ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₂₁ to R₂₄ may each be a hydrogen atom.

In Formula 6-2, n21 and n22 may each independently be an integer from 0 to 5. If n21 and n22 are each 0, the amine compound may not be substituted with R₂₁ and R₂₂. A case where n21 and n22 are each 5 and R₂₁ groups and R₂₂ groups are each hydrogen atoms may be the same as a case where n21 and n22 are each 0. When n21 and n22 are each 2 or more, multiple R₂₁ groups and multiple R₂₂ groups may be the same as each other, or at least one thereof may be different from the others.

In Formula 6-3, n23 and n24 may each independently be an integer from 0 to 4. If n23 and n24 are each 0, the amine compound may not be substituted with R₂₃ and R₂₄. A case where n23 and n24 are each 4 and R₂₃ groups and R₂₄ groups are each hydrogen atoms may be the same as a case where n23 and n24 are each 0. When n23 and n24 are each 2 or more, multiple R₂₃ groups and multiple R₂₄ groups may be the same as each other, or at least one thereof may be different from the others.

In Formula 6-1 to Formula 6-6, Ar₁, Ar₂, Ar₄, R₁, R₂, n1, n2, and m are the same as described in Formula 1 and R₁₁ to R₁₈, R_{c}, and n11 to n18 are the same as described in Formula 5-1 to Formula 5-4.

In an embodiment, at least one of Ar₁ to Ar₄ may each independently be a group represented by any one of Formula 7-1 to Formula 7-7:

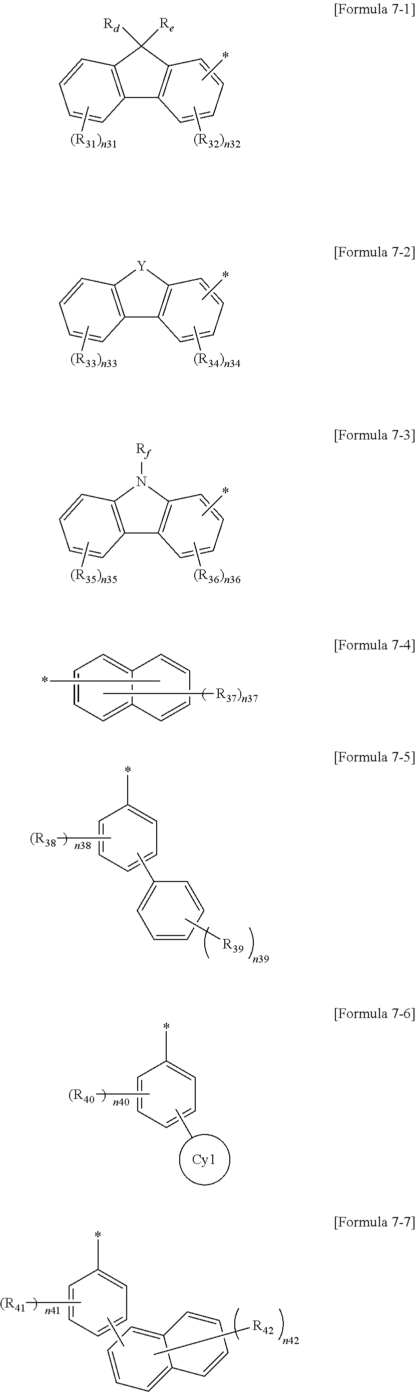

In Formula 7-2, Y may be O or S.

In Formula 7-1 to Formula 7-7, R₃₁ to R₄₂ may each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₃₁ to R₄₂ may each independently be a hydrogen atom, a substituted or unsubstituted t-butyl group, a substituted or unsubstituted cyclohexyl group, a substituted or unsubstituted phenyl group, or a substituted or unsubstituted biphenyl group.

In Formula 7-1 and Formula 7-3, R_{d} to R_{f} may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, R_{d} to R_{f} may each independently be a substituted or unsubstituted methyl group or a substituted or unsubstituted phenyl group. As another example, R_{d} and Rₑ may be bonded to each other to form a ring. When R_{d} and Rₑ are bonded to each other to form a ring, the substituent represented by Formula 7-1 may have a spiro structure.

In Formula 7-6, ring Cy1 may be a substituted or unsubstituted cycloalkyl group having 3 to 20 ring-forming carbon atoms. In an embodiment, ring Cy1 may be a substituted or unsubstituted cyclohexyl group, a substituted or unsubstituted bicycloheptanyl group, or a substituted or unsubstituted adamantyl group. For example, ring Cy1 may be a substituted or unsubstituted cyclohexyl group, a substituted or unsubstituted bicyclo[2,3,1]heptanyl group, or a substituted or unsubstituted adamantyl group.

In Formula 7-1 to Formula 7-7, n31, n33, n35, n38, n40, and n41 may each independently be an integer from 0 to 4; n32, n34, and n36 may each independently be an integer from 0 to 3; n37 and n42 may each independently be an integer from 0 to 7; and n39 may be an integer from 0 to 5.

If n31, n33, n35, n38, n40, and n41 are each 0, the amine compound may not be substituted with R₃₁, R₃₃, R₃₅, R₃₈, R₄₀, and R₄₁. A case where n31, n33, n35, n38, n40, and n41 are each 4 and R₃₁ groups, R₃₃ groups, R₃₅ groups, R₃₈ groups, R₄₀ groups, and R₄₁ groups are each hydrogen atoms may be the same as a case where n31, n33, n35, n38, n40, and n41 are each 0. If n31, n33, n35, n38, n40, and n41 are each 2 or greater, multiple groups of each of R₃₁, R₃₃, R₃₅, R₃₈, R₄₀, and R₄₁ may be the same as each other, or at least one thereof may be different from the others.

If n32, n34, and n36 are each 0, the amine compound may not be substituted with R₃₂, R₃₄, and R₃₆. A case where n32, n34, and n36 are each 3 and R₃₂ groups, R₃₄ groups, and R₃₆ groups are each hydrogen atoms may be the same as a case where n32, n34, and n36 are each 0. If n32, n34, and n36 are each 2 or more, multiple groups of each of R₃₂, R₃₄, and R₃₆ may be the same as each other, or at least one thereof may be different from the others.

If n37 and n42 are each 0, the amine compound may not be substituted with R₃₇ and R₄₂. A case where n37 and n42 are each 7 and R₃₇ groups and R₄₂ groups are each hydrogen atoms may be the same as a case where n37 and n42 are each 0. If n37 and n42 are each 2 or more, multiple R₃₇ groups and multiple R₄₂ groups may be the same as each other, or at least one thereof may be different from the others.

If n39 is 0, the amine compound may not be substituted with R₃₉. A case where n39 is 5 and R₃₉ groups are all hydrogen atoms may be the same as a case where n39 is 0. If n39 is 2 or more, multiple R₃₉ groups may all be the same, or at least one thereof may be different from the others.

In Formula 7-1 to Formula 7-7, -* represents a bonding site to a nitrogen atom in Formula 1.

In an embodiment, the amine compound represented by Formula 1 may be represented by Formula 8-1 or Formula 8-2:

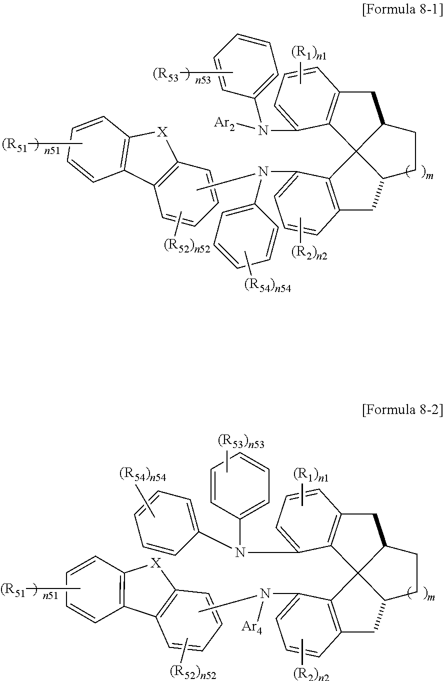

Formula 8-1 and Formula 8-2 each represent a case where the types of substituents linked to the first amine group and the second amine group are specified in the structure of Formula 1. Formula 8-1 and Formula 8-2 represent the cases where at least three of Ar₁ to Ar₄ are specified in the structure of Formula 1.

In Formula 8-1 and Formula 8-2, X may be N(R₅₅), C(R₅₆)(R₅₇), O, or S.

In Formula 8-1 and Formula 8-2, R₅₁ to R₅₇ may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, R₅₁ to R₅₇ may each independently be a hydrogen atom, a substituted or unsubstituted methyl group, or a substituted or unsubstituted phenyl group. As another example, R₅₆ and R₅₇ may be bonded to each other to form a ring.

In Formula 8-1 and Formula 8-2, Ar₂, Ar₄, R₁, R₂, n1, n2, and m are the same as described in Formula 1.

In an embodiment, the amine compound represented by Formula 1 may be represented by any one of Formula 9-1 to Formula 9-4:

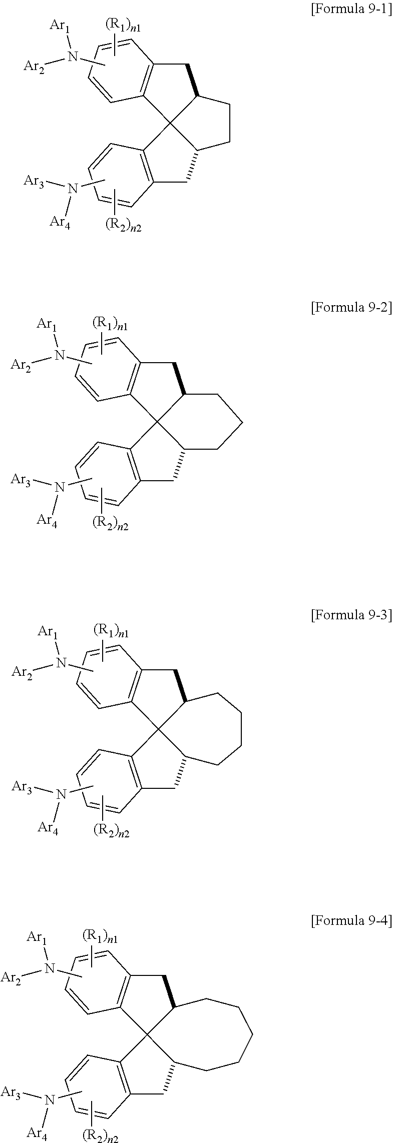

Formula 9-1 to Formula 9-4 each represent a case where the number of carbon atoms of the alkyl substituent is specified in the structure of Formula 1. Formula 9-1 to Formula 9-4 each represent a case where m is specified in the structure of Formula 1. Formula 9-1 represents a case where m is 1 in Formula 1. Formula 9-2 represents a case where m is 2 in Formula 1. Formula 9-3 represents a case where m is 3 in Formula 1. Formula 9-4 represents a case where m is 4 in Formula 1.

In Formula 9-1 to Formula 9-4, Ar₁ to Ar₄, R₁, R₂, n1, and n2 are the same as described in Formula 1.

In an embodiment, the amine compound represented by Formula 1 may be represented by any one of Formula 10-1 to Formula 10-4:

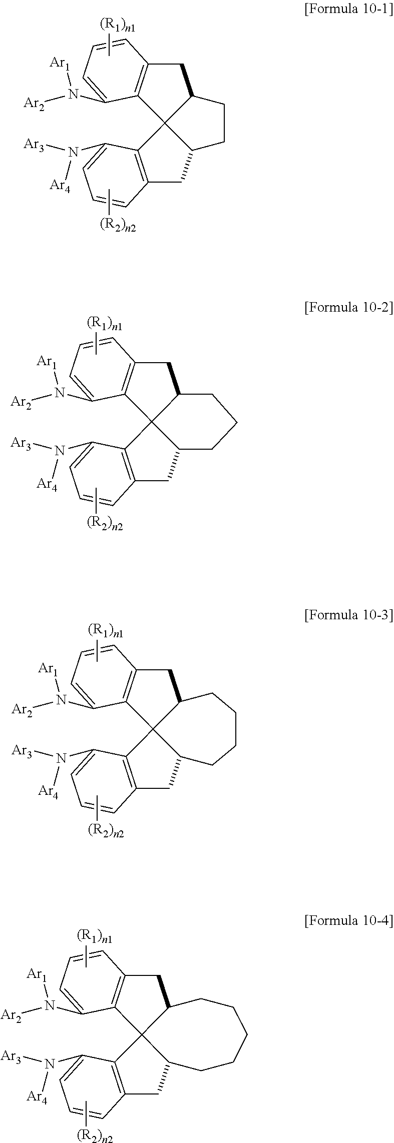

In Formula 10-1 to Formula 10-4, Ar₁ to Ar₄, R₁, R₂, n1, and n2 are the same as described in Formula 1.

The amine compound may be any compound selected from Compound Group 1. The light emitting device ED of an embodiment may include at least one compound selected from Compound Group 1 (for example, the hole transport region HTR may include at least one compound selected from Compound Group 1):

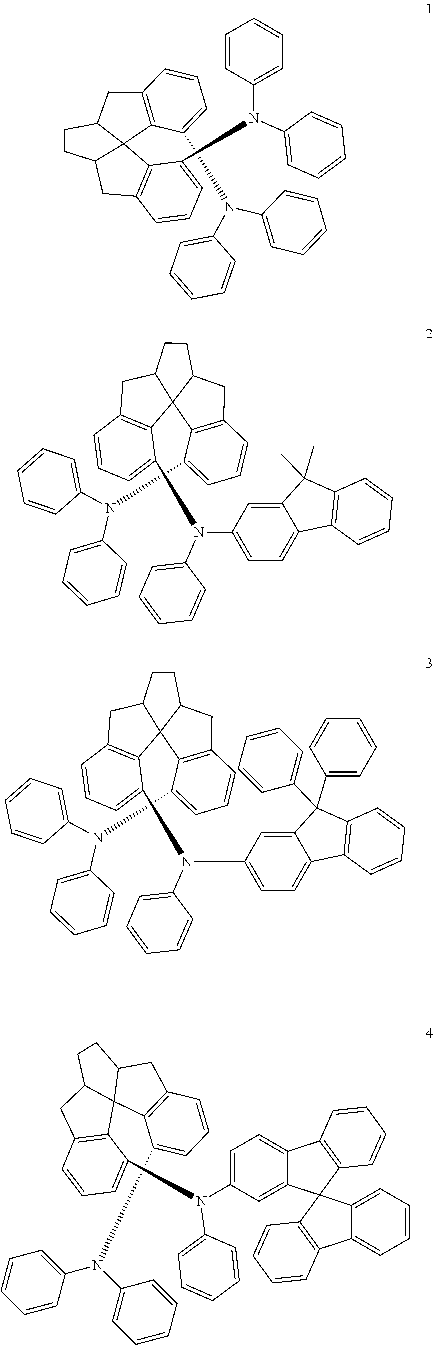

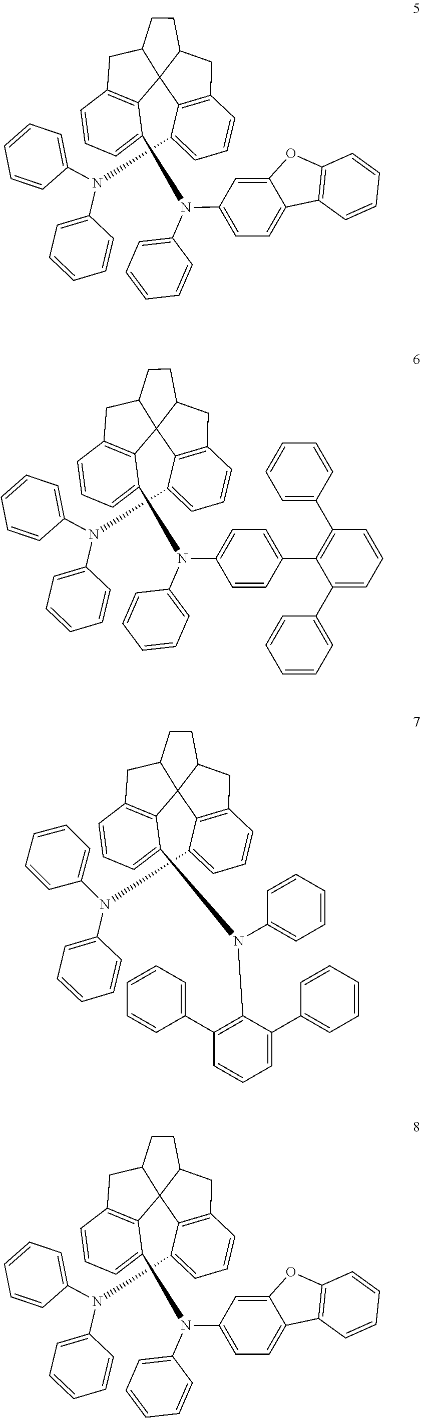

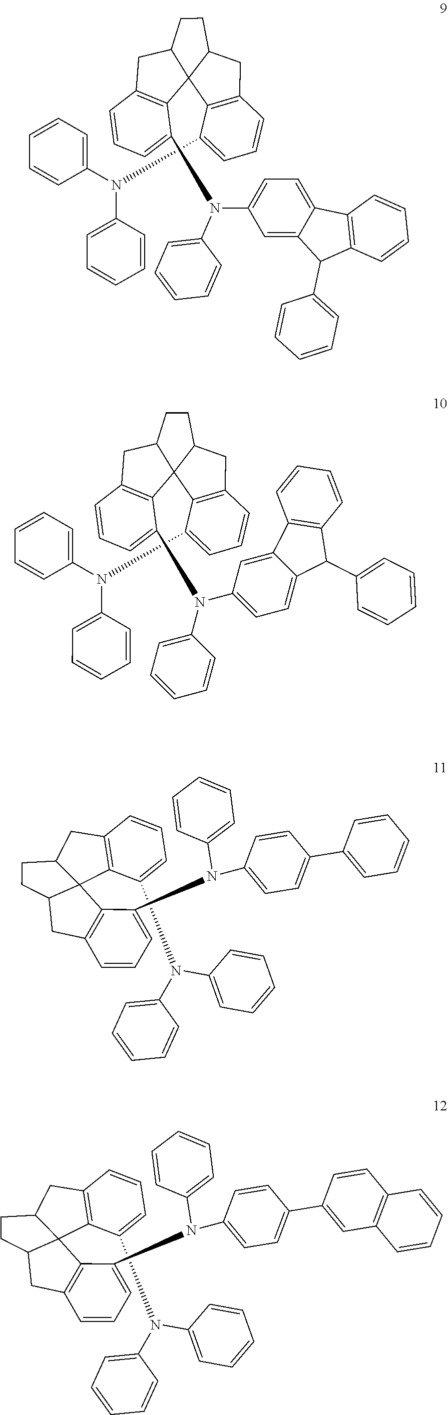

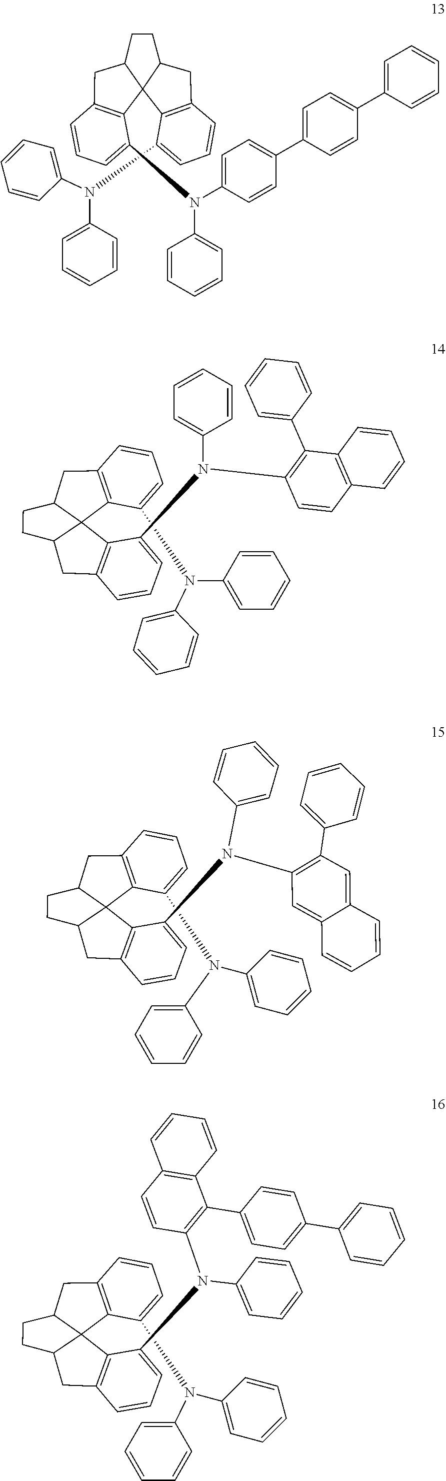

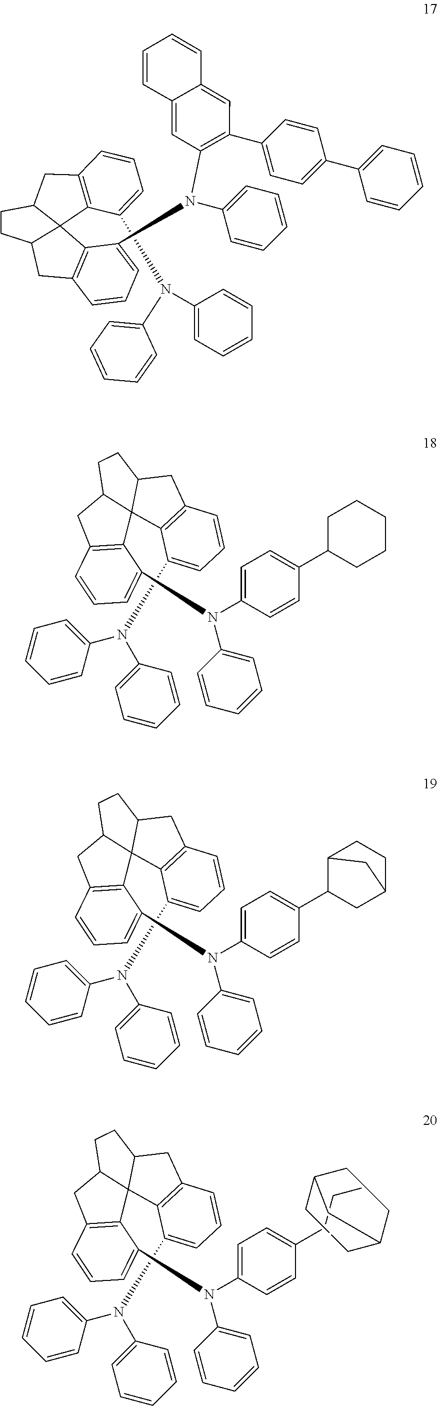

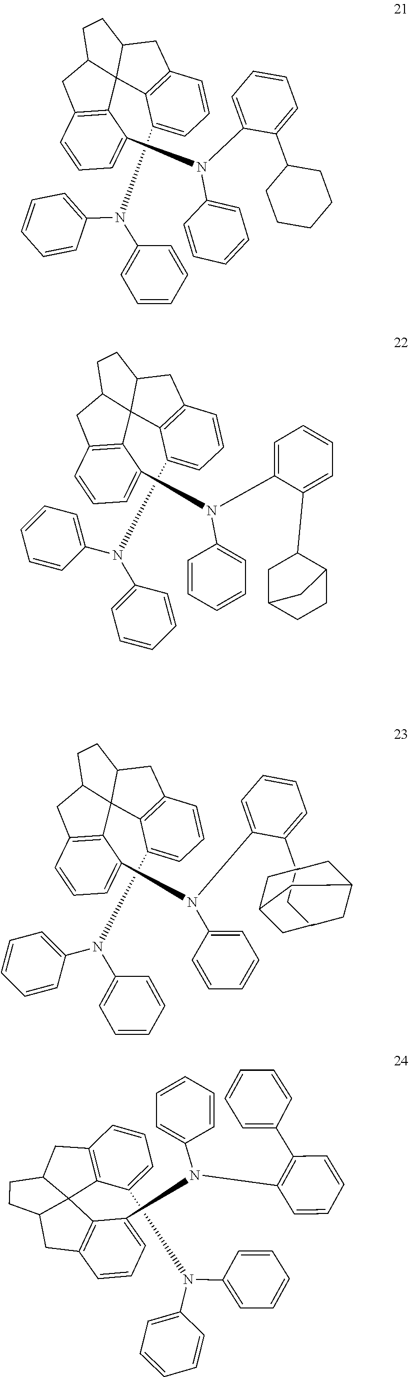

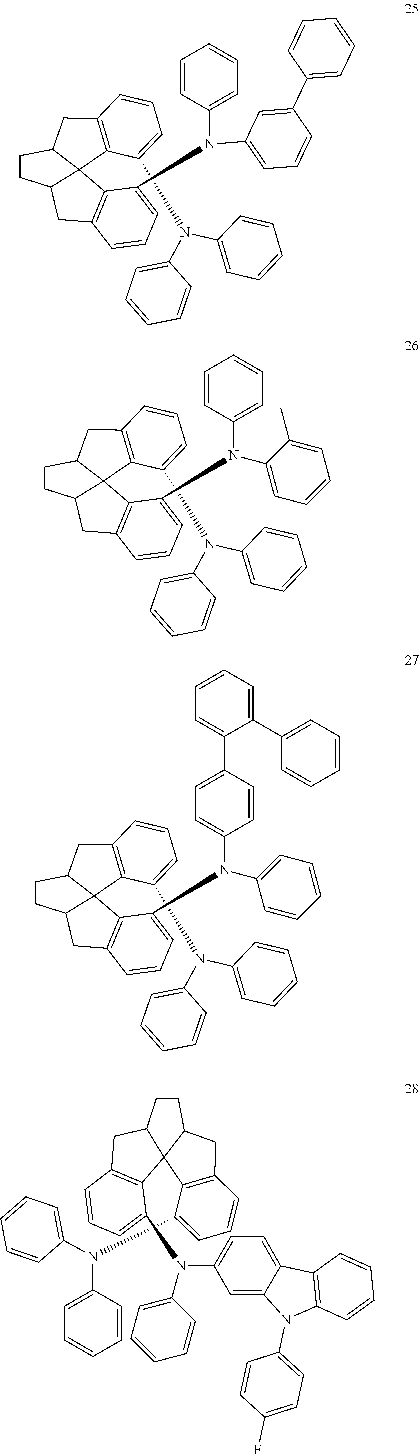

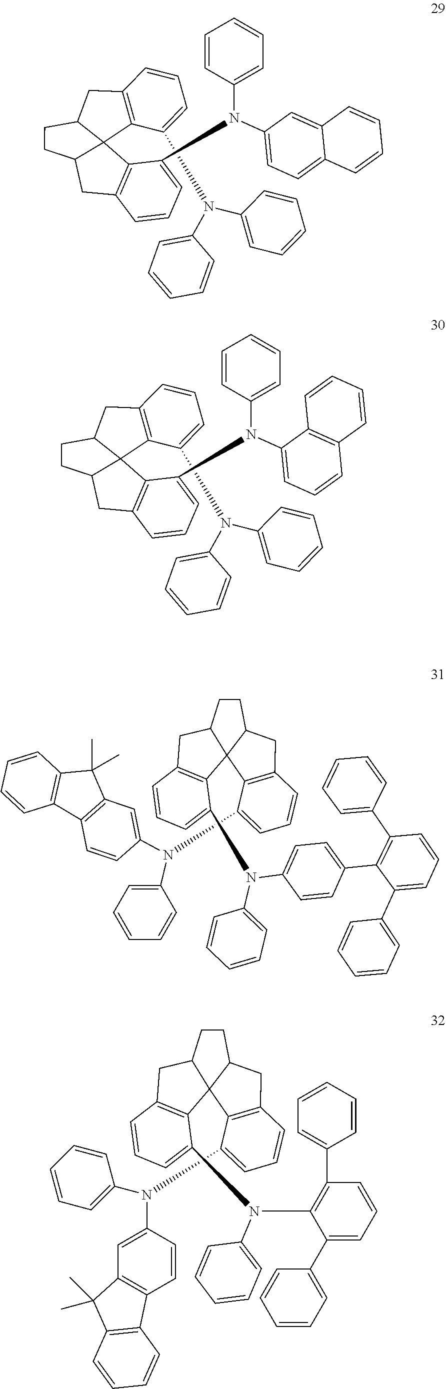

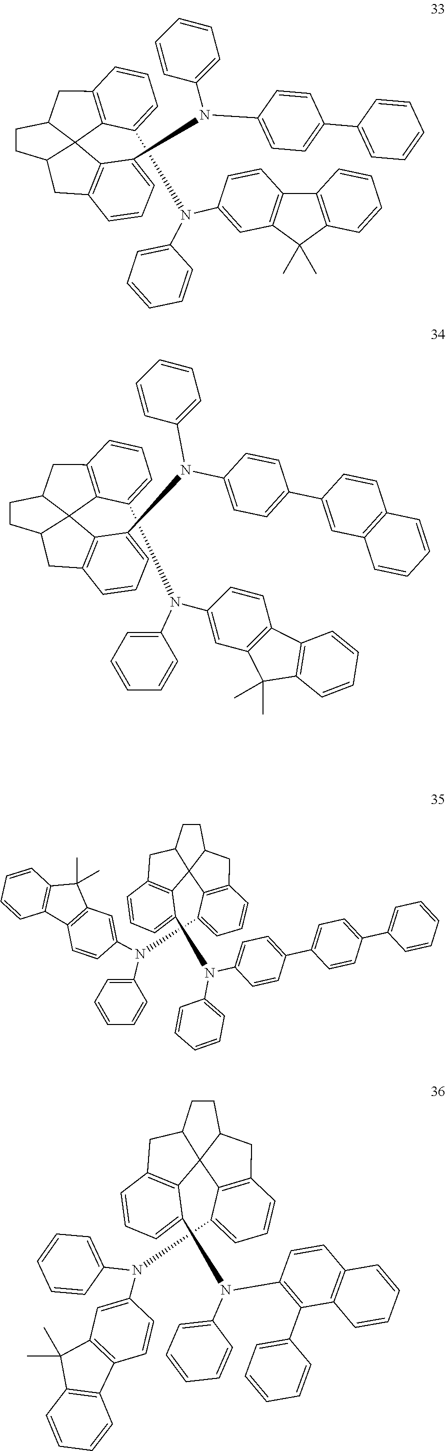

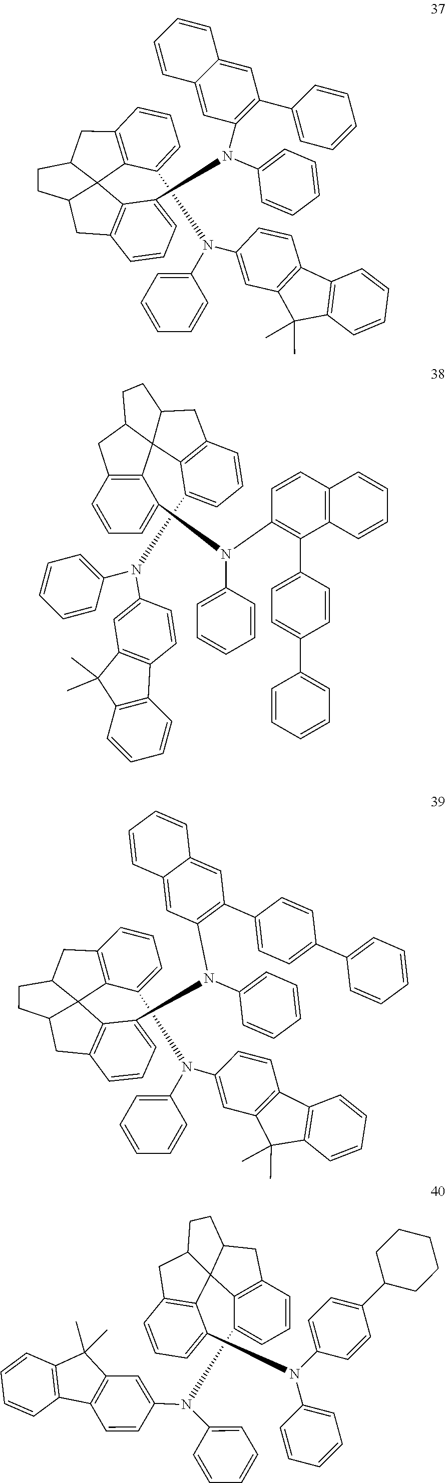

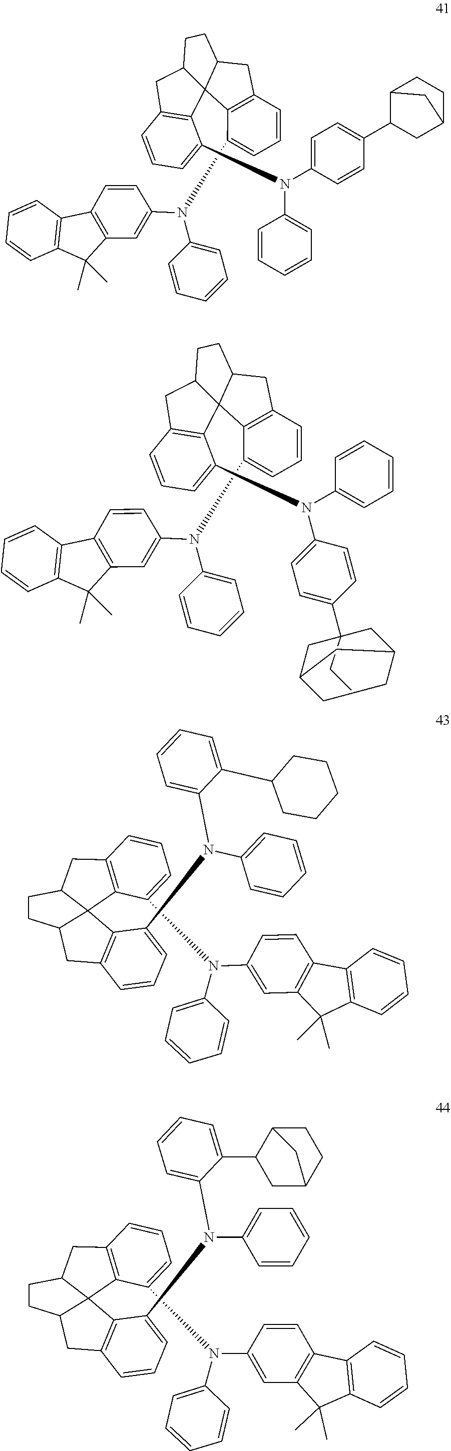

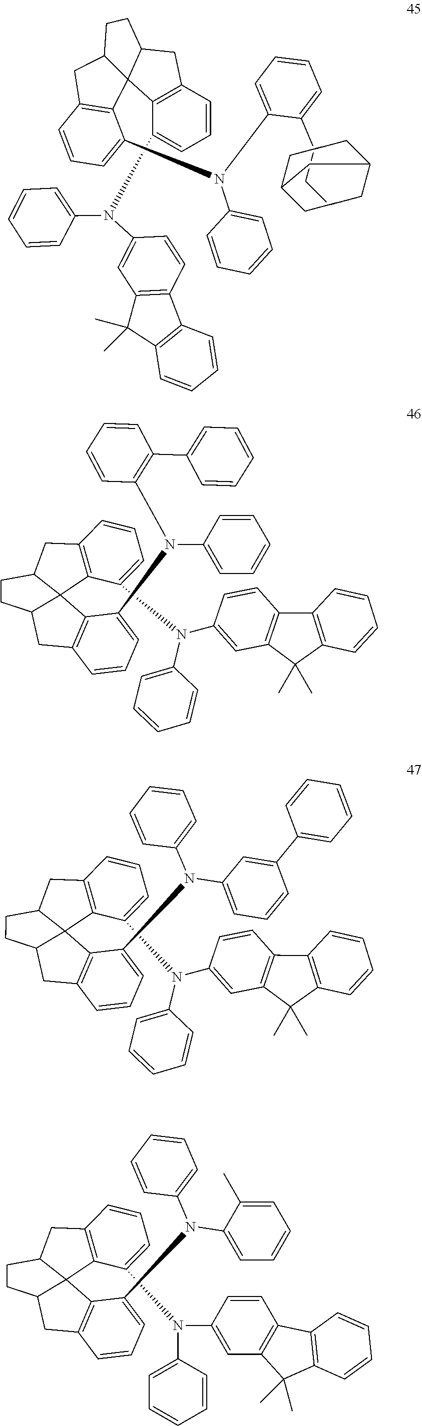

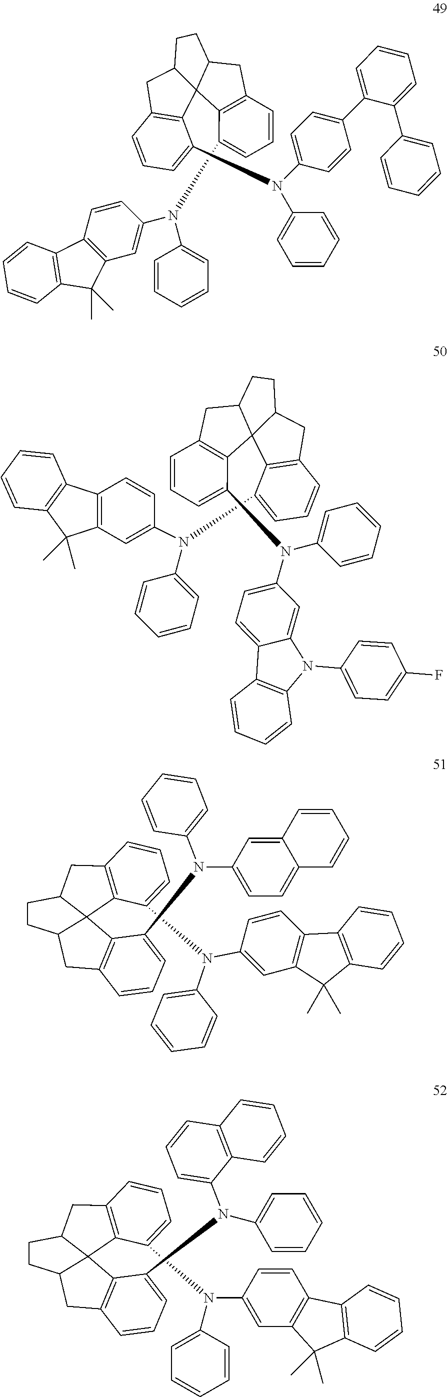

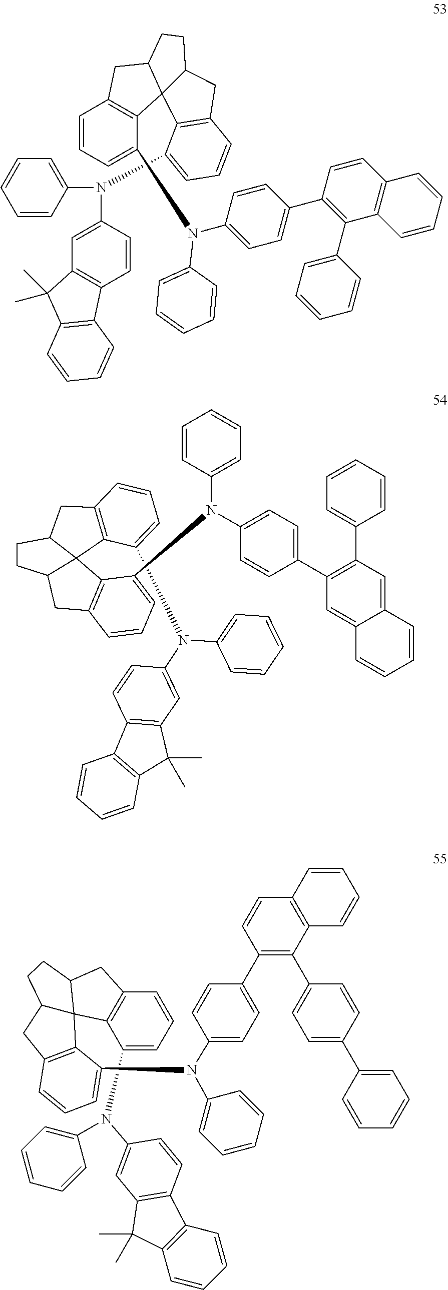

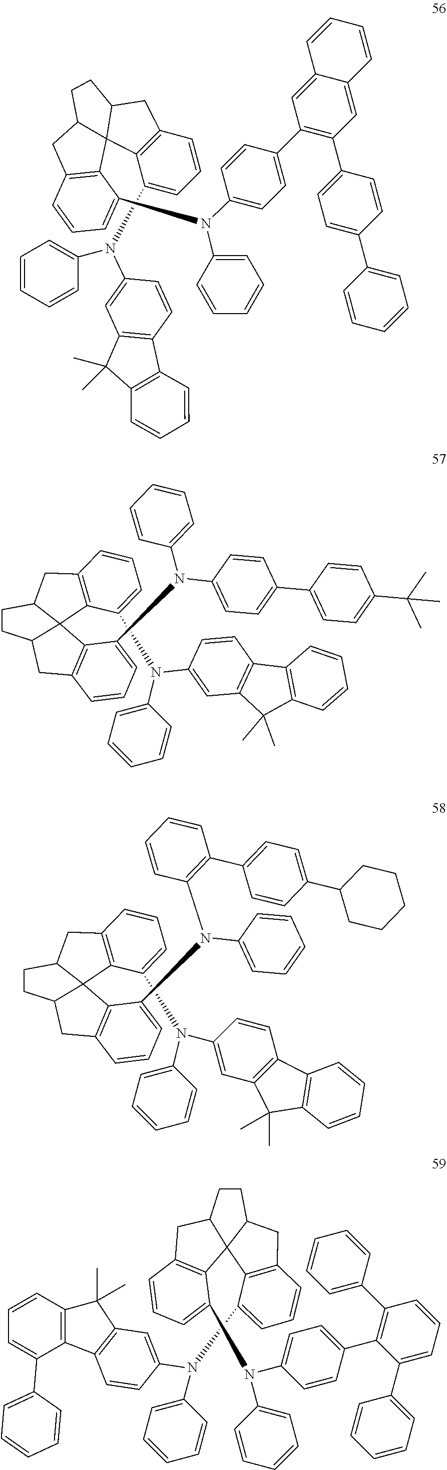

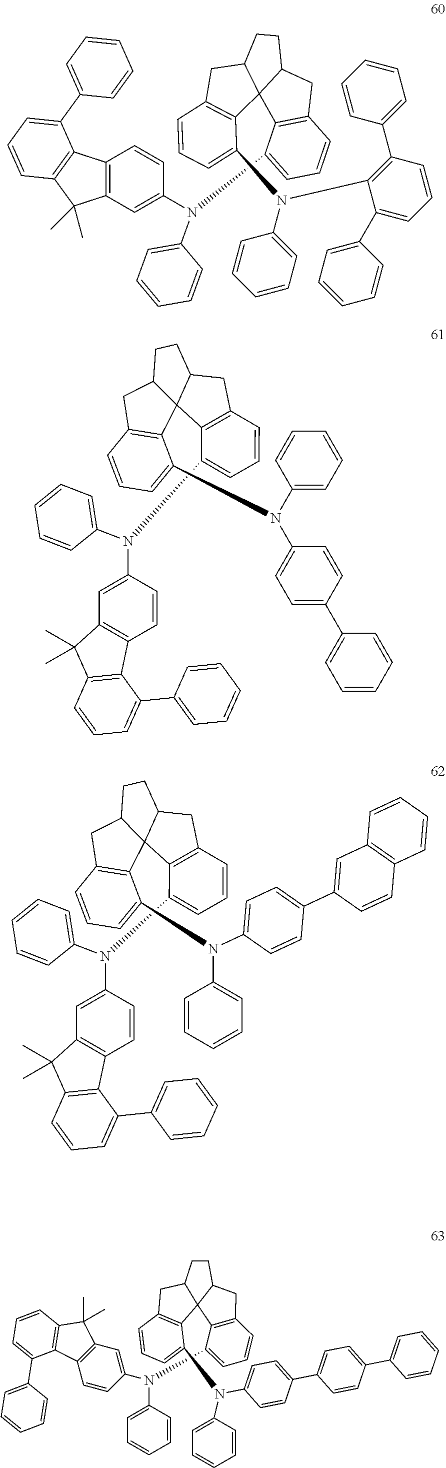

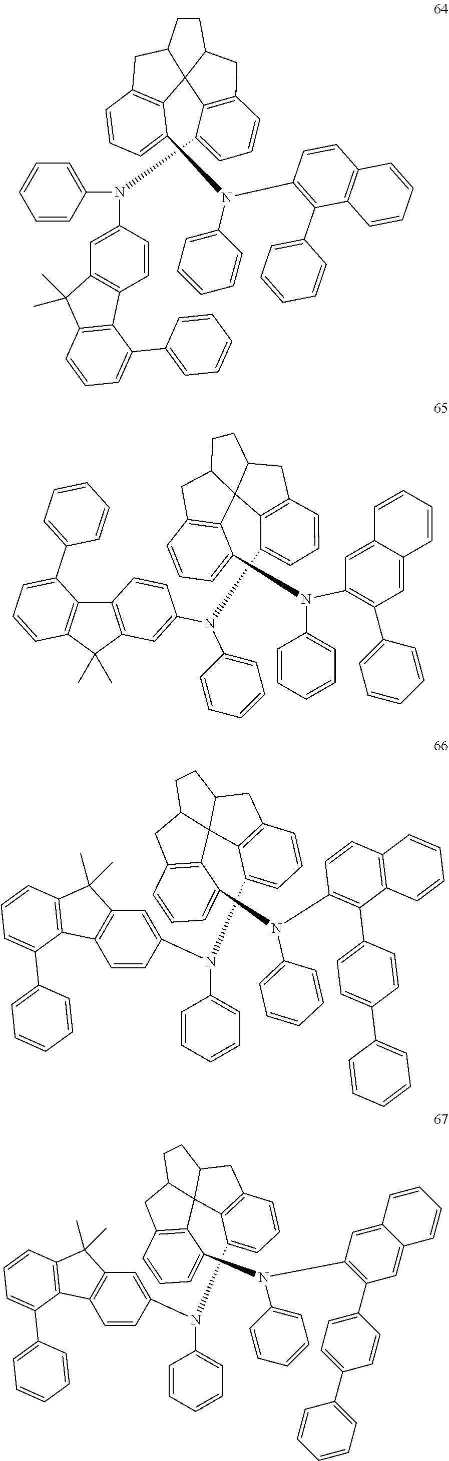

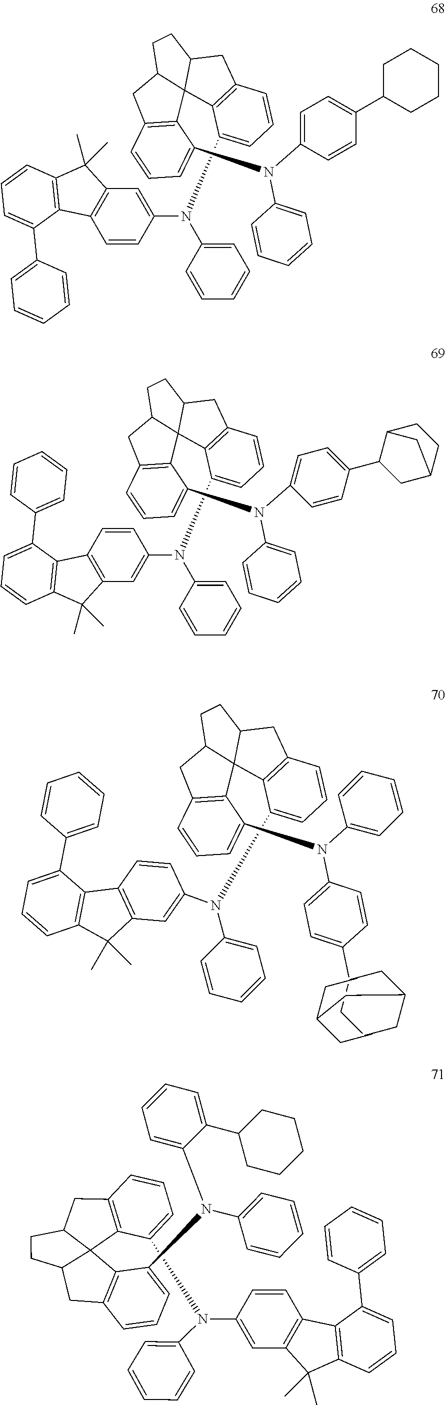

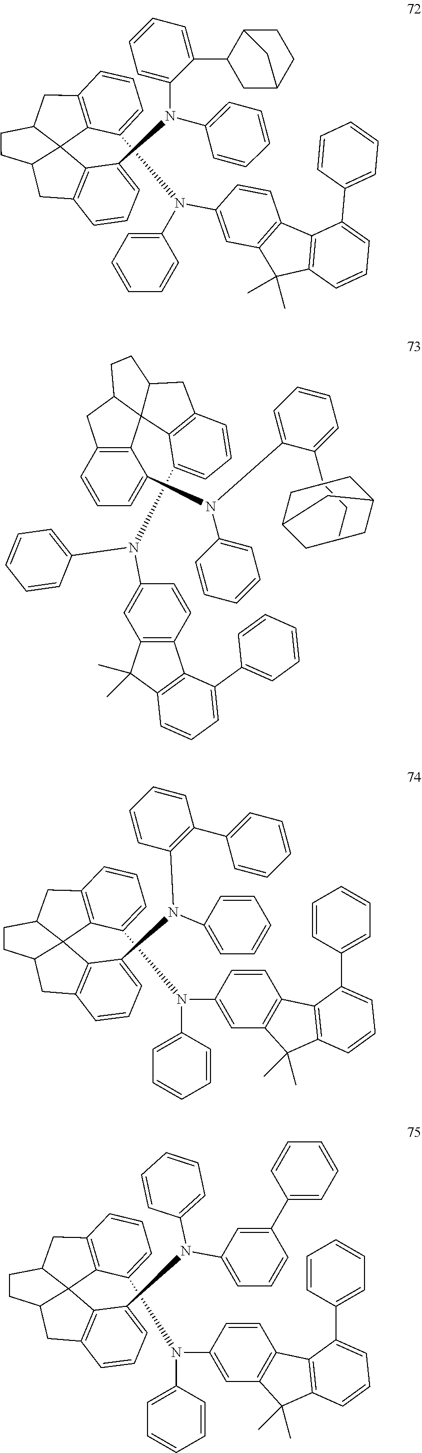

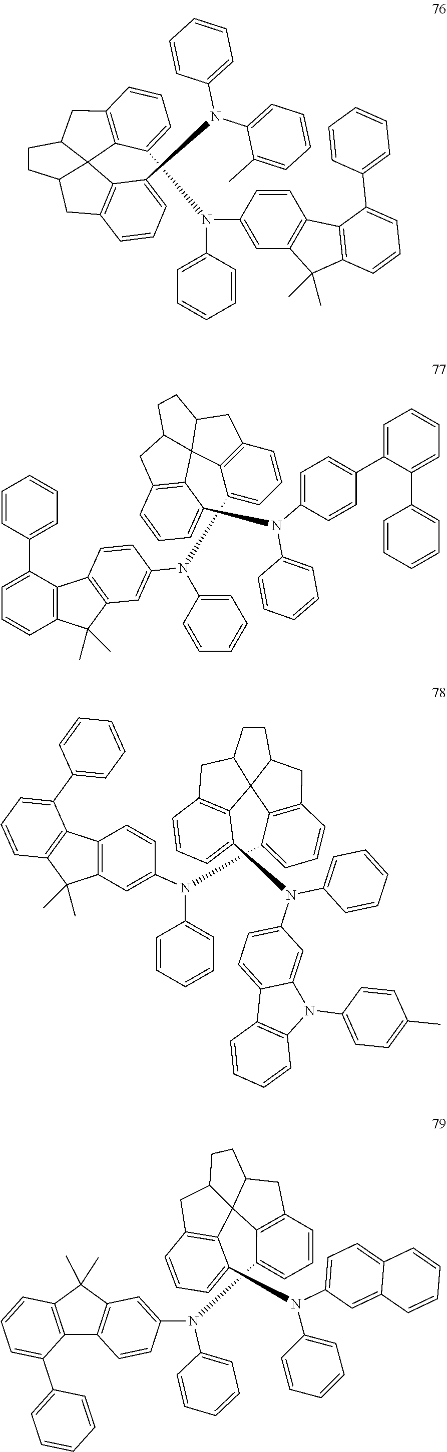

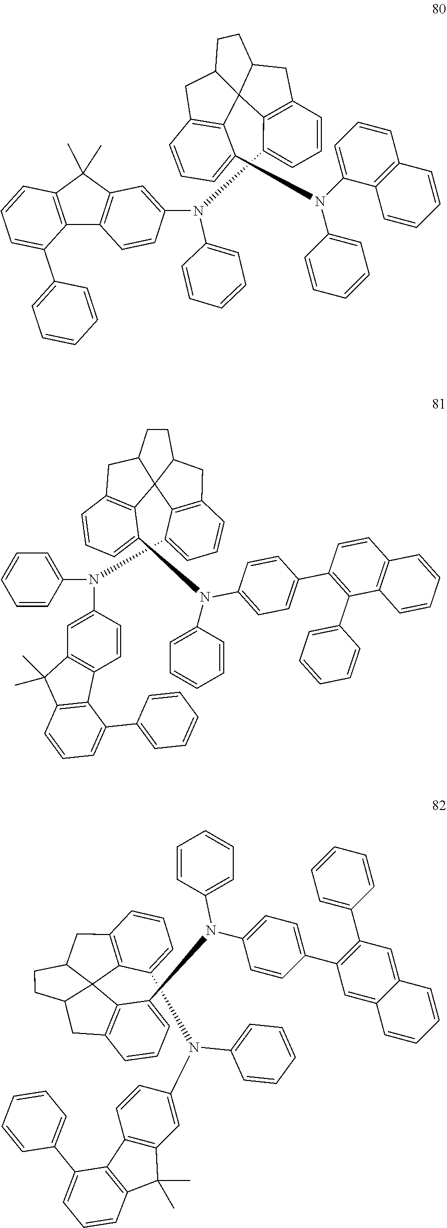

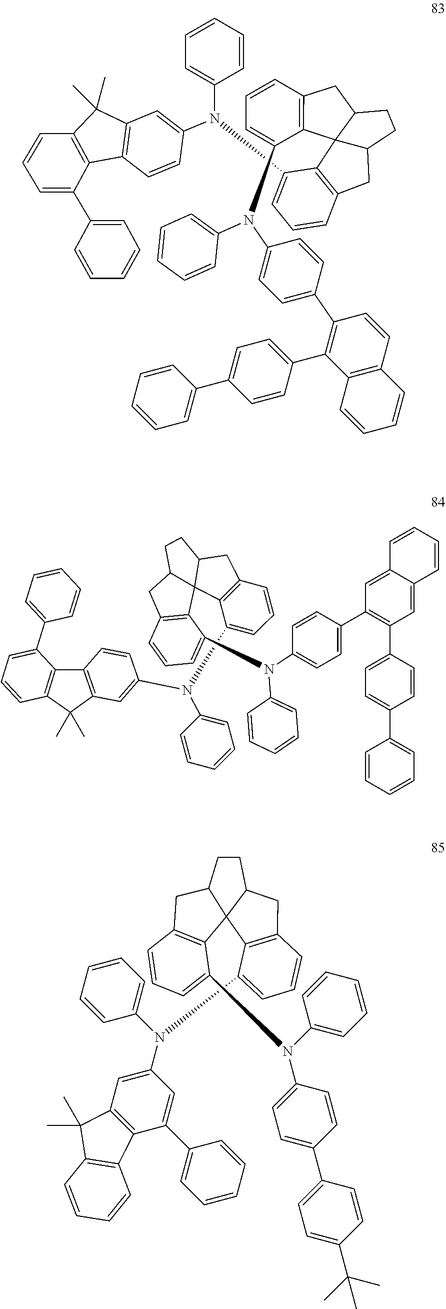

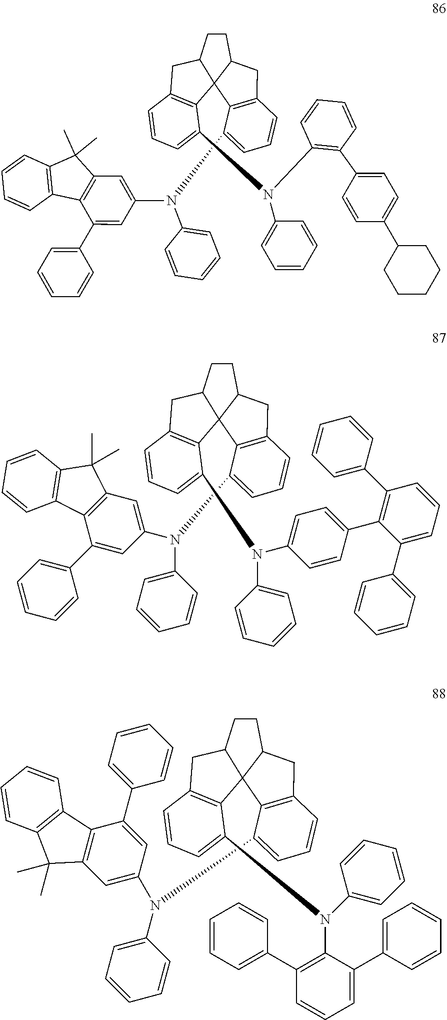

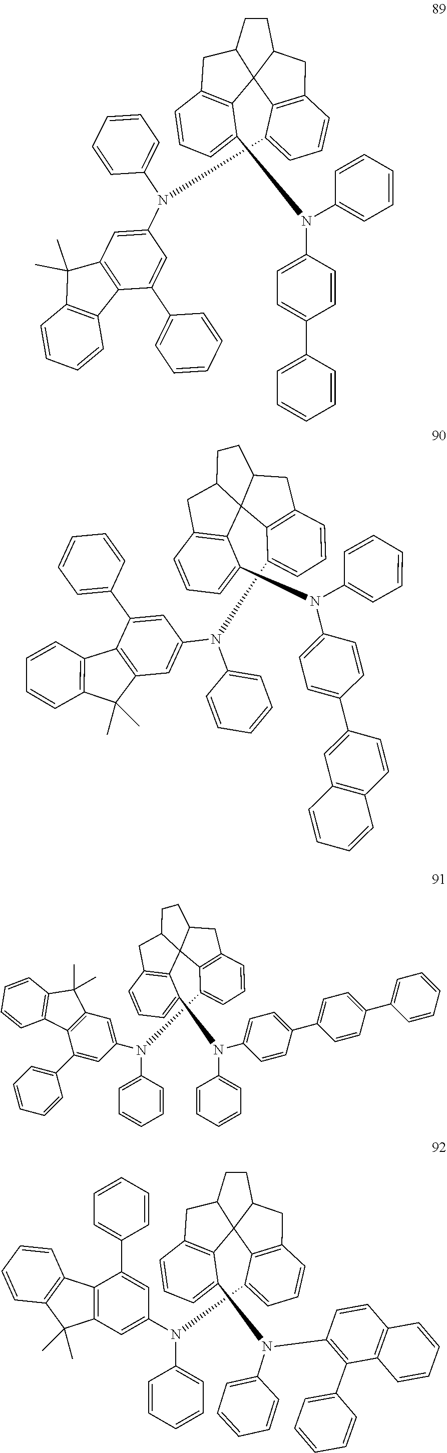

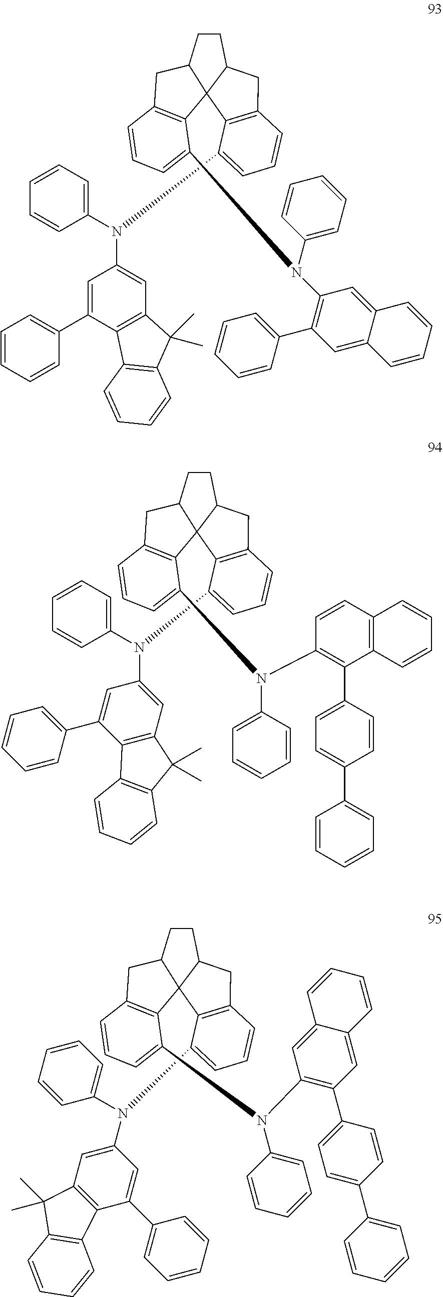

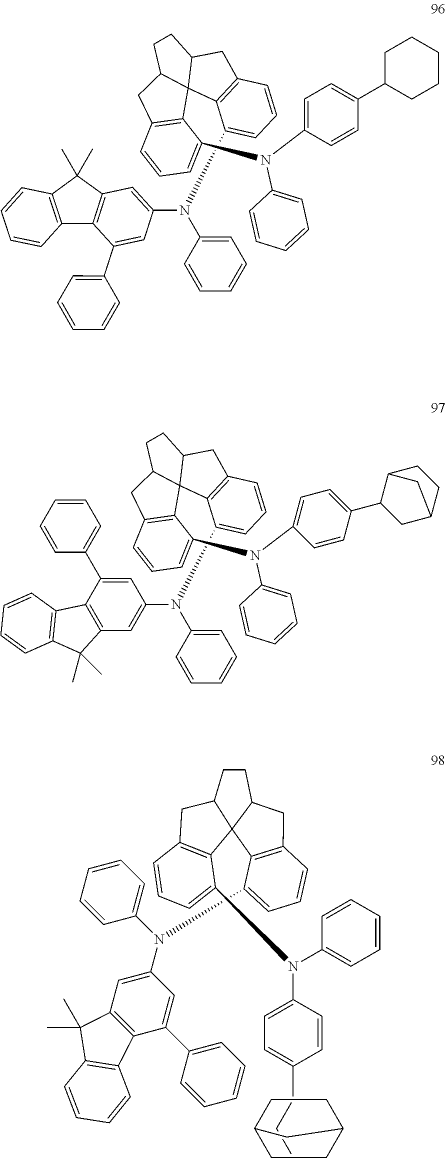

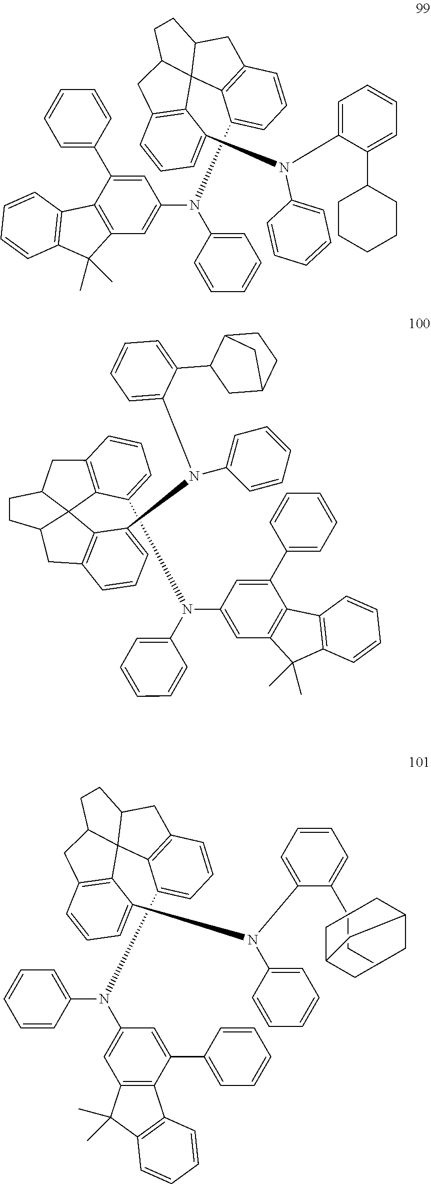

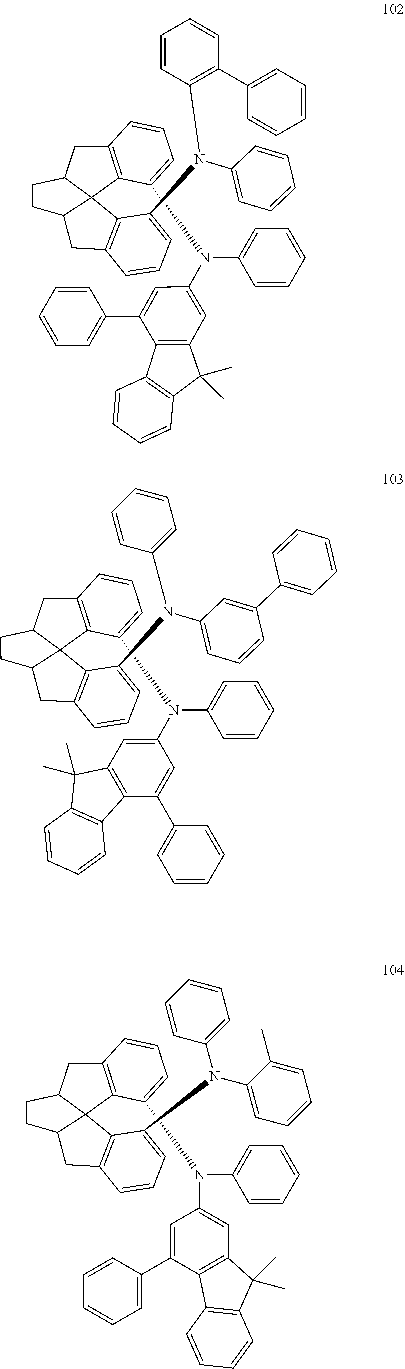

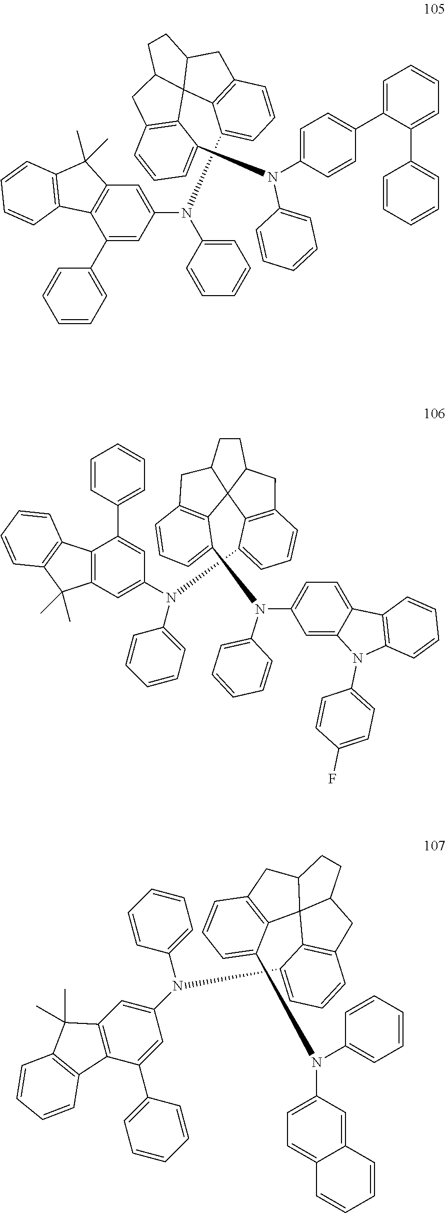

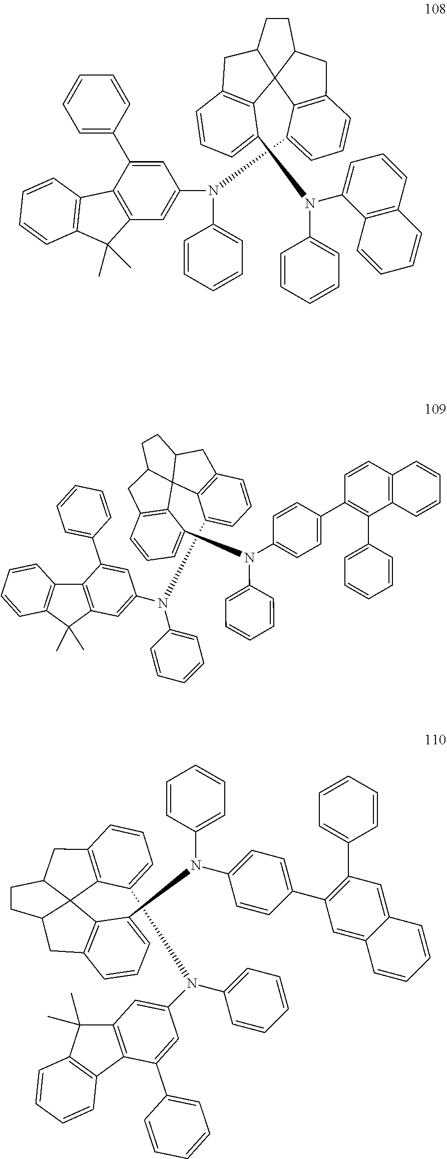

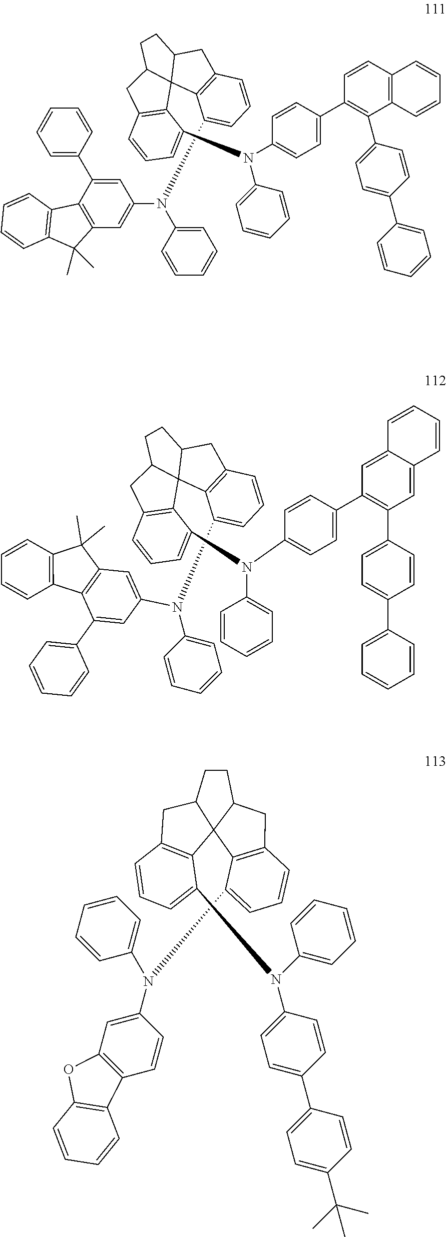

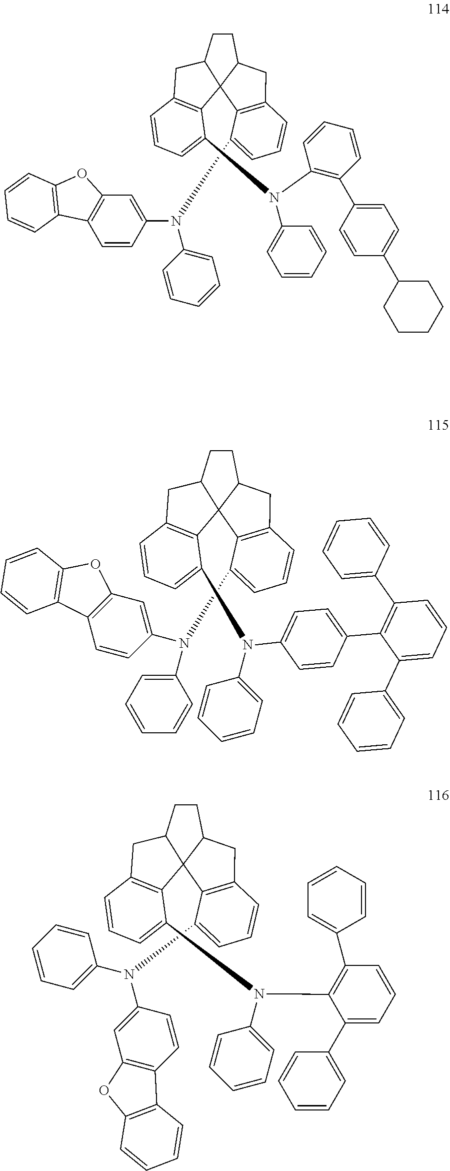

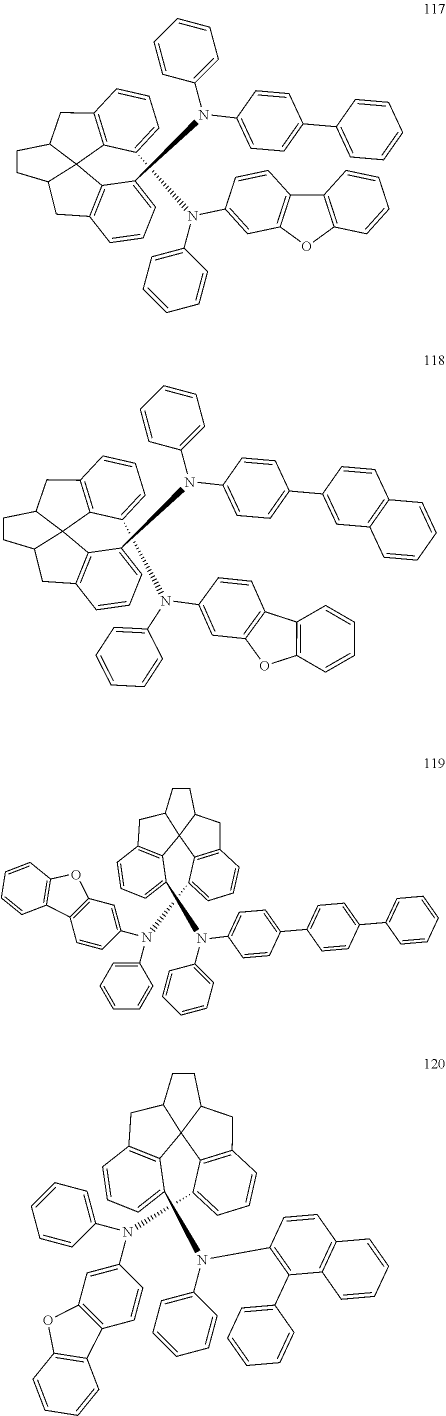

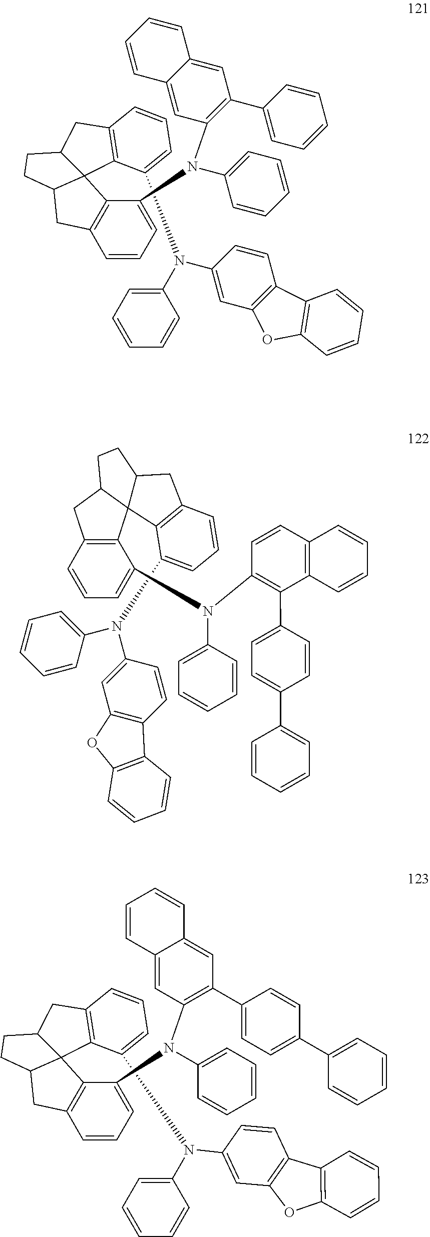

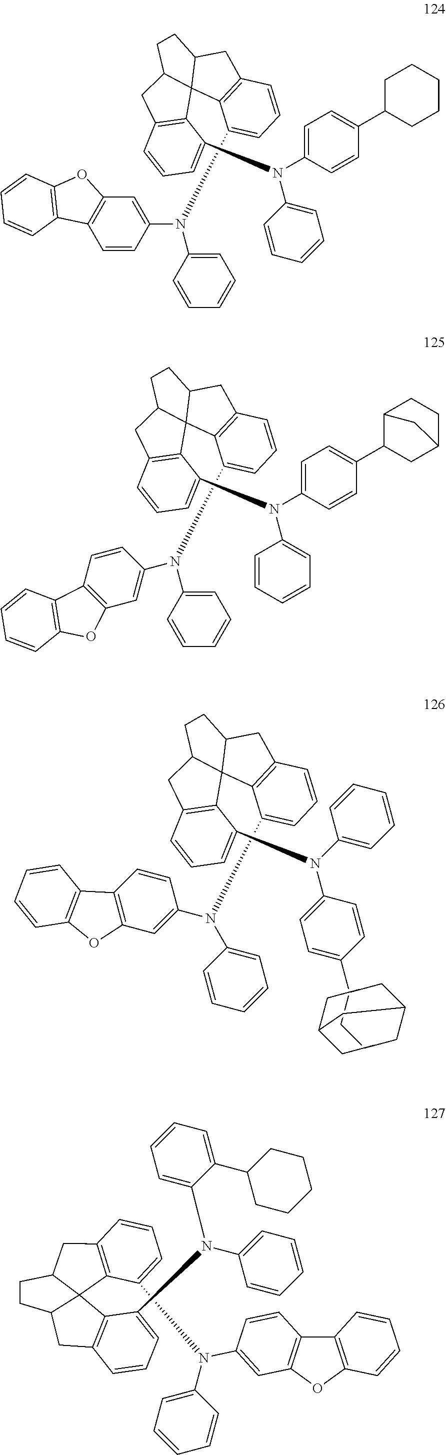

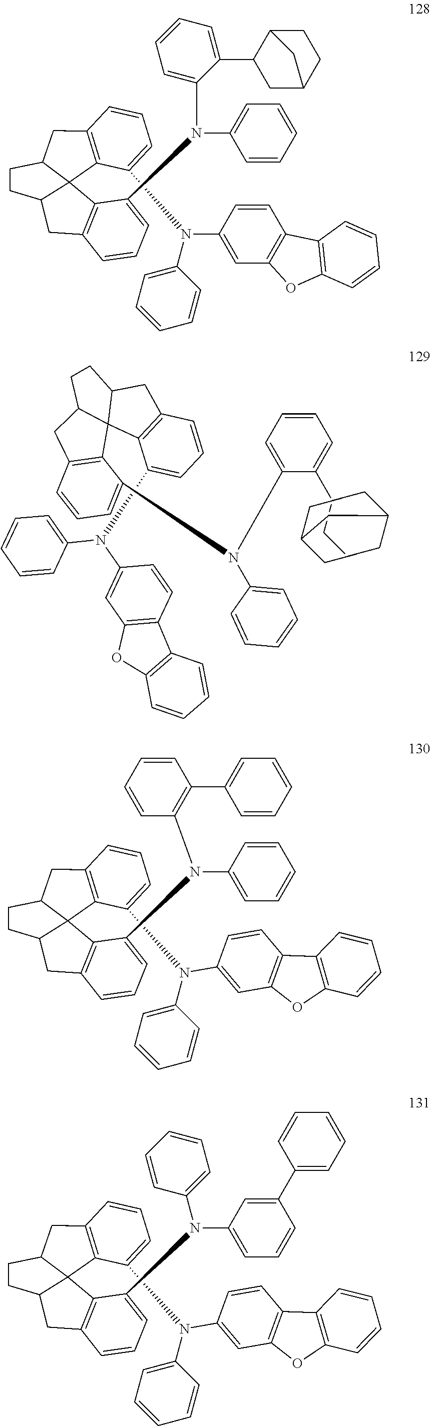

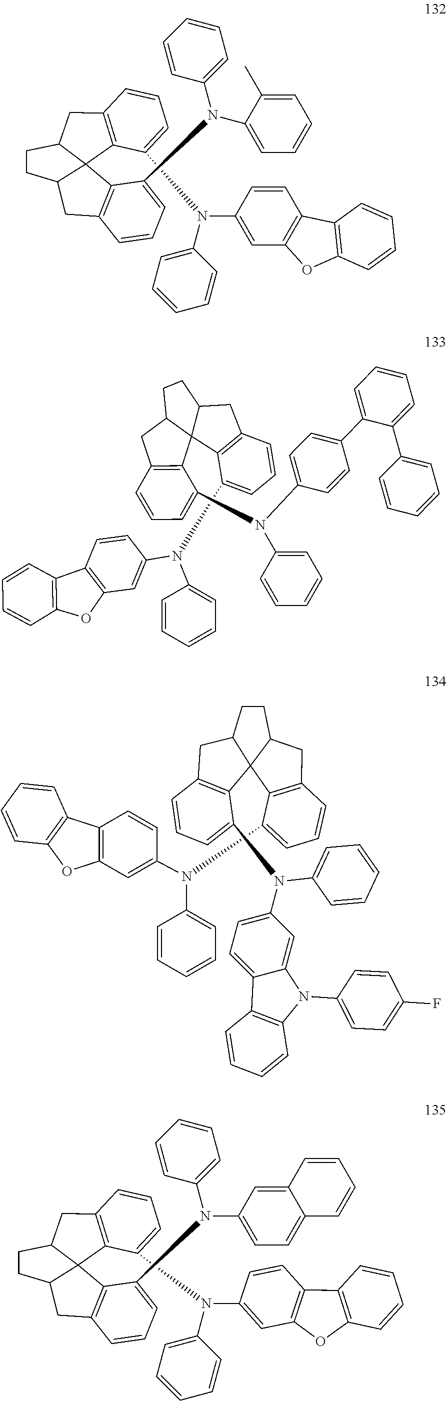

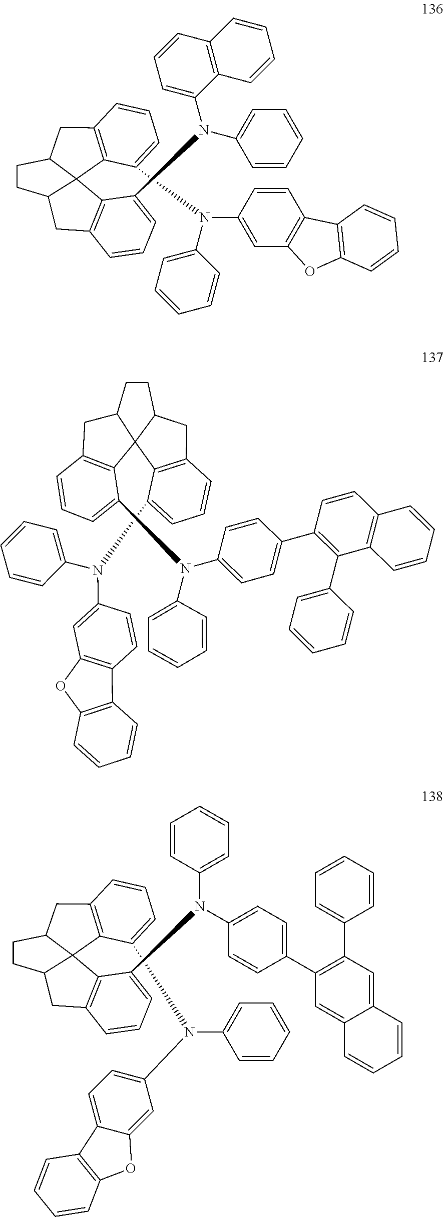

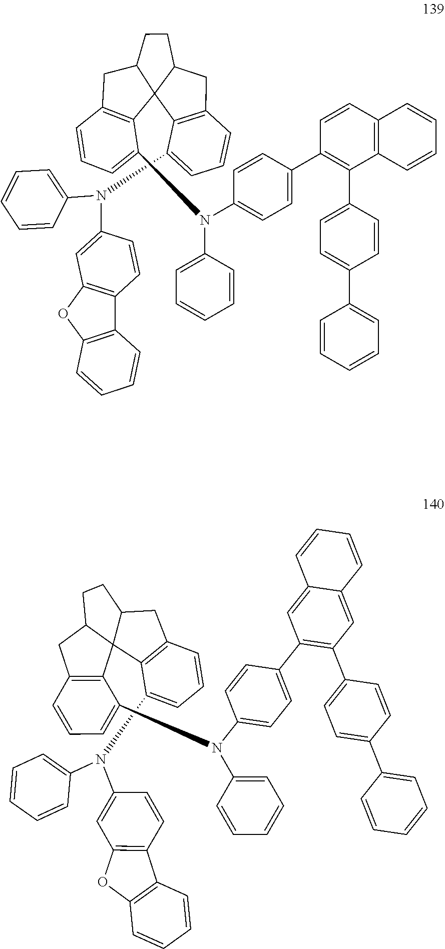

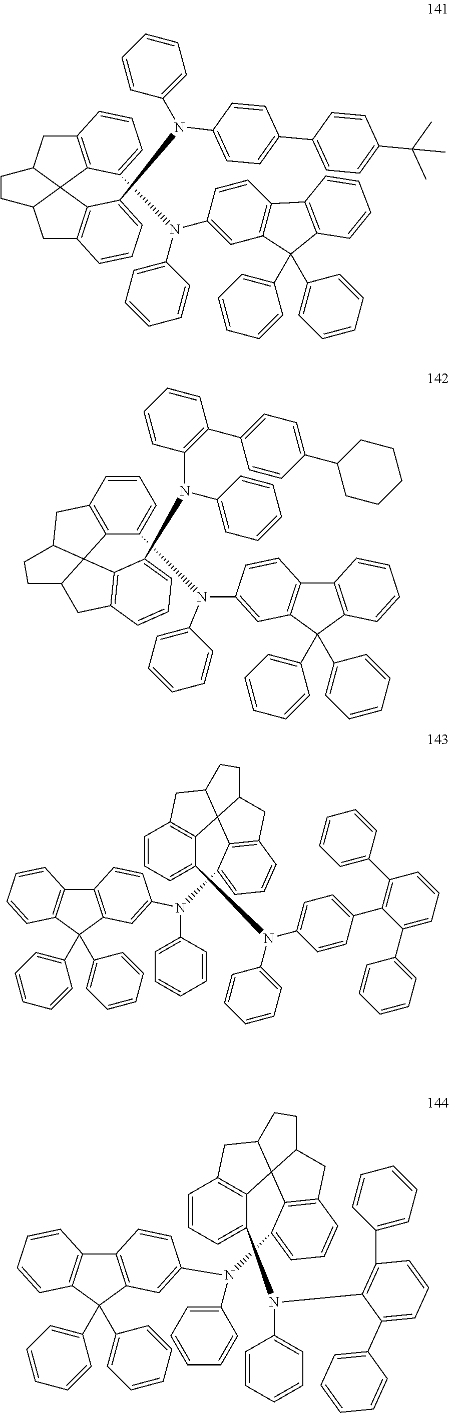

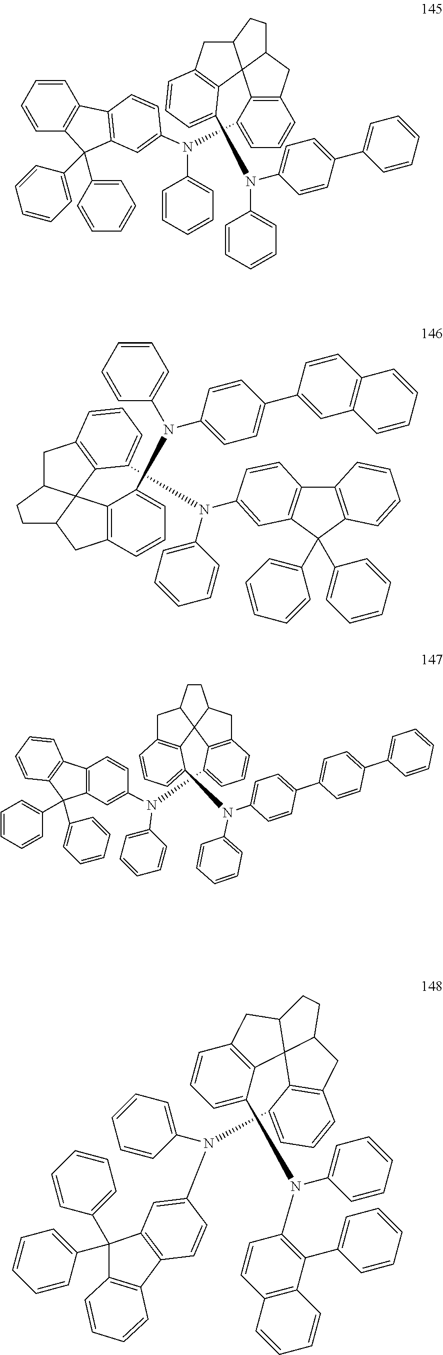

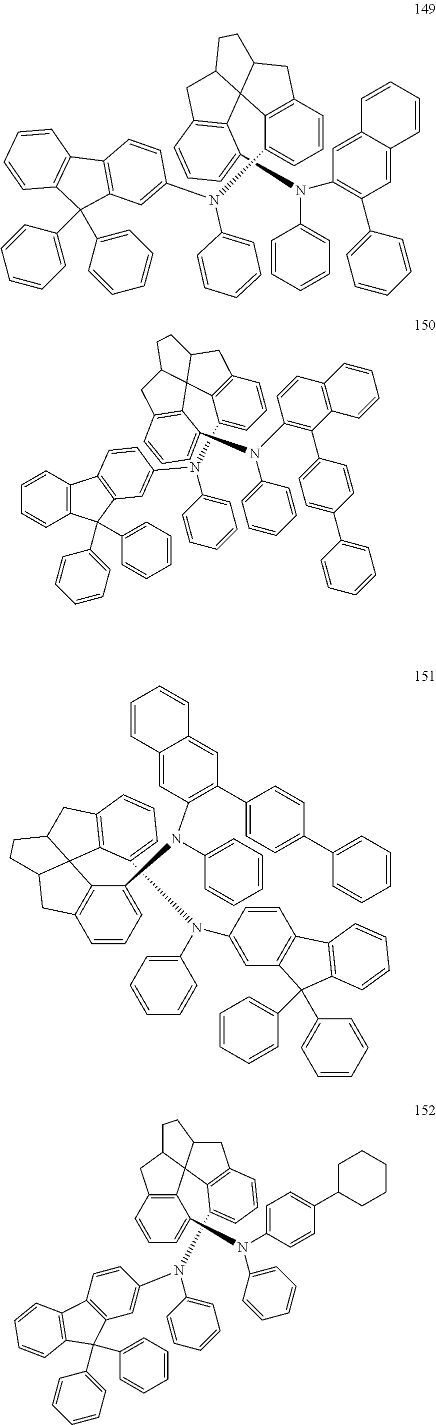

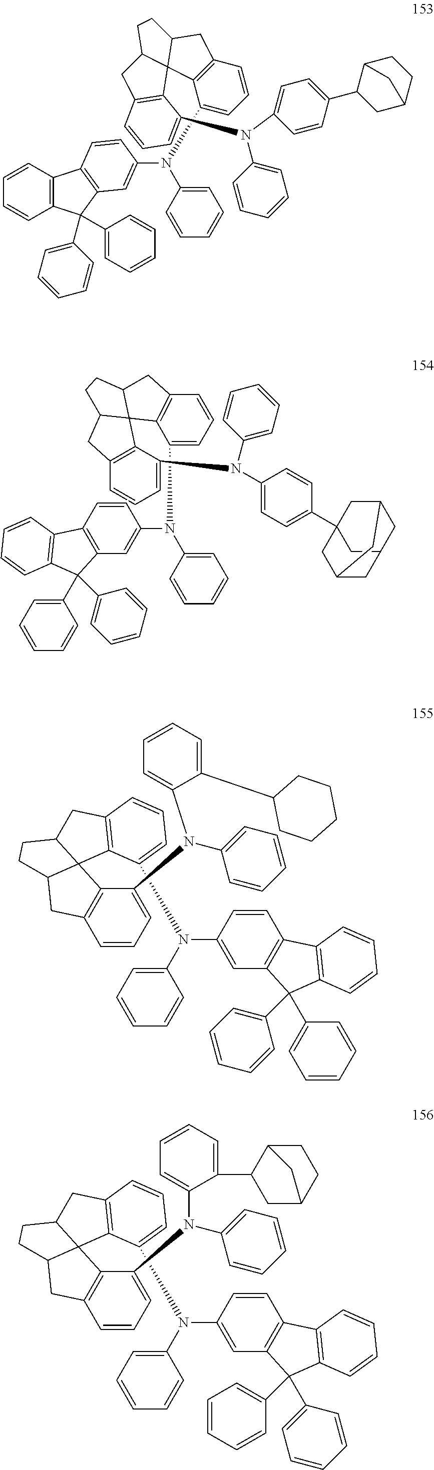

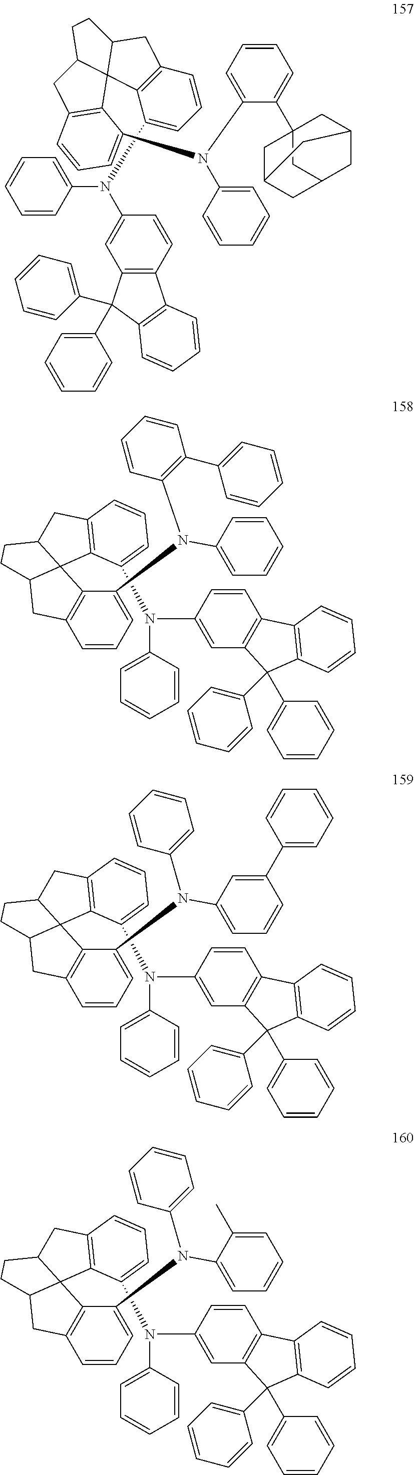

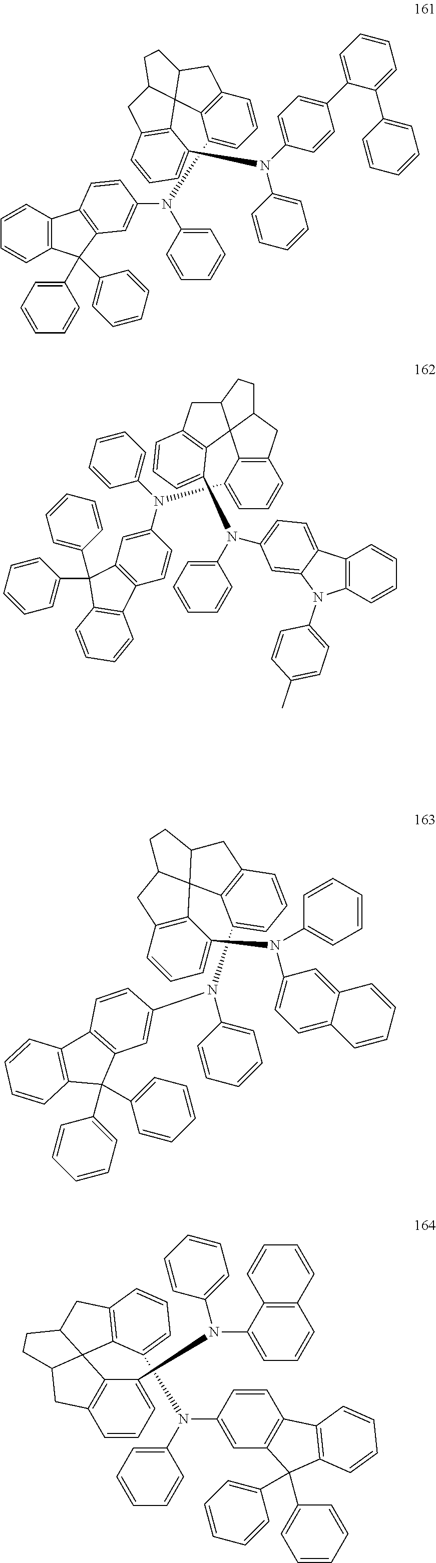

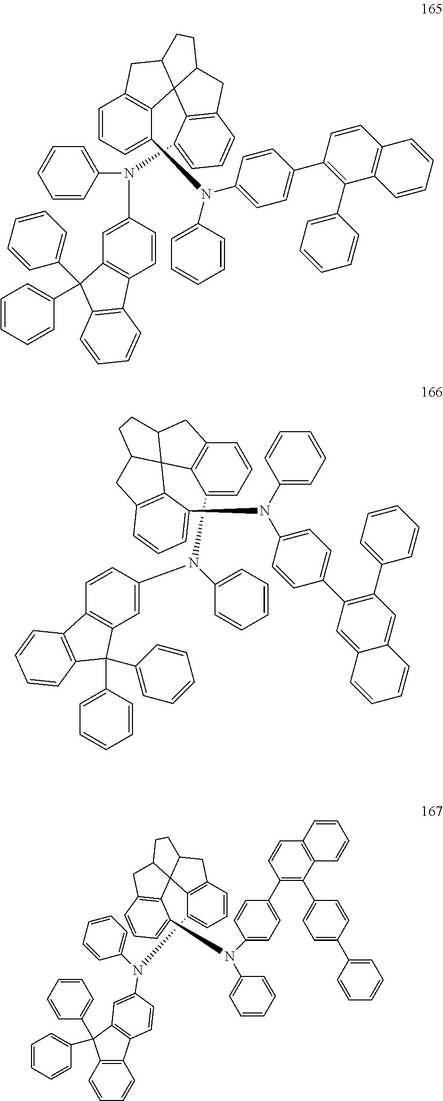

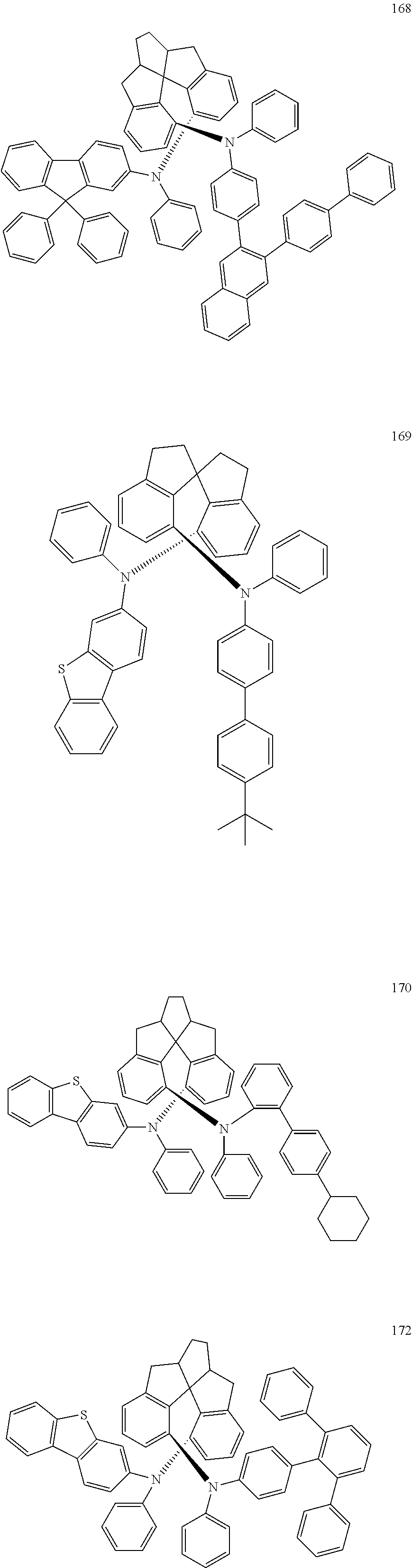

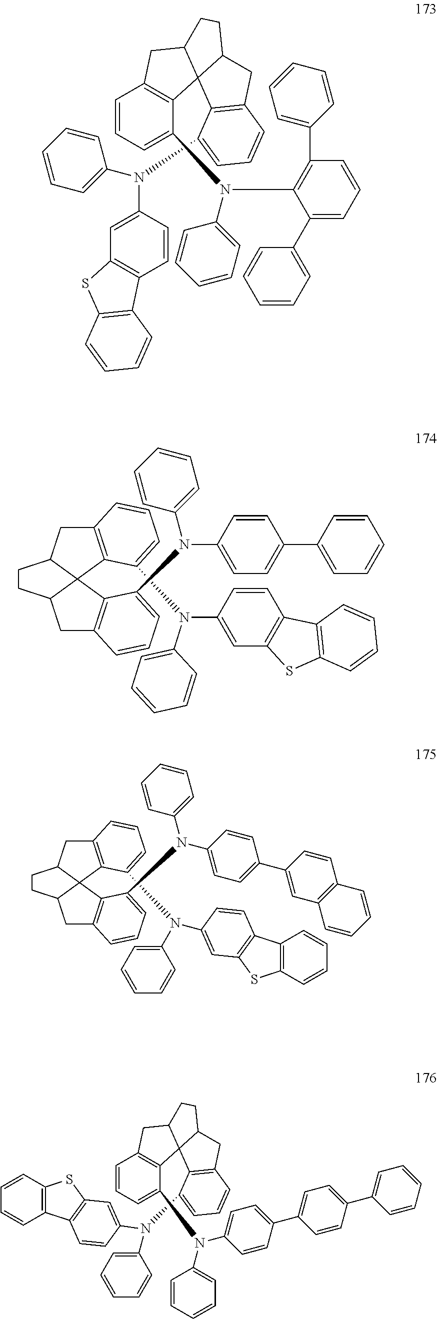

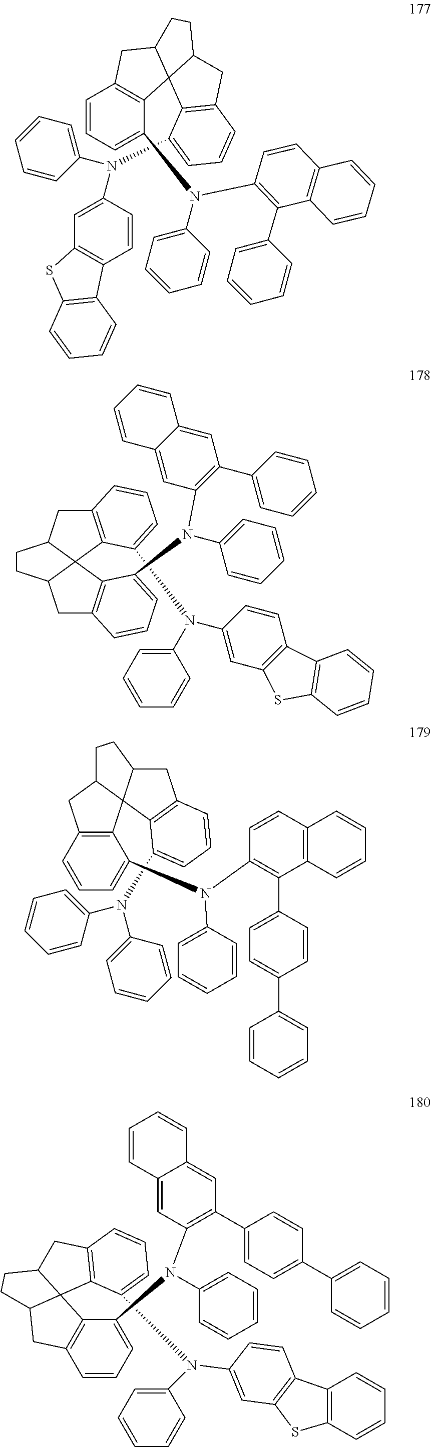

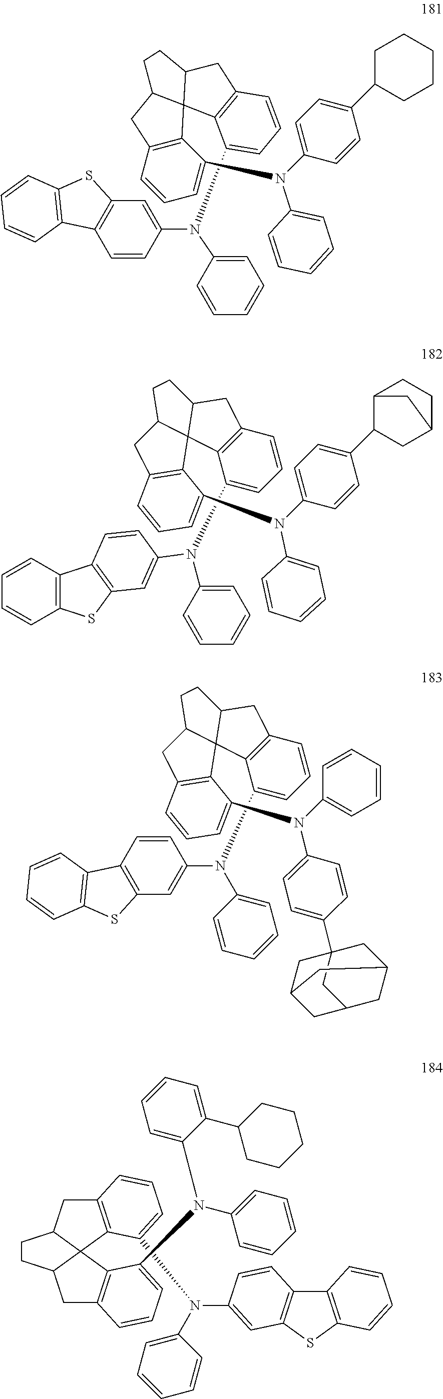

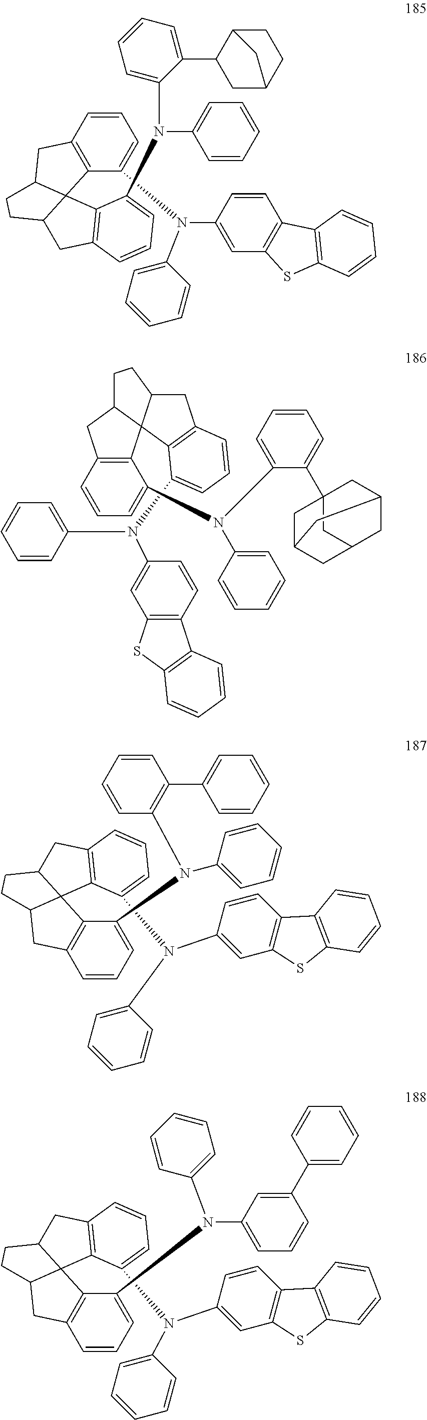

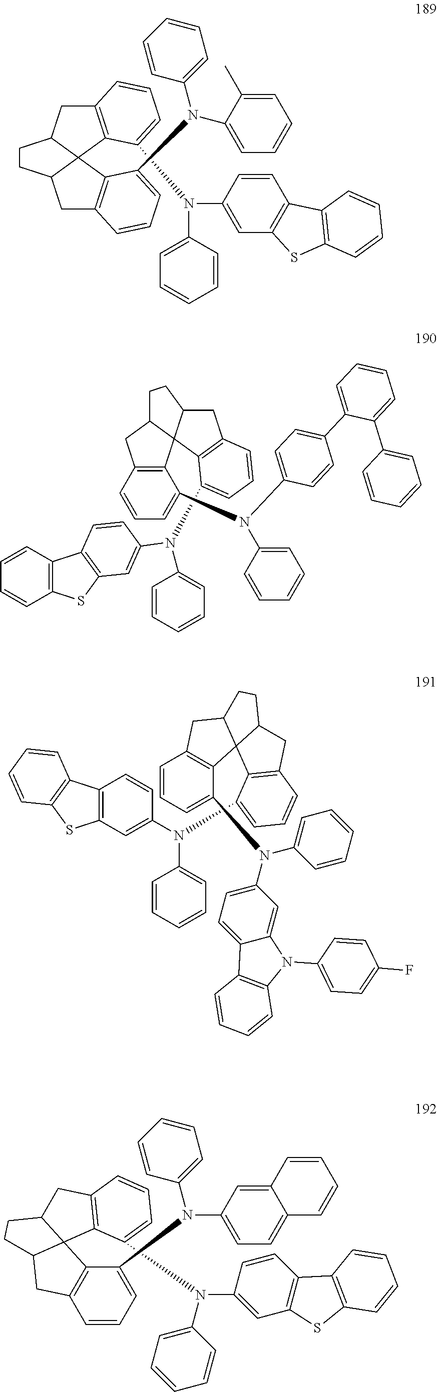

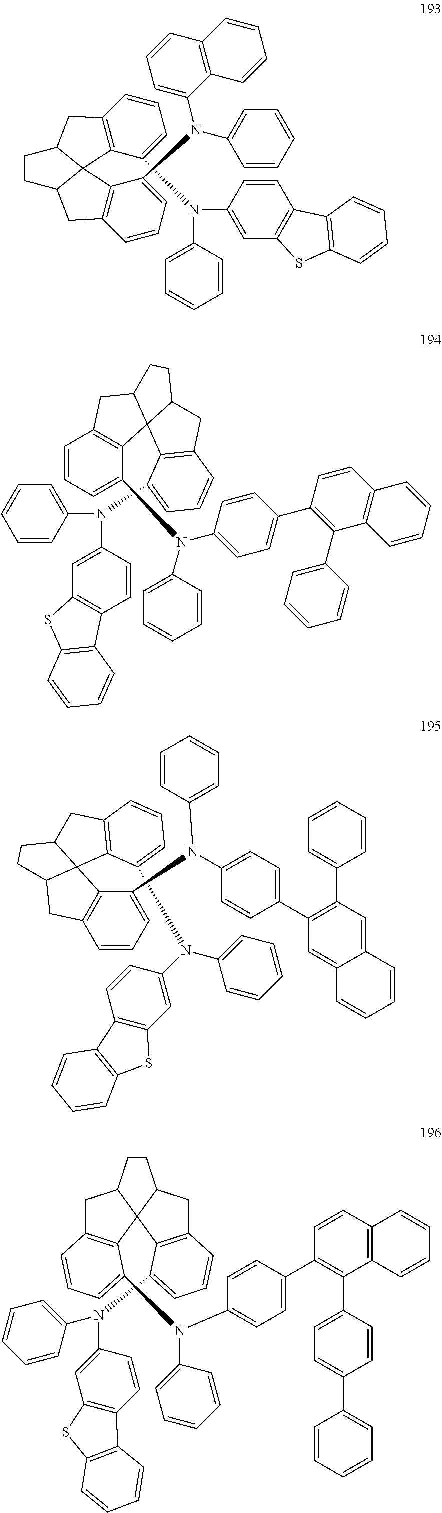

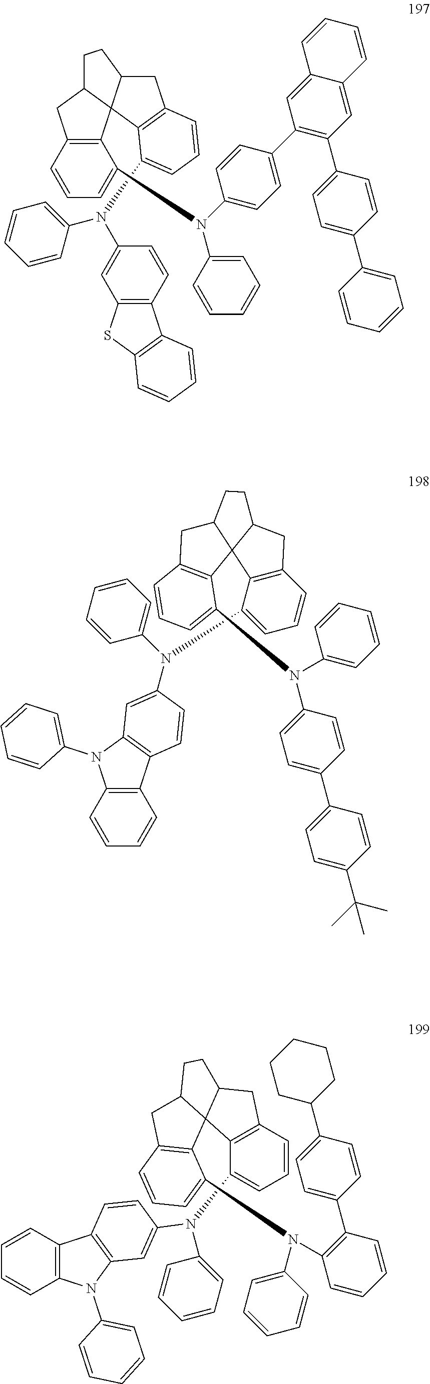

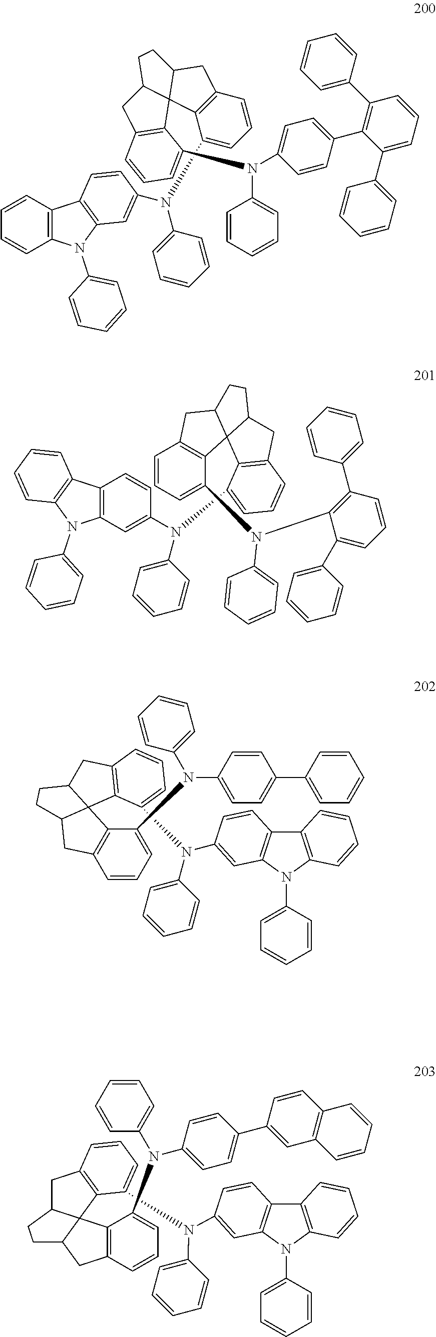

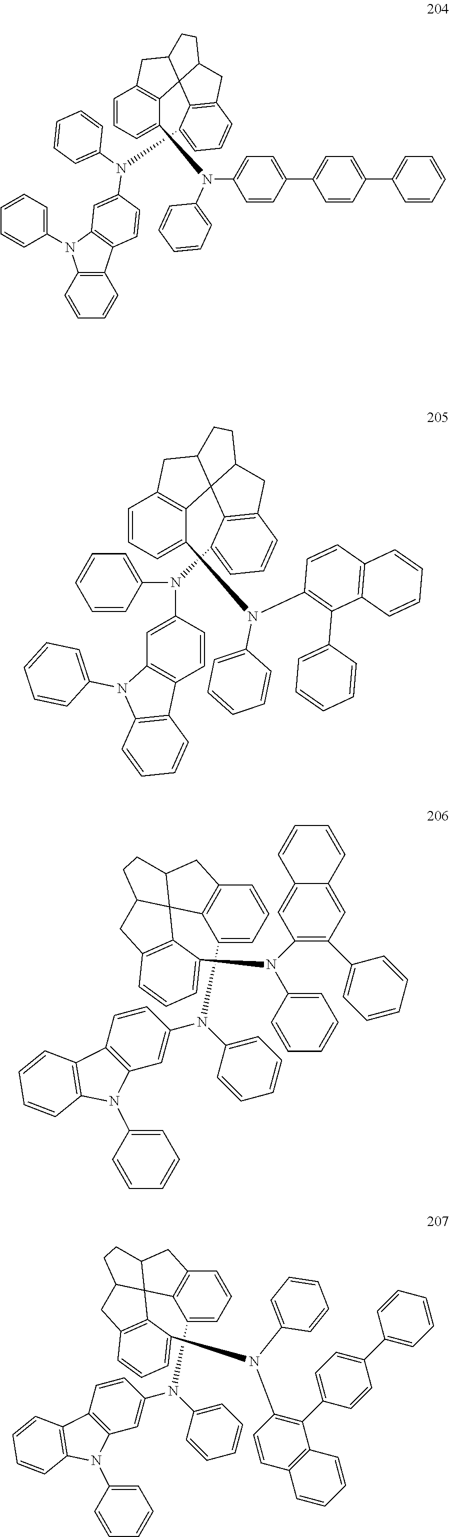

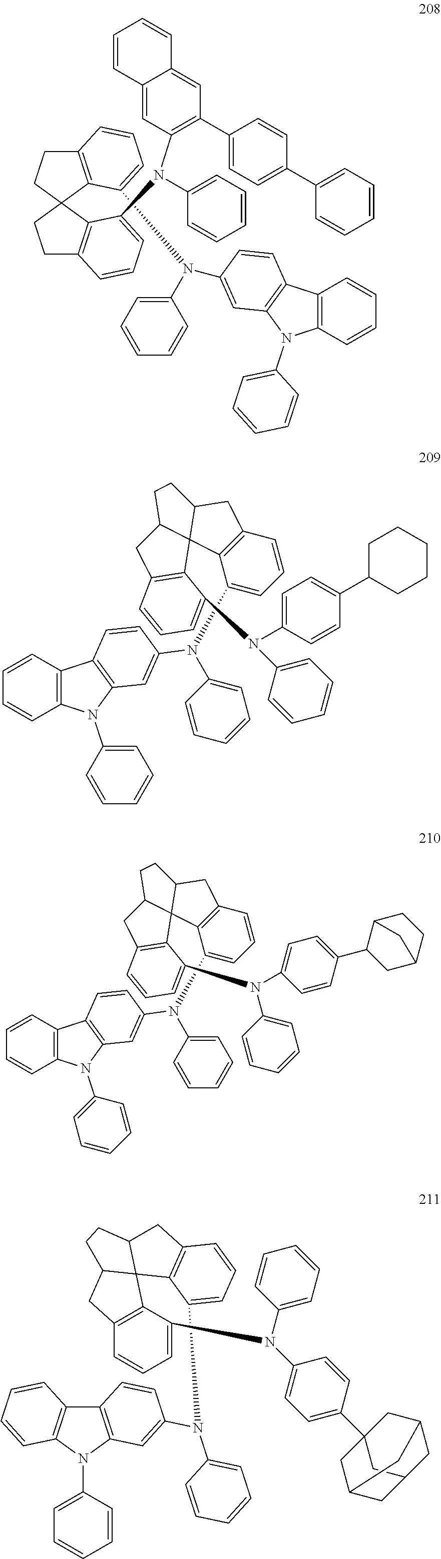

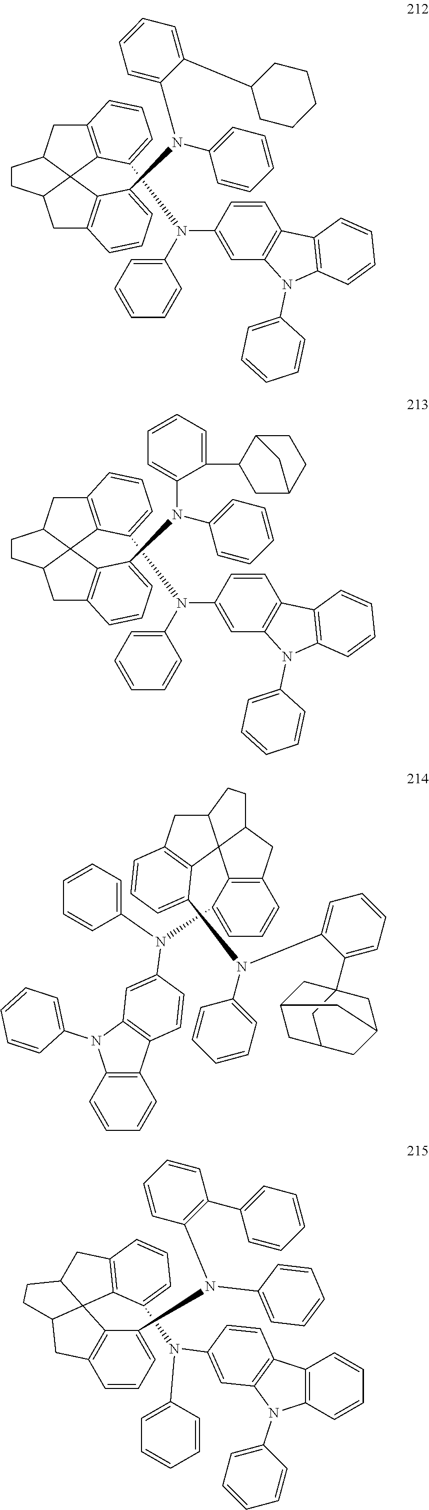

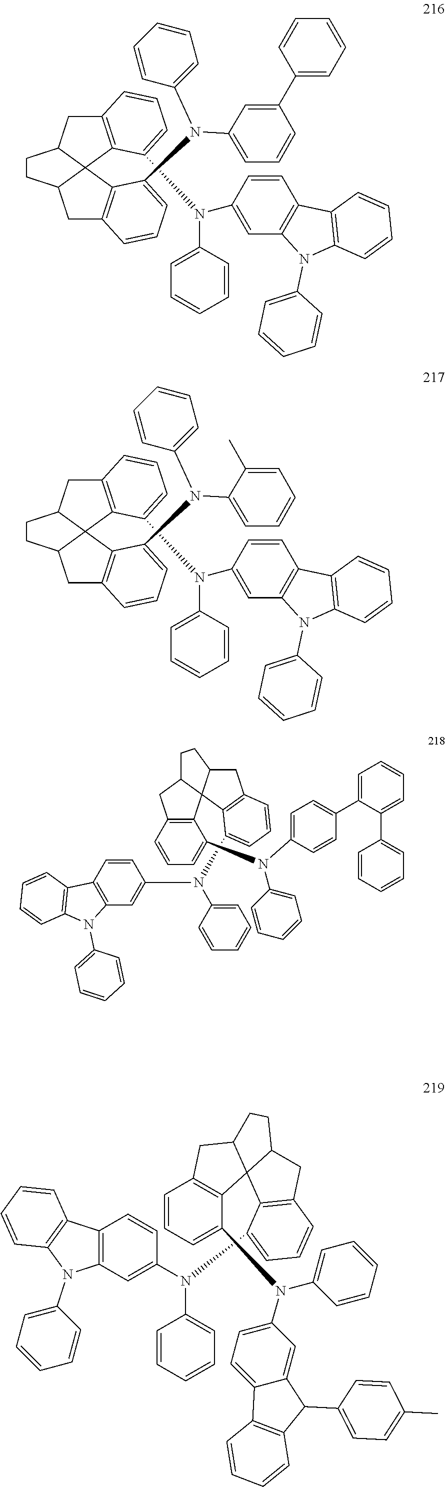

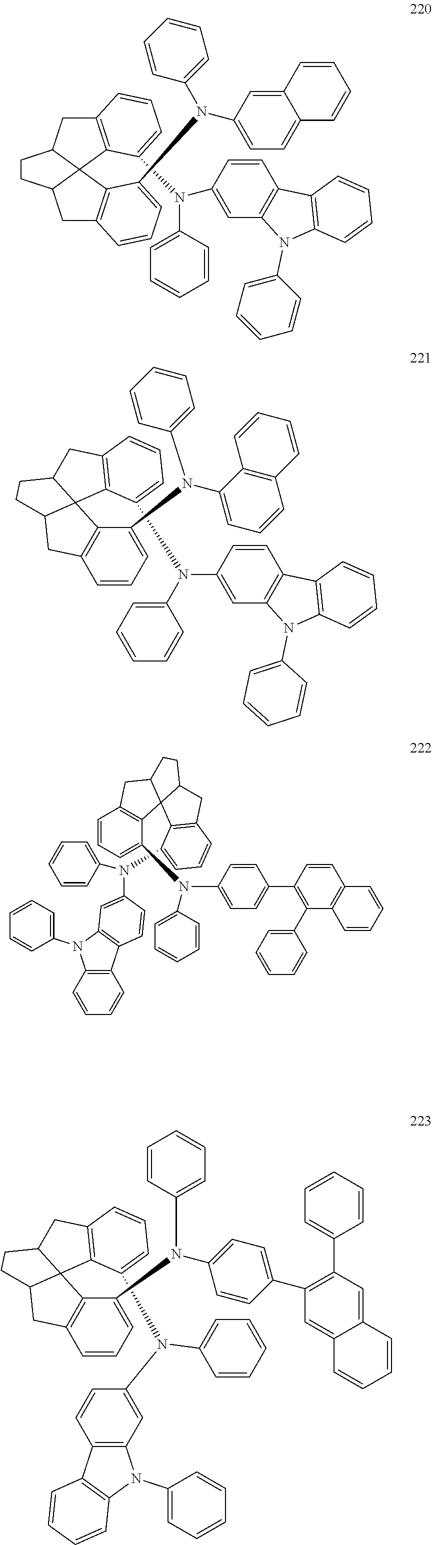

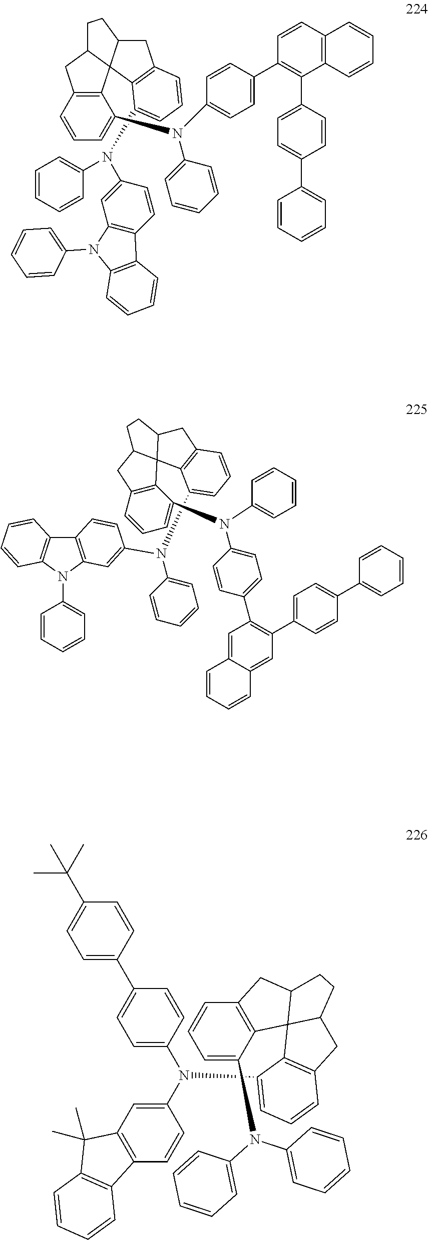

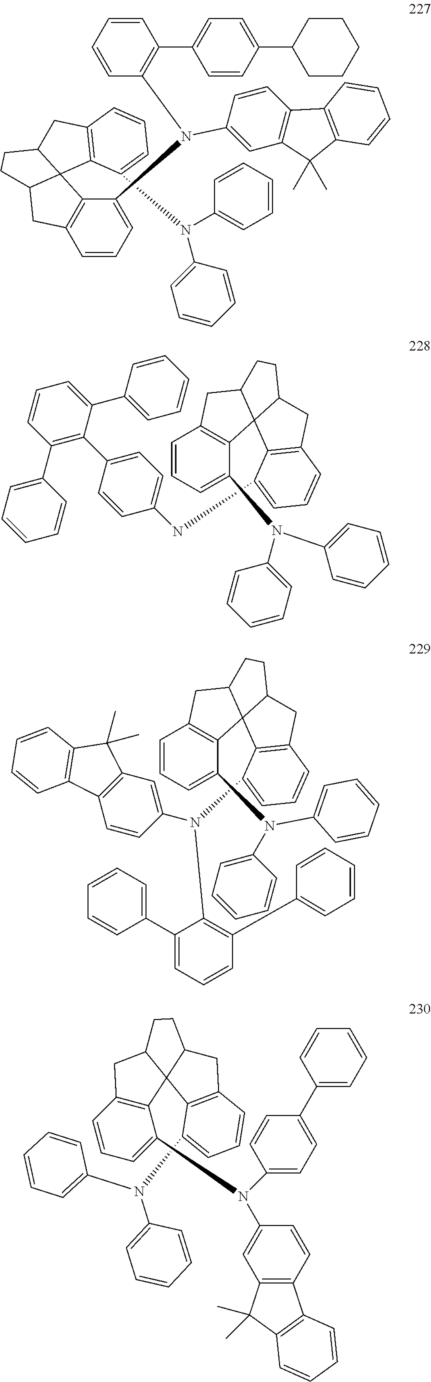

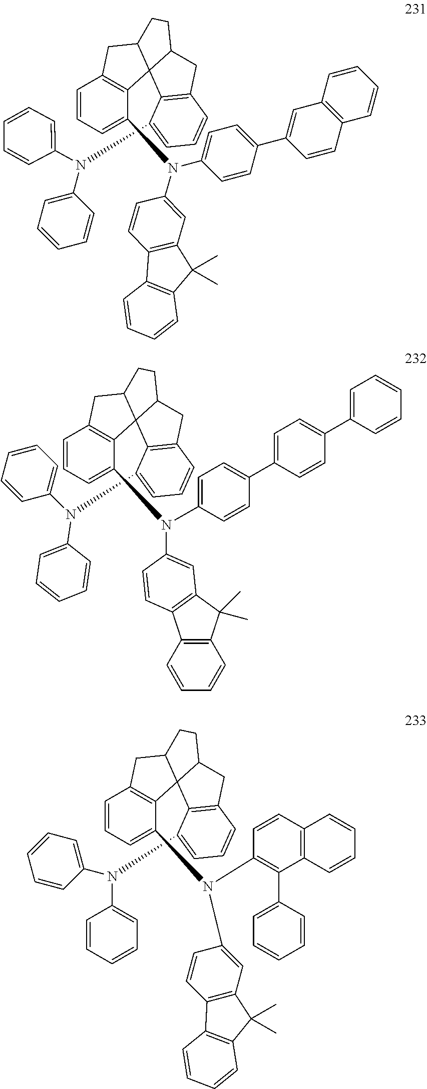

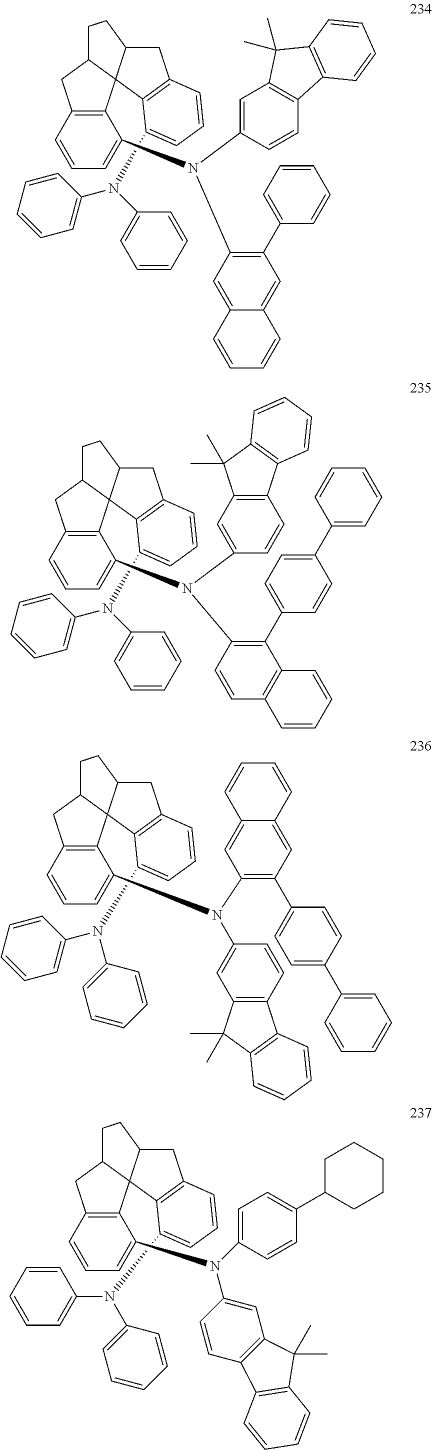

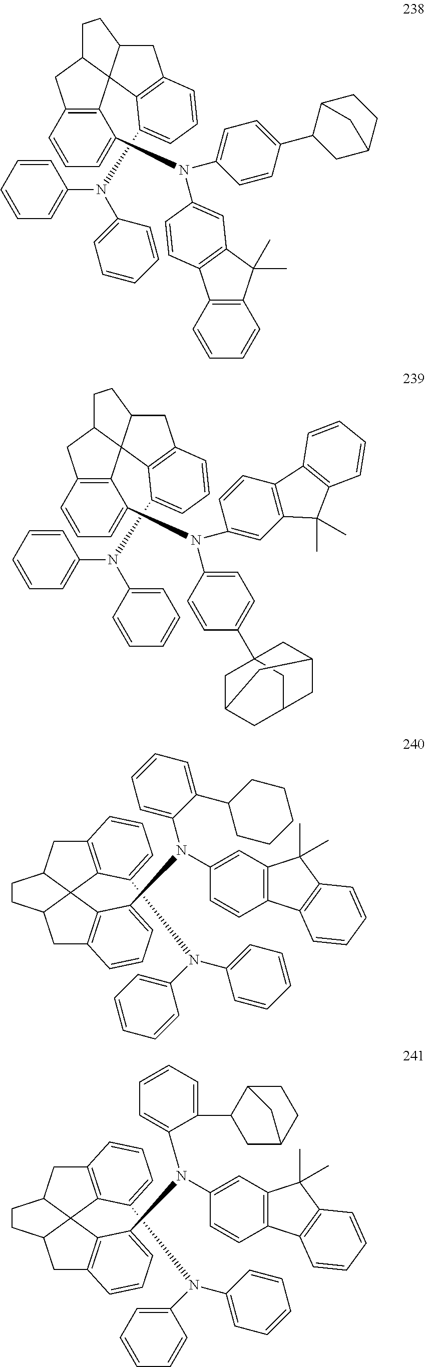

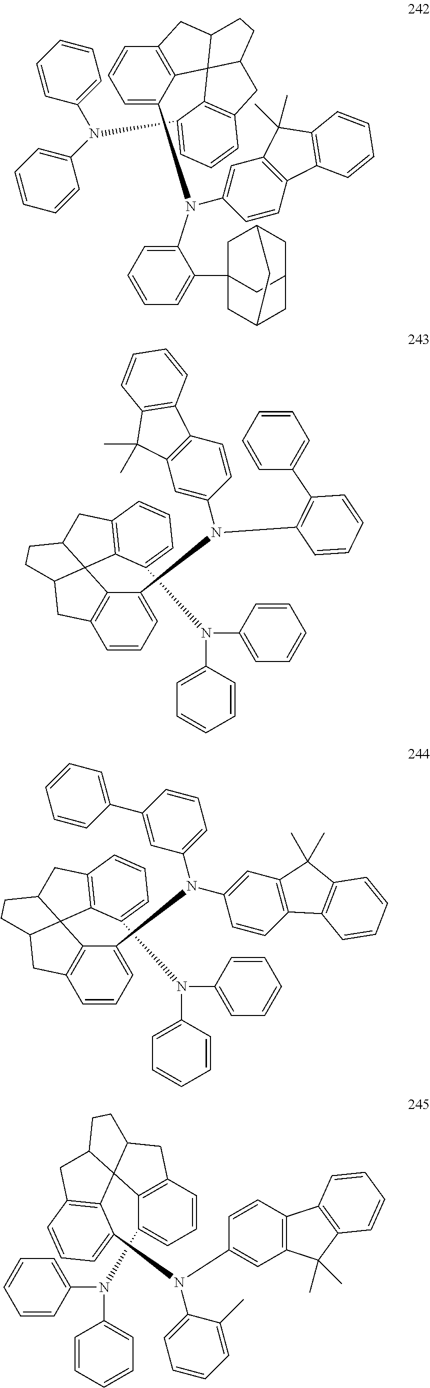

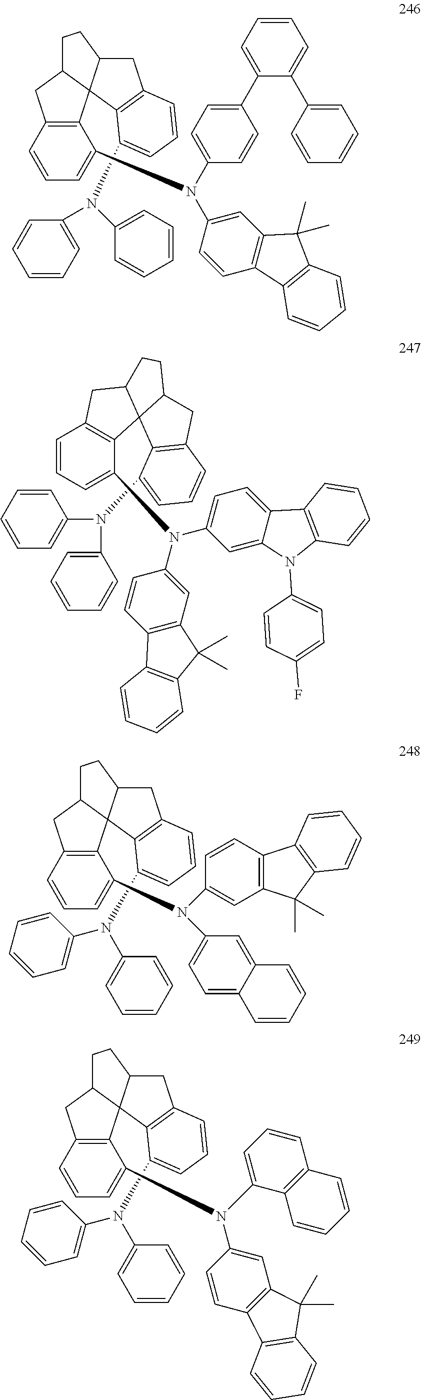

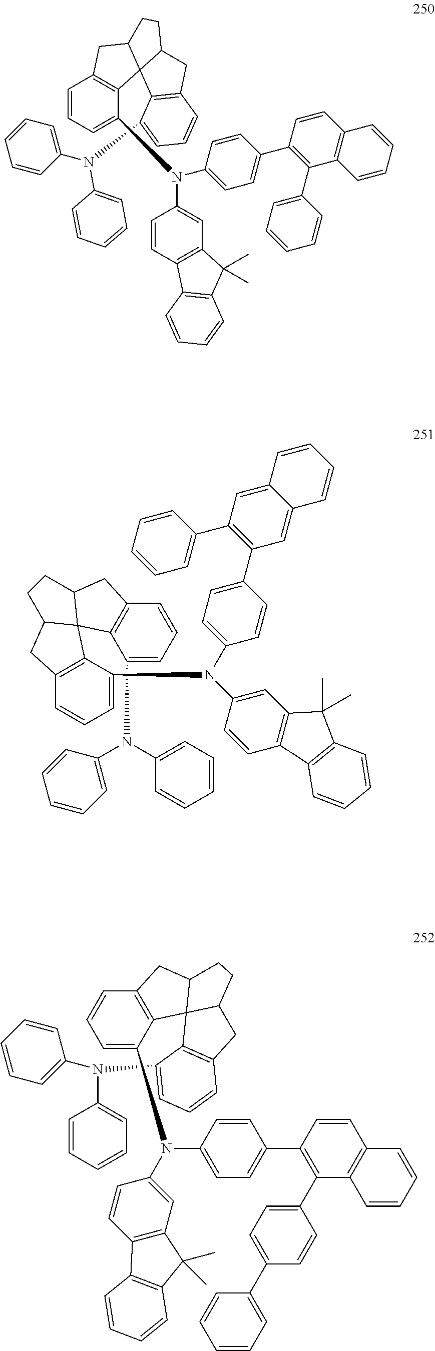

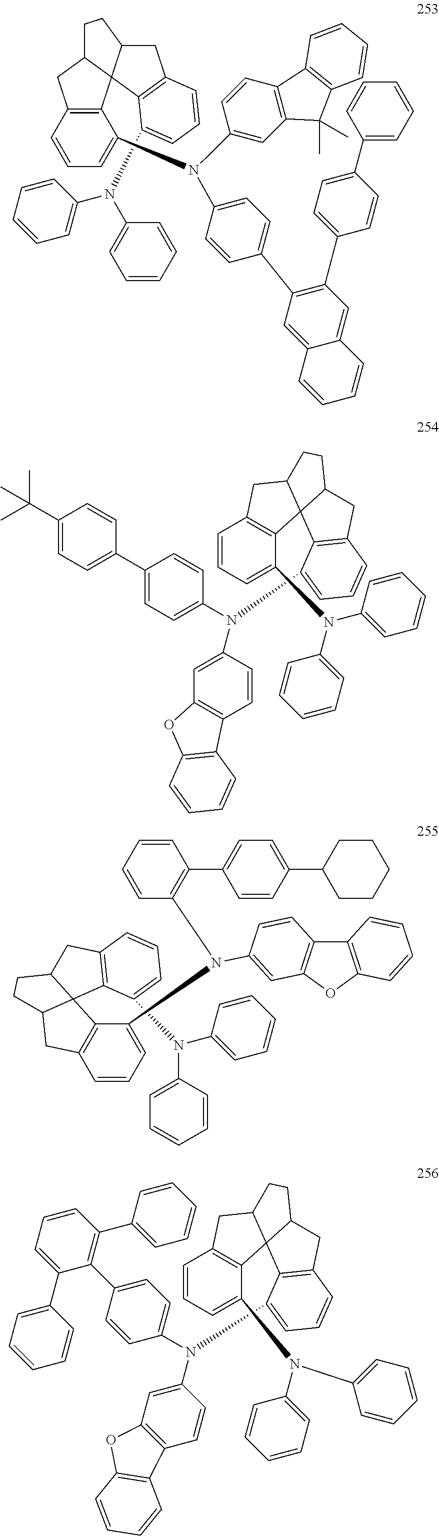

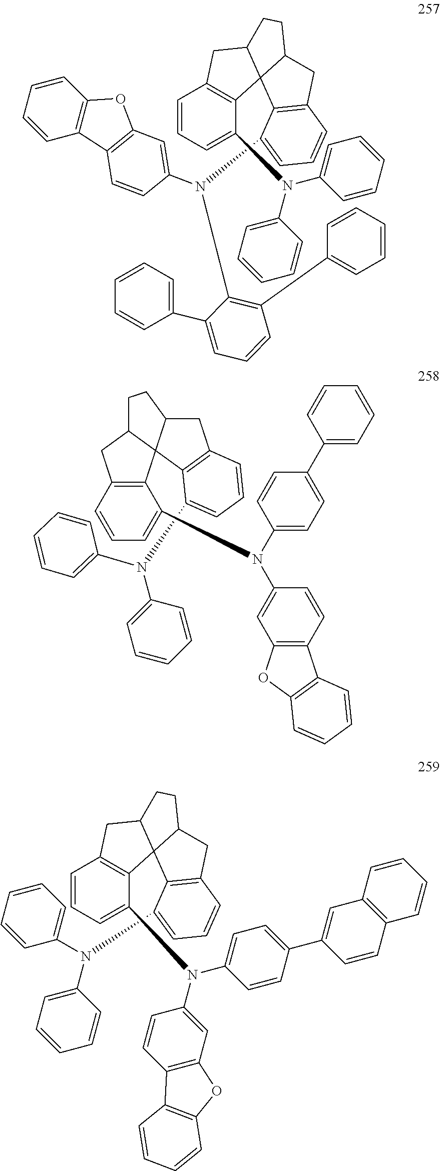

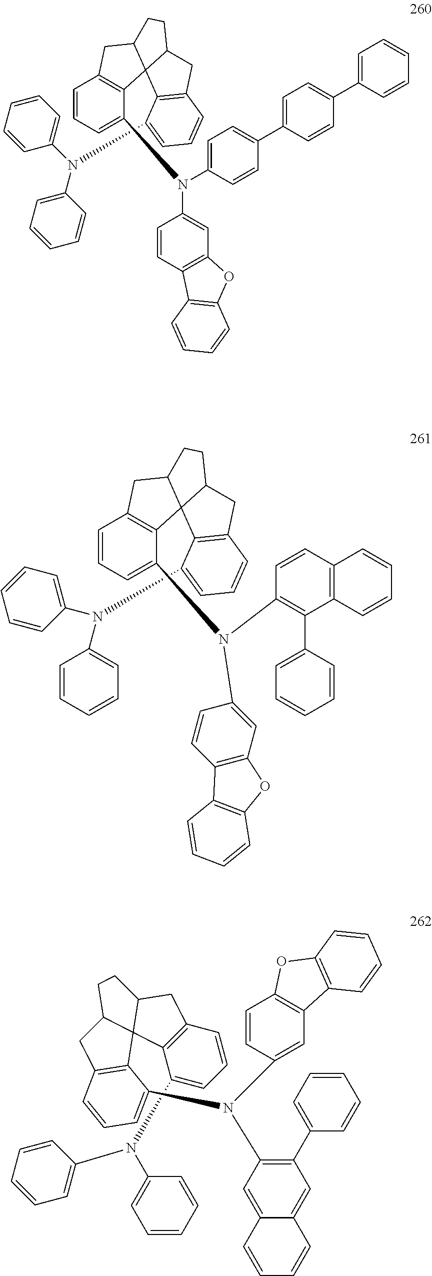

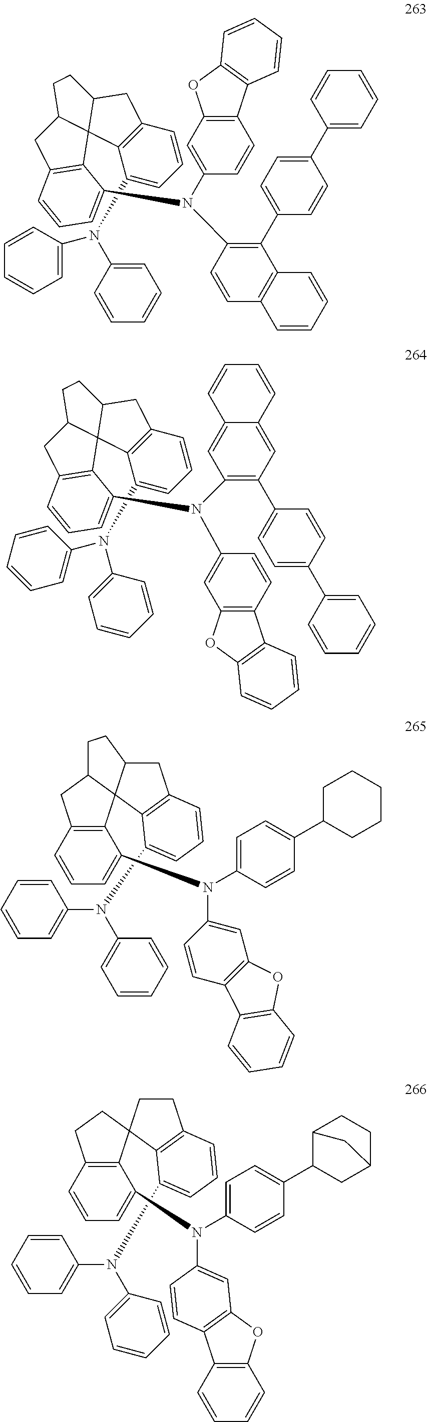

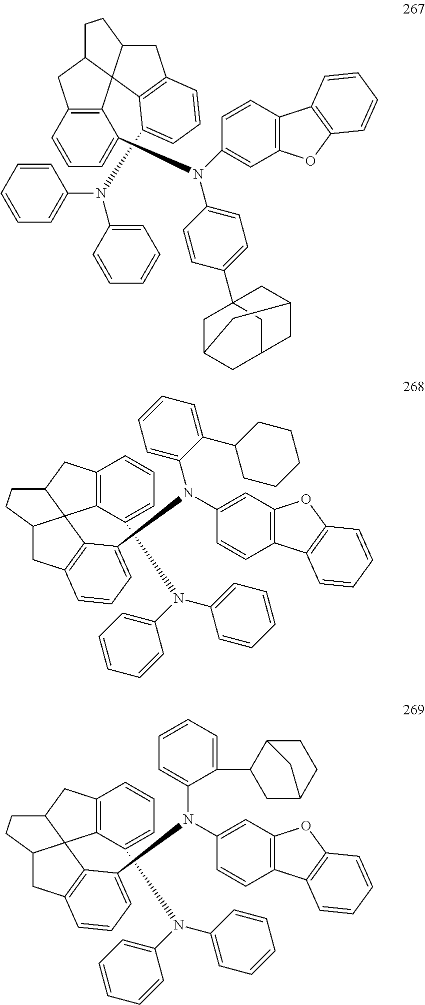

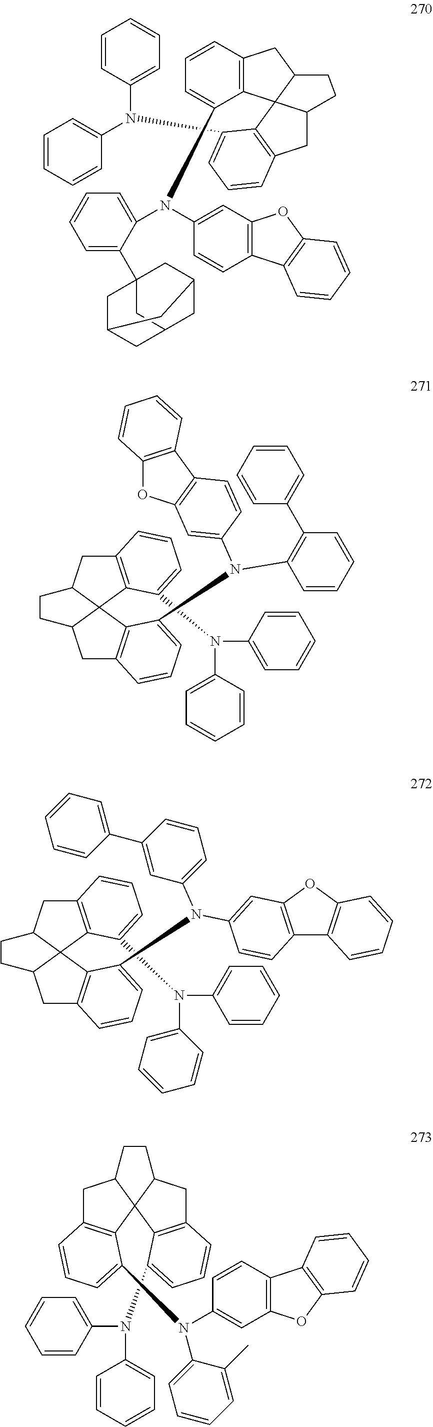

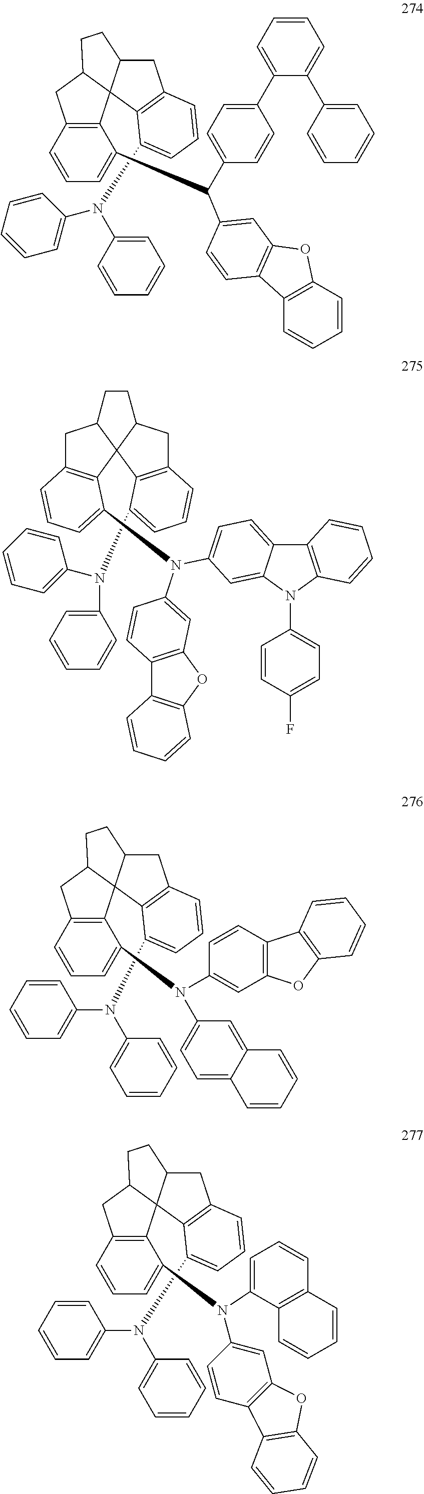

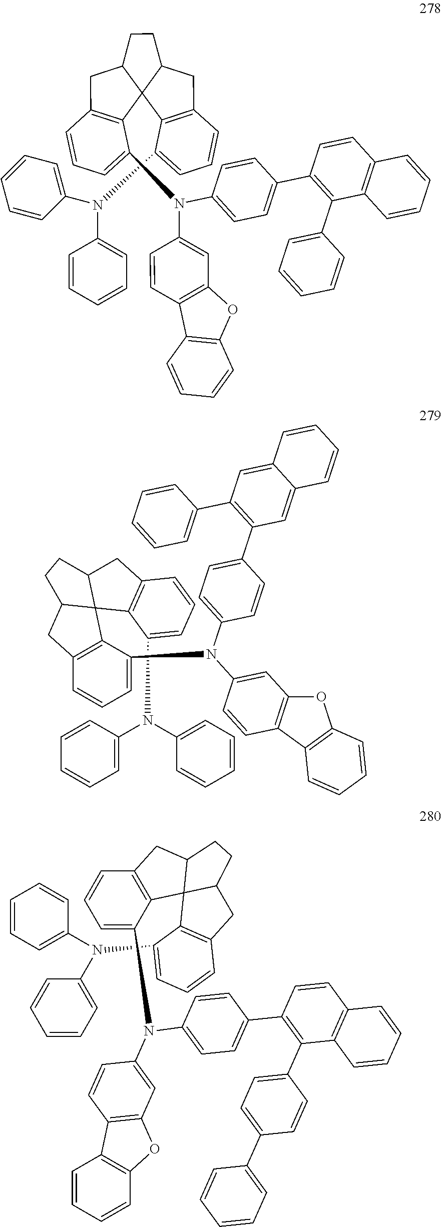

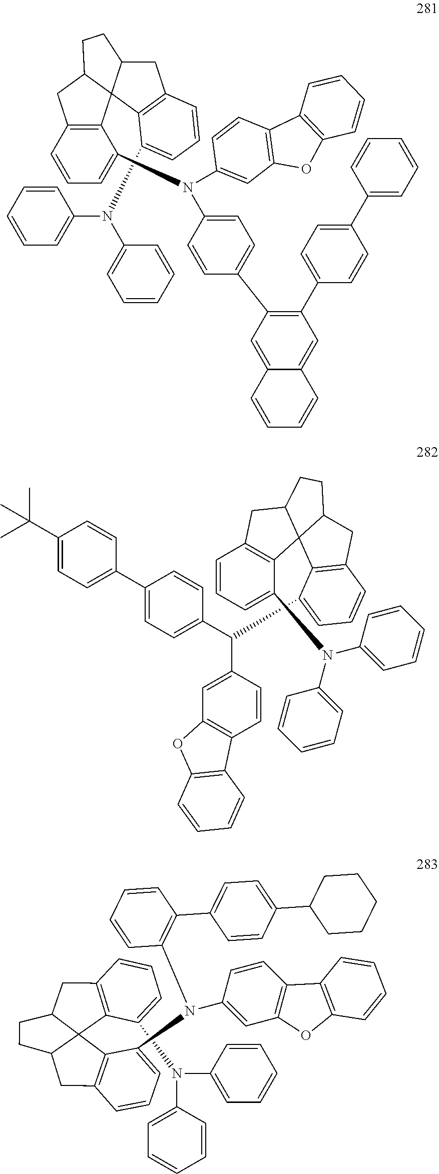

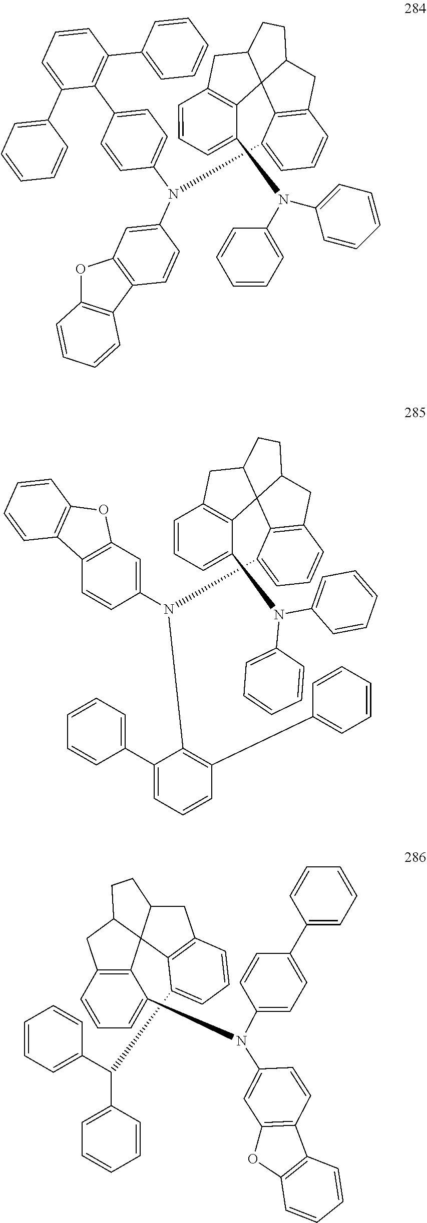

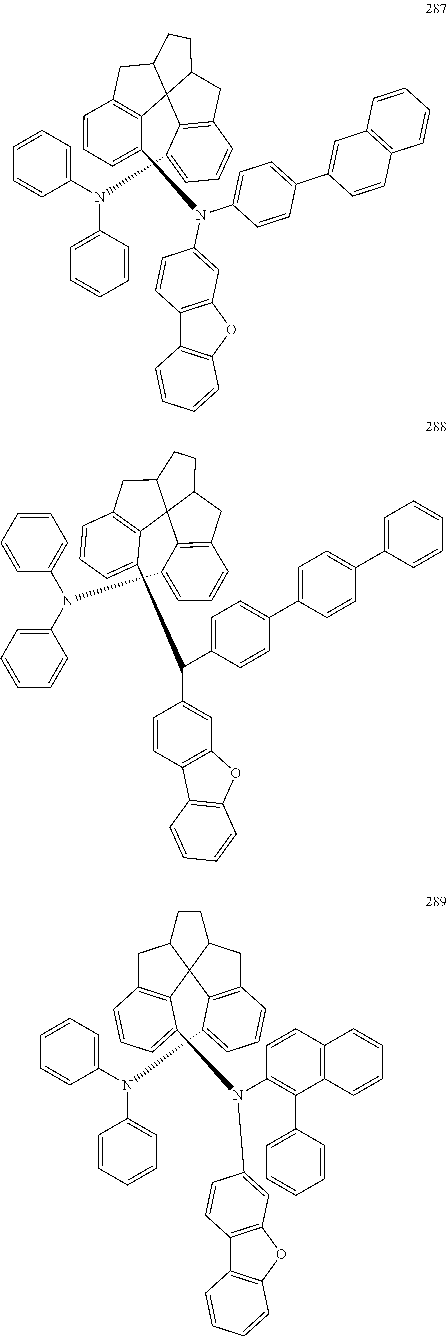

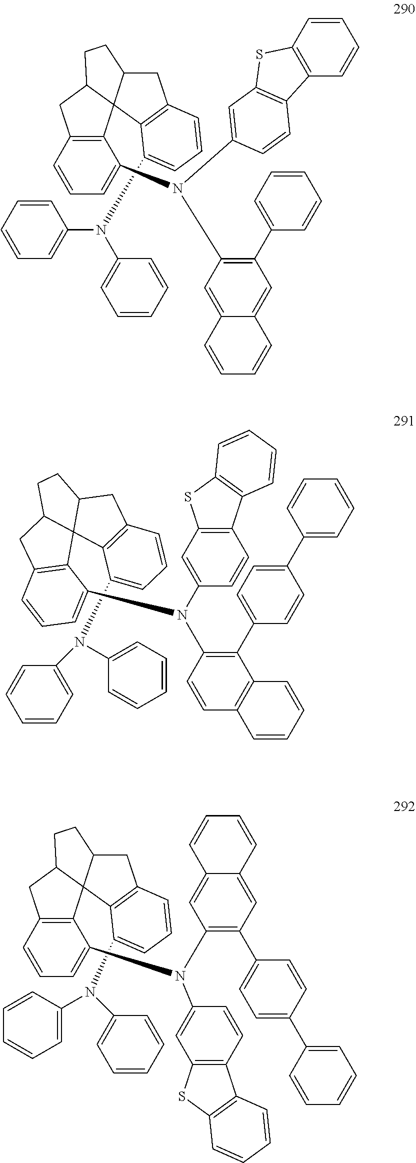

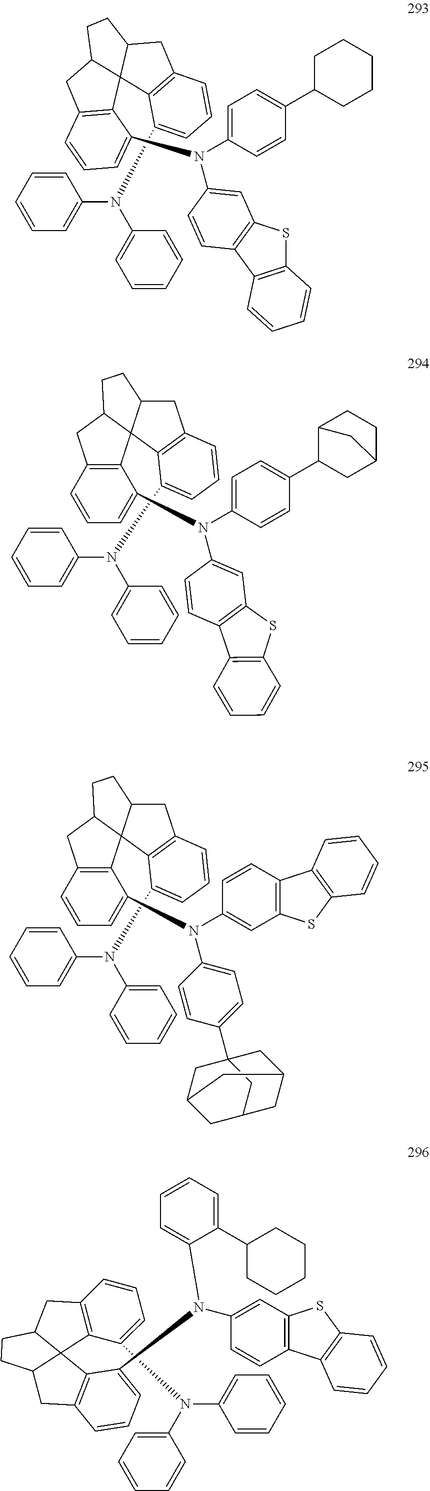

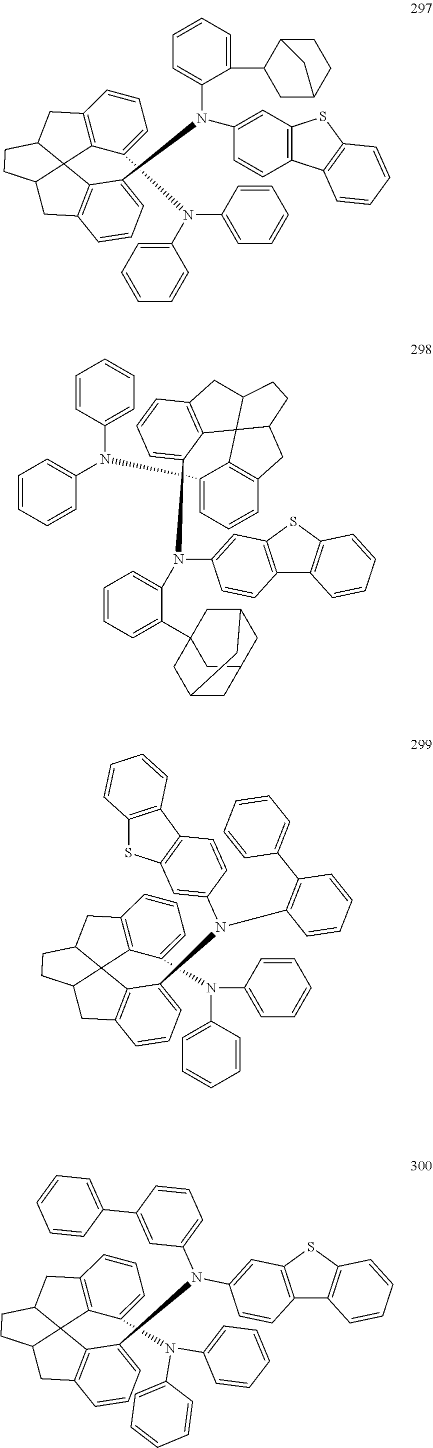

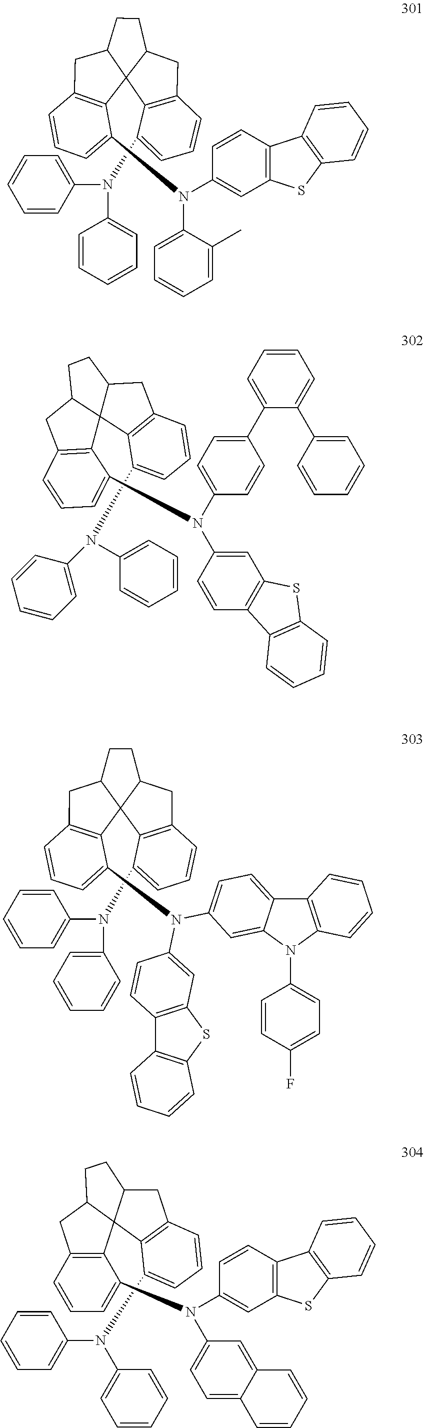

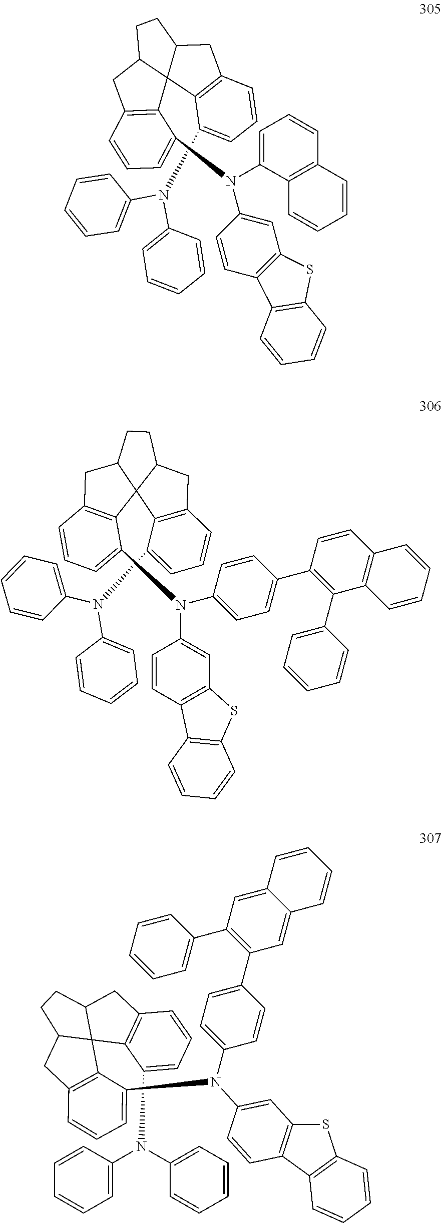

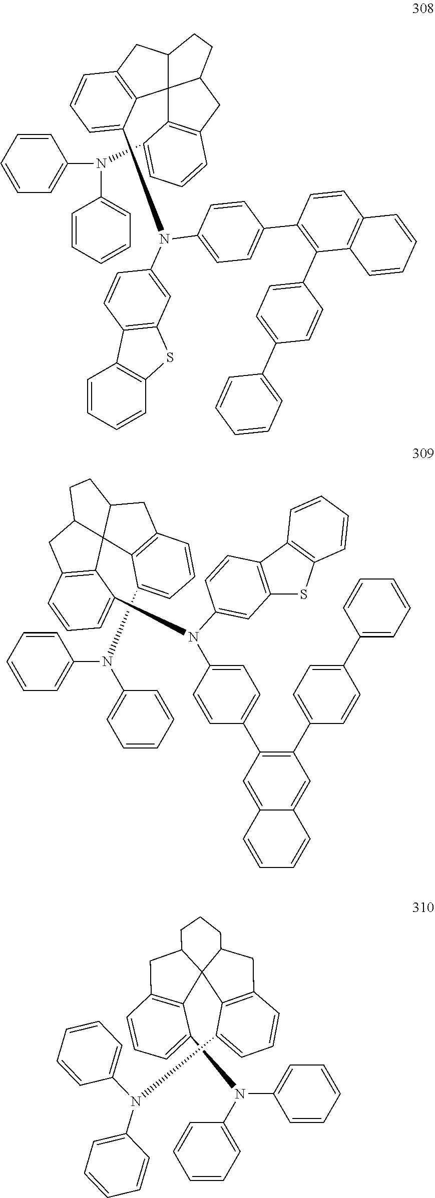

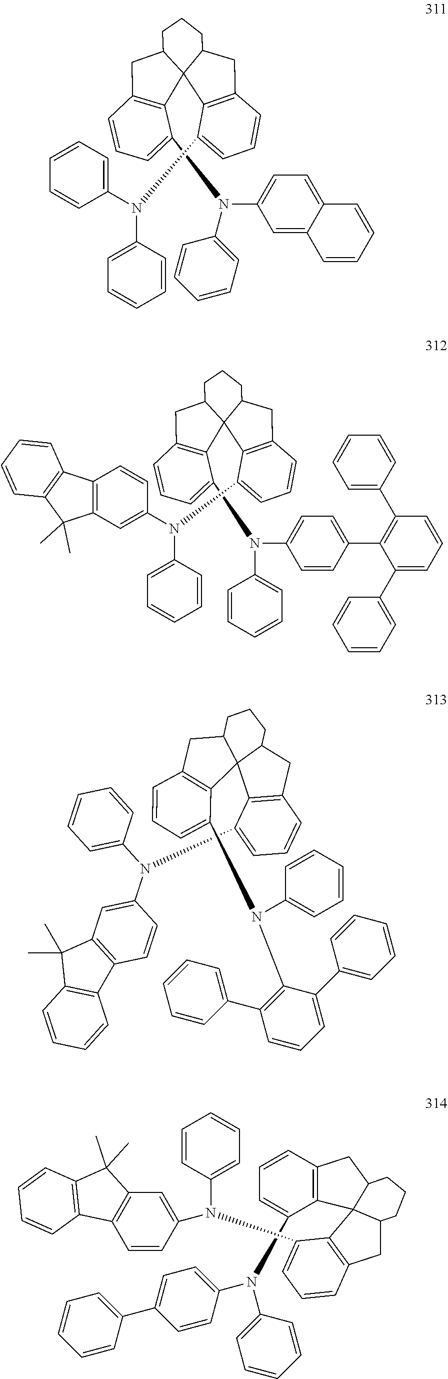

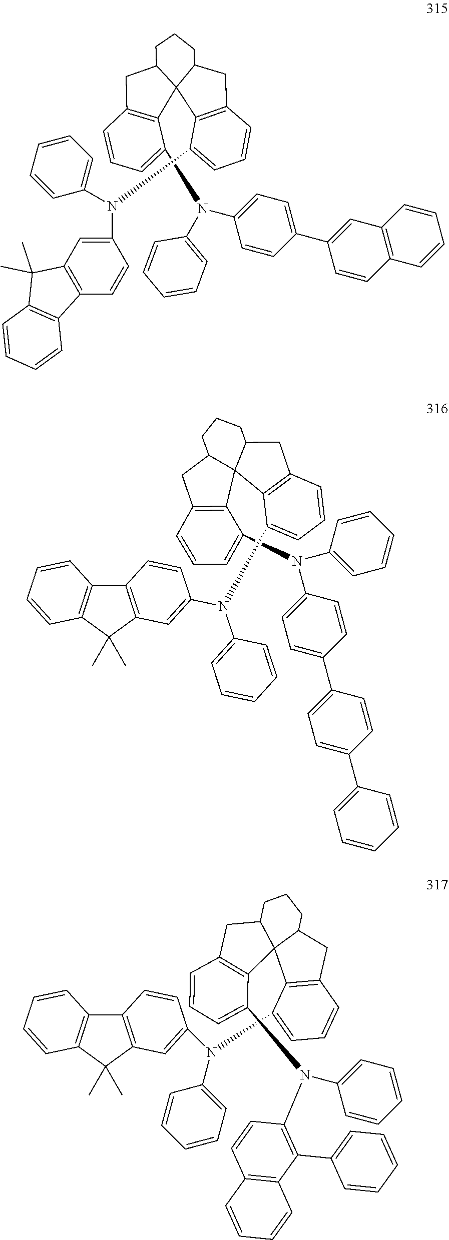

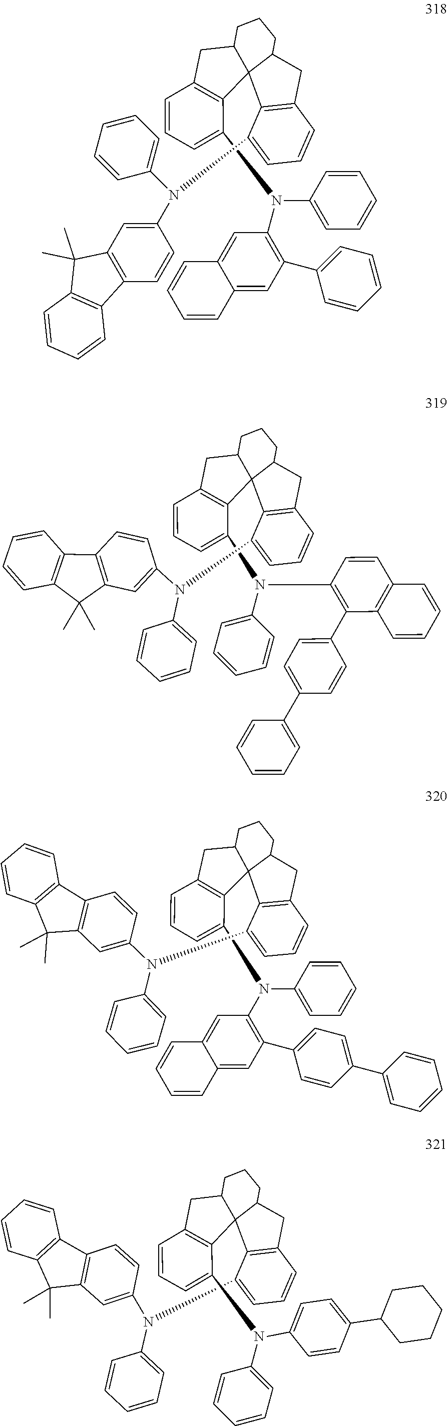

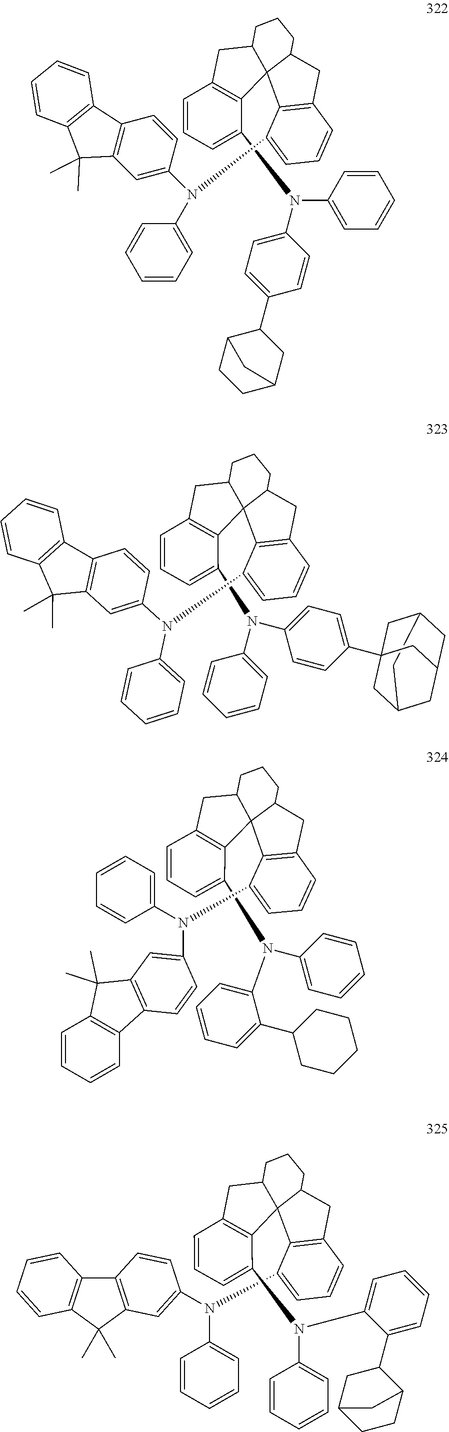

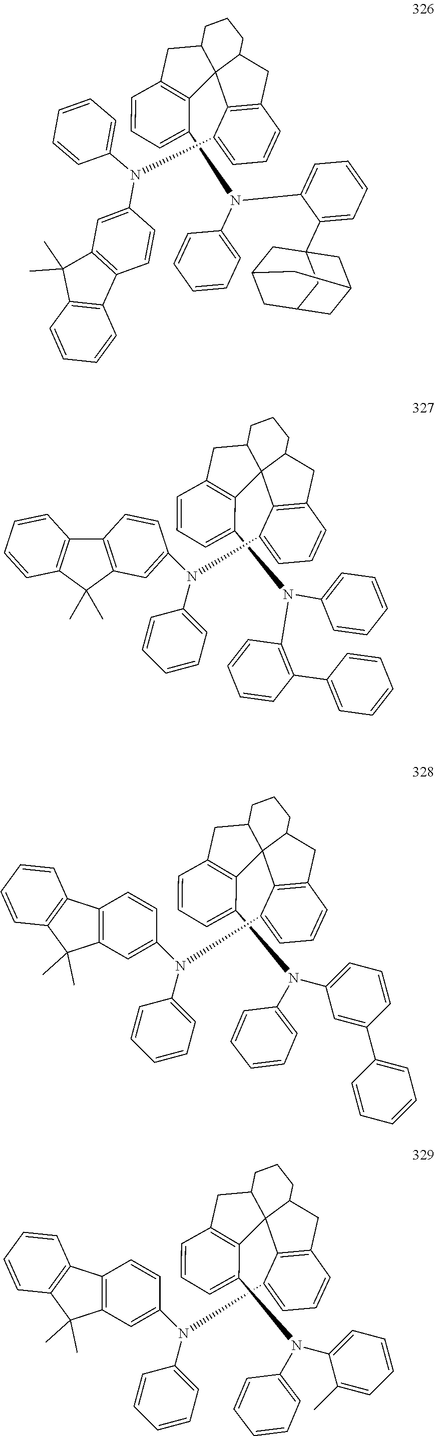

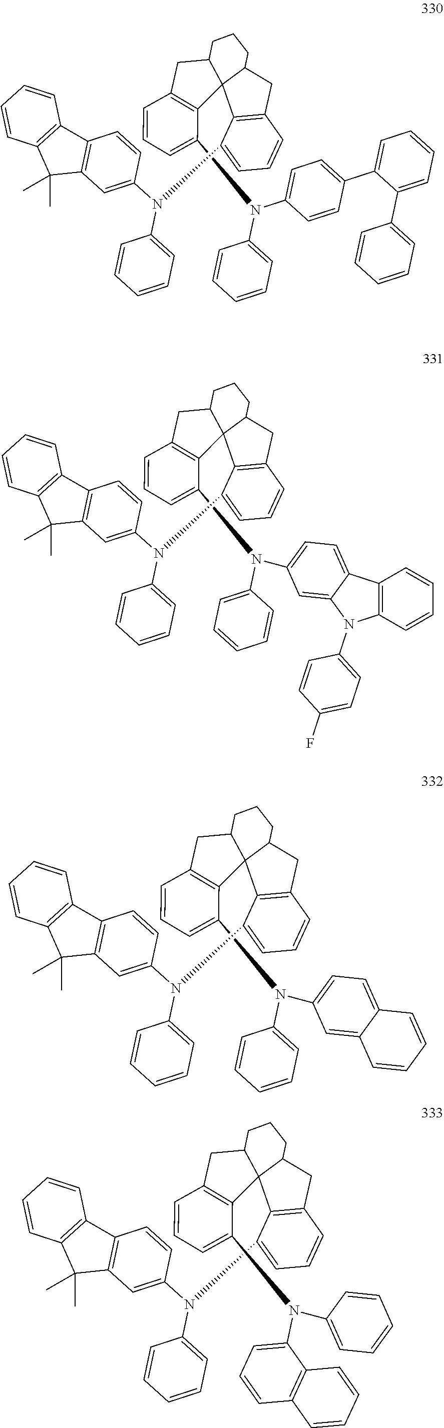

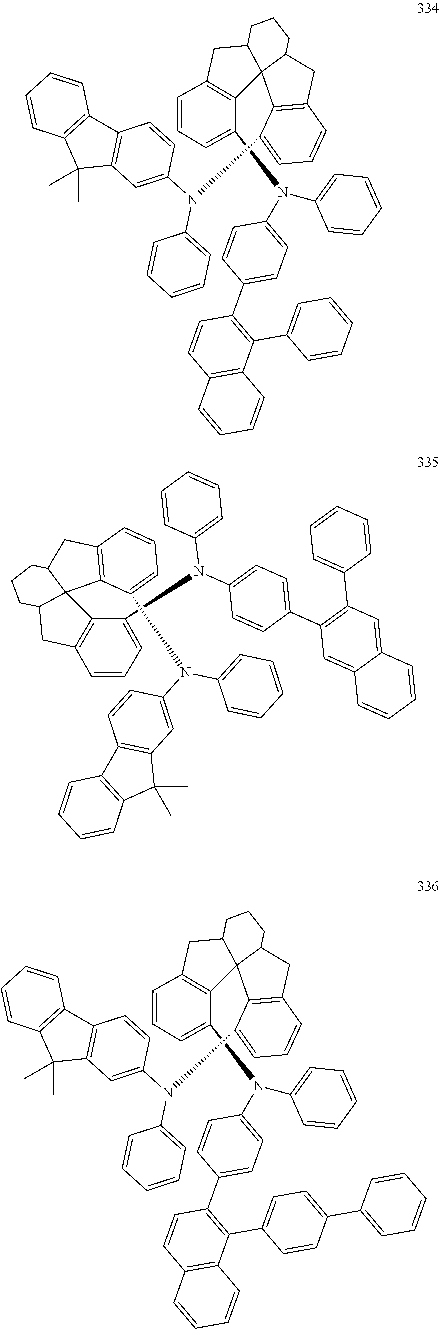

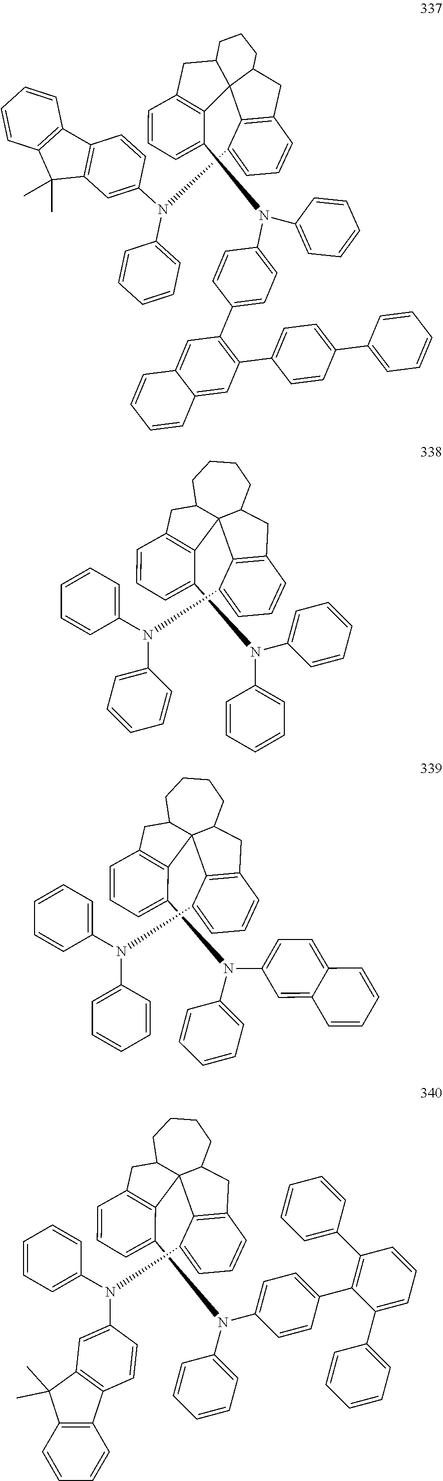

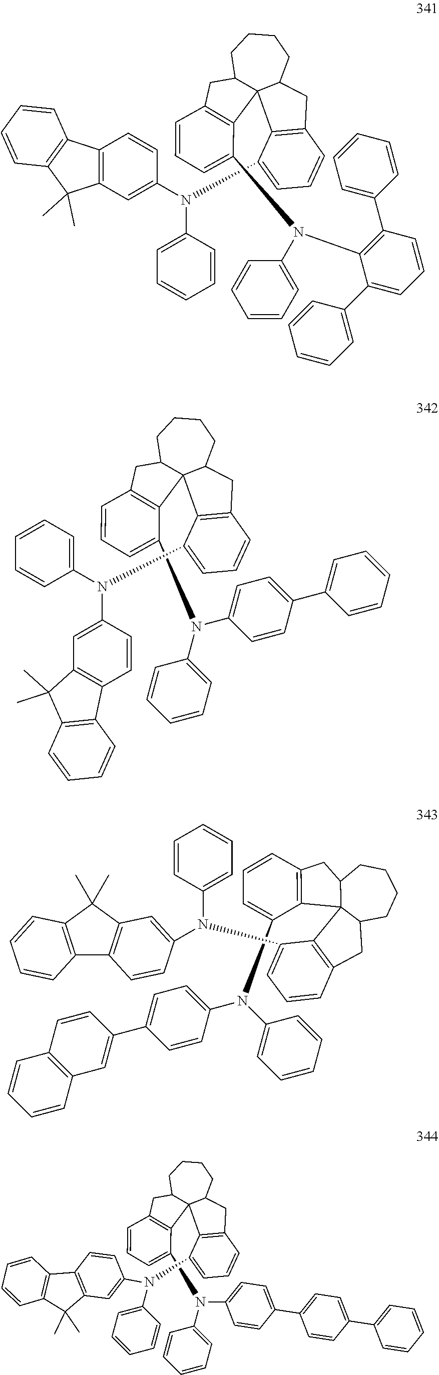

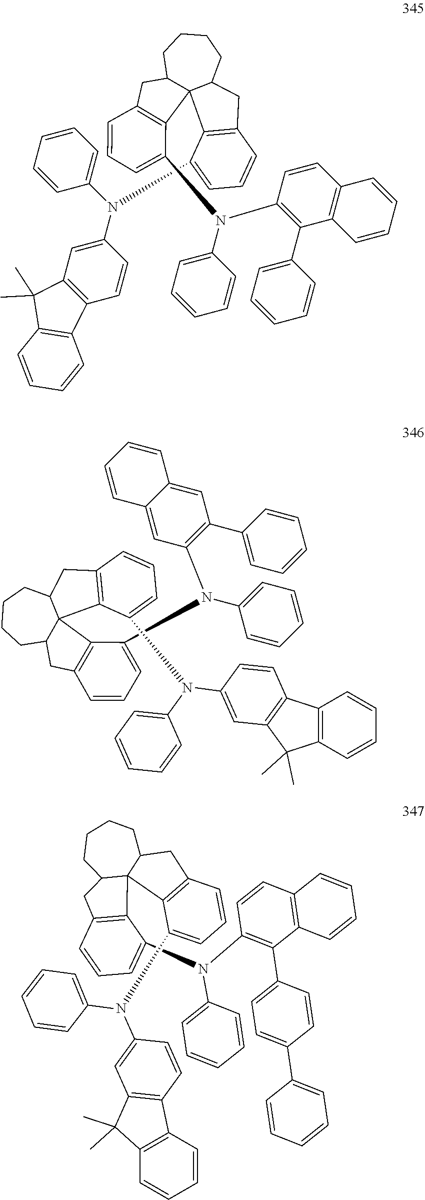

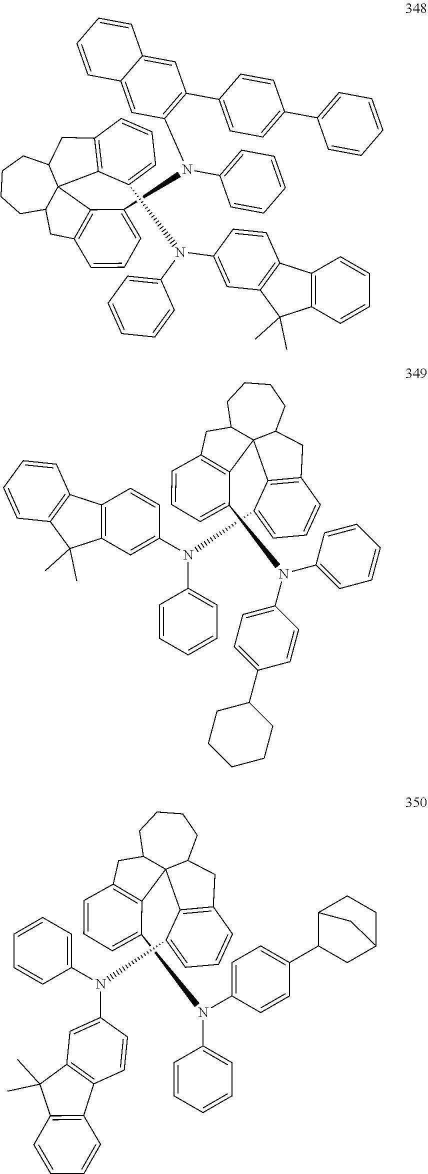

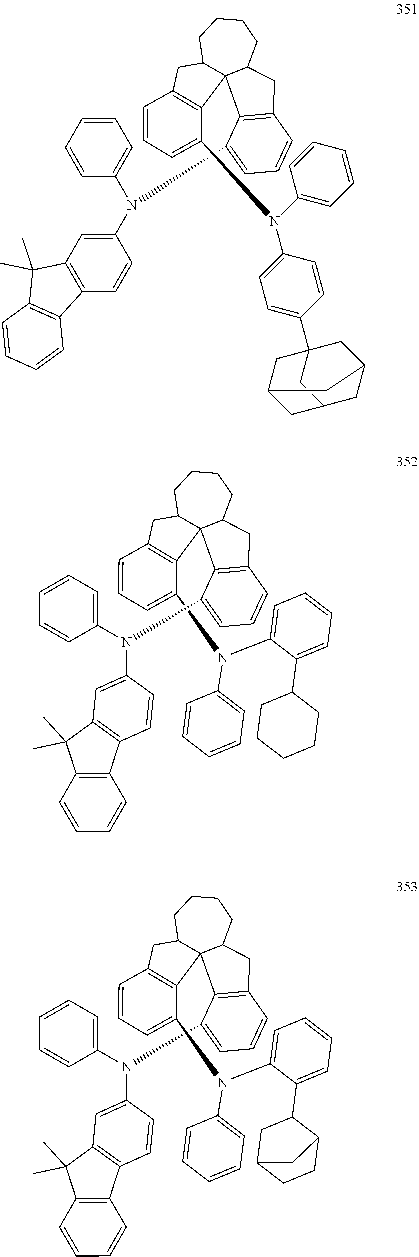

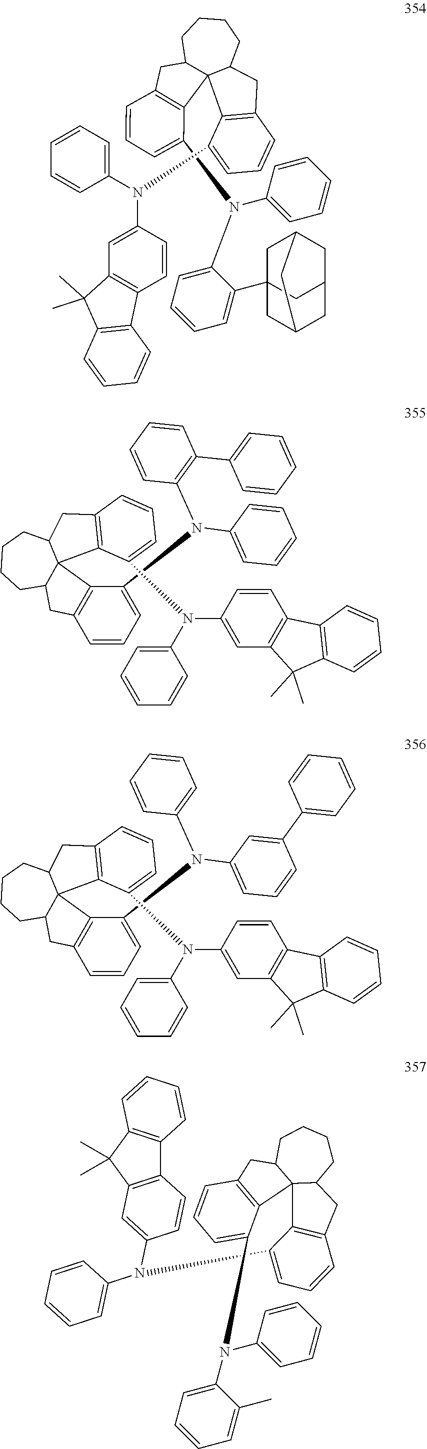

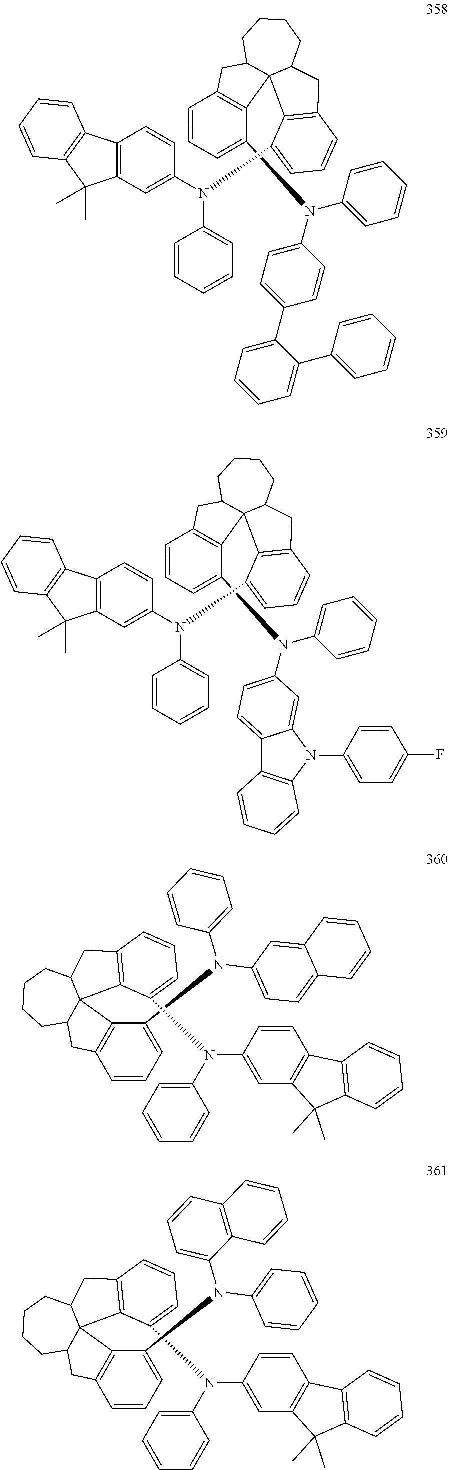

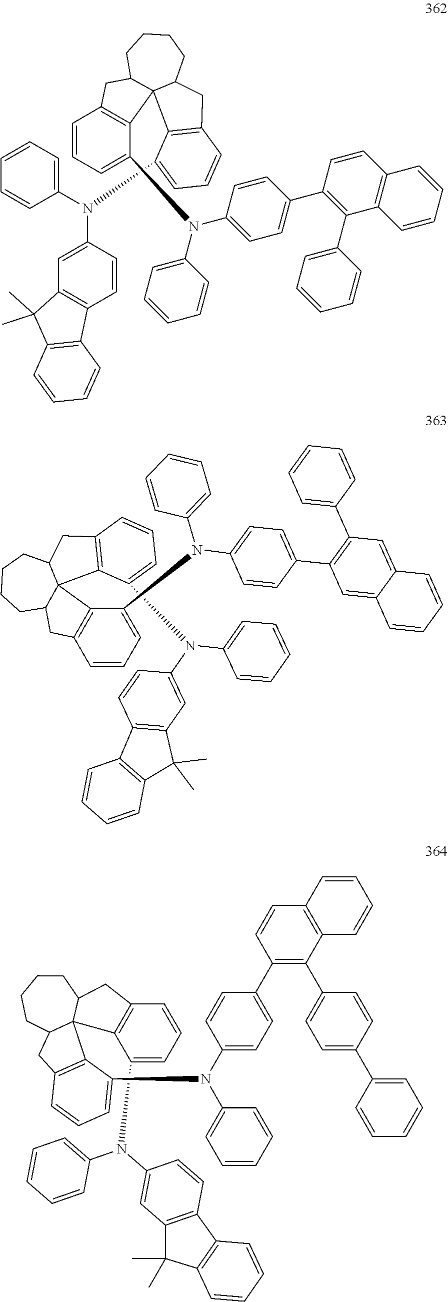

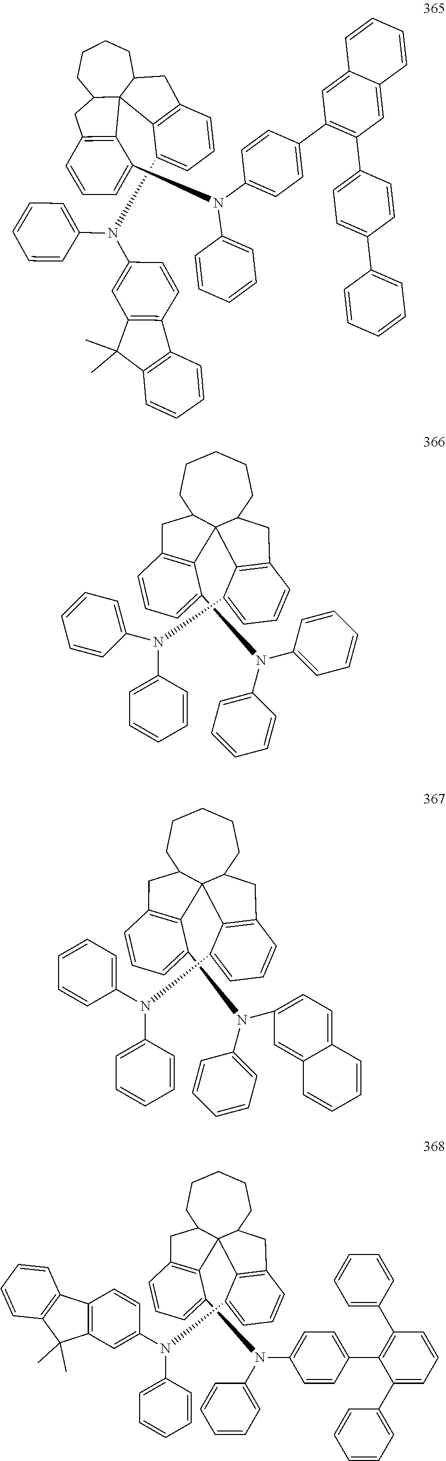

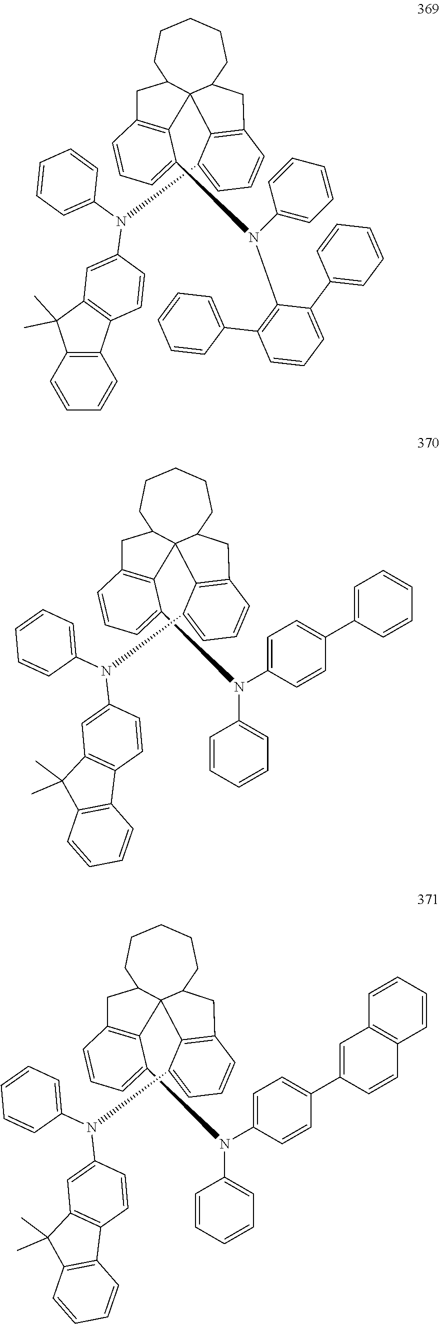

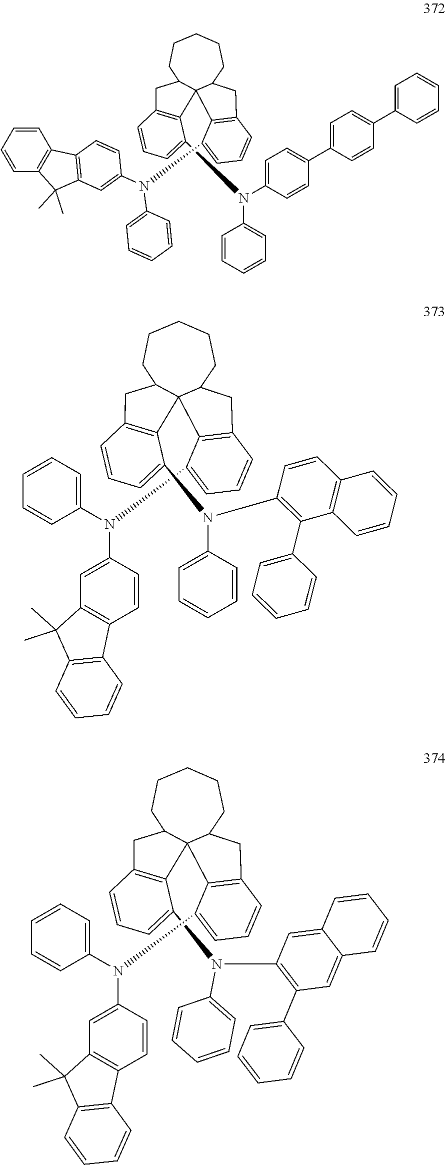

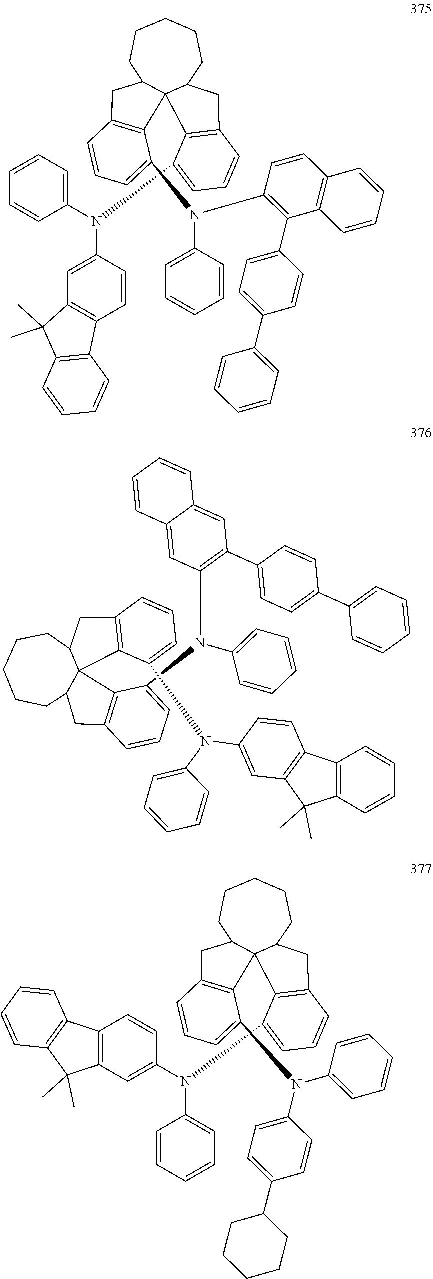

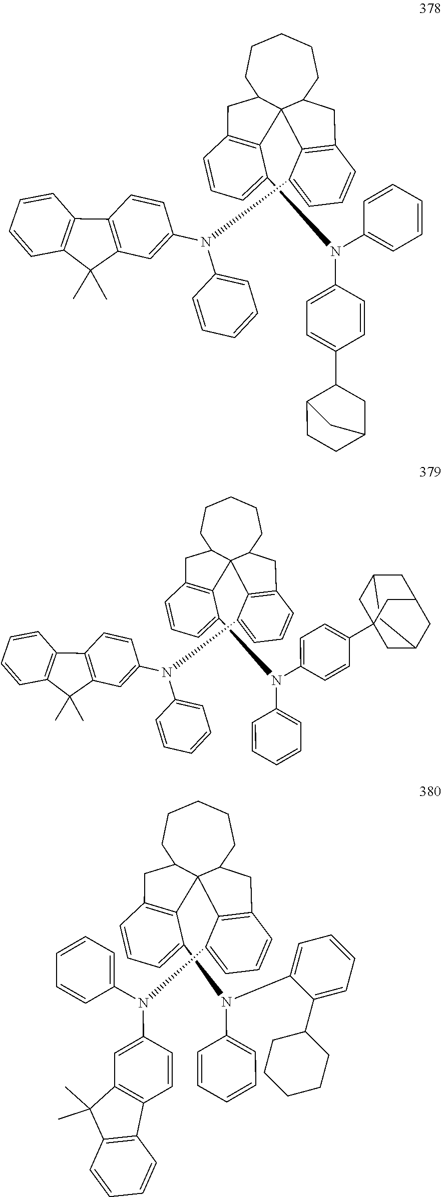

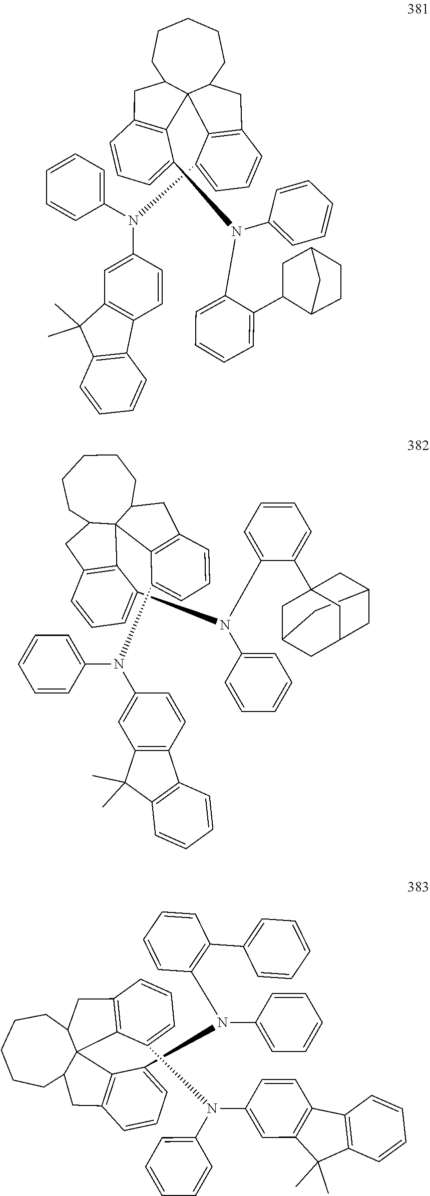

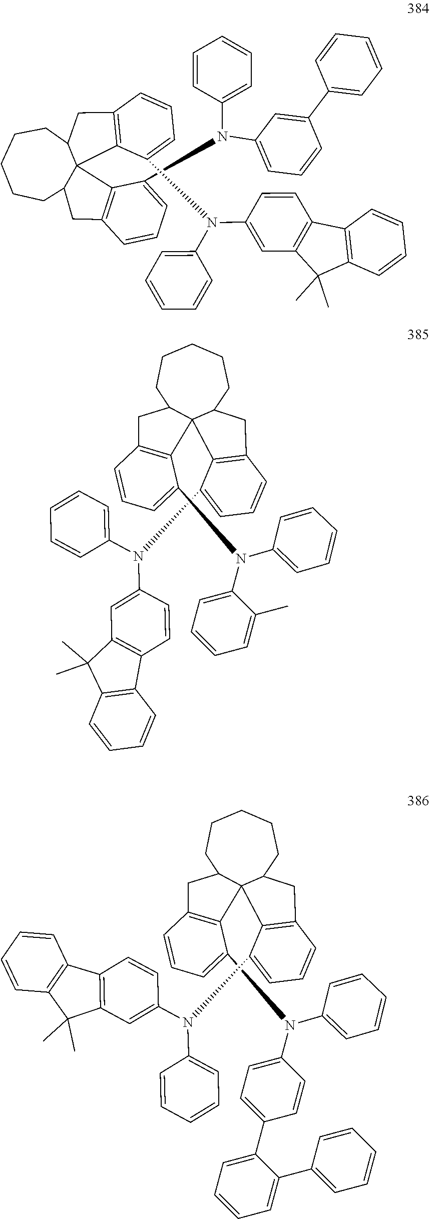

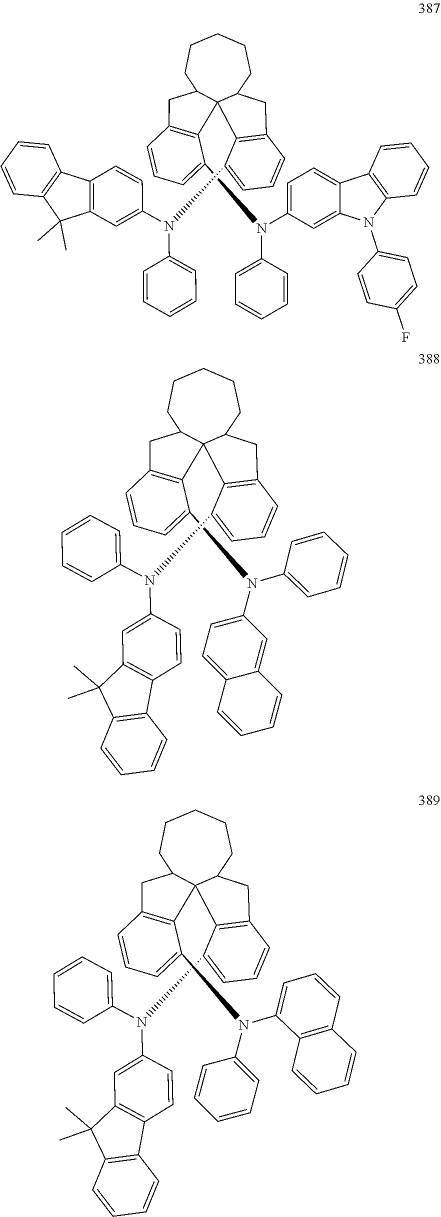

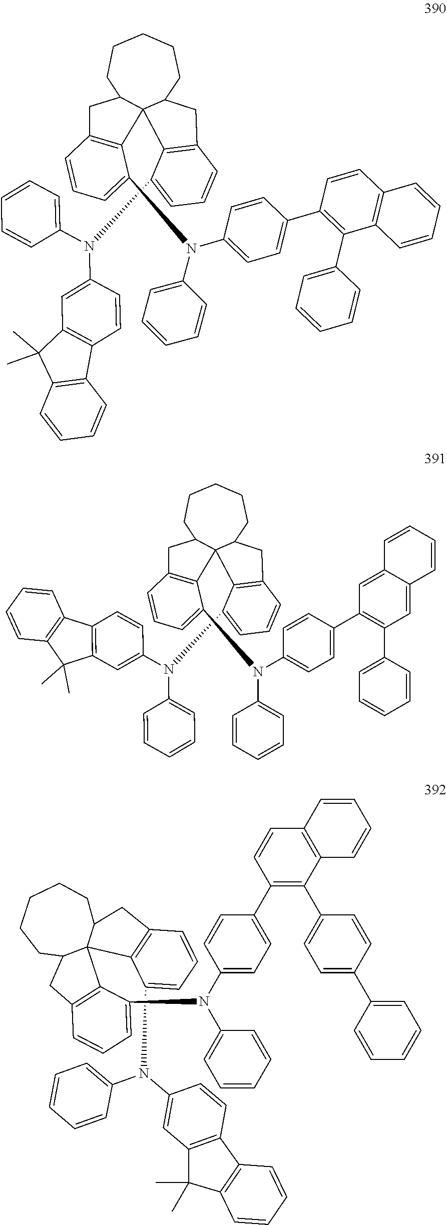

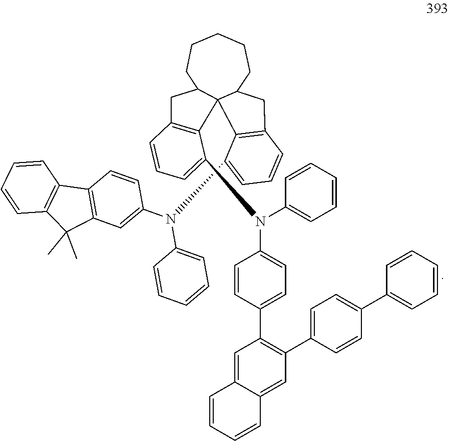

The amine compound according to an embodiment necessarily includes the spirobiindane moiety, and has a structure in which the first amine group and the second amine group are respectively linked to the first benzene ring and the second benzene ring included in the spirobiindane moiety. The first amine group and the second amine group may be directly linked to the first benzene ring and the second benzene ring, respectively. The amine compound includes an alkyl substituent linking carbon 2 and carbon 2′ of the spirobiindane moiety. The alkyl substituent may form a cyclopentyl ring, a cyclohexyl ring, a cycloheptyl ring, or a cyclooctyl ring, according to the number of carbon atoms of the alkyl substituent linked to the spirobiindane moiety. The amine compound having this structure may have a wide band gap, and the types of the substituents linked to the first amine group and linked to the second amine group may be modified, thereby variously changing a highest occupied molecular orbital (HOMO) energy level of the molecule. Accordingly, a hole injection barrier between the first electrode EL1 and the hole transport region HTR may be variously modified, and the amine compound may have a suitable energy level between the hole transport region HTR and the emission layer EML, thereby adjusting to increase exciton generation efficiency in the emission layer EML. Therefore, when the amine compound according to an embodiment is applied to a hole transport region HTR of the light emitting device ED, the light emitting device may achieve high efficiency, a low voltage, high brightness, and a long service life.

The amine compound according to an embodiment has the advantage of a significantly increased glass transition temperature due to a large molecular weight. The amine compound may exhibit excellent heat resistance and durability characteristics due to such a high glass transition temperature. The amine compound has a low refractive characteristic due to the alkyl substituent which links carbon 2 and carbon 2′ of the spirobiindane moiety, and the combination of the substituents linked to the first and second amine groups and/or the number of carbon atoms of the alkyl substituent may be variously modified, thereby changing a refractive index of the molecule. Thus, when the amine compound of an embodiment is used in the hole transport region HTR, the refractive index and the light extraction mode may be changed between the first electrode EL1 and the second electrode EL2, and thus external quantum efficiency may be increased. Accordingly, when the amine compound is used in the hole transport region HTR, the luminous efficiency of the light emitting device ED may be increased and the service life of the light emitting device ED may be improved. As described above, the amine compound has excellent heat resistance and durability, and thus the light emitting device of an embodiment may have an improvement in the service life and luminous efficiency by including the amine compound of an embodiment as a material for the light emitting device ED.

The hole transport region HTR may include a compound represented by Formula H-2:

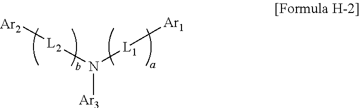

In Formula H-2, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-2, a and b may each independently be an integer from 0 to 10. When a or b is 2 or greater, multiple L₁ groups or multiple L₂ groups may each independently be a substituted or unsubstituted arylene group having 6 to 30 ring-forming carbon atoms or a substituted or unsubstituted heteroarylene group having 2 to 30 ring-forming carbon atoms.

In Formula H-2, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. In Formula H-2, Ar₃ may be a substituted or unsubstituted aryl group having 6 to 30 ring-forming carbon atoms or a substituted or unsubstituted heteroaryl group having 2 to 30 ring-forming carbon atoms.

In an embodiment, a compound represented by Formula H-2 may be a monoamine compound. In another embodiment, a compound represented by Formula H-2 may be a diamine compound in which at least one of Ar₁ to Ar₃ includes an amine group as a substituent. In still other embodiments, a compound represented by Formula H-2 may be a carbazole-based compound in which at least one of Ar₁ or Ar₂ includes a substituted or unsubstituted carbazole group, or may be a fluorene-based compound in which at least one of Ar₁ or Ar₂ includes a substituted or unsubstituted fluorene group.

The compound represented by Formula H-2 may be any compound selected from Compound Group H. However, the compounds listed in Compound Group H are only examples, and compounds represented by Formula H-2 are not limited to Compound Group H:

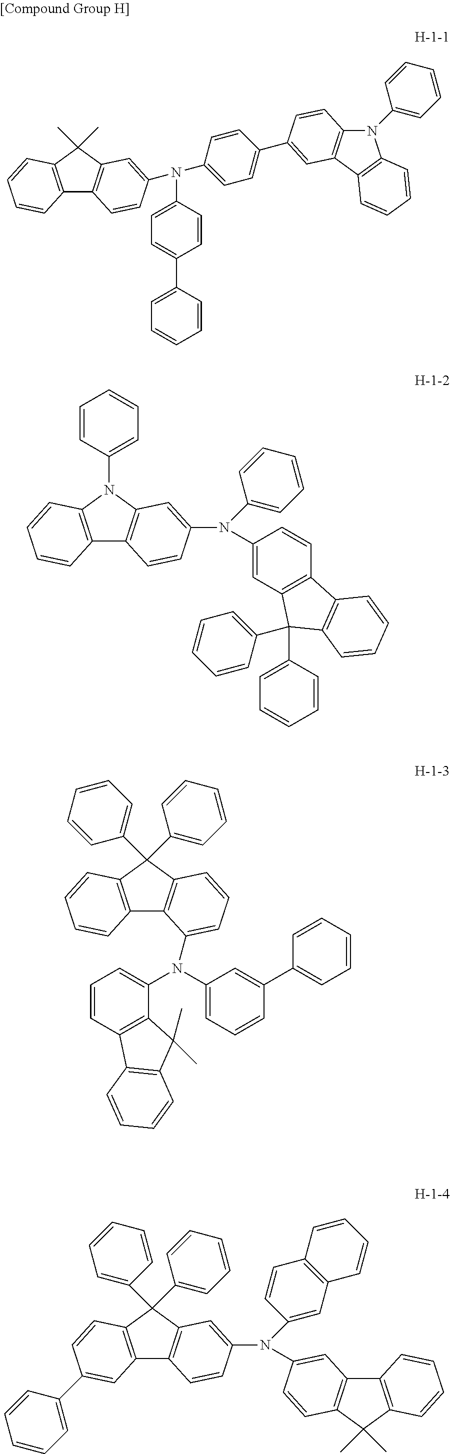 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

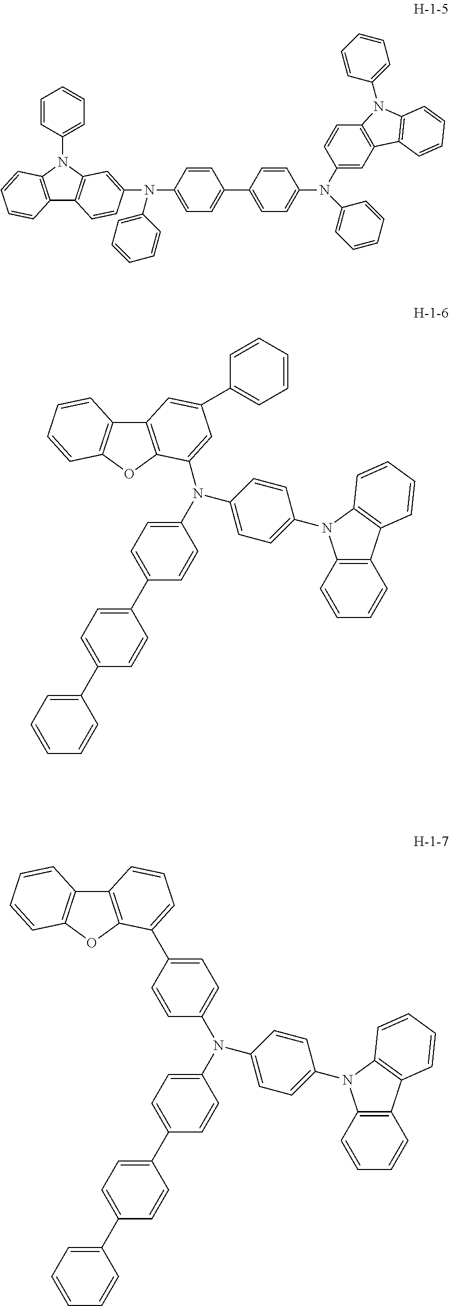 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

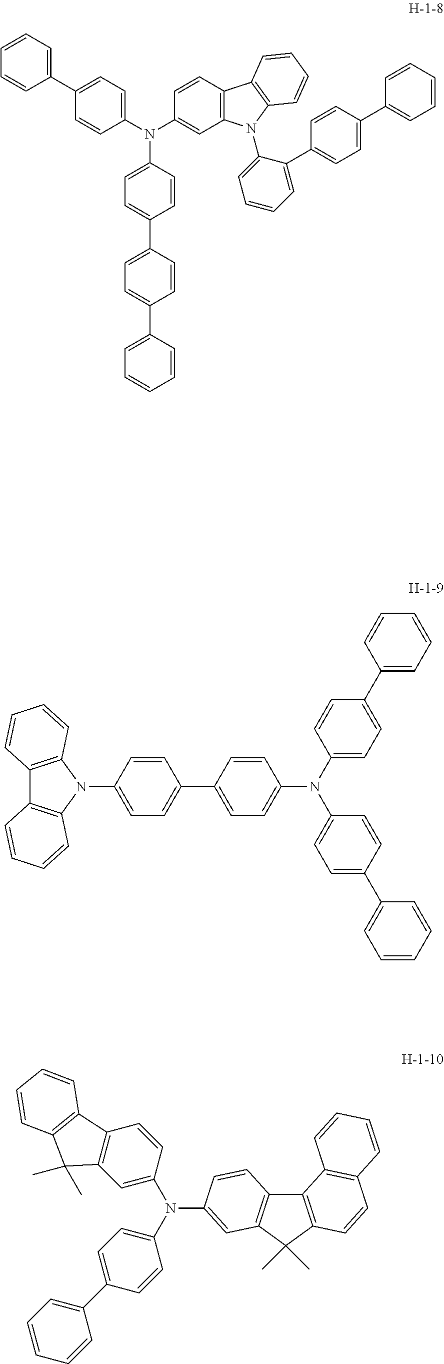 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

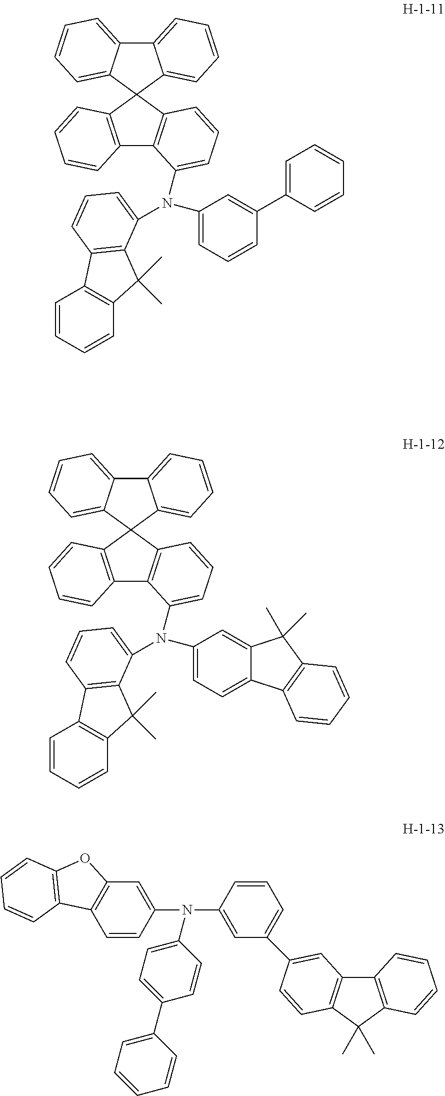 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

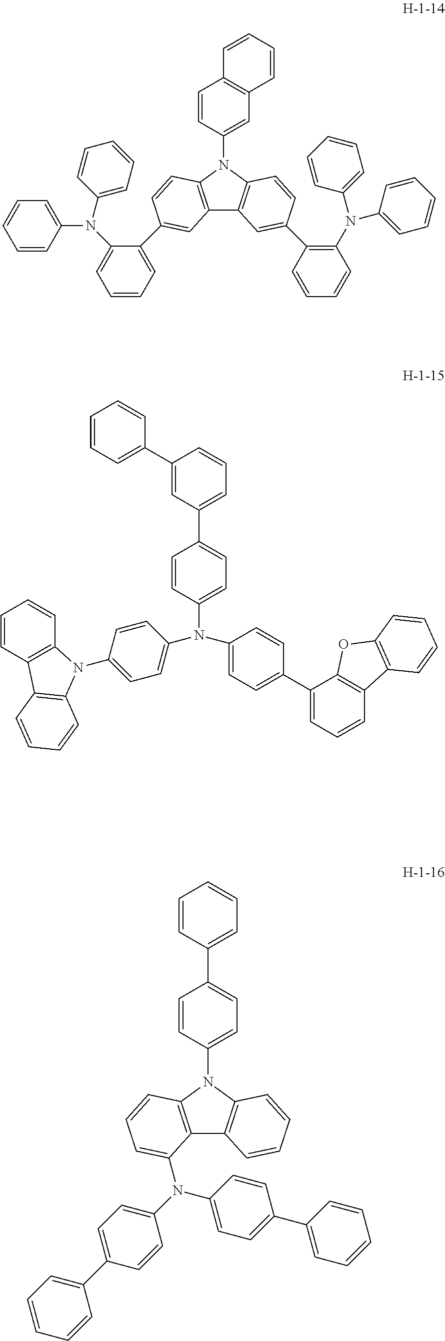 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

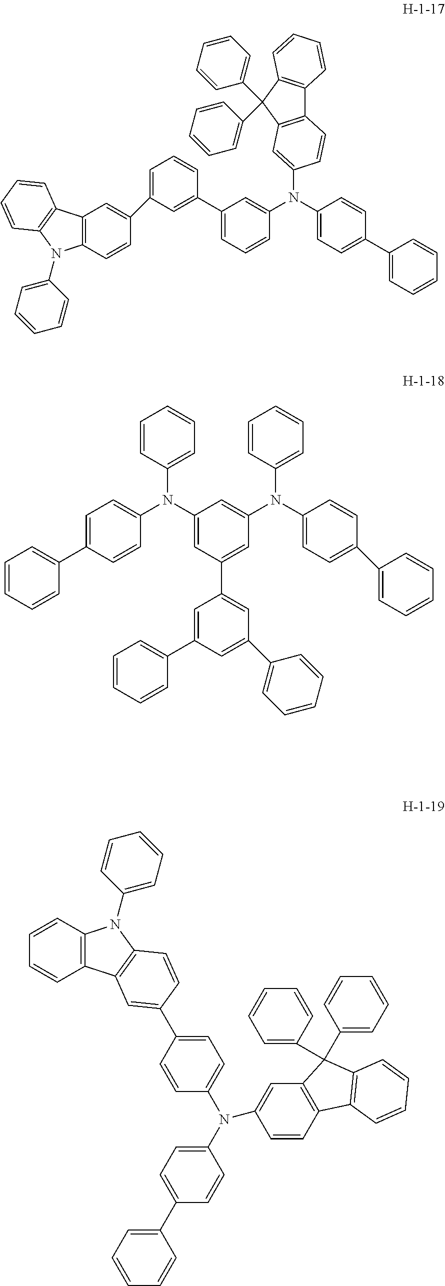 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, or an electron blocking layer EBL.

A thickness of the hole transport region HTR may be in a range of about 100 Å to about 10,000 Å. For example, the thickness of the hole transport region HTR may be in a range of about 100 Å to about 5,000 Å. When the hole transport region HTR includes a hole injection layer HIL, the hole injection layer HIL may have, for example, a thickness in a range of about 30 Å to about 1,000 Å. When the hole transport region HTR includes a hole transport layer HTL, the hole transport layer HTL may have a thickness in a range of about 250 Å to about 1,000 Å. When the hole transport region HTR includes an electron blocking layer EBL, the electron blocking layer EBL may have a thickness in a range of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments are not limited thereto. For example, the p-dopant may include a metal halide compound such as CuI or RbI, a quinone derivative such as tetracyanoquinodimethane (TCNQ) or 2,3,5,6-tetrafluoro-7,7′8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments are not limited thereto.

As described above, the hole transport region HTR may further include at least one of a buffer layer (not shown) or an electron blocking layer EBL, in addition to a hole injection layer HIL and a hole transport layer HTL. The buffer layer (not shown) may compensate for a resonance distance according to a wavelength of light emitted from the emission layer EML and may thus increase light emission efficiency. A material that may be included in the hole transport region HTR may be used as a material included in the buffer layer (not shown). The electron blocking layer EBL may prevent electron injection from an electron transport region ETR to a hole transport region HTR.

The emission layer EML is provided on the hole transport region HTR. The emission layer EML may have a thickness, for example, in a range of about 100 Å to about 1,000 Å. For example, the emission layer EML may have a thickness in a range of about 100 Å to about 300 Å. The emission layer EML may be a layer consisting of a single material, a layer including different materials, or a structure including multiple layers including different materials.

In the light emitting device ED according to an embodiment, the emission layer EML may include an anthracene derivative, a pyrene derivative, a fluoranthene derivative, a chrysene derivative, a dihydrobenzanthracene derivative, or a triphenylene derivative. For example, the emission layer EML may include an anthracene derivative or a pyrene derivative.

In the light emitting device ED according to embodiments illustrated in FIGS. 3 to 6, the emission layer EML may include a host and a dopant, and the emission layer EML may include a compound represented by Formula E-1. The compound represented by Formula E-1 may be used as a fluorescent host material.

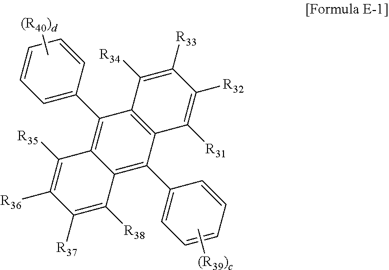

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, in Formula E-1, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

The compound represented by Formula E-1 may be any compound selected from Compound E1 to Compound E19:

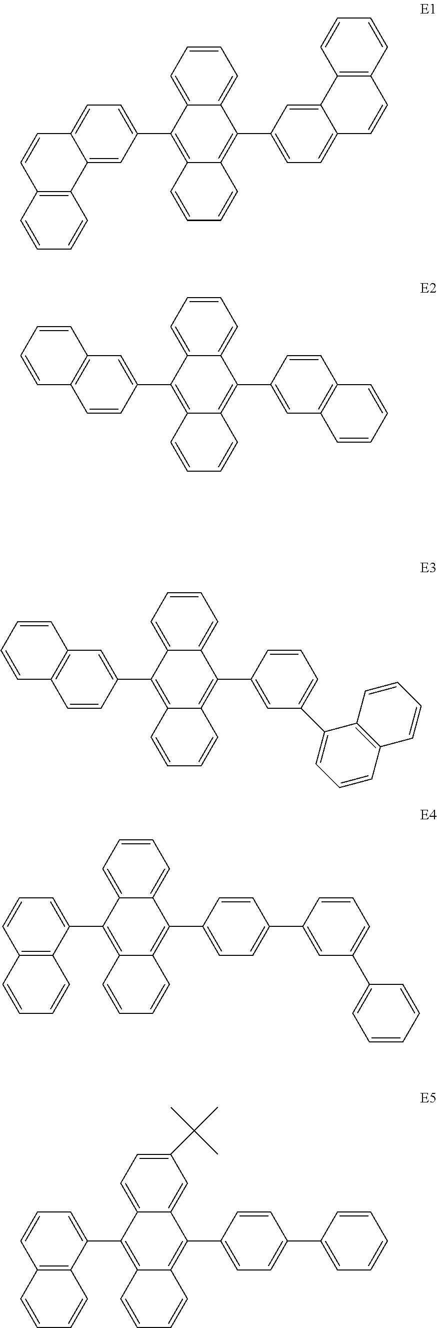

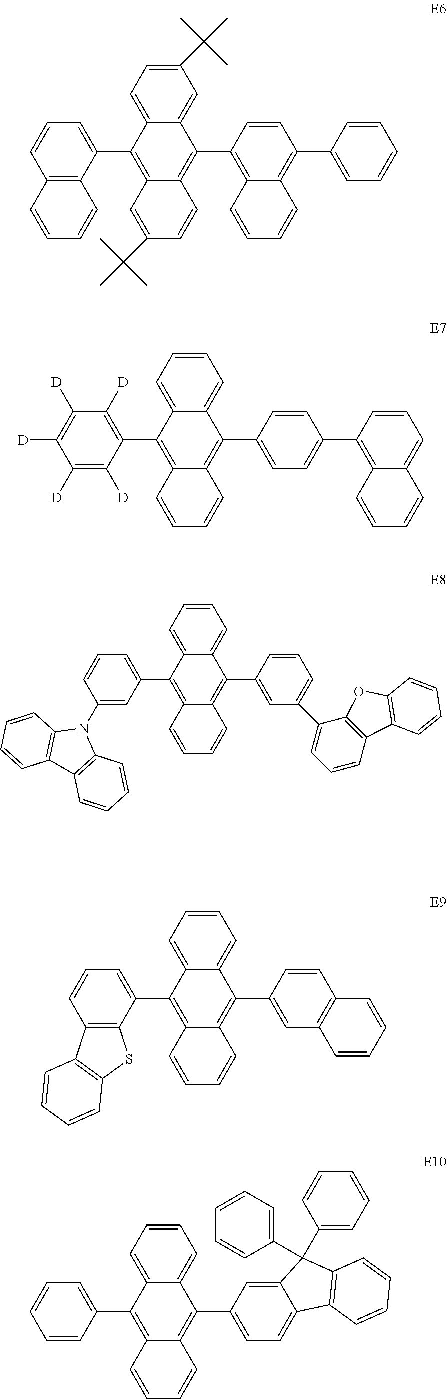

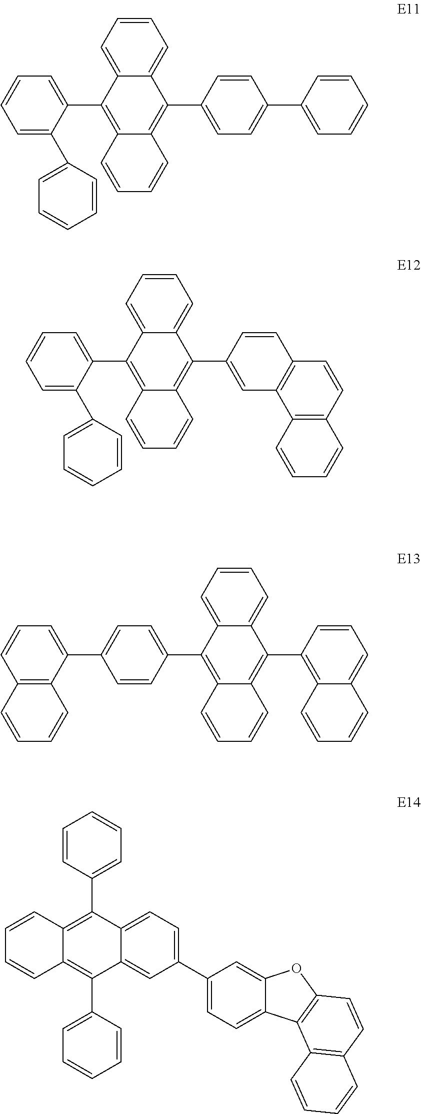

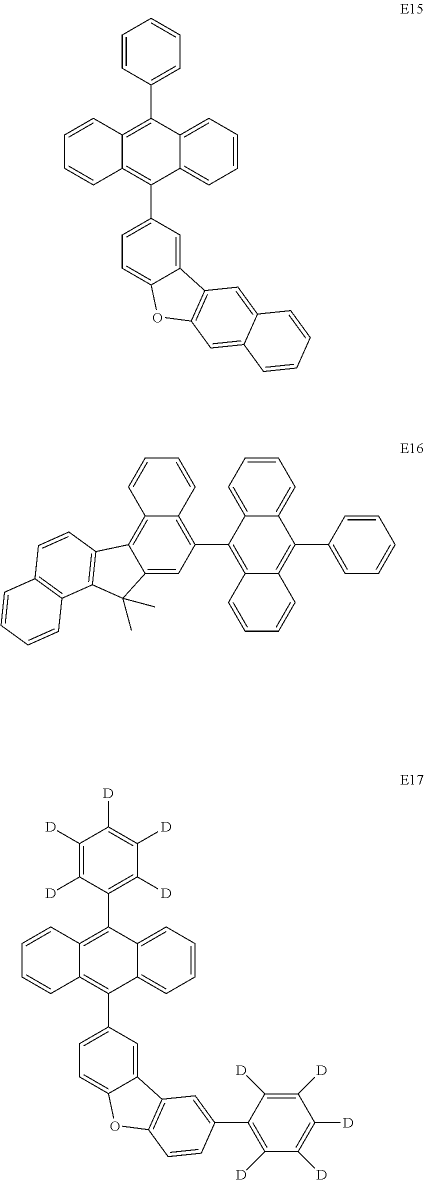

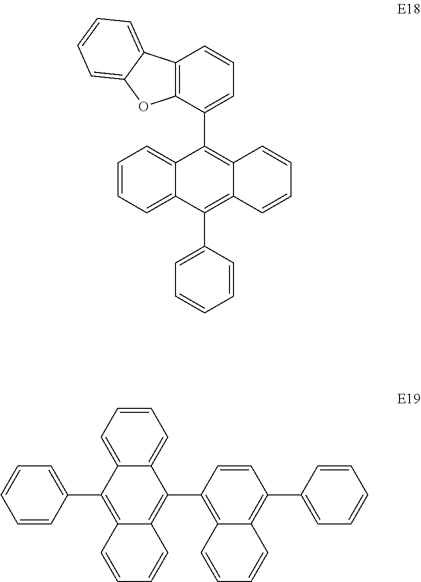

In an embodiment, the emission layer EML may include at least one of a first compound represented by Formula HT-1, or a second compound represented by Formula ET-1.

In an embodiment, the first compound represented by Formula HT-1 may be used as a hole transporting host material for the emission layer EML.

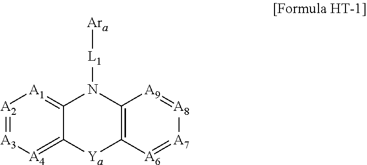

In Formula HT-1, A₁ to A₄ and A₆ to A₉ may each independently be N or C(R₄1). For example, in an embodiment, A₁ to A₄ and A₆ to A₉ may each independently be C(R₄₁). In another embodiment, one of A₁ to A₄ and A₆ to A₉ may be N, and the remainder of A₁ to A₄ and A₆ to A₉ may each independently be C(R₄₁).

In Formula HT-1, L₁ may be a direct linkage, a substituted or unsubstituted arylene group having 6 to 30 ring-forming carbon atoms, or a substituted or unsubstituted heteroarylene group having 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, etc., but embodiments are not limited thereto.

In Formula HT-1, Yₐ may be a direct linkage, C(R₄₂)(R₄₃), or Si(R₄₄)(R₄₅). For example, the two benzene rings linked to the nitrogen atom in Formula HT-1 may be linked via a direct linkage,

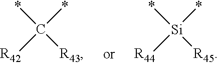

In Formula HT-1, when Yₐ is a direct linkage, the first compound represented by Formula HT-1 may include a carbazole moiety.

In Formula HT-1, Arₐ may be a substituted or unsubstituted aryl group having 6 to 30 ring-forming carbon atoms or a substituted or unsubstituted heteroaryl group having 2 to 30 ring-forming carbon atoms. For example, Arₐ may be a substituted or unsubstituted carbazole group, a substituted or unsubstituted dibenzofuran group, a substituted or unsubstituted dibenzothiophene group, a substituted or unsubstituted biphenyl group, etc., but embodiments are not limited thereto.

In Formula HT-1, R₄₁ to R₄₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms, or may be bonded to an adjacent group to form a ring. For example, R₄₁ to R₄₅ may each independently be a hydrogen atom or a deuterium atom. For example, R₄₁ to R₄₅ may each independently be an unsubstituted methyl group or an unsubstituted phenyl group.

The first compound represented by Formula HT-1 may be any compound selected from Compound Group 2. The emission layer EML in the light emitting device ED may include at least one compound selected from Compound Group 2 as a hole transporting host material. In Compound Group 2, D represents a deuterium atom.

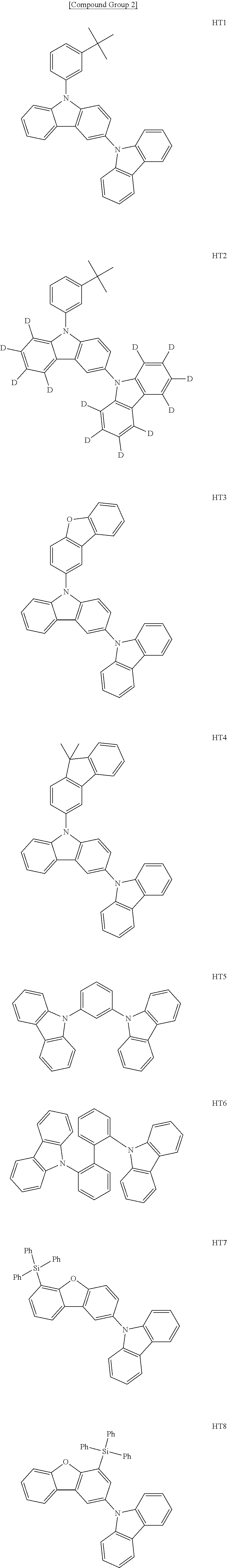

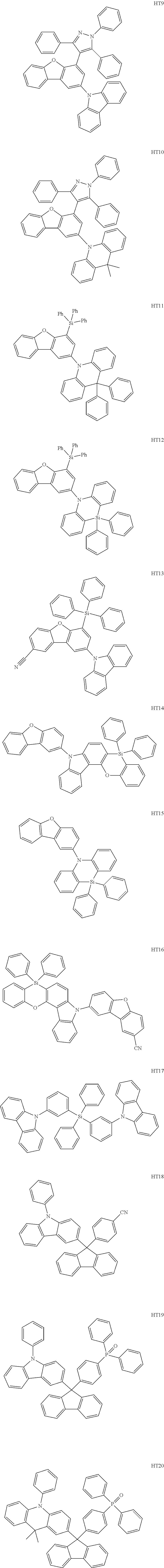

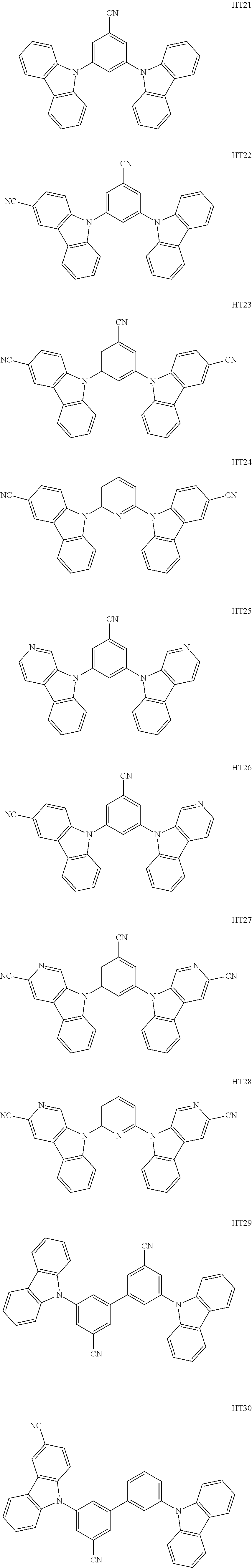

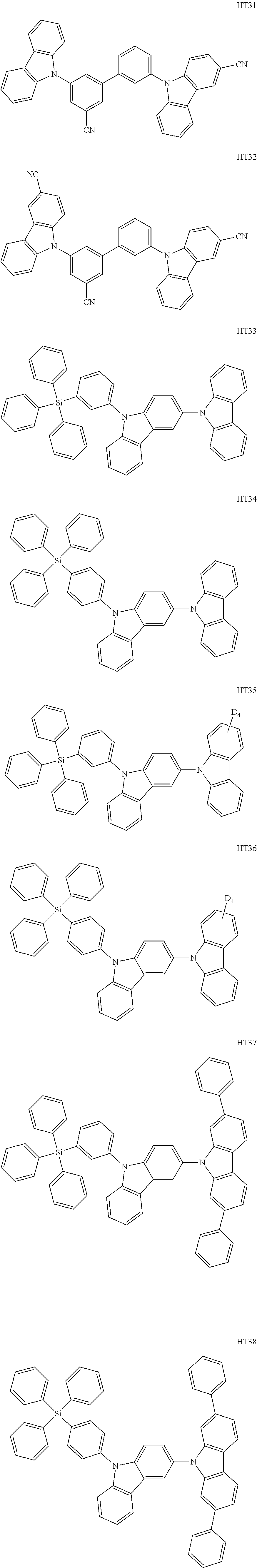

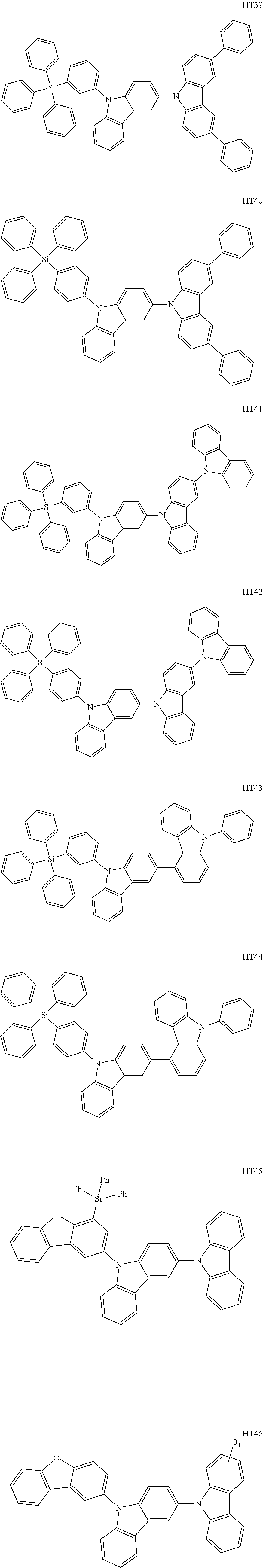

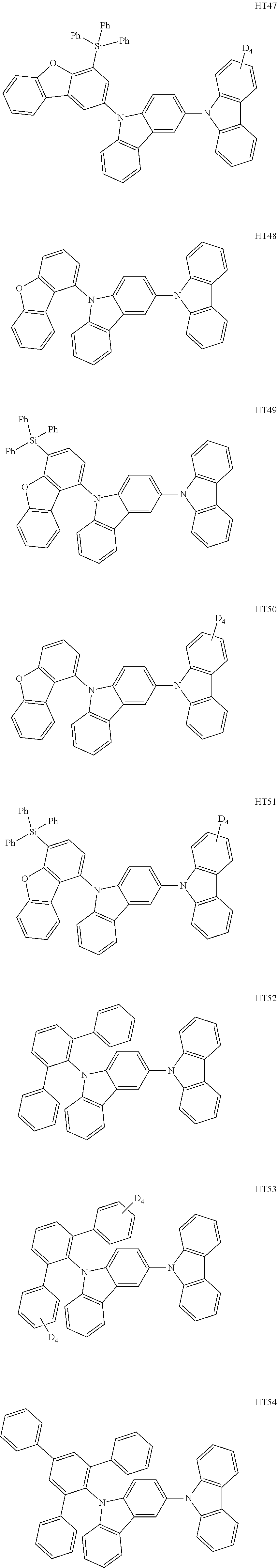

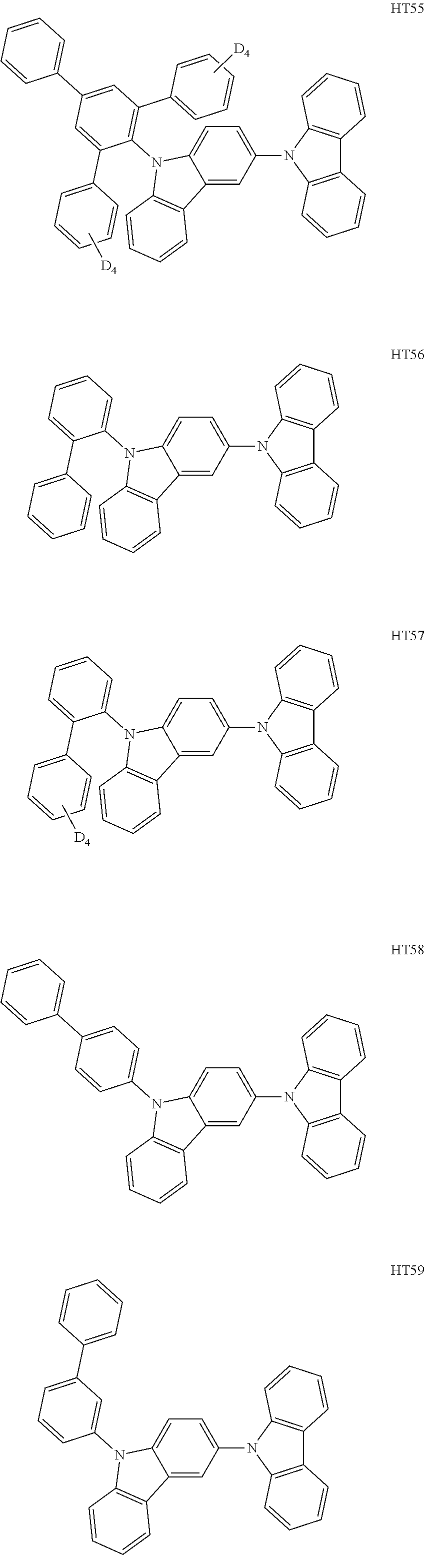

In an embodiment, the emission layer EML may include a second compound represented by Formula ET-1. The second compound represented by Formula ET-1 may be used as an electron transport host material for the emission layer EML.

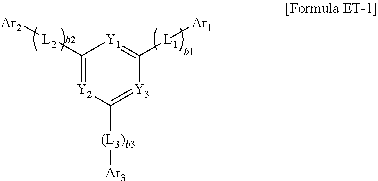

In Formula ET-1, at least one of Y₁ to Y₃ may each be N, and the remainder of Y₁ to Y₃ may each independently be C(Rₐ); and Rₐ may be a hydrogen atom, a deuterium atom, a substituted or unsubstituted alkyl group having 1 to 20 carbon atoms, a substituted or unsubstituted aryl group having 6 to 60 ring-forming carbon atoms, or a substituted or unsubstituted heteroaryl group having 2 to 60 ring-forming carbon atoms.

In Formula ET-1, b1 to b3 may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms.

In Formula ET-1, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Ar₁ to Ar₃ may each independently be a substituted or unsubstituted phenyl group or a substituted or unsubstituted carbazole group.

The second compound represented by Formula ET-1 may be any compound selected from Compound Group 3. The emission layer EML in the light emitting device ED may include at least one compound selected from Compound Group 3. In Compound Group 3, D represents a deuterium atom.

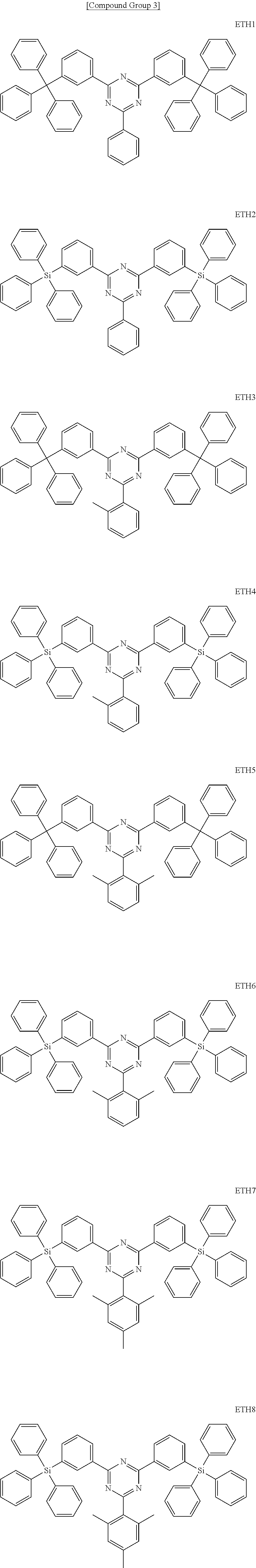

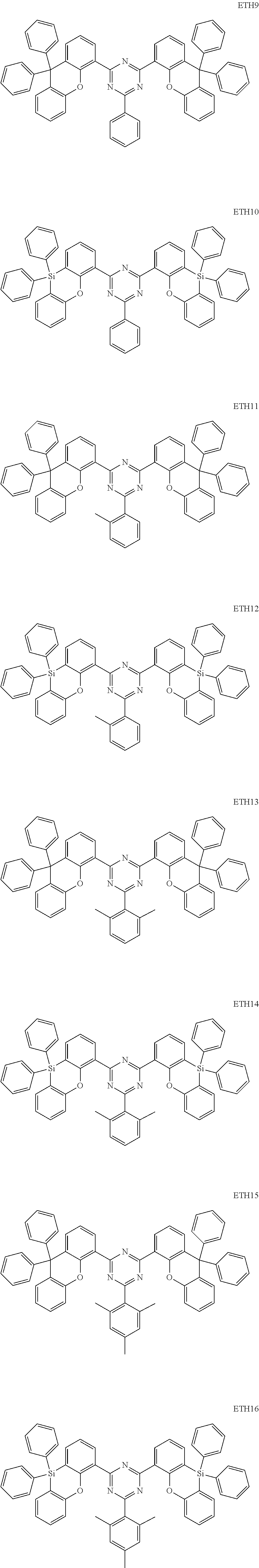

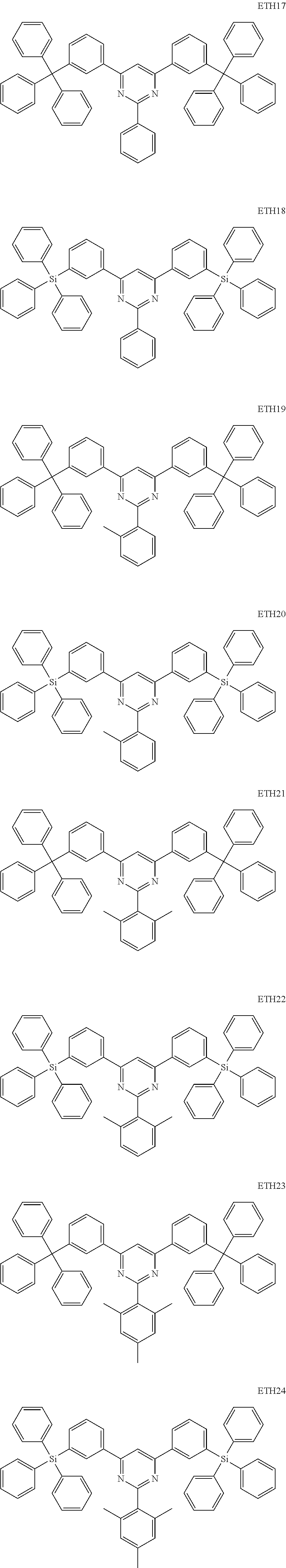

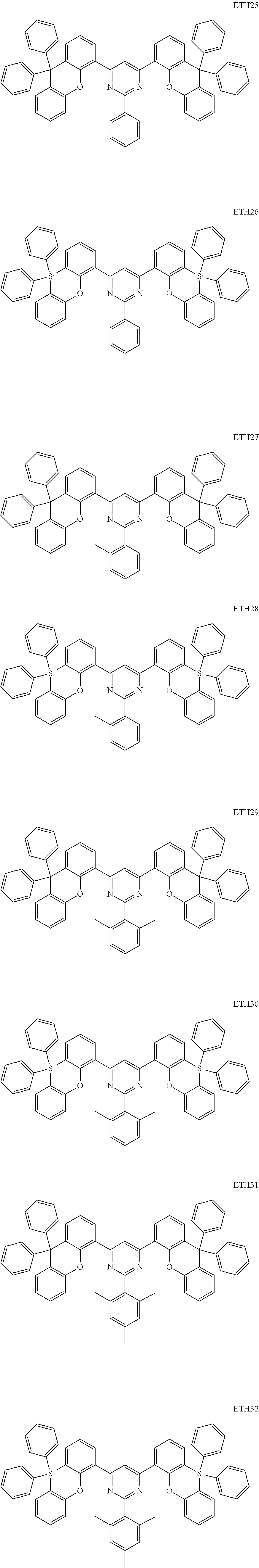

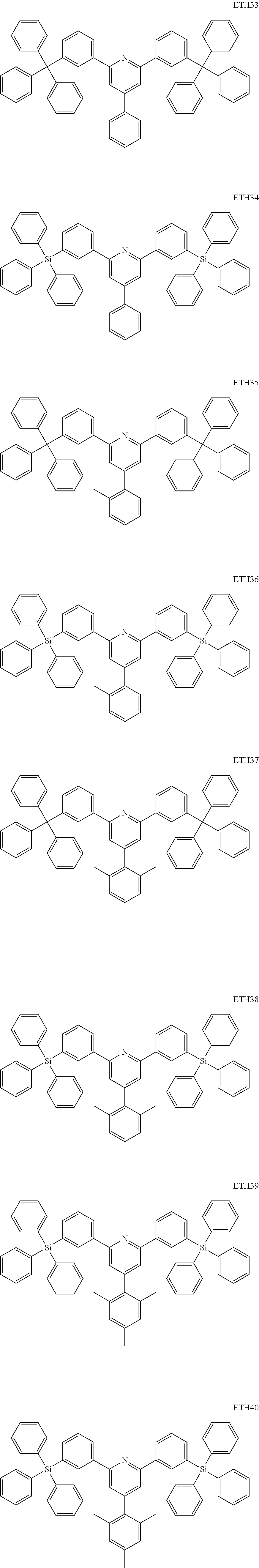

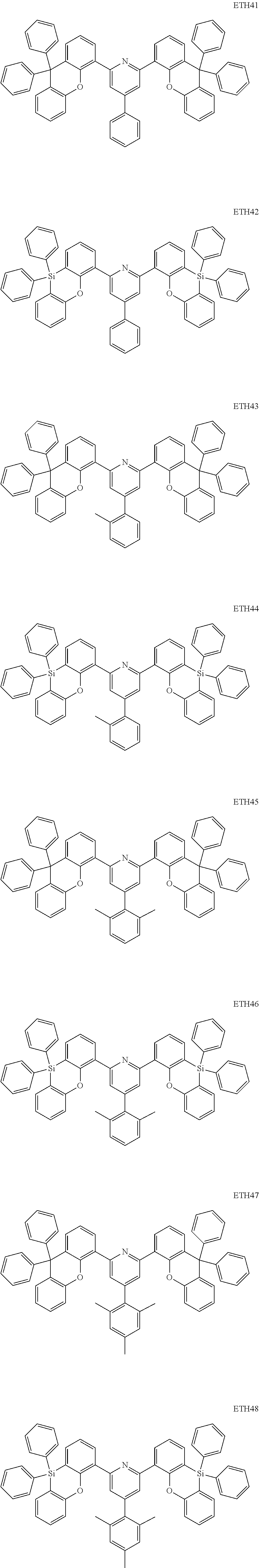

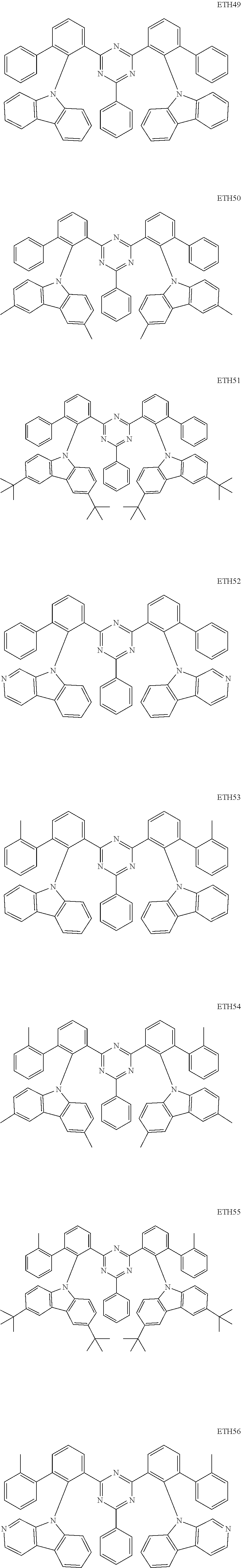

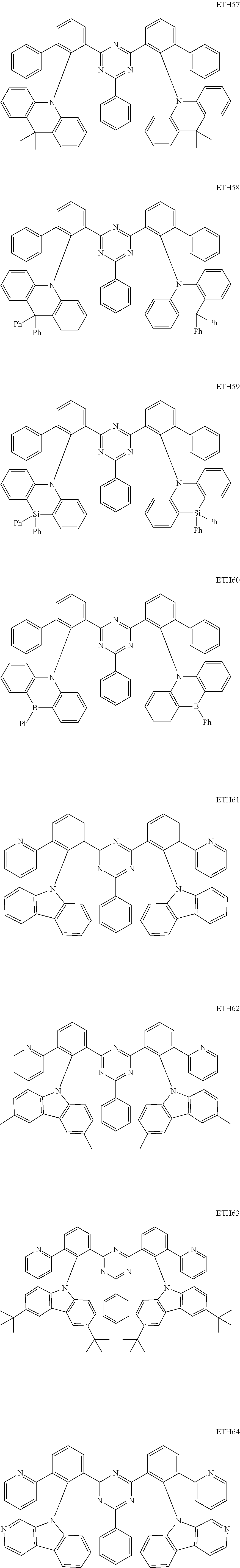

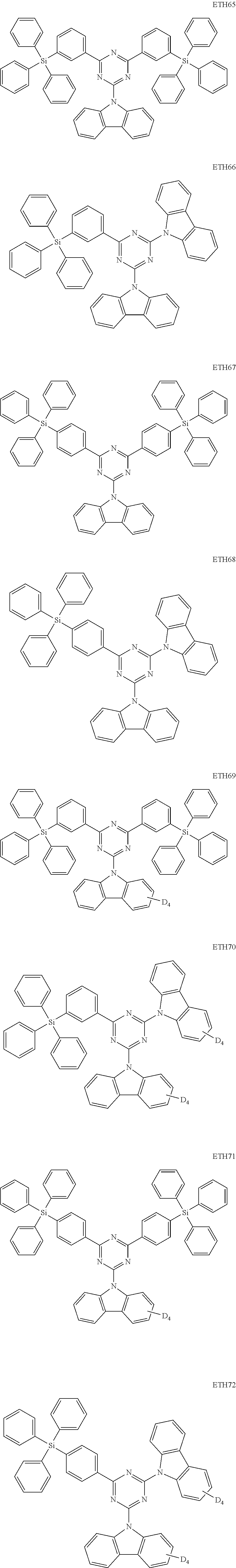

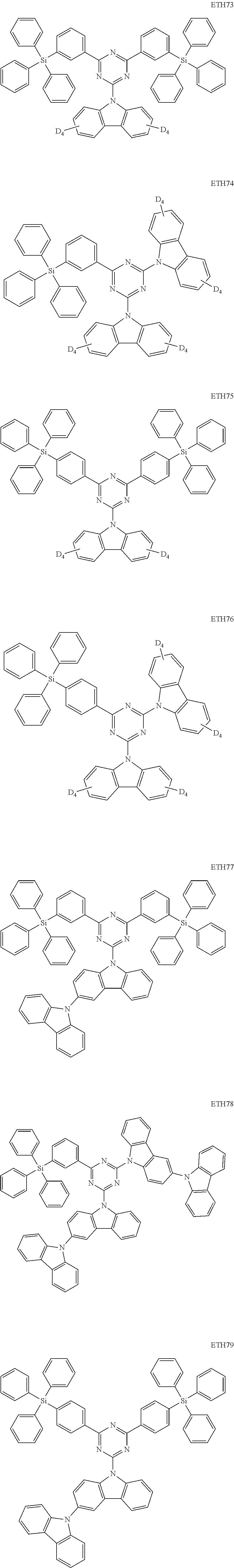

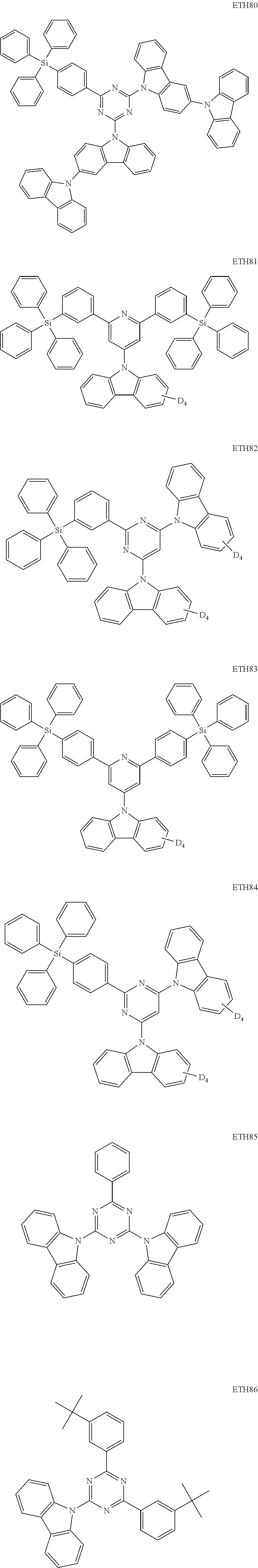

In an embodiment, the emission layer EML may include a compound represented by Formula E-2a or Formula E-2b. The compound represented by Formula E-2a or Formula E-2b may be used as a phosphorescent host material.

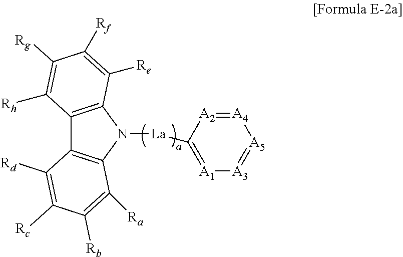

In Formula E-2a, a may be an integer from 0 to 10; and Lₐ may be a direct linkage, a substituted or unsubstituted arylene group having 6 to 30 ring-forming carbon atoms, or a substituted or unsubstituted heteroarylene group having 2 to 30 ring-forming carbon atoms. When a is 2 or more, multiple Lₐ groups may each independently be a substituted or unsubstituted arylene group having 6 to 30 ring-forming carbon atoms or a substituted or unsubstituted heteroarylene group having 2 to 30 ring-forming carbon atoms.

In Formula E-2a, A₁ to A₅ may each independently be N or C(R₁). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, Rₐ to Rᵢ may be bonded to an adjacent group to form a hydrocarbon ring or a heterocycle containing N, O, S, etc., as a ring-forming atom.

In Formula E-2a, two or three of A₁ to A₅ may each be N, and the remainder of A₁ to A₅ may each independently be C(R₁).

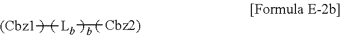

In Formula E-2b, Cbz1 and Cbz2 may each independently be an unsubstituted carbazole group, or a carbazole group substituted with an aryl group having 6 to 30 ring-forming carbon atoms. In Formula E-2b, L_{b} may be a direct linkage, a substituted or unsubstituted arylene group having 6 to 30 ring-forming carbon atoms, or a substituted or unsubstituted heteroarylene group having 2 to 30 ring-forming carbon atoms. In Formula E-2b, b may be an integer from 0 to 10, and when b is 2 or more, multiple L_{b} groups may each independently be a substituted or unsubstituted arylene group having 6 to 30 ring-forming carbon atoms or a substituted or unsubstituted heteroarylene group having 2 to 30 ring-forming carbon atoms.

The compound represented by Formula E-2a or Formula E-2b may be any compound selected from Compound Group E-2. However, the compounds listed in Compound Group E-2 are only examples, and the compound represented by Formula E-2a or Formula E-2b is not limited to Compound Group E-2.

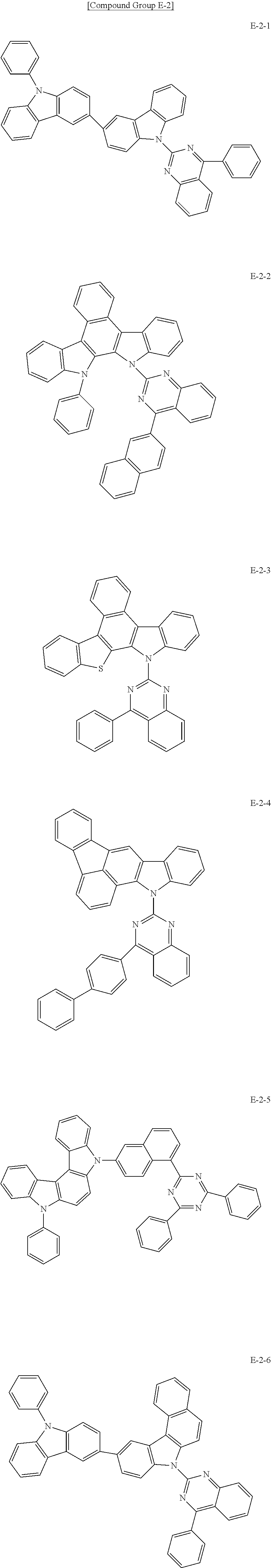 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

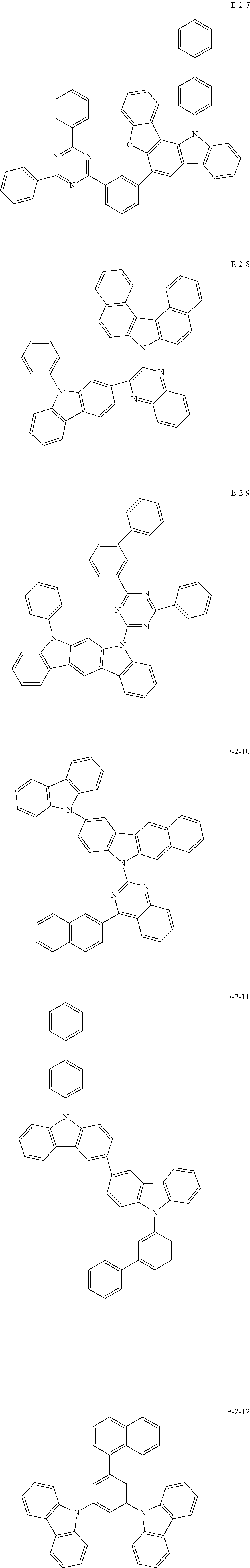

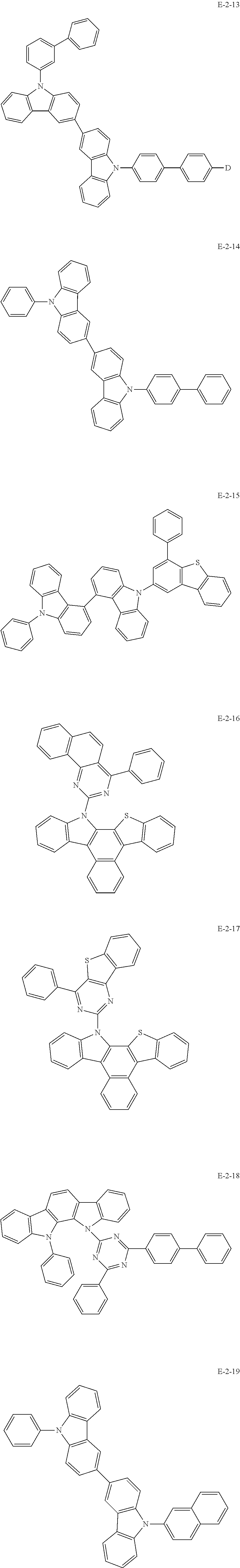

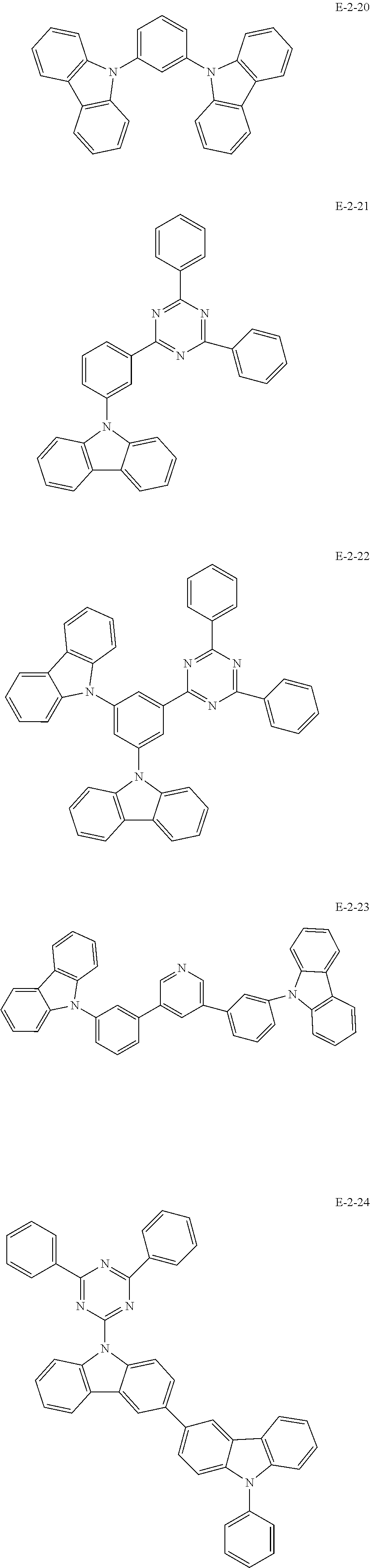 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material of the related art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments are not limited thereto. For example, tris(8-hydroxyquinolinato)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as a host material.

The emission layer EML may include a compound represented by Formula M-a or Formula M-b. The compound represented by Formula M-a or Formula M-b may be used as a phosphorescent dopant material.

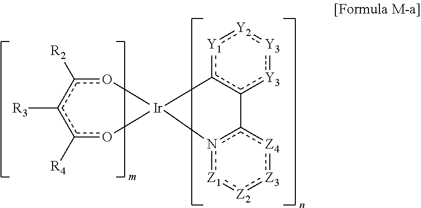

In Formula M-a, Y₁ to Y₄ and Z₁ to Z₄ may each independently be C(R₁) or N; and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m may be 0 or 1, and n may be 2 or 3. In Formula M-a, when m is 0, n may be 3, and when m is 1, n may be 2.

The compound represented by Formula M-a may be any compound selected from Compound M-a1 to Compound M-a21. However, Compounds M-a1 to M-a21 are only examples, and the compound represented by Formula M-a is not limited to Compounds M-a1 to M-a21.

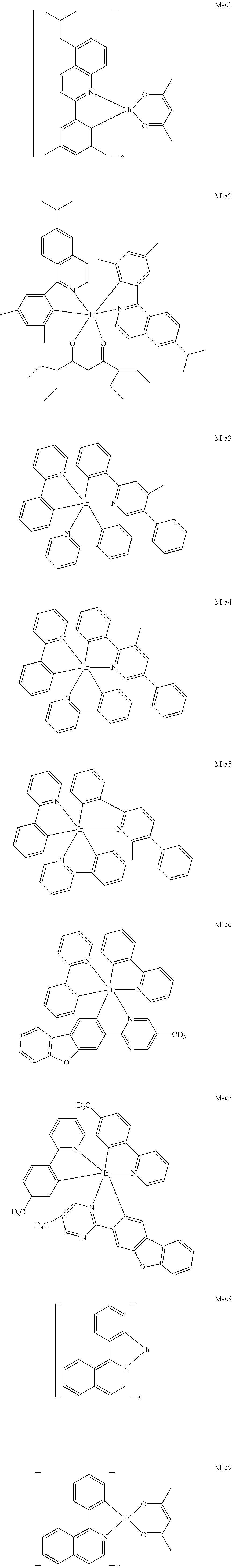

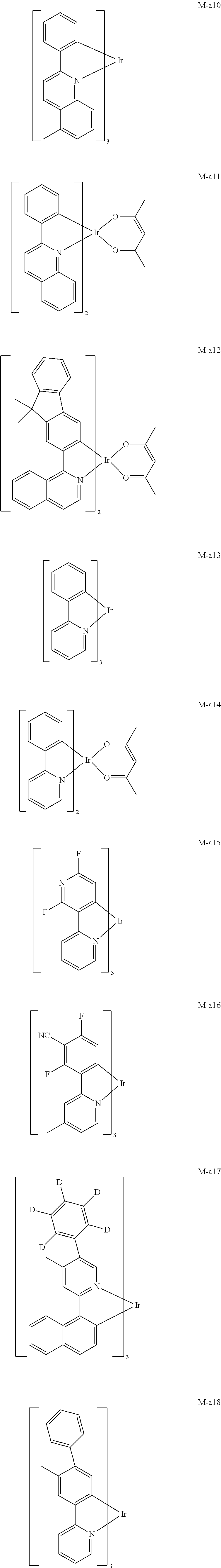

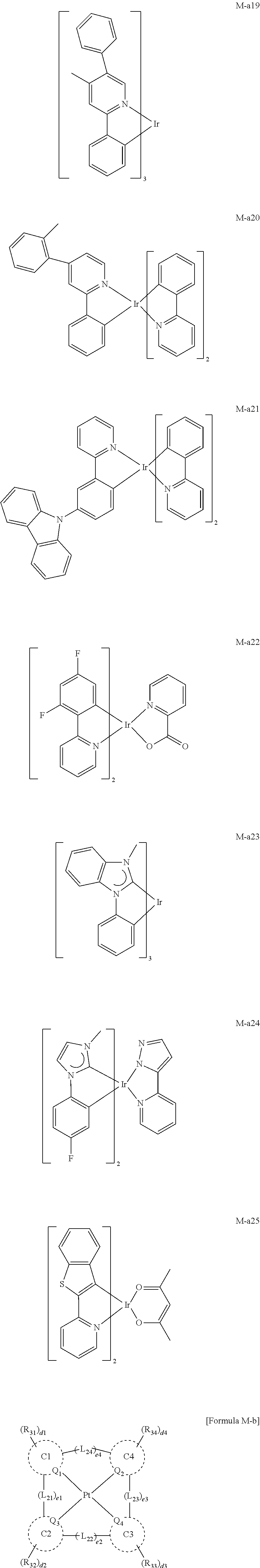

In Formula M-b, Q₁ to Q4 may each independently be C or N; and C1 to C4 may each independently be a substituted or unsubstituted hydrocarbon ring having 5 to 30 ring-forming carbon atoms or a substituted or unsubstituted heterocycle having 2 to 30 ring-forming carbon atoms. In Formula M-b, L₂₁ to L₂₄ may each independently be a direct linkage, *—O— *, *—S—*,

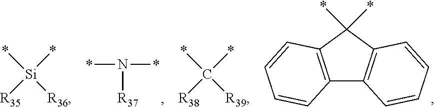

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms; and el to e4 may each independently be 0 or 1. In Formula M-b, R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring; and d1 to d4 may each independently be an integer from 0 to 4.

The compound represented by Formula M-b may be used as a blue phosphorescent dopant or a green phosphorescent dopant.

The compound represented by Formula M-b may be any compound selected from Compound M-b-1 to Compound M-b-11. However, Compound M-b-1 to Compound M-b-11 are only examples, and the compound represented by Formula M-b is not limited to Compound M-b-1 to Compound M-b-11.

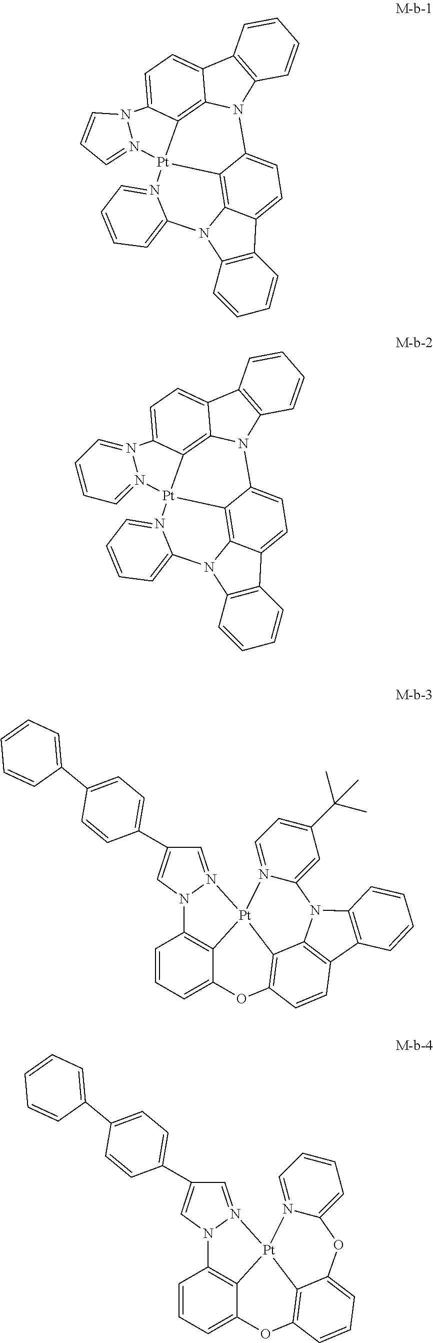

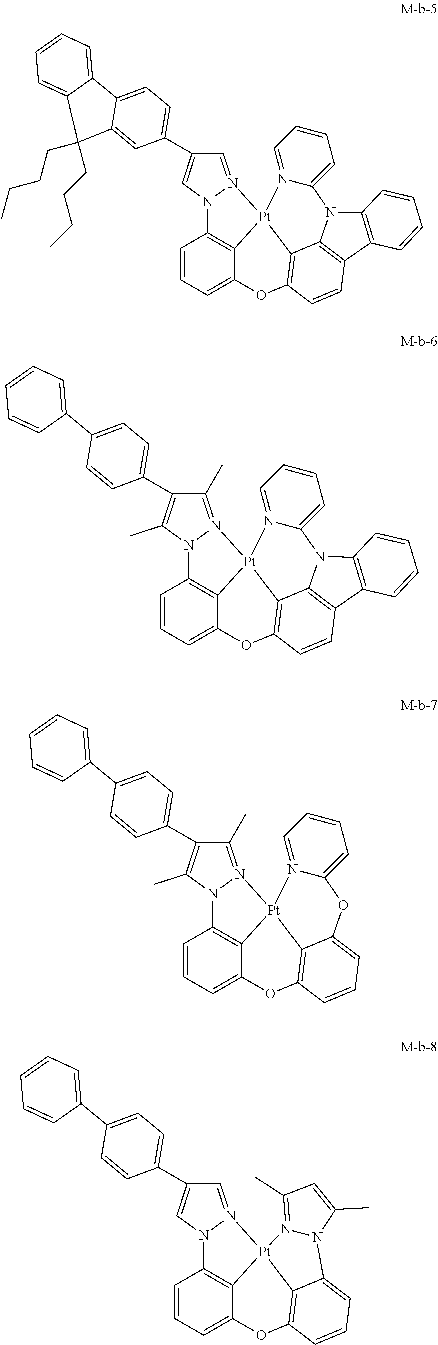

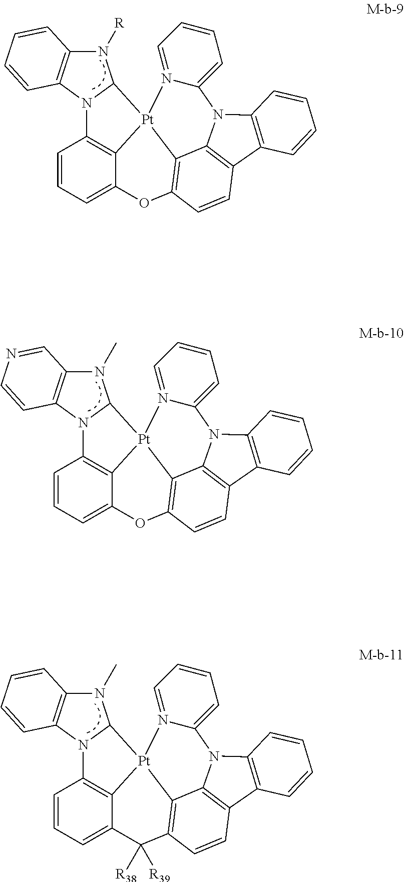

In Compound M-b-1 to Compound M-b-11, R, R_{3M}, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one of Formula F-a to Formula F-c. The compound represented by one of Formula F-a to Formula F-c may be used as a fluorescence dopant material.

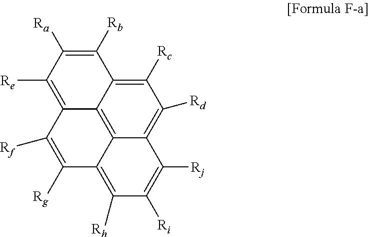

In Formula F-a, two of Rₐ to Rⱼ may each independently be substituted with a group represented by *—NAr₁Ar₂. The remainder of Rₐ to Rⱼ which are not substituted with the group represented by *—NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In the group represented by *—NAr₁Ar₂, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. For example, at least one of Ar₁ or Ar₂ may be a heteroaryl group containing O or S as a ring-forming atom.

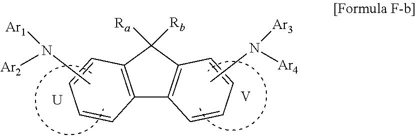

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b, Ar₁ to Ar₄ may each independently be a substituted or unsubstituted aryl group having 6 to 30 ring-forming carbon atoms or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms or a substituted or unsubstituted heterocycle having 2 to 30 ring-forming carbon atoms.

In Formula F-b, the number of rings represented by U and V may each independently be 0 or 1. For example, in Formula F-b, when the number of U or V is 1, a fused ring may be present at a portion indicated by U or V, and when the number of U or V is 0, a fused ring may not be present at the portion indicated by U or V. When the number of U is 0 and the number of V is 1, or when the number of U is 1 and the number of V is 0, a fused ring having a fluorene core of Formula F-b may be a cyclic compound having four rings. When U and V are each 0, a fused ring having a fluorene core of Formula F-b may be a cyclic compound having three rings. When U and V are each 1, a fused ring having a fluorene core of Formula F-b may be a cyclic compound having five rings.

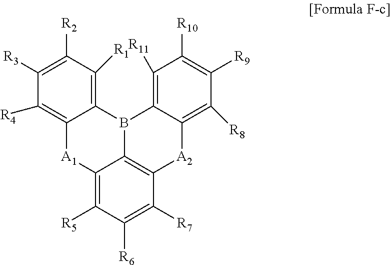

In Formula F-c, A₁ and A₂ may each independently be O, S, Se, or N(Rₘ); and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-c, A₁ and A₂ may each independently be bonded to substituents of an adjacent ring to form a condensed ring. For example, when A₁ and A₂ are each independently N(Rₘ), Ai may be bonded to R₄ or R₅ to form a ring. For example, A₂ may be bonded to R₇ or R₈ to form a ring.

In an embodiment, the emission layer EML may include, as a dopant material of the related art, a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), etc.

The emission layer EML may include a phosphorescent dopant material of the related art. For example, a metal complex containing iridium (Ir), platinum (Pt), osmium (Os), gold (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as a phosphorescent dopant. However, embodiments are not limited thereto.

In an embodiment, the emission layer EML may include a quantum dot. The quantum dot may be a Group II-VI compound, a Group III-VI compound, a Group 1-II-VI compound, a Group III-V compound, a Group III—II-V compound, a Group IV-VI compound, a Group IV element, a Group IV compound, or any combination thereof.

The Group II-VI compound may include: a binary compound selected from the group consisting of CdSe, CdTe, CdS, ZnS, ZnSe, ZnTe, ZnO, HgS, HgSe, HgTe, MgSe, MgS, and any mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and any mixture thereof; a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and any mixture thereof; or any combination thereof.

The Group III-VI compound may include: a binary compound such as In₂S₃ or In₂Se₃: a ternary compound such as InGaS₃ or InGaSe₃; or any combination thereof.

The Group I-III-VI compound may include: a ternary compound selected from the group consisting of AgInS, AgInS₂, CuInS, CuInS₂, AgGaS₂, CuGaS₂, CuGaO₂, AgGaO₂, AgAlO₂, and any mixture thereof; a quaternary compound such as AgInGaS₂ or CuInGaS₂; or any combination thereof.

The Group III-V compound may include: a binary compound selected from the group consisting of GaN, GaP, GaAs, GaSb, AlN, AlP, AlAs, AlSb, InN, InP, InAs, InSb, and any mixture thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and any mixture thereof; a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and any mixture thereof; or any combination thereof. In an embodiment, the Group III-V compound may further include a Group II metal. For example, InZnP, etc., may be selected as a Group III-II-V compound.

The Group IV-VI compound may include: a binary compound selected from the group consisting of SnS, SnSe, SnTe, PbS, PbSe, PbTe, and any mixture thereof; a ternary compound selected from the group consisting of SnSeS, SnSeTe, SnSTe, PbSeS, PbSeTe, PbSTe, SnPbS, SnPbSe, SnPbTe, and any mixture thereof; a quaternary compound selected from the group consisting of SnPbSSe, SnPbSeTe, SnPbSTe, and any mixture thereof; or any combination thereof. The Group IV element may be Si, Ge, or any mixture thereof. The Group IV compound may include a binary compound selected from the group consisting of SiC, SiGe, and any mixture thereof.

A binary compound, a ternary compound, or a quaternary compound may be present in a particle at a uniform concentration distribution, or may be present in a particle at a partially different concentration distribution. In an embodiment, the quantum dot may have a core/shell structure in which a quantum dot surrounds another quantum dot. A quantum dot having a core/shell structure may have a concentration gradient in which the concentration of a material that is present in the shell decreases toward the core.

In embodiments, the quantum dot may have the above-described core/shell structure including a core containing nanocrystals and a shell surrounding the core. The shell of the quantum dot may serve as a protection layer to prevent the chemical deformation of the core to maintain semiconductor properties, and/or may serve as a charging layer to impart electrophoresis properties to the quantum dot. The shell may be a single layer or a multilayer.

Examples of a shell of a quantum dot may include a metal oxide, a non-metal oxide, a semiconductor compound, or any combination thereof.

Examples of a metal oxide or a non-metal oxide may include: a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; or a ternary compound such as MgAl₂O₄, CoFe₂O₄, NiFe₂O₄, or CoMn₂O₄; or any combination thereof. However, embodiments are not limited thereto.

Examples of a semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width at half maximum (FWHM) of an emission wavelength spectrum equal to or less than about 45 nm. For example, the quantum dot may have a FWHM of an emission wavelength spectrum equal to or less than about 40 nm. For example, the quantum dot may have a FWHM of an emission wavelength spectrum equal to or less than about 30 nm. Color purity or color reproducibility may be improved in the above ranges. Light emitted through a quantum dot may be emitted in all directions, so that a wide viewing angle may be improved.

The form of a quantum dot may be any form that is used in the related art. For example, a quantum dot may have a spherical shape, a pyramidal shape, a multi-arm shape, or a cubic shape, or the quantum dot may be in the form of nanoparticles, nanotubes, nanowires, nanofibers, nanoparticles, etc.

A quantum dot may control the color of emitted light according to a particle size thereof, and thus the quantum dot may have various light emission colors such as green, red, etc.

In the light emitting device ED according to an embodiment illustrated in each of FIGS. 3 to 6, the electron transport region ETR is provided on the emission layer EML. The electron transport region ETR may include at least one of a hole blocking layer HBL, an electron transport layer ETL, or an electron injection layer EIL, but embodiments are not limited thereto.

The electron transport region ETR may be a layer consisting of a single material, a layer including different materials, or a structure including multiple layers including different materials.

For example, the electron transport region ETR may have a single layer structure of an electron injection layer EIL or an electron transport layer ETL, or may have a single layer structure formed of an electron injection material and an electron transport material. In other embodiment, the electron transport region ETR may have a single layer structure including different materials, or may have a structure in which an electron transport layer ETL/electron injection layer EIL, or a hole blocking layer HBL/electron transport layer ETL/electron injection layer EIL are stacked in its respective stated order from an emission layer EML, but embodiments are not limited thereto. The electron transport region ETR may have a thickness, for example, in a range of about 1,000 Å to about 1,500 Å.

The electron transport region ETR may be formed by using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-2:

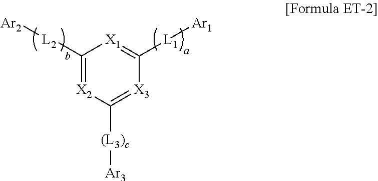

In Formula ET-2, at least one of X₁ to X₃ may each be N, and the remainder of X₁ to X₃ may each independently be C(Rₐ). In Formula ET-2, Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula ET-2, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer from 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When a to c are each 2 or more, multiple groups of each of L₁ to L₃ may each independently be a substituted or unsubstituted arylene group having 6 to 30 ring-forming carbon atoms or a substituted or unsubstituted heteroarylene group having 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), berylliumbis(benzoquinolin-10-olate (Bebg₂), 9,10-di(naphthalene-2-yl)anthracene (ADN), 1,3-Bis[3,5-di(pyridin-3-yl)phenyl]benzene (BmPyPhB), or any mixture thereof.

In an embodiment, the electron transport region ETR may include at least one compound selected from Compound ETl to Compound ET36:

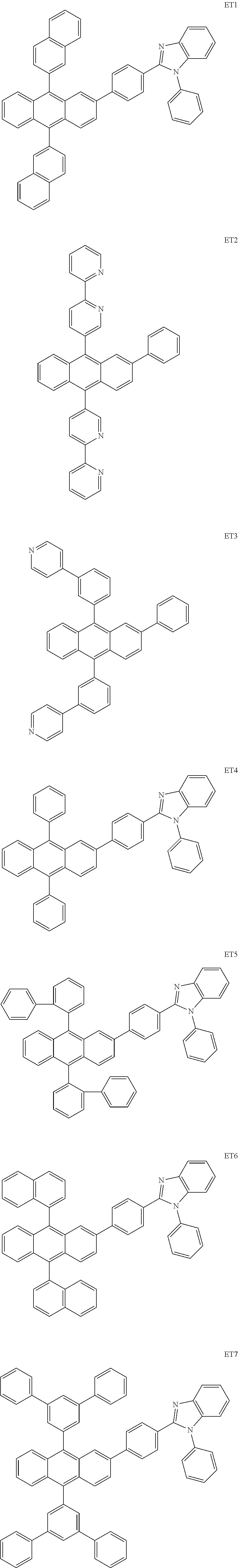

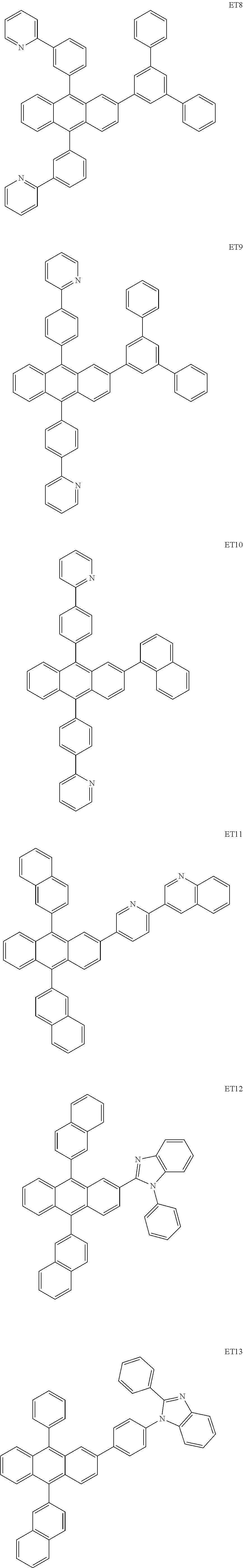

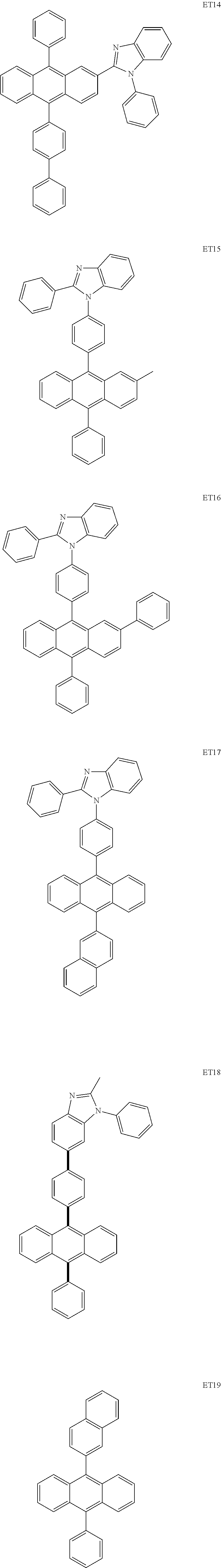

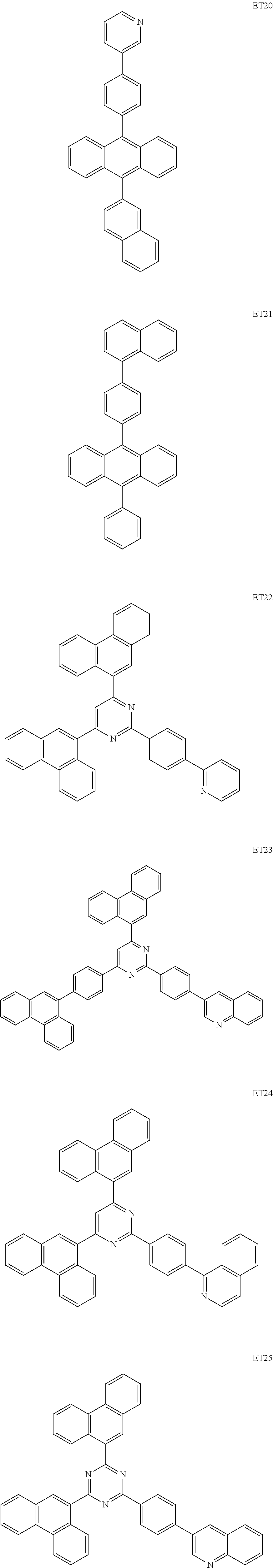

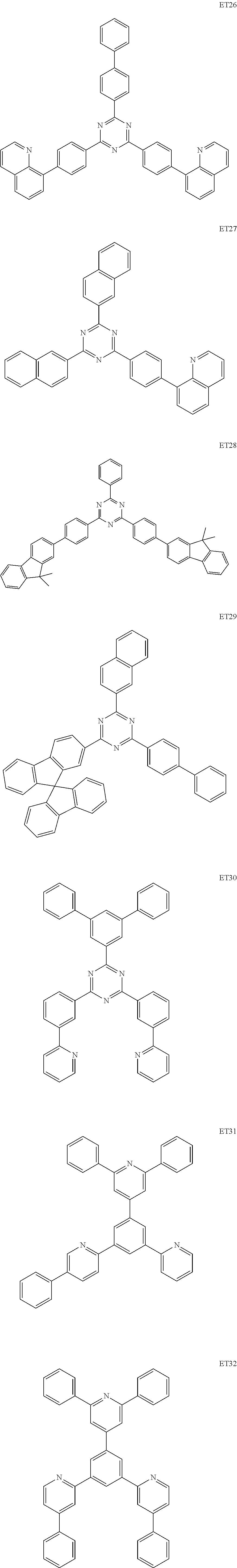

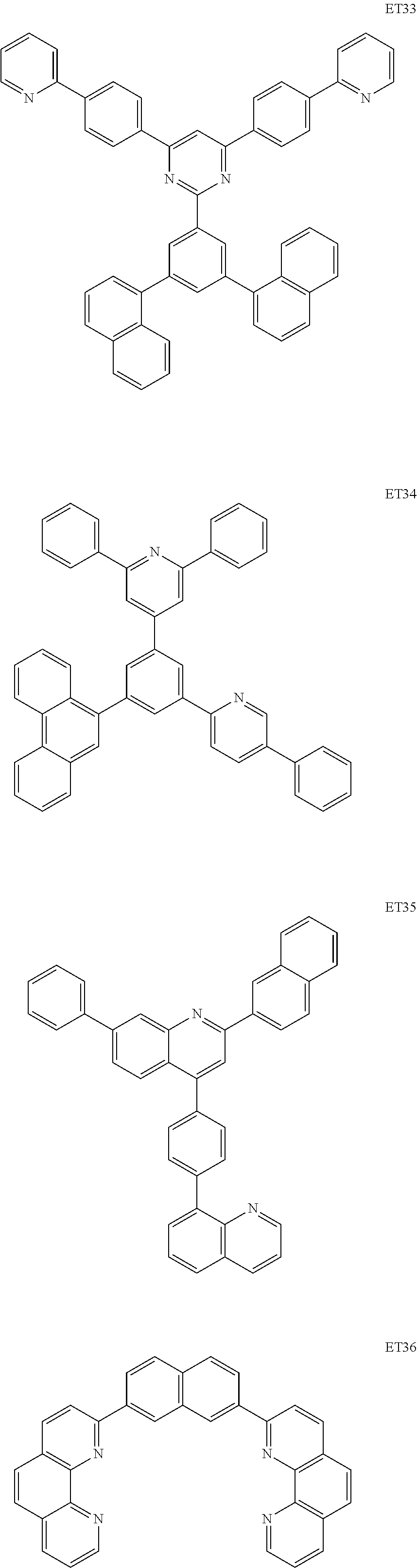 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In an embodiment, the electron transport regions ETR may include: a metal halide such as LiF, NaCl, CsF, RbCl, RbI, CuI, or KI; a lanthanide metal such as Yb; or a co-deposited material of a metal halide and a lanthanide metal. For example, the electron transport region ETR may include KI:Yb, RbI:Yb, LiF:Yb, etc., as a co-deposited material. The electron transport region ETR may be formed of a metal oxide such as Li₂O or BaO, or 8-hydroxyl-lithium quinolate (Liq), etc., but embodiments are not limited thereto. The electron transport region ETR may also be formed of a mixture material of an electron transport material and an insulating organometallic salt. The organometallic salt may be a material having an energy band gap equal to or greater than about 4 eV. For example, the organometallic salt may include a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but embodiments are not limited thereto.

The electron transport region ETR may include the above-described compounds of the hole transport region in at least one of an electron injection layer EIL, an electron transport layer ETL, or a hole blocking layer HBL.

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. If the thickness of the electron transport layer ETL satisfies any of the aforementioned ranges, satisfactory electron transport characteristics may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. If the thickness of the electron injection layer EIL satisfies any of the above-described ranges, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgAg). In another embodiment, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, or the like.

Although not shown in the drawings, the second electrode EL2 may be electrically connected to an auxiliary electrode. If the second electrode EL2 is electrically connected to the auxiliary electrode, the resistance of the second electrode EL2 may decrease.

In an embodiment, the light emitting device ED may further include a capping layer CPL disposed on the second electrode EL2. The capping layer CPL may be a multilayer or a single layer.

In an embodiment, the capping layer CPL may include an organic layer or an inorganic layer. For example, when the capping layer CPL contains an inorganic material, the inorganic material may include an alkaline metal compound (e.g., LiF), an alkaline earth metal compound (e.g., MgF₂), SiON, SiNₓ, SiOy, etc.

For example, when the capping layer CPL includes an organic material, the organic material may include 2,2′-dimethyl-N,N′-di-[(1-naphthyl)-N,N′-diphenyl]-1,1′-biphenyl-4,4′-diamine (a-NPD), NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or an epoxy resin, or an acrylate such as methacrylate. However, embodiments are not limited thereto. In an embodiment, the capping layer CPL may include at least one of Compounds P1 to P5:

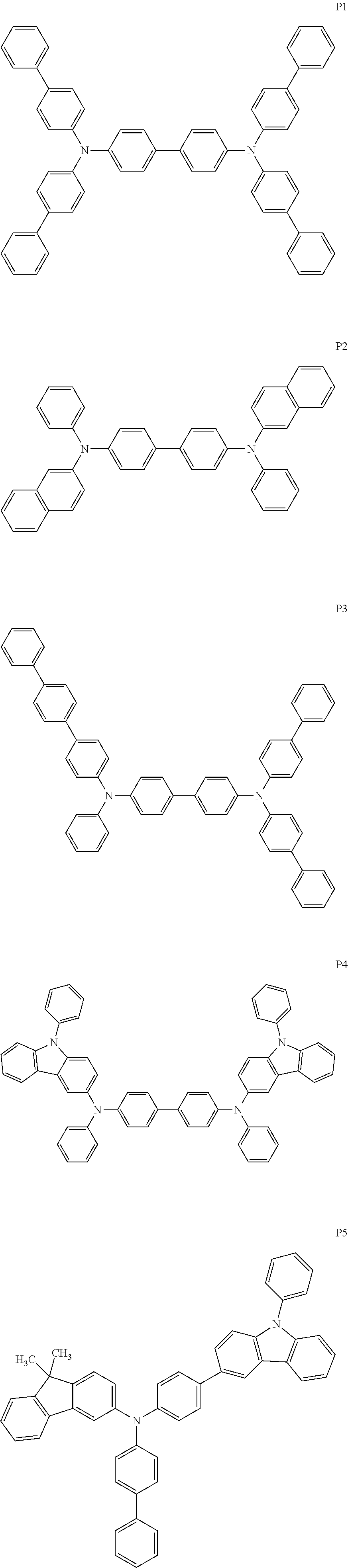 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, a refractive index of the capping layer CPL may be equal to or greater than 1.6 with respect to light in a wavelength range of about 550 nm to about 660 nm.

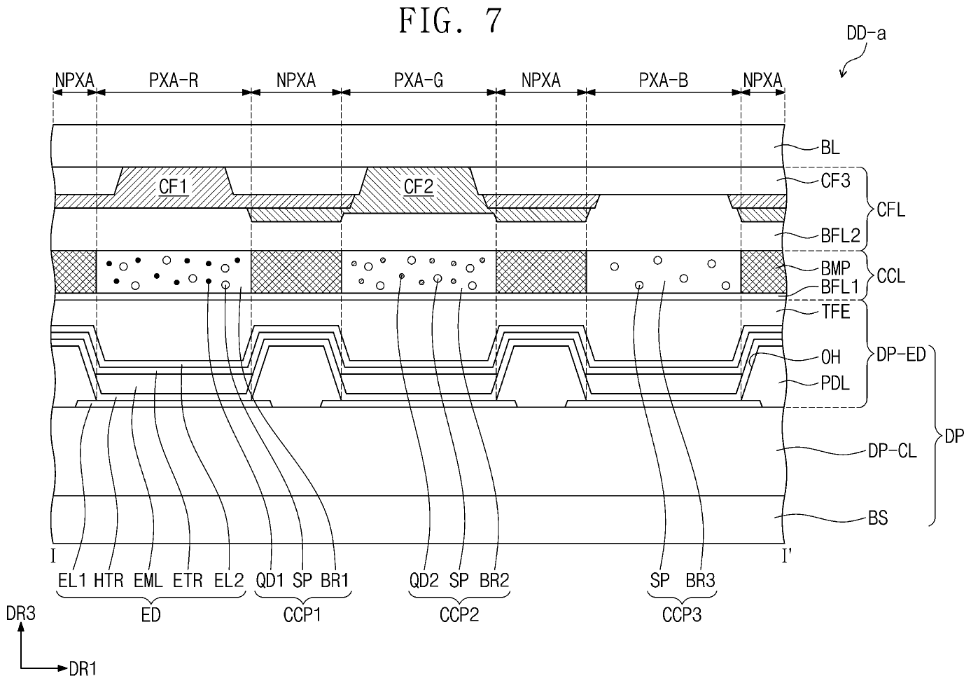

FIG. 7 to FIG. 10 are each a schematic cross-sectional view of a display apparatus according to embodiments. In the explanation on the display apparatuses according to embodiments with reference to FIGS. 7 to 10, the features which have been described above with respect to FIGS. 1 to 6 will not be explained again, and the differing features will be described.

Referring to FIG. 7, a display apparatus DD-a according to an embodiment may include a display panel DP including a display device layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and a display device layer DP-ED, and the display device layer DP-ED may include a light emitting device ED.

The light emitting device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In embodiments, a structure of the light emitting device ED shown in FIG. 7 may be the same as a structure of a light emitting device according to one of FIGS. 3 to 6 as described herein.

In the display apparatus DD-a, the emission layer EML of the light emitting device ED may include the amine compound as described herein.

Referring to FIG. 7, the emission layer EML may be disposed in an opening OH defined in a pixel defining film PDL. For example, the emission layer EML, which is divided by the pixel defining film PDL and provided corresponding to each of the light emitting regions PXA-R, PXA-G, and PXA-B may each emit light in a same wavelength range. In the display apparatus DD-a, the emission layer EML may emit blue light. Although not shown in the drawings, in an embodiment, the emission layer EML may be provided as a common layer for all of the light emitting regions PXA-R, PXA-G, and PXA-B.

The light control layer CCL may be disposed on the display panel DP. The light control layer CCL may include a light conversion body. The light conversion body may be a quantum dot, a phosphor, or the like. The light conversion body may convert the wavelength of a provided light and may emit the resulting light. For example, the light control layer CCL may be a layer including a quantum dot or a layer including a phosphor.

The light control layer CCL may include light control parts CCP1, CCP2, and CCP3. The light control parts CCP1, CCP2, and CCP3 may be spaced apart from each other.

Referring to FIG. 7, divided patterns BMP may be disposed between the light control parts CCP1, CCP2, and CCP3 which are spaced apart from each other, but embodiments are not limited thereto. In FIG. 7, it is shown that the divided patterns BMP do not overlap the light control parts CCP1, CCP2, and CCP3, but at least a portion of the edges of the light control parts CCP1, CCP2, and CCP3 may overlap the divided patterns BMP.

The light control layer CCL may include a first light control part CCP1 including a first quantum dot QD1 that converts first color light provided from the light emitting device ED into second color light, a second light control part CCP2 including a second quantum dot QD2 that converts the first color light into third color light, and a third light control part CCP3 that transmits the first color light.

In an embodiment, the first light control part CCP1 may provide red light which may be the second color light, and the second light control part CCP2 may provide green light which may be the third color light. The third light control part CCP3 may provide blue light by transmitting a blue light which may be the first color light provided from the light emitting device ED. For example, the first quantum dot QD1 may be a red quantum dot, and the second quantum dot QD2 may be a green quantum dot. The quantum dots QD1 and QD2 may each be a quantum dot as described herein.

The light control layer CCL may further include a scatterer SP. The first light control part CCP1 may include the first quantum dot QD1 and a scatterer SP, the second light control part CCP2 may include the second quantum dot QD2 and a scatterer SP, and the third light control part CCP3 may not include any quantum dot but may include a scatterer SP.

The scatterer SP may be inorganic particles. For example, the scatterer SP may include at least one of TiO₂, ZnO, Al₂O₃, SiO₂, or hollow silica. The scatterer SP may include any one of TiO₂, ZnO, Al₂O₃, SiO₂, and hollow silica, or the scatterer SP may be a mixture of at least two materials selected from TiO₂, ZnO, Al₂O₃, SiO₂, and hollow silica.

The first light control part CCP1, the second light control part CCP2, and the third light control part CCP3 may each include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of various resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light control layer CCL may include a barrier layer BFL1. The barrier layer BFL1 may prevent the penetration of moisture and/or oxygen (hereinafter, referred to as ‘moisture/oxygen’). The barrier layer BFL1 may be disposed on the light control parts CCP1, CCP2, and CCP3 to block the light control parts CCP1, CCP2, and CCP3 from exposure to moisture/oxygen. The barrier layer BFL1 may cover the light control parts CCP1, CCP2, and CCP3. A barrier layer BFL2 may be provided between the light control parts CCP1, CCP2, and CCP3 and the filters CF1, CF2, and CF3.

The barrier layers BFL1 and BFL2 may each independently include at least one inorganic layer. For example, the barrier layers BFL1 and BFL2 may each independently include an inorganic material. For example, the barrier layers BFL1 and BFL2 may each independently include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures transmittance, etc. The barrier layers BFL1 and BFL2 may each independently further include an organic film. The barrier layers BFL1 and BFL2 may each independently be formed of a single layer or of multiple layers.

In the display apparatus DD-a, the color filter layer CFL may be disposed on the light control layer CCL. In an embodiment, the color filter layer CFL may be directly disposed on the light control layer CCL. For example, the barrier layer BFL2 may be omitted.

The color filter layer CFL may include a light shielding part (not shown) and filters CF1, CF2, and CF3. The color filter layer CFL may include a first filter CF1 that transmits second color light, a second filter CF2 that transmits third color light, and a third filter CF3 that transmits first color light. For example, the first filter CF1 may be a red filter, the second filter CF2 may be a green filter, and the third filter CF3 may be a blue filter. The filters CF1, CF2, and CF3 may each include a polymeric photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may not be separated and may be provided as one filter.

The light shielding part (not shown) may be a black matrix. The light shielding part (not shown) may include an organic light shielding material or an inorganic light shielding material that includes a black pigment or dye. The light shielding part (not shown) may prevent light leakage, and may separate boundaries between the adjacent filters CF1, CF2, and CF3. In an embodiment, the light shielding part (not shown) may be formed of a blue filter.

The first to third filters CF1, CF2, and CF3 may be disposed to respectively correspond to the red light emitting region PXA-R, the green light emitting region PXA-G, and the blue light emitting region PXA-B.

A base substrate BL may be disposed on the color filter layer CFL. The base substrate BL may provide a base surface on which the color filter layer CFL, the light control layer CCL, and the like are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

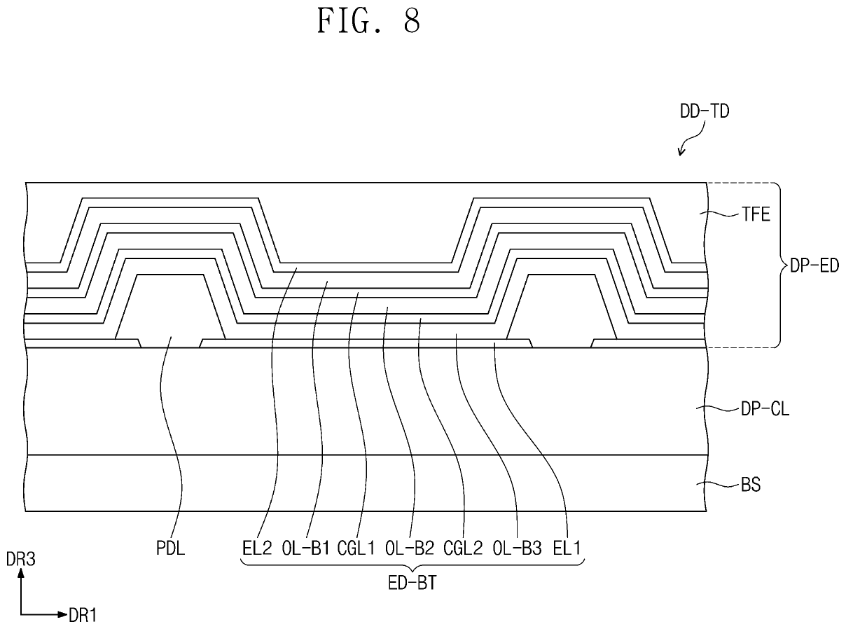

FIG. 8 is a schematic cross-sectional view illustrating a portion of a display apparatus according to an embodiment that corresponds to the display panel DP of FIG. 7. In the display apparatus DD-TD according to an embodiment, the light emitting device ED-BT may include light emitting structures OL-B1, OL-B2, and OL-B3. The light emitting device ED-BT may include a first electrode EL1 and a second electrode EL2 which face each other, and the light emitting structures OL-B1, OL-B2, and OL-B3 stacked in a thickness direction between the first electrode EL1 and the second electrode EL2. The light emitting structures OL-B1, OL-B2, and OL-B3 may each include an emission layer EML (FIG. 7) and a hole transport region HTR and an electron transport region ETR disposed with the emission layer EML (FIG. 7) arranged therebetween.

For example, the light emitting device ED-BT included in the display apparatus DD-TD may be a light emitting device having a tandem structure and including multiple emission layers.

In an embodiment illustrated in FIG. 8, light emitted from the light emitting structures OL-B1, OL-B2, and OL-B3 may each be blue light. However, embodiments are not limited thereto, and the light respectively emitted from the light emitting structures OL-B1, OL-B2, and OL-B3 may have wavelength ranges that are different from each other. For example, the light emitting device ED-BT, which includes the light emitting structures OL-B1, OL-B2, and OL-B3 that emit light having different wavelength ranges from each other, may emit white light.

Charge generation layers CGL1 and CGL2 may each be disposed between neighboring light emitting structures OL-B1, OL-B2, and OL-B3. Charge generation layers CGL1 and CGL2 may each independently include a p-type charge generation layer and/or an n-type charge generation layer.

In an embodiment, at least one of the light emitting structures OL-B1, OL-B2, or OL-B3 included in the display apparatus DD-TD may include the amine compound according to an embodiment, as described herein.

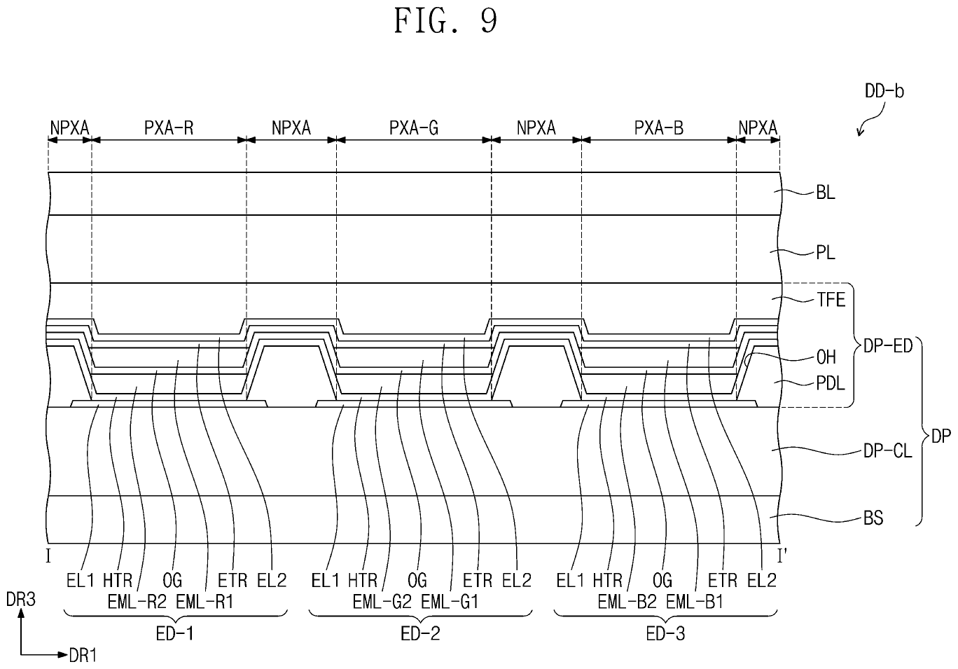

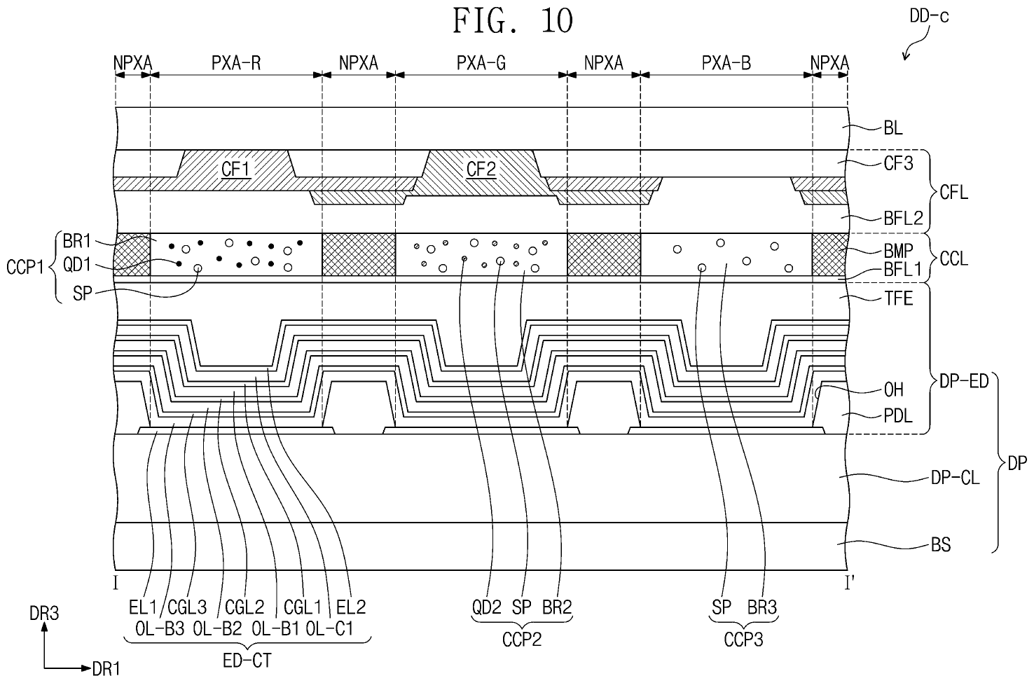

FIG. 9 is a schematic cross-sectional view illustrating a display apparatus according to an embodiment; and FIG. 10 is a schematic cross-sectional view illustrating a display apparatus according to an embodiment.

Referring to FIG. 9, a display apparatus DD-b according to an embodiment may include light emitting devices ED-1, ED-2, and ED-3, which may each include two emission layers that are stacked. In comparison to the display apparatus DD illustrated in FIG. 2, the embodiment illustrated in FIG. 9 is different at least in that the first to third light emitting devices ED-1, ED-2, and ED-3 each include two emission layers that are stacked in a thickness direction. In each of the first to third light emitting devices ED-1, ED-2, and ED-3, the two emission layers may emit light in a same wavelength region.

The first light emitting device ED-1 may include a first red emission layer EML-R1 and a second red emission layer EML-R2. The second light emitting device ED-2 may include a first green emission layer EML-G1 and a second green emission layer EML-G2. The third light emitting device ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may be a single layer or a multilayer. The emission auxiliary part OG may include a charge generation layer. For example, the emission auxiliary part OG may include an electron transport region, a charge generation layer, and a hole transport region, which may be stacked in that order. The emission auxiliary part OG may be provided as a common layer for all of the first to third light emitting devices ED-1, ED-2, and ED-3. However, embodiments are not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may each be disposed between the electron transport region ETR and the emission auxiliary part OG. The second red emission layer EML-R2, the second green emission layer EML-G2, and the second blue emission layer EML-B2 may each be disposed between the emission auxiliary part OG and the hole transport region HTR.

For example, the first light emitting device ED-1 may include the first electrode EL1, the hole transport region HTR, the second red emission layer EML-R2, the emission auxiliary part OG, the first red emission layer EML-R1, the electron transport region ETR, and the second electrode EL2, which are stacked in that order. The second light emitting device ED-2 may include the first electrode EL1, the hole transport region HTR, the second green emission layer EML-G2, the emission auxiliary part OG, the first green emission layer EML-G1, the electron transport region ETR, and the second electrode EL2, which are stacked in that order. The third light emitting device ED-3 may include the first electrode EL1, the hole transport region HTR, the second blue emission layer EML-B2, the emission auxiliary part OG, the first blue emission layer EML-B1, the electron transport region ETR, and the second electrode EL2, which are stacked in that order.

An optical auxiliary layer PL may be disposed on the display device layer DP-ED. The optical auxiliary layer PL may include a polarizing layer. The optical auxiliary layer PL may be disposed on the display panel DP and may control light reflected at the display panel DP from an external light. Although not shown in the drawings, in an embodiment, the optical auxiliary layer PL may be omitted from the display apparatus DD-b.

At least one emission layer included in the display apparatus DD-b illustrated in FIG. 9 may include the amine compound as described herein. For example, in an embodiment, at least one of the first blue emission layer EML-B1 or the second blue emission layer EML-B2 may include the amine compound.

In contrast to FIGS. 8 and 9, FIG. 10 illustrates a display apparatus DD-c that is different at least in that it includes four light emitting structures OL-B1, OL-B2, OL-B3, and OL-C1. A light emitting device ED-CT may include a first electrode EL1 and a second electrode EL2 which face each other, and first to fourth light emitting structures OL-B1, OL-B2, OL-B3, and OL-C1 that are stacked in a thickness direction between the first electrode EL1 and the second electrode EL2. Charge generation layers CGL1, CGL2, and CGL3 may each be disposed between adjacent light emitting structures among the first to fourth light emitting structures OL-B1, OL-B2, OL-B3, and OL-C1. Among the four light emitting structures, the first to third light emitting structures OL-B1, OL-B2, and OL-B3 may each emit blue light, and the fourth light emitting structure OL-C1 may emit green light. However, embodiments are not limited thereto, and the first to fourth light emitting structures OL-B1, OL-B2, OL-B3, and OL-C1 may emit light having different wavelength regions from each other.

The charge generation layers CGL1, CGL2, and CGL3 which are disposed between adjacent light emitting structures OL-B1, OL-B2, OL-B3, and OL-C1 may each independently include a p-type charge generation layer and/or an n-type charge generation layer.

In the display apparatus DD-c, at least one of the light emitting structures OL-B1, OL-B2, OL-B3, and OL-C1 may include the amine compound as described herein.

The light emitting device ED according to an embodiment may include the amine compound according to an embodiment in at least one functional layer disposed between the first electrode EL1 and the second electrode EL2, thereby exhibiting improved luminous efficiency and service life characteristics. The light emitting device ED may include the amine compound in at least one of a hole transport region HTR, an emission layer EML, or an electron transport region ETR disposed between the first electrode EL1 and the second electrode EL2, or in a capping layer CPL.

In an embodiment, the hole transport region HTR of the light emitting device ED may include the amine compound according to an embodiment, and the light emitting device of an embodiment may exhibit excellent luminous efficiency and long service life characteristics. In an embodiment, the hole transport region HTR may include a hole injection layer HIL disposed on the first electrode EL1 and a hole transport layer HTL disposed on the hole injection layer HIL, and the hole transport layer HTL may include the amine compound according to an embodiment.

Hereinafter, an amine compound according to an embodiment and a light emitting device according to an embodiment will be described in detail with reference to the Examples and the Comparative Examples. The Examples described below are only provided as illustrations to assist in understanding the disclosure, and the scope thereof is not limited thereto.

### EXAMPLES

### 1. Synthesis of Amine Compound

A synthesis method of an amine compound according to an embodiment will be described in detail by illustrating synthesis methods of Compounds 1, 2, 33, 37, 40, 54, 61, 230, 310, 338, and 366. In the following descriptions, the synthesis methods of the amine compounds are provided as examples, and the synthesis methods of the amine compound are not limited to the Examples.

### (1) Synthesis of Compound 1

Compound 1 according to an example may be synthesized by, for example, the reaction below.

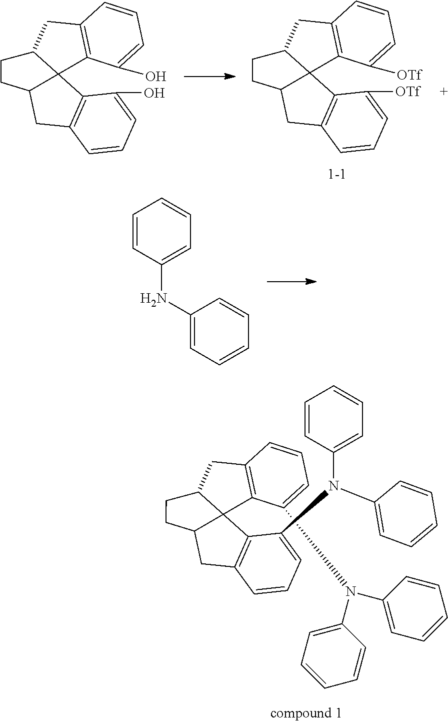

### Synthesis of Intermediate 1-1

(5aR,7aR)-5,5a,6,7,7a,8-hexahydrocyclopenta[1,2-a:1,5-a′]diindene-1,12-diol 2.8 g (10 mmol) were dissolved in dichloromethane (100 mL) and diisopropylethylamine (DIPEA), and 5 mL of trifluoromethanesulfonic acid was added dropwise thereto at 0° C., and the reaction solution was stirred at room temperature for about 1 hour. Water (40 mL) was added to the reaction solution, and the mixture was extracted three times with 50 mL of ethyl ether. The collected ethyl ether was dried over MgSO₄, and residues obtained by evaporating the solvent were separated and purified by silica gel column chromatography to obtain Intermediate 1-1 (3.78 g, yield 70%). The resultant intermediate was identified with LC-MS. C₂₁H₁₆F₆O₆S₂ M⁺: 542.0

### Synthesis of Compound 1

Intermediate 1-1 (3.78 g, 7.0 mmol), diphenyl amine (2.70 g, 16 mmol), P(t-Bu)₃ (1.60 g, 0.8 mmol), tris(dibenzylideneacetone)dipalladium(0) (Pd₂dba₃) (0.36 g, 0.4 mmol), and sodium tert-butoxide (2.3 g, 24 mmol) were dissolved in 200 mL of toluene and the reaction solution was stirred at about 80° C. for about 3 hours. The reaction solution was cooled to room temperature, 60 mL of water was added thereto, and the mixture was extracted three times with 80 mL of ethyl ether. The collected ethyl ether was dried over MgSO₄, and residues obtained by evaporating the solvent were separated and purified by silica gel column chromatography to obtain Compound 1 (2.28 g, yield 56%). The resultant compound was identified through HRMS. (Yield: 56%, HRMS (EI): calcd.: 580.2878; found: 580.2876).

### (2) Synthesis of Compound 2

Compound 2 was synthesized in the same manner as in Synthesis of Compound 1 by using diphenyl amine and 9,9-dimethyl-N-phenyl-9H-fluoren-2-amine. The resultant compound was identified through HRMS. (HRMS (EI): calcd.: 696.3504; found: 696.3506).

### (3) Synthesis of Compound 33

Compound 33 was synthesized in the same manner as in Synthesis of Compound 1 by using 9,9-dimethyl-N-phenyl-9H-fluoren-2-amine and N-phenyl-[1,1′-biphenyl]-4-amine instead of diphenyl amine. The resultant compound was identified through HRMS. (HRMS (EI): calcd.: 772.3817; found: 772.3815).

### (4) Synthesis of Compound 37

Compound 37 was synthesized in the same manner as in Synthesis of Compound 1 by using 9,9-dimethyl-N-phenyl-9H-fluoren-2-amine and N,3-diphenylnaphthalen-2-amine instead of diphenyl amine. The resultant compound was identified through HRMS. (HRMS (EI): calcd.: 838.4287; found: 838.4289).

### (5) Synthesis of Compound 40

Compound 40 was synthesized in the same manner as in Synthesis of Compound 1 by using 9,9-dimethyl-N-phenyl-9H-fluoren-2-amine and 4-cyclohexyl-N-phenylaniline instead of diphenyl amine. The resultant compound was identified through HRMS. (HRMS (EI): calcd.: 778.4287; found: 778.4285).

### (6) Synthesis of Compound 54

Compound 54 was synthesized in the same manner as in Synthesis of Compound 1 by using 9,9-dimethyl-N-phenyl-9H-fluoren-2-amine and N-phenyl-4-(3-phenylnaphthalen-2-yl)aniline instead of diphenyl amine. The resultant compound was identified through HRMS. (HRMS (EI): calcd.: 898.4287; found: 898.4280).

### (7) Synthesis of Compound 61

Compound 61 was synthesized in the same manner as in Synthesis of Compound 1 by using N-phenyl-[1,1′-biphenyl]-4-amine and 9,9-dimethyl-N,5-diphenyl-9H-fluoren-2-amine instead of diphenyl amine. The resultant compound was identified through HRMS. (HRMS (EI): calcd.: 848.4130; found: 848.4136).

### (8) Synthesis of Compound 230

Compound 230 was synthesized in the same manner as in Synthesis of Compound 1 by using N-([1,1′-biphenyl]-4-yl)-9,9-dimethyl-9H-fluoren-2-amine instead of diphenyl amine.

The resultant compound was identified through HRMS. (HRMS (EI): calcd.: 772.3817; found: 772.3815).

### (9) Synthesis of Compound 310

Compound 310 was synthesized in the same manner as in Synthesis of Compound 1 by using (5aR,8aR)-5a,6,7,8,8a,9-hexahydro-5H-indeno[2,1-d]fluorene-1,13-diol instead of (5aR,7aR)-5,5a,6,7,7a,8-hexahydrocyclopenta[1,2-a:1,5-a′]diindene-1,12-diol. The resultant compound was identified through HRMS. (HRMS (EI): calcd.: 594.3035; found: 594.3033).

### (10) Synthesis of Compound 338

Compound 338 was synthesized in the same manner as in Synthesis of Compound 1 by using (5aR,9aR)-5,5a,6,7,8,9,9a,10-octahydrobenzo[a]indeno[2,1-i]azulene-1,14-diol instead of (5aR,7aR)-5,5a,6,7,7a,8-hexahydrocyclopenta[1,2-a:1,5-a′]diindene-1,12-diol.

The resultant compound was identified through HRMS. (HRMS (EI): calcd.: 608.3191; found: 608.3193).

### (11) Synthesis of Compound 366

Compound 338 was synthesized in the same manner as in Synthesis of Compound 1 by using (1aR,6aR)-1a,2,3,4,5,6,6a,7-octahydro-1H-cycloocta[1,2-a:1,8-a′]diindene-11,12-diol instead of (5aR,7aR)-5,5a,6,7,7a,8-hexahydrocyclopenta[1,2-a:1,5-a′]diindene-1,12-diol.

The resultant compound was identified through HRMS. (HRMS (EI): calcd.: 622.3348; found: 622.3346).

### 2. Manufacture and Evaluation of Light Emitting Device Including Amine Compound

(Manufacture of Light Emitting Device)

A light emitting device including an amine compound according to an embodiment in a hole transport layer was manufactured as follows. Compounds 1, 2, 33, 37, 40, 54, 61, 230, 310, 338, and 366, which are Example Compounds as described above, were used as hole transport layer materials to manufacture the light emitting devices of Examples 1 to 11, respectively. Comparative Example 1 to Comparative Example 3 correspond to the light emitting device manufactured by using Comparative Compound C1 to Comparative Compound C3 as a hole transport layer material.

### Example Compounds

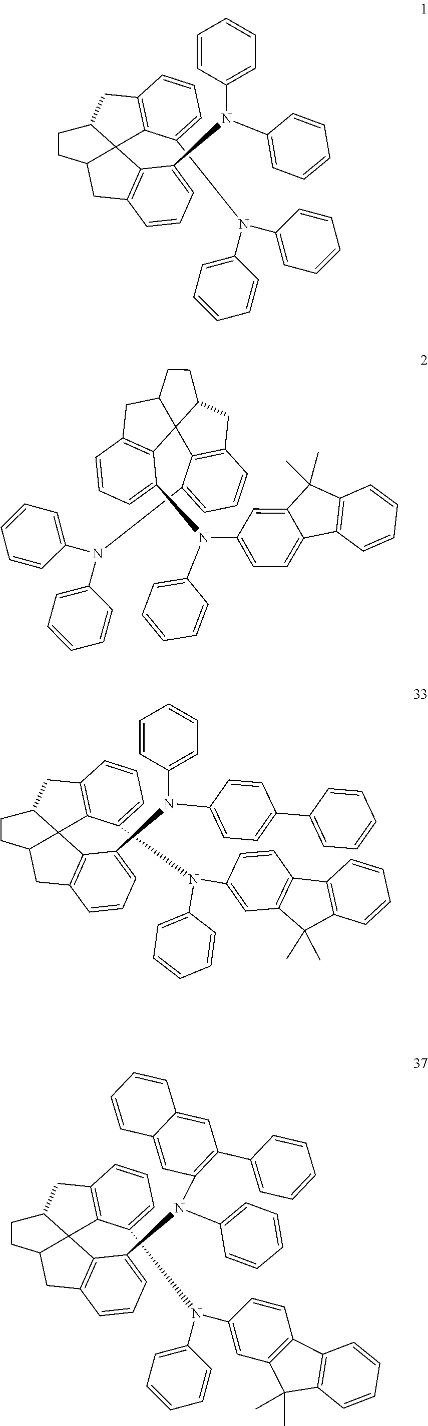

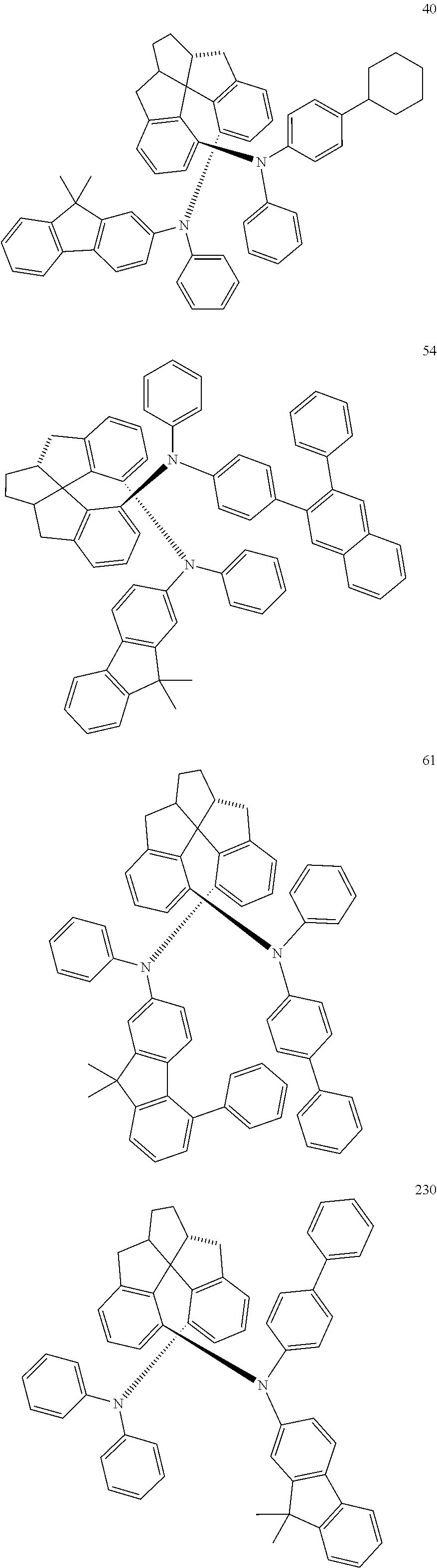

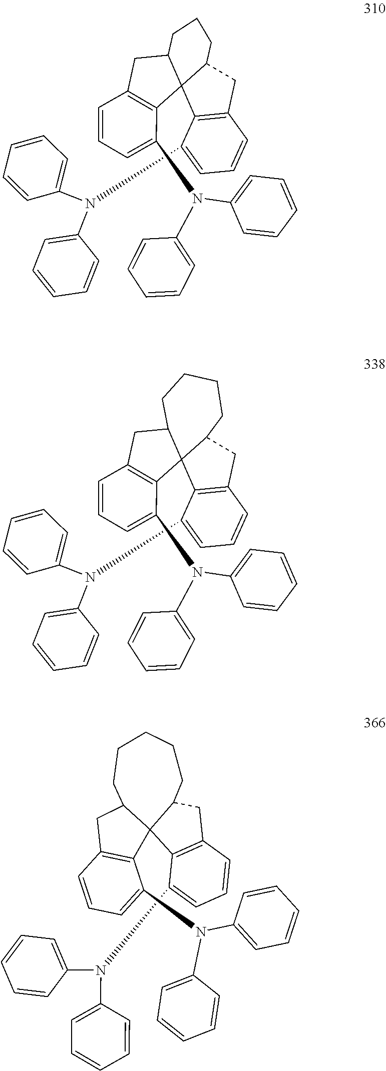

### Comparative Example Compounds

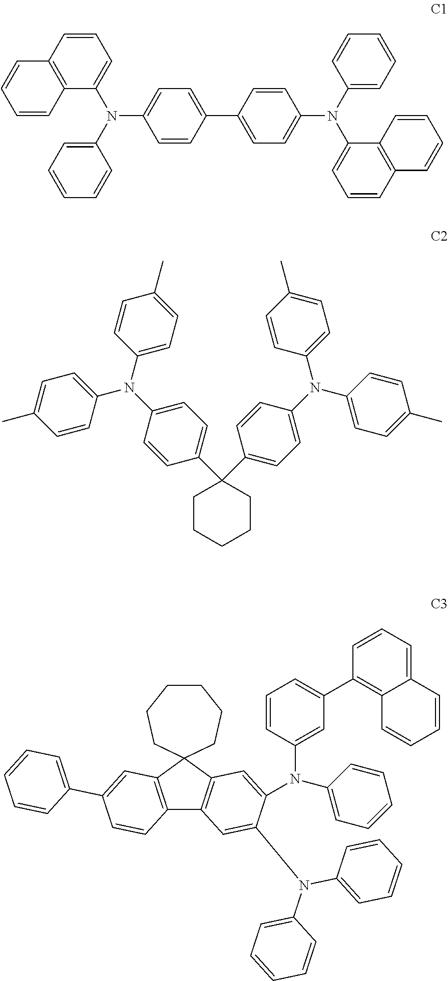

The light emitting devices of the Examples and the Comparative Examples were manufactured by the following method. With respect to the light emitting devices of the Examples and the Comparative Examples, an ITO glass substrate of about 15 Q/cm² (about 1,200 Å) made by Corning Co. was cut to a size of about 50 mm×50 mm×0.7 mm, washed by ultrasonic waves using isopropyl alcohol and distilled water for about 5 minutes, respectively, and irradiated with ultraviolet rays for about 30 minutes and cleansed by exposing to ozone, and installed on a vacuum deposition apparatus.

On the ITO glass substrate, 2-TNATA was deposited in vacuum to form a 600-Å-thick hole injection layer, and Example Compound or Comparative Compound was deposited in vacuum to form a 300-Å-thick hole transport layer.

On the hole transport layer, 9,10-di(naphthalen-2-yl)anthracene (hereinafter, ADN) as a blue fluorescent host and 4,4′-bis[2-(4-(N,N-diphenylamino)phenyl)vinyl]biphenyl (hereinafter, DPAVBi) as a blue fluorescent dopant were co-deposited at a weight ratio of 98:2 to form a 300-Å-thick emission layer.

On the emission layer, Alq₃ was deposited to form a 300-Å-thick electron transport layer, and on the electron transport layer, LiF that is an alkaline metal halide was deposited to form a 10-Å-thick electron injection layer. On the electron injection layer, Al was deposited in vacuum to form a 3,000-Å-thick second electrode.

Compounds used for manufacturing the light emitting devices of Examples and Comparative Examples are disclosed below. The materials below were used to manufacture the devices by subjecting commercial products to sublimation purification.

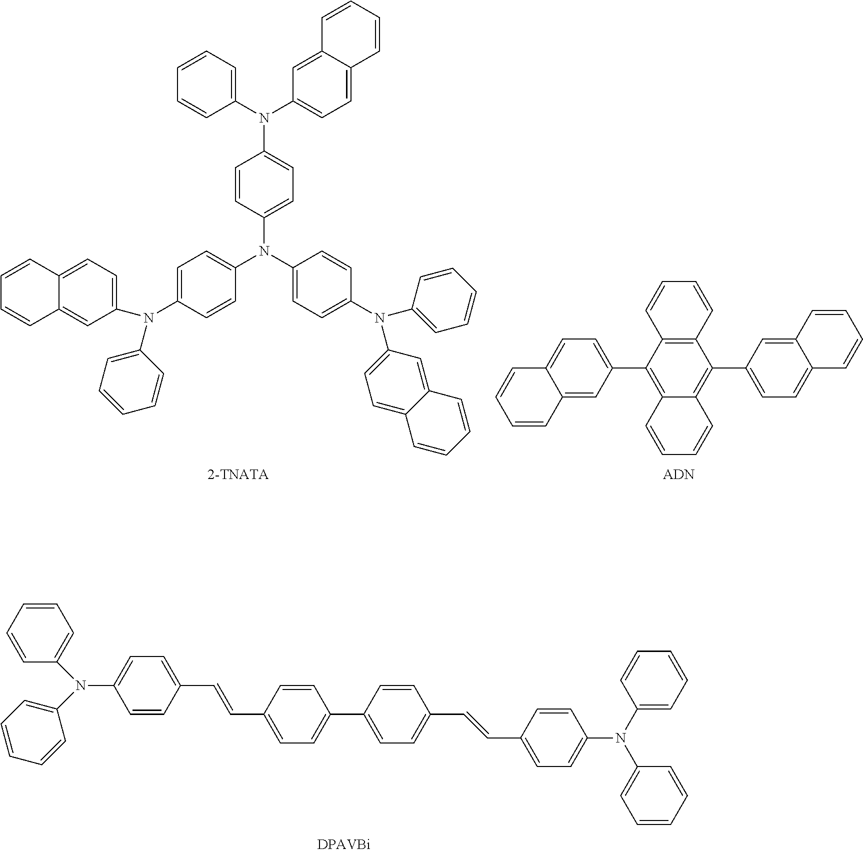

(Evaluation of Light Emitting Device Characteristics)

Driving voltage, brightness, luminous efficiency, and a half service life of each of the light emitting devices manufactured with Example Compounds 1, 2, 33, 37, 40, 54, 61, 230, 310, 338, and 366, and Comparative Example Compound C1 to C3 as described above were evaluated. Evaluation results of the light emitting devices of Examples 1 to 11, and Comparative Example 1 to 3 are listed in Table 1. In the characteristic evaluation results of the Examples and the Comparative Example listed in Table 1, driving voltages and current densities were measured by using V7000 OLED IVL Test System (Polaronix). To evaluate the characteristics of the light emitting devices manufactured in Examples 1 to 11 and Comparative Example 1 to 3, driving voltages and efficiencies (cd/A) at a current density of 50 mA/cm² were measured, and the deterioration time from an initial value to 50% brightness when the device was continuously operated at a current density of 100 mA/cm² was set as a half service life, and the evaluation was conducted.

TABLE 1

| | | Driving | Current | | | | |
|---|---|---|---|---|---|---|---|
| | Hole transport | voltage | density | Brightness | Efficiency | Emission | Half- |
| | layer material | (V) | (mA/cm²) | (cd/m²) | (cd/A) | color | lifespan (hr) |
| Example 1 | Compound 1 | 5.13 | 50 | 3200 | 6.40 | Blue | 650 |
| Example 2 | Compound 2 | 5.08 | 50 | 3150 | 6.30 | Blue | 620 |
| Example 3 | Compound 33 | 4.99 | 50 | 3100 | 6.20 | Blue | 580 |
| Example 4 | Compound 37 | 5.19 | 50 | 3140 | 6.28 | Blue | 530 |
| Example 5 | Compound 40 | 5.20 | 50 | 3270 | 6.54 | Blue | 480 |
| Example 6 | Compound 54 | 4.95 | 50 | 3190 | 6.38 | Blue | 530 |
| Example 7 | Compound 61 | 5.07 | 50 | 3155 | 6.31 | Blue | 580 |
| Example 8 | Compound 230 | 5.08 | 50 | 3200 | 6.40 | Blue | 540 |
| Example 9 | Compound 310 | 5.23 | 50 | 3115 | 6.23 | Blue | 570 |
| Example 10 | Compound 338 | 4.98 | 50 | 3200 | 6.40 | Blue | 620 |
| Example 11 | Compound 366 | 5.05 | 50 | 3245 | 6.49 | Blue | 650 |
| Comparative | Comparative | 7.01 | 50 | 2645 | 5.29 | Blue | 258 |
| Example 1 | Example | | | | | | |
| | Compound C1 | | | | | | |
| Comparative | Comparative | 5.85 | 50 | 2700 | 5.50 | Blue | 340 |
| Example 2 | Example | | | | | | |
| | Compound C2 | | | | | | |
| Comparative | Comparative | 6.01 | 50 | 2810 | 5.37 | Blue | 350 |
| Example 3 | Example | | | | | | |
| | Compound C3 | | | | | | |

Referring to the results of Table 1, it may be confirmed that the Examples of the light emitting devices, in which the amine compounds according to embodiments are used as a material for the hole transport layer, emit the same blue light, exhibit lower driving voltage, and have improved luminous efficiency and service life characteristics as compared with the Comparative Examples. The amine compound according to an embodiment includes a 1,1-spirobiindane moiety. The 1,1-spirobiindane moiety may have a rigid core because two indane rings are linked by sharing carbon 1 that is an sp³ carbon. The amine compound of an embodiment having this structure may have a high glass transition temperature and high melting point characteristics due to a large molecular weight and improved rigidity at the core, thereby exhibiting excellent heat resistance and durability characteristics.

The amine compound according to an embodiment has a structure in which the first and second amine groups are linked to the two indane rings respectively with having the 1,1-spirobiindane moiety at the center, and the alkyl substituent links the two indane rings linked with the first and second amine groups. Accordingly, the amine compound of an embodiment may have a wide band gap, and the types of the substituents linked to the first amine group and the second amine group may be modified, thereby variously changing a highest occupied molecular orbital (HOMO) energy level of the molecule. Therefore, when the amine compound of an embodiment is applied in the hole transport region, the hole transport properties may be increased, thus improving recombination probability of holes and electrons in the emission layer, thereby increasing the luminous efficiency.

Comparative Examples 1 to Comparative Example 3 do not include the 1,1-spirobiindane moiety, and thus exhibit low thermal stability and have a decrease in the hole transport property, thereby exhibiting reduced both luminous efficiency and service life of the device as compared with Examples.

The light emitting device of an embodiment may exhibit improved device characteristics with a low driving voltage, high efficiency, and a long service life.

The amine compound of an embodiment may be included in a hole transport region of the light emitting device to contribute to high efficiency and a long service life of the light emitting device.

Embodiments have been disclosed herein, and although terms are employed, they are used and are to be interpreted in a generic and descriptive sense only and not for the purposes of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light emitting device comprising:
a first electrode;
a second electrode facing the first electrode; and
a plurality of functional layers disposed between the first electrode and the second electrode, wherein
at least one of the functional layers comprises an amine compound represented by Formula 1: 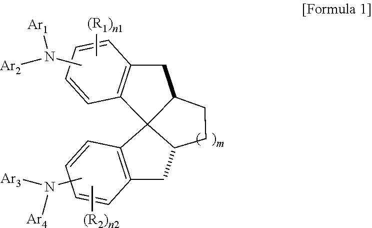
wherein in Formula 1,
Ar₁ to Ar₄ are each independently a substituted or unsubstituted aryl group having 6 to 30 ring-forming carbon atoms or a substituted or unsubstituted heteroaryl group having 2 to 30 ring-forming carbon atoms,
R₁ and R₂ are each independently a hydrogen atom, a deuterium atom, a halogen atom, a substituted or unsubstituted alkyl group having 1 to 20 carbon group, a substituted or unsubstituted aryl group having 6 to 30 ring-forming carbon atoms, or a substituted or unsubstituted heteroaryl group having 2 to 30 ring-forming carbon atoms,
n1 and n2 are each independently an integer from 0 to 3, and
m is an integer from 1 to 4.

2. The light emitting device of claim 1, wherein
the plurality of functional layers comprises:
a hole transport region disposed on the first electrode;
an emission layer disposed on the hole transport region; and
an electron transport region disposed on the emission layer, and
the hole transport region comprises the amine compound.

3. The light emitting device of claim 2, wherein
the hole transport region comprises:
a hole injection layer disposed on the first electrode; and
a hole transport layer disposed on the hole injection layer, and
the hole transport layer comprises the amine compound.

4. The light emitting device of claim 1, wherein the amine compound represented by Formula 1 is represented by Formula 2: 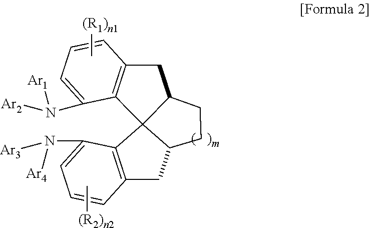
wherein in Formula 2,
Ar₁ to Ar₄, R₁, R₂, n1, n2, and m are the same as defined in Formula 1.

5. The light emitting device of claim 1, wherein the amine compound represented by Formula 1 is represented by one of Formula 3-1 to Formula 3-4: 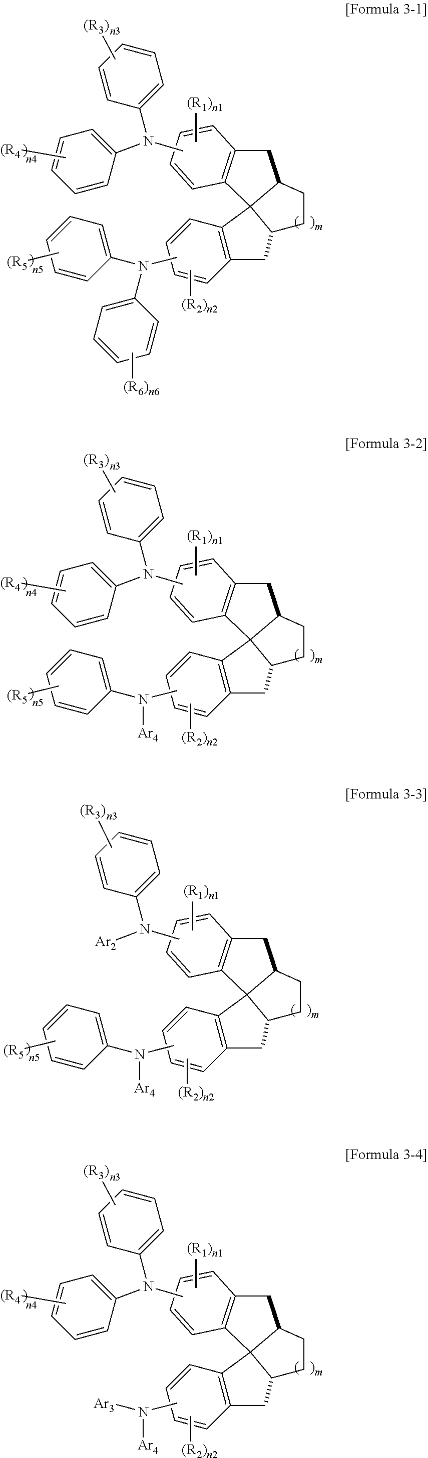
wherein in Formula 3-1 to Formula 3-4,
R₃ to R₆ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n3 to n6 are each independently an integer from 0 to 5, and
Ar₂ to Ar₄, R₁, R₂, n1, n2, and m are the same as defined in Formula 1.

6. The light emitting device of claim 5, wherein the amine compound represented by Formula 1 is represented by one of Formula 4-1 to Formula 4-4: 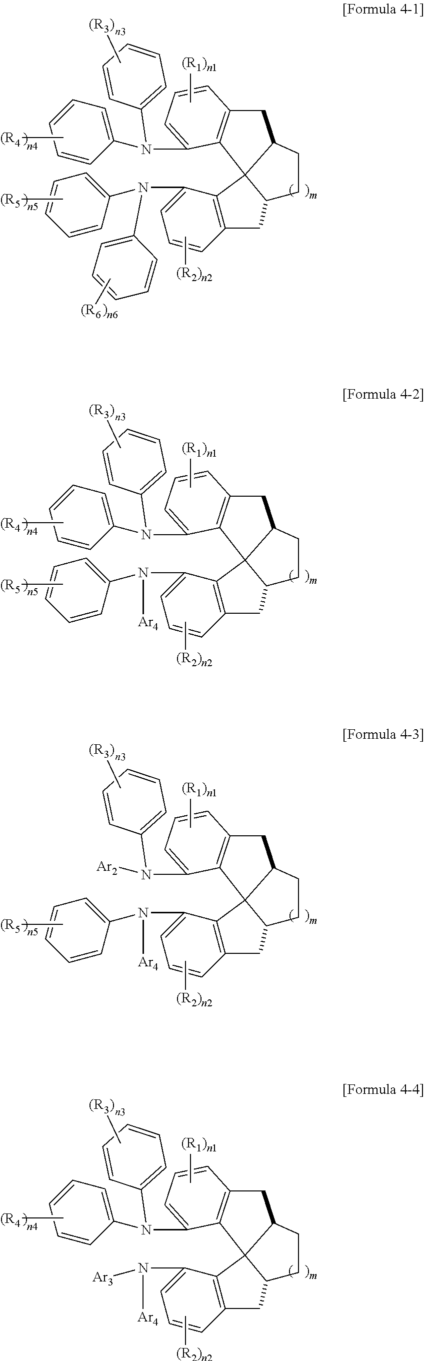
wherein in Formula 4-1 to Formula 4-4,
Ar₂ to Ar₄, R₁, R₂, n1, n2, and m are the same as defined in Formula 1, and
R₃ to R₆ and n3 to n6 are the same as defined in Formula 3-1 to Formula 3-4.

7. The light emitting device of claim 1, wherein the amine compound represented by Formula 1 is represented by one of Formula 5-1 to Formula 5-4: 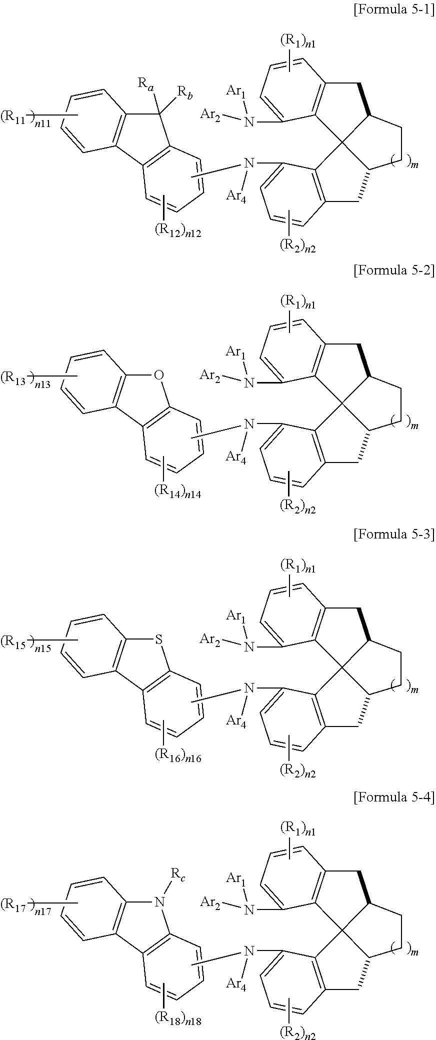
wherein in Formula 5-1 to Formula 5-4,
R₁ to R₁₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Rₐ to R_{c} are each independently a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring,
n11, n13, n15, and n17 are each independently an integer from 0 to 4,
n12, n14, n16, and n18 are each independently an integer from 0 to 3, and
Ar₁, Ar₂, Ar₄, R₁, R₂, n1, n2, and m are the same as defined in Formula 1.

8. The light emitting device of claim 7, wherein the amine compound represented by Formula 1 is represented by one of Formula 6-1 to Formula 6-6: 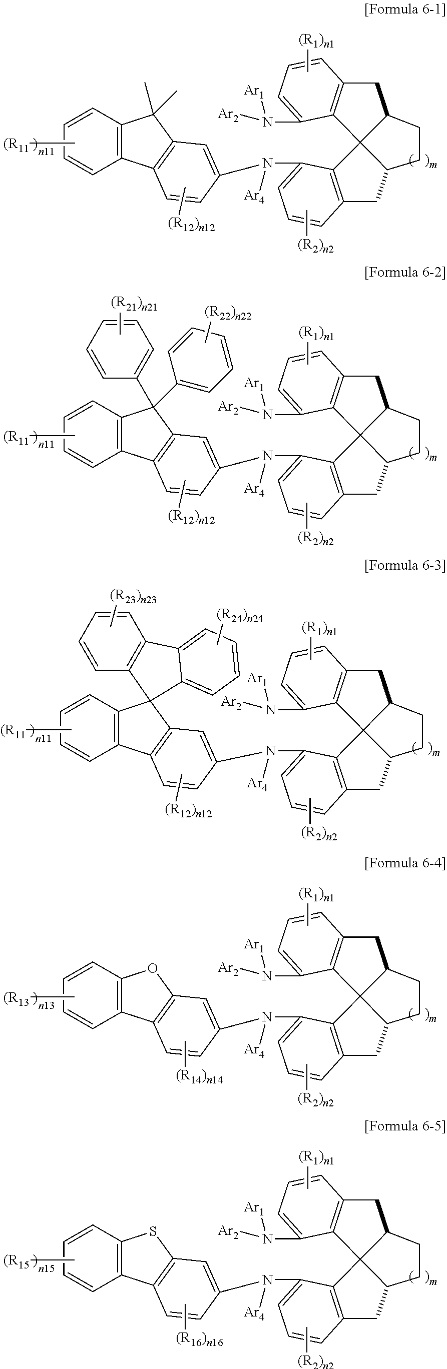 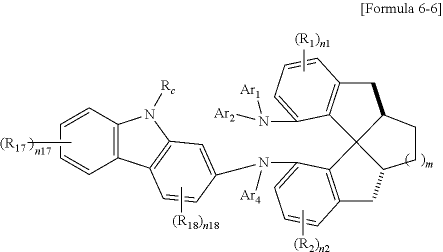
wherein in Formula 6-1 to Formula 6-6,
R₂₁ to R₂₄ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n21 and n22 are each independently an integer from 0 to 5,
n23 and n24 are each independently an integer from 0 to 4,
Ar₁, Ar₂, Ar₄, R₁, R₂, n1, n2, and m are the same as defined in Formula 1, and
R₁₁ to R₁₈, R_{c}, and n11 to n18 are the same as defined in Formula 5-1 to Formula 5-4.

9. The light emitting device of claim 1, wherein at least one of Ar₁ to Ar₄ is each independently a group represented by one of Formula 7-1 to Formula 7-7: 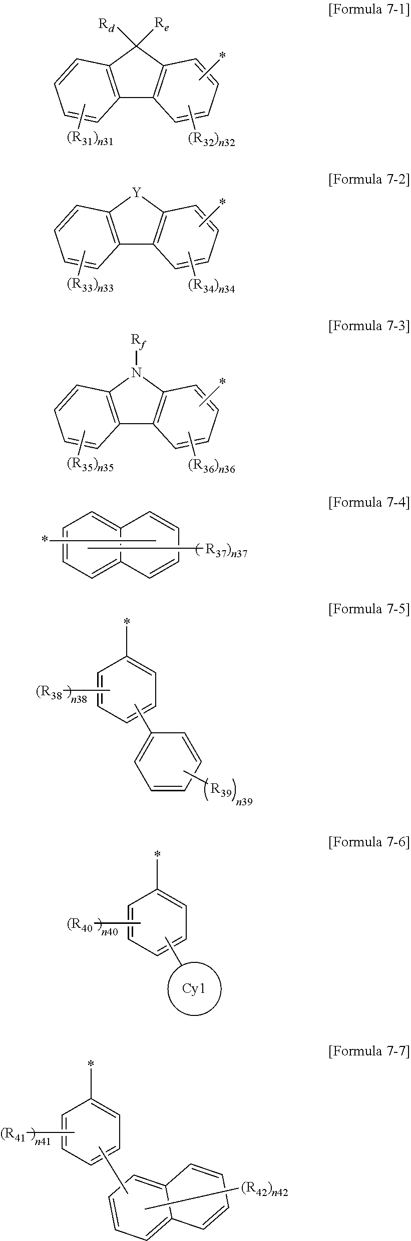
wherein in Formula 7-1 to Formula 7-7,
Y is O or S,
R₃₁ to R₄₂ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
R_{d} to R_{f} are each independently a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring,
ring Cy1 is a substituted or unsubstituted cycloalkyl group having 3 to 20 ring-forming carbon atoms,
n31, n33, n35, n38, n40, and n41 are each independently an integer from 0 to 4,
n32, n34 and n36 are each independently an integer from 0 to 3,
n37 and n42 are each independently an integer from 0 to 7,
n39 is an integer from 0 to 5, and
-* represents a bonding site to a nitrogen atom in Formula 1.

10. The light emitting device of claim 1, wherein the amine compound represented by Formula 1 is represented by Formula 8-1 or Formula 8-2: 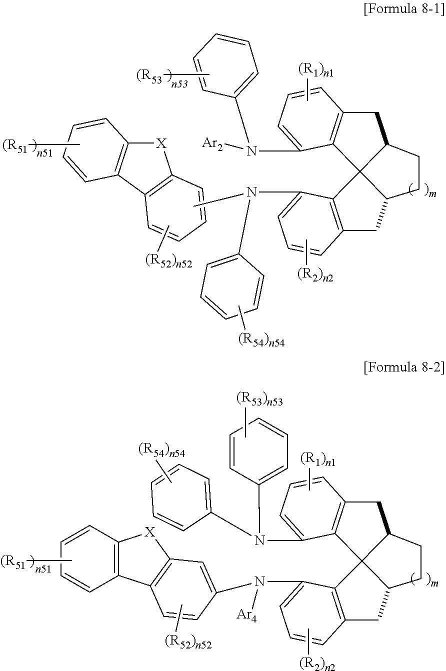
wherein in Formula 8-1 and Formula 8-2,
X is N(R₅₅), C(R₅₆)(R₅₇), O, or S,
R₅₁ to R₅₇ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and
Ar₂, Ar₄, R₁, R₂, n1, n2, and m are the same as defined in Formula 1.

11. The light emitting device of claim 1, wherein the amine compound represented by Formula 1 is represented by one of Formula 9-1 to Formula 9-4: 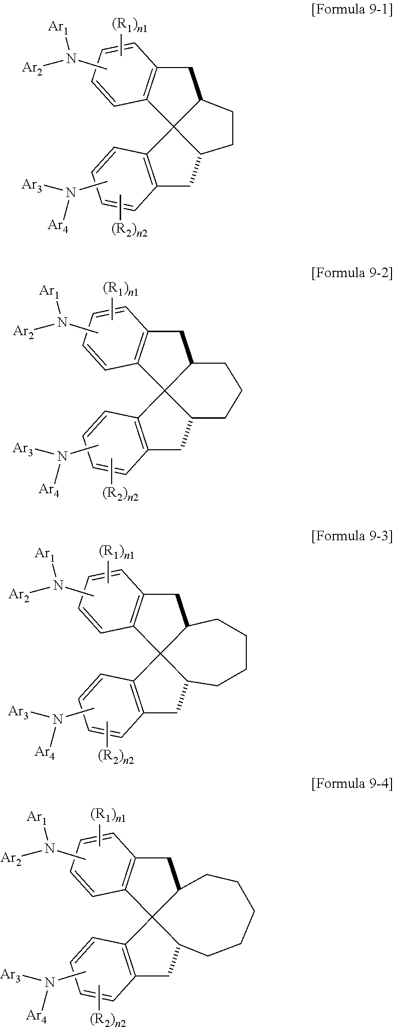
wherein in Formula 9-1 to Formula 9-4,
Ar₁ to Ar₄, R₁, R₂, n1, and n2 are the same as defined in Formula 1.

12. The light emitting device of claim 1, wherein the amine compound comprises at least one compound selected from Compound Group 1:

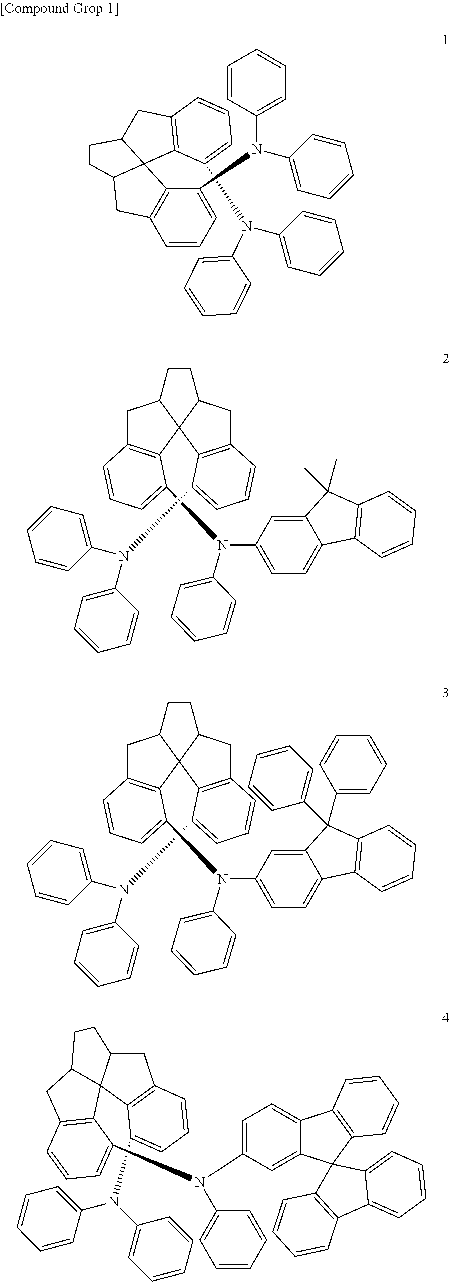

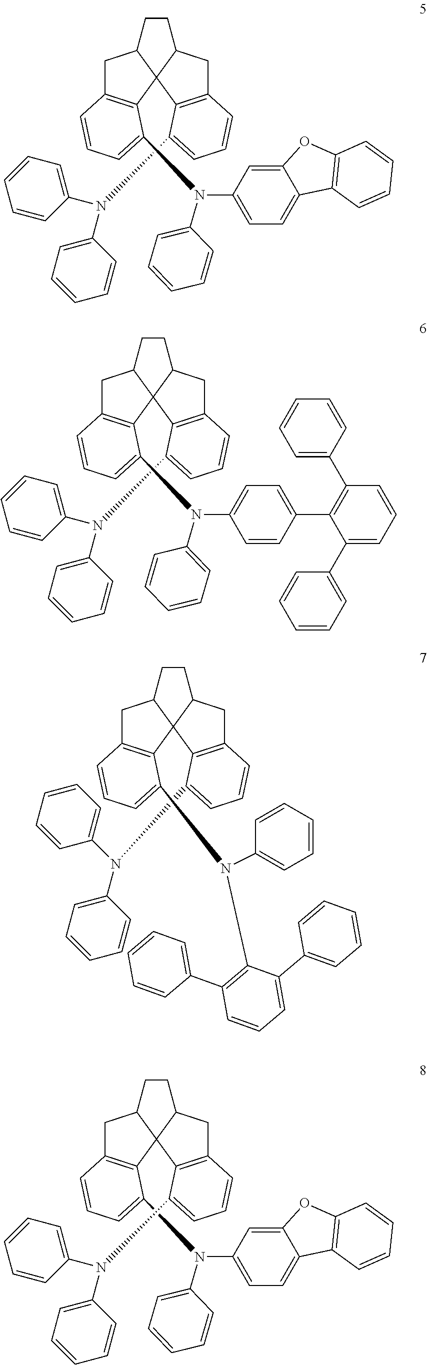

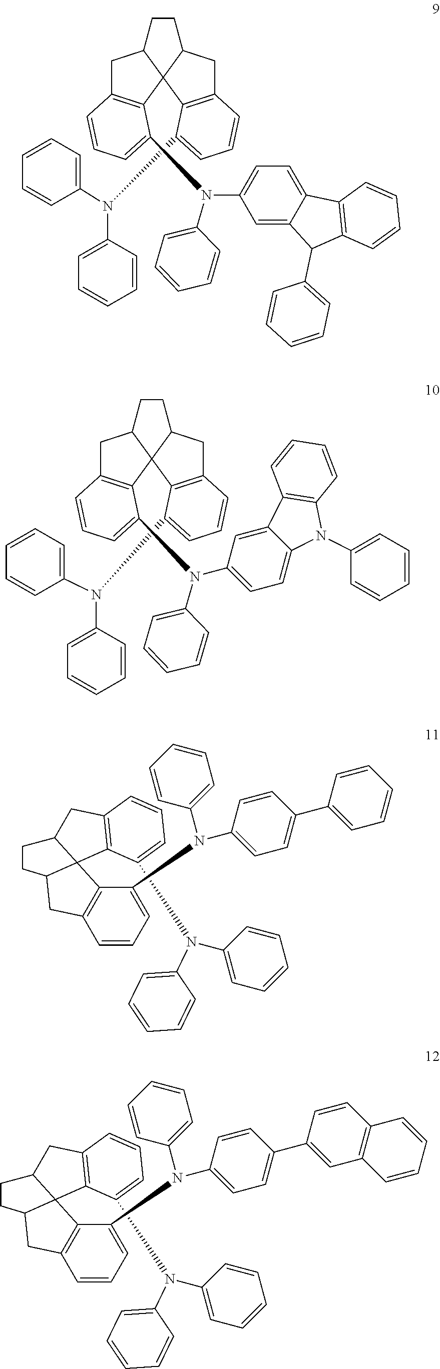

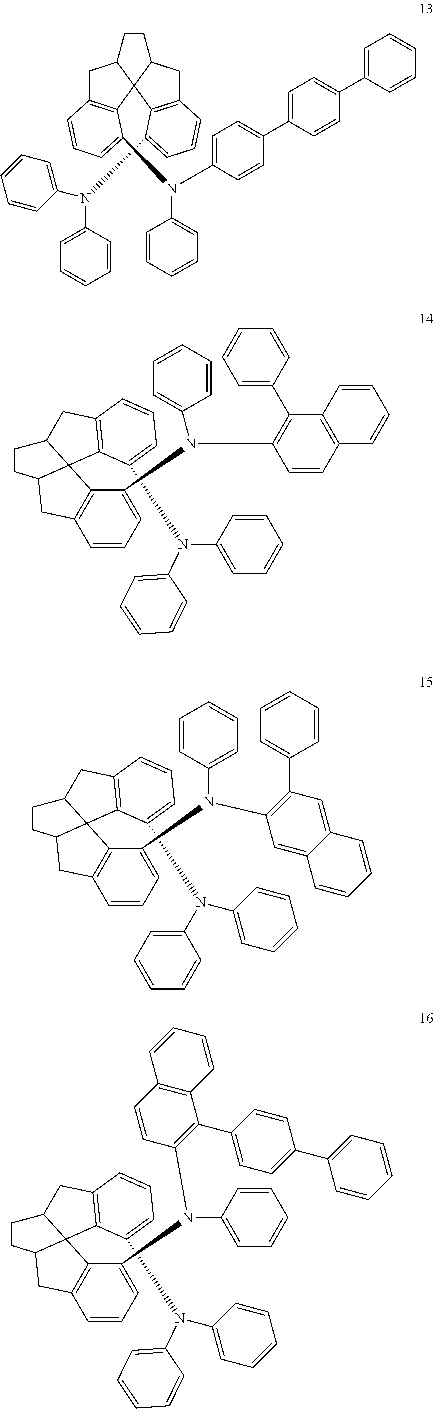

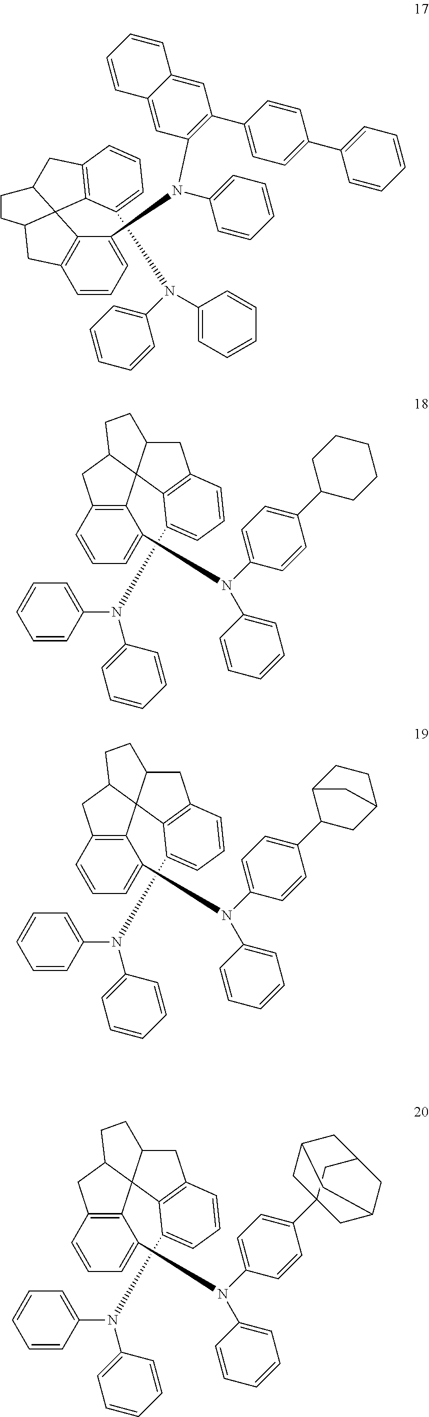

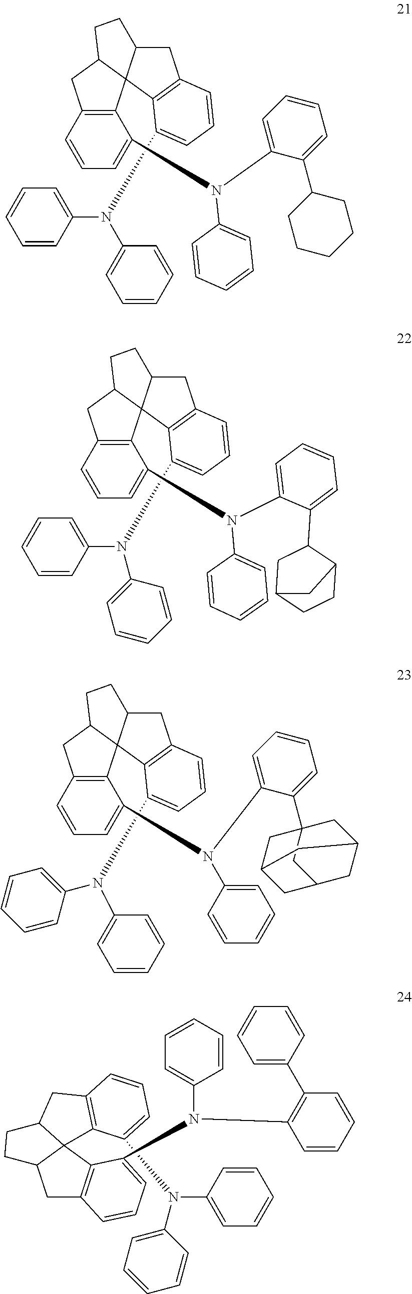

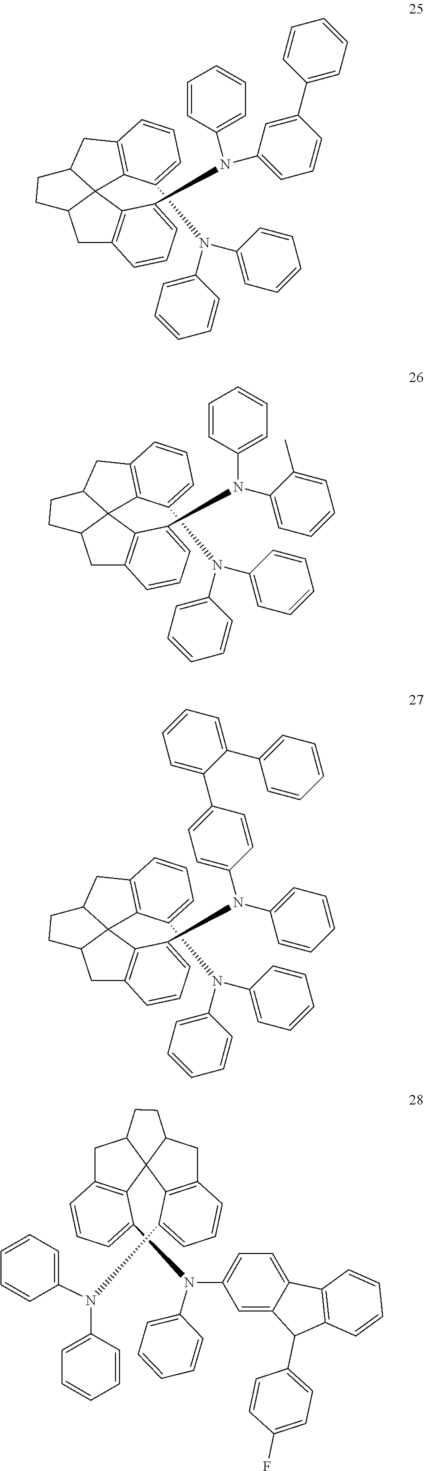

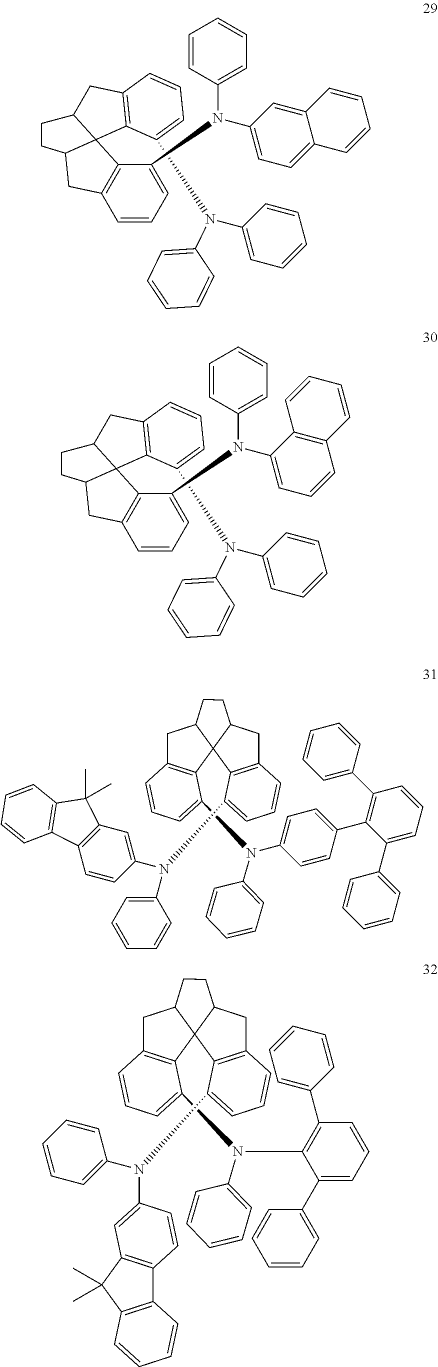

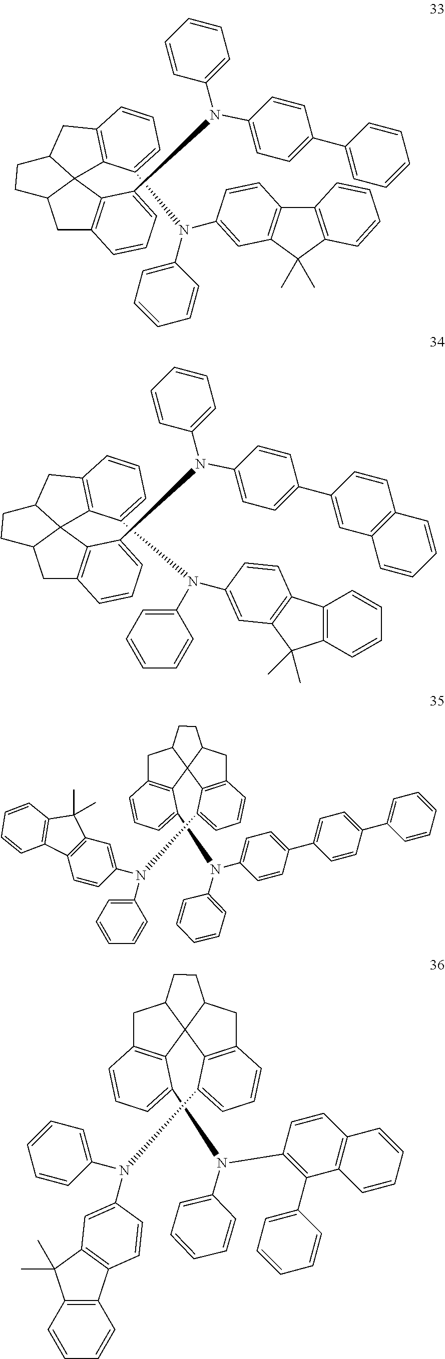

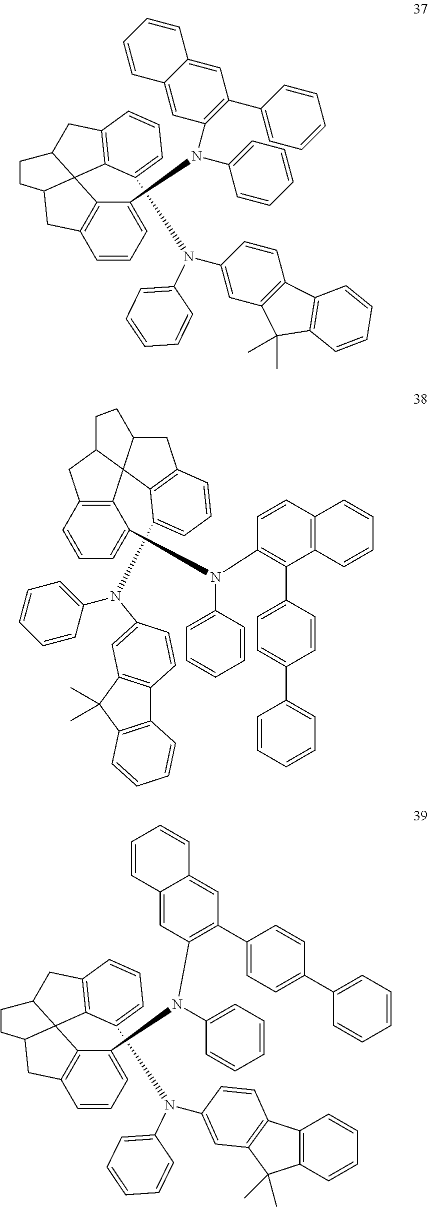

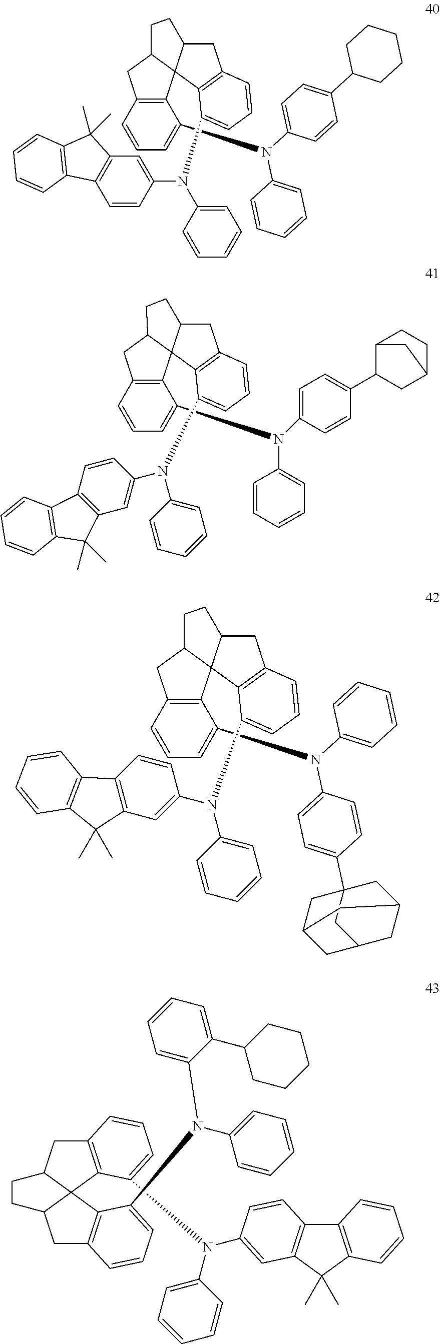

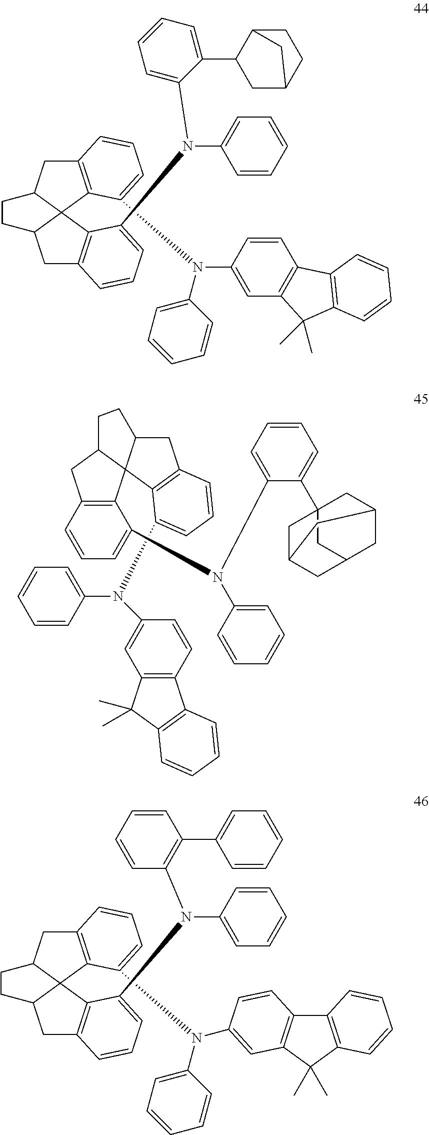

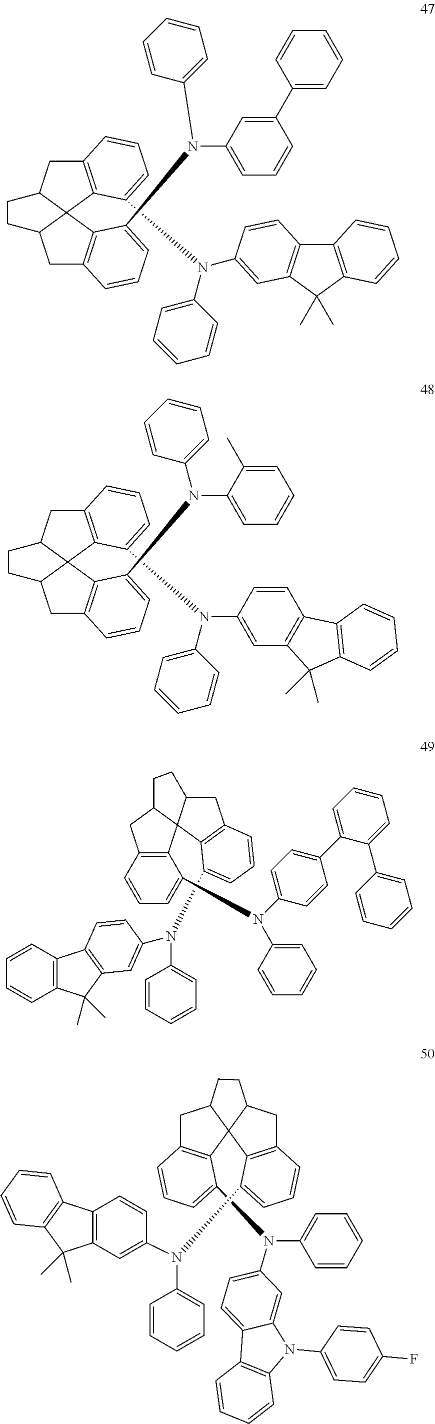

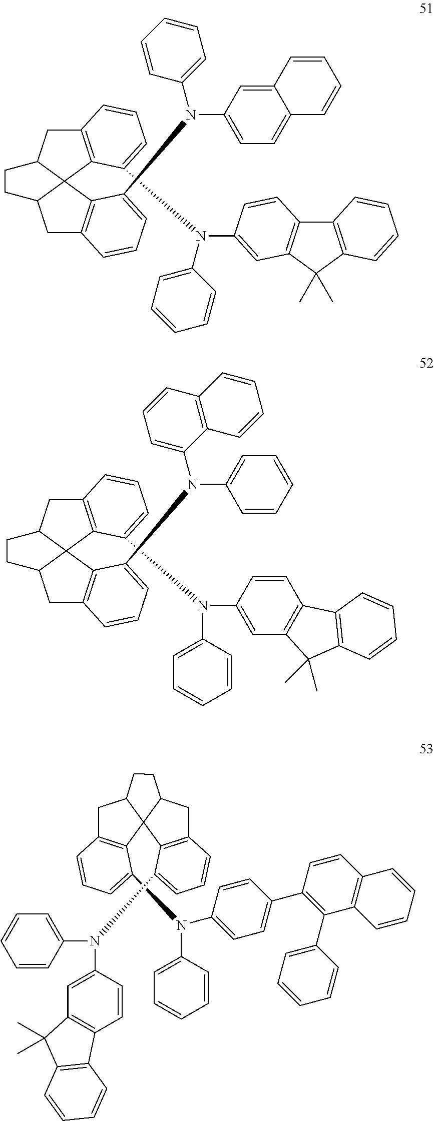

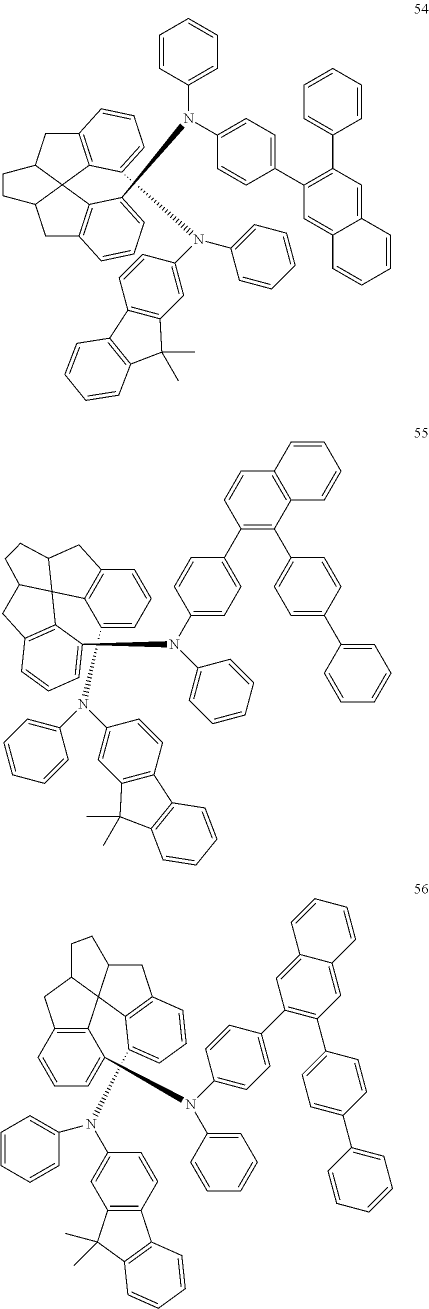

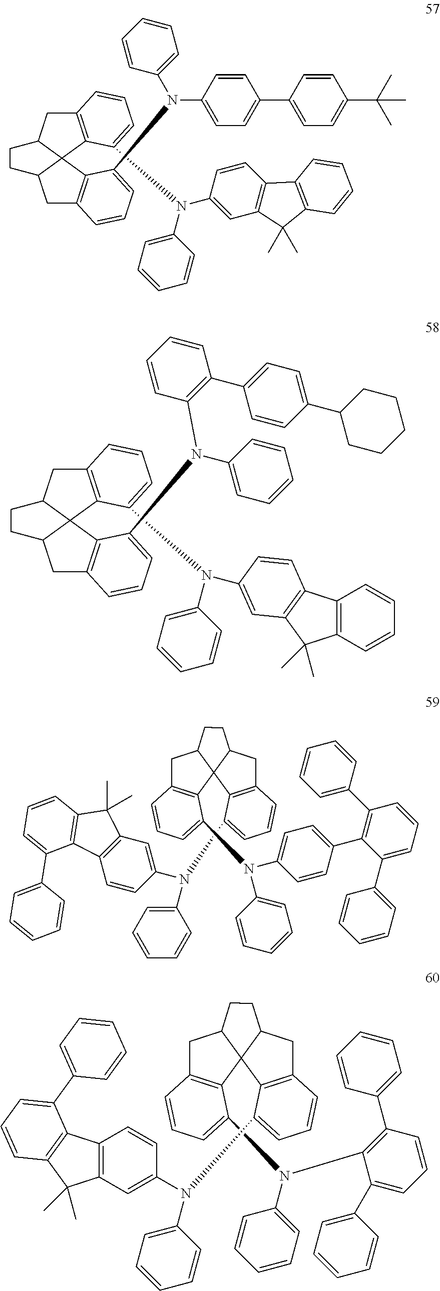

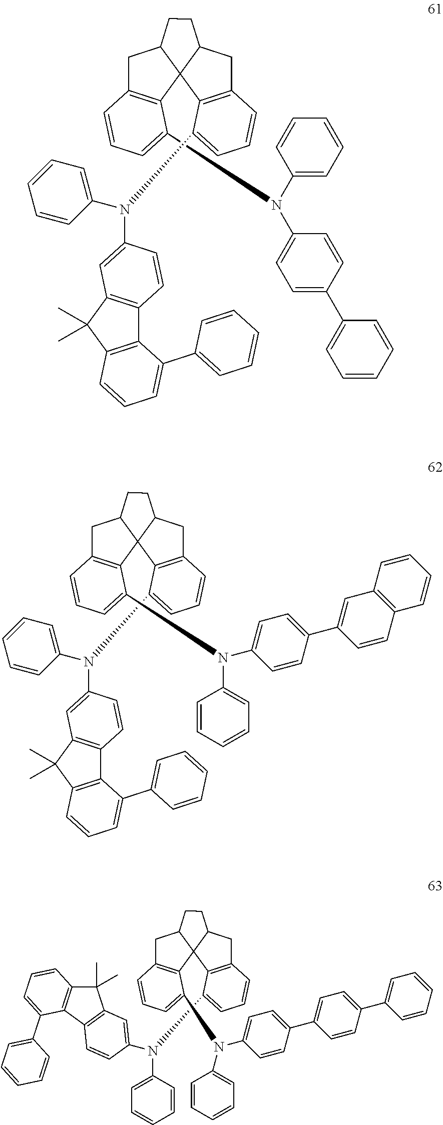

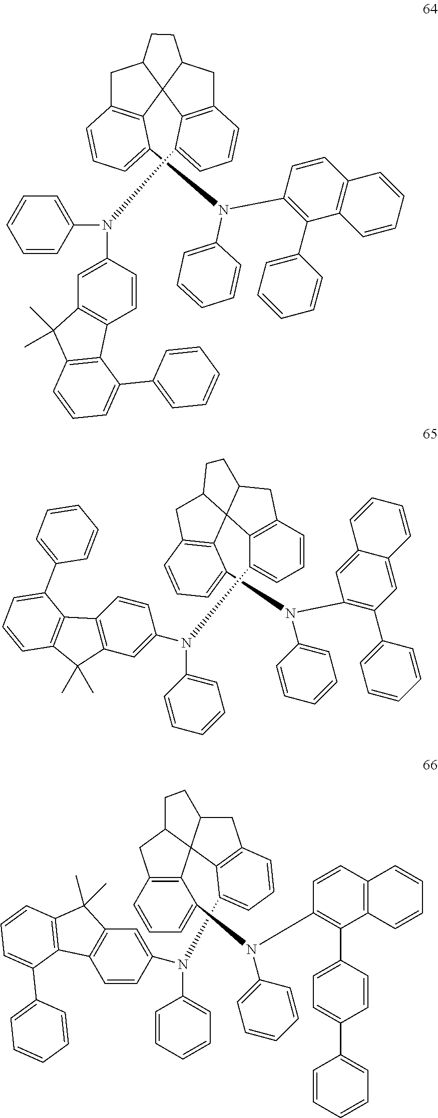

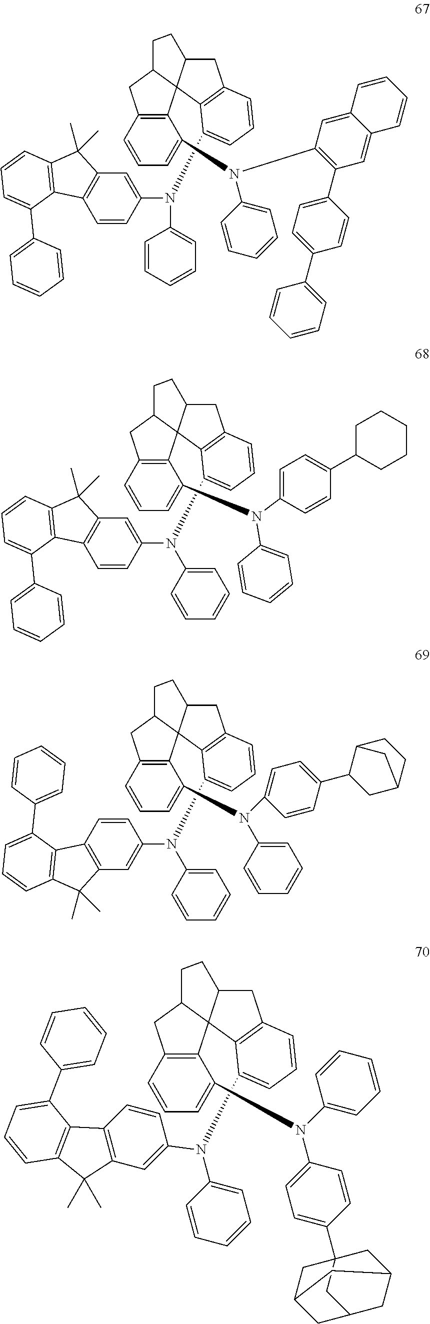

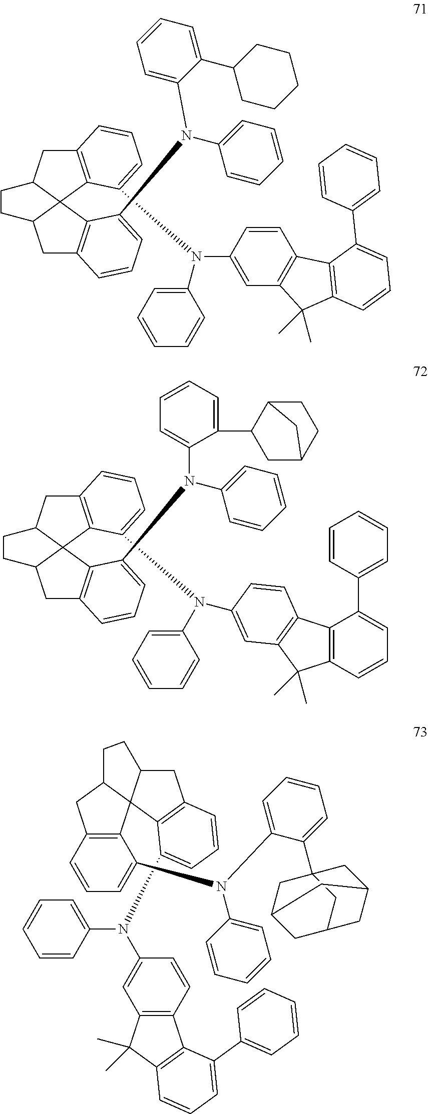

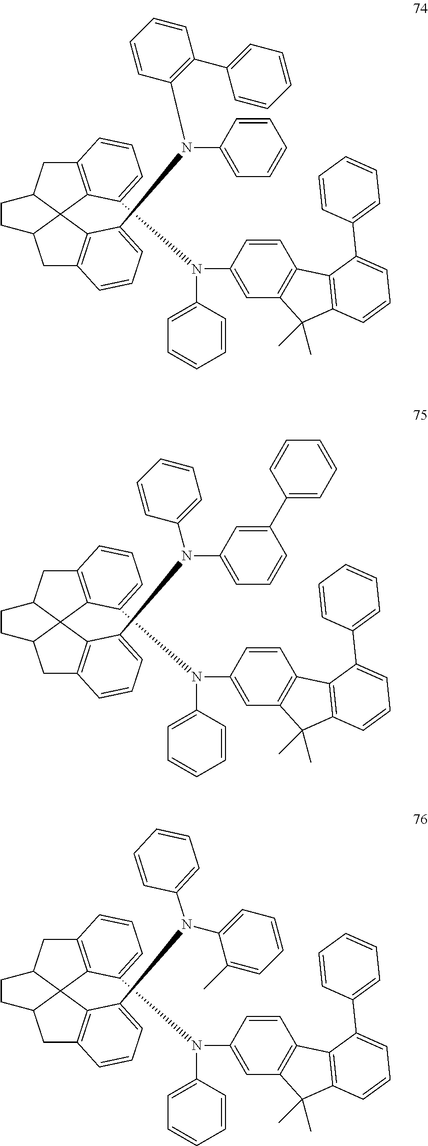

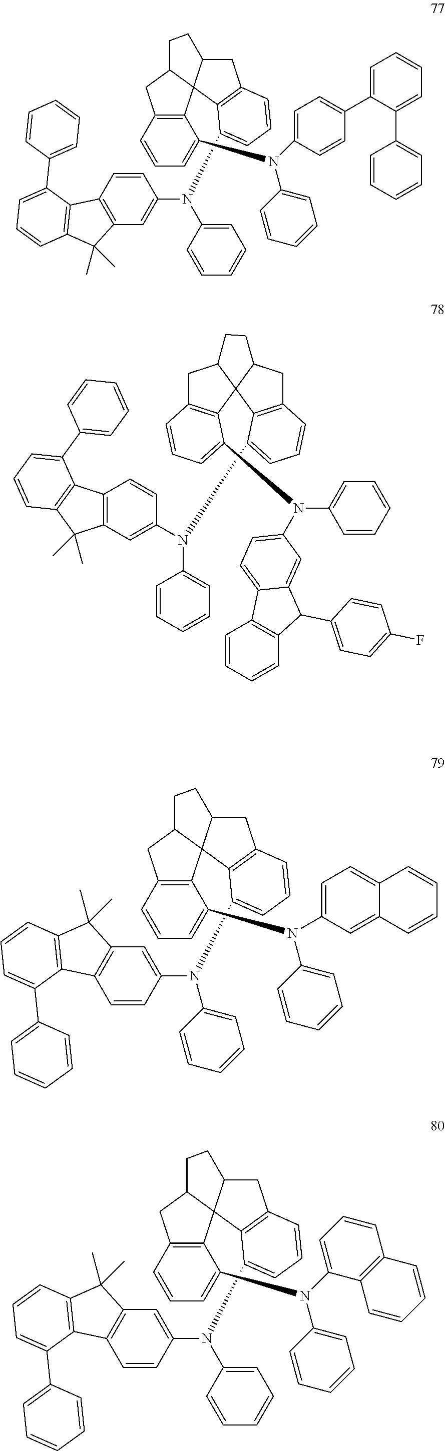

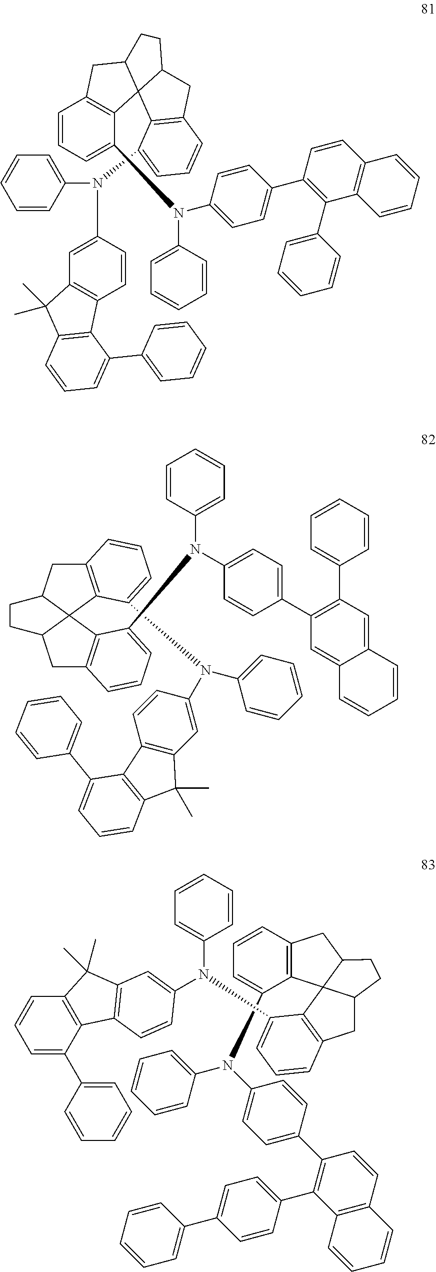

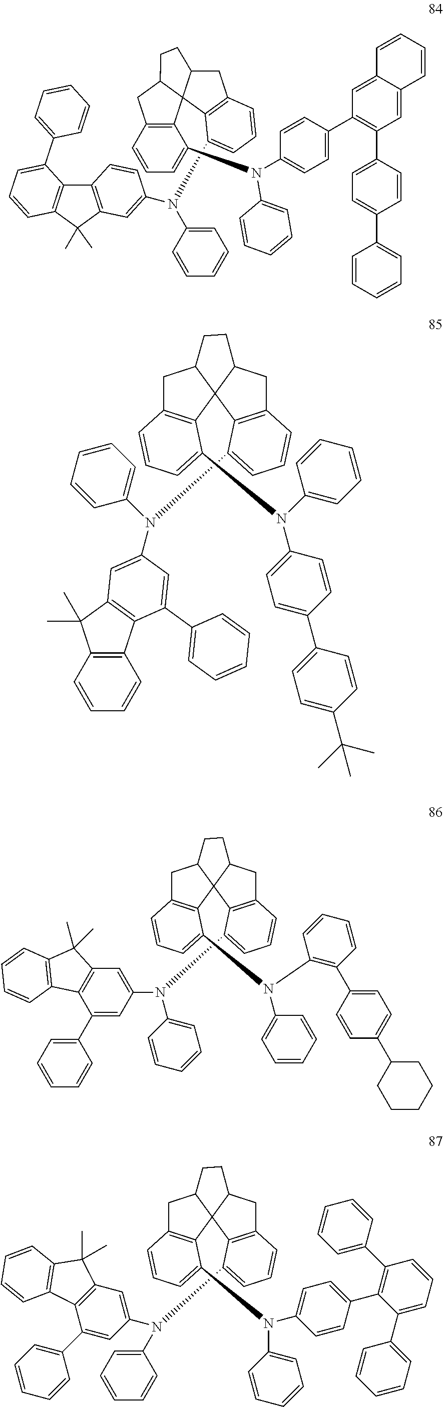

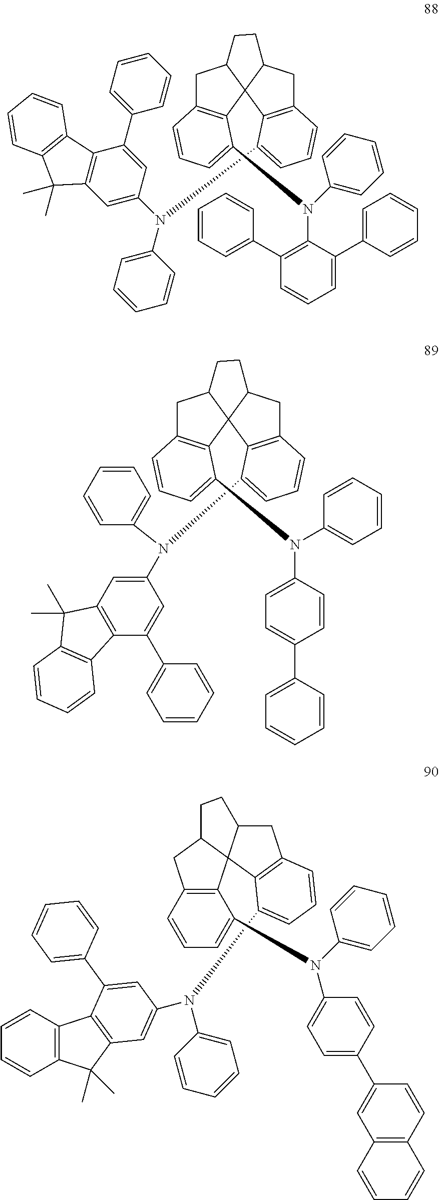

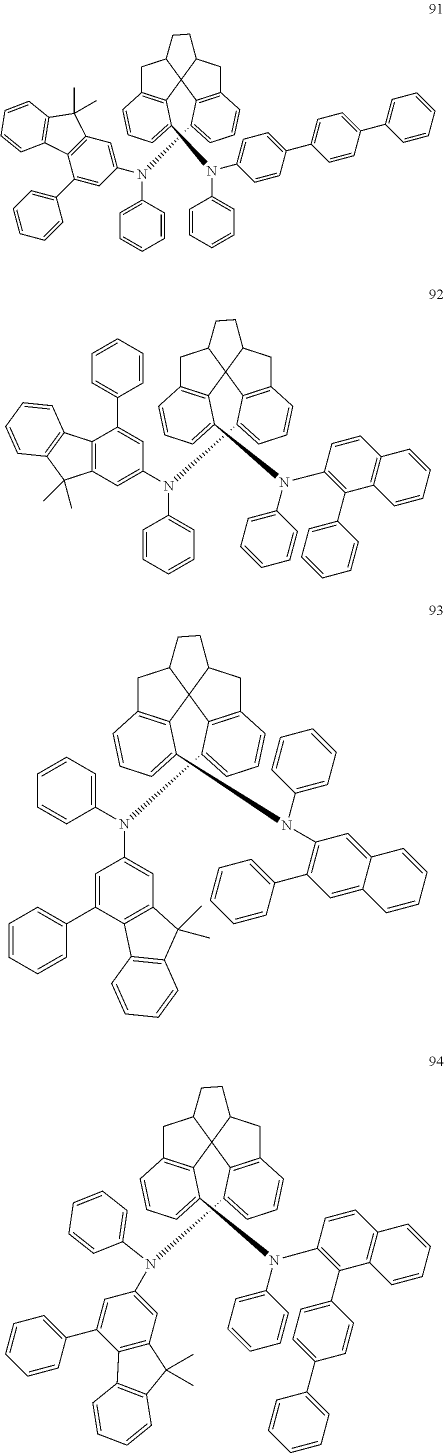

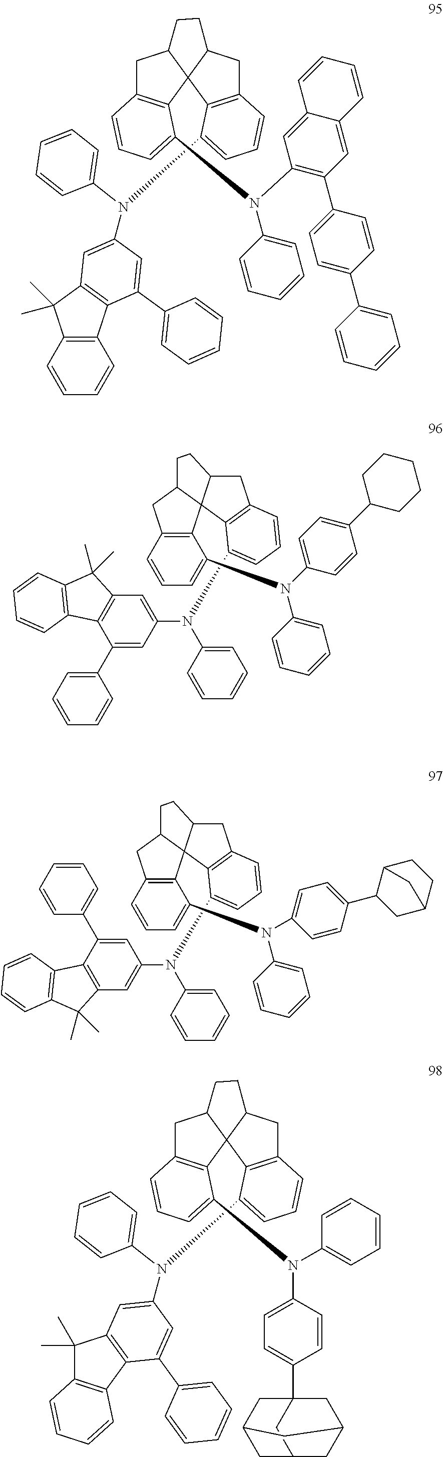

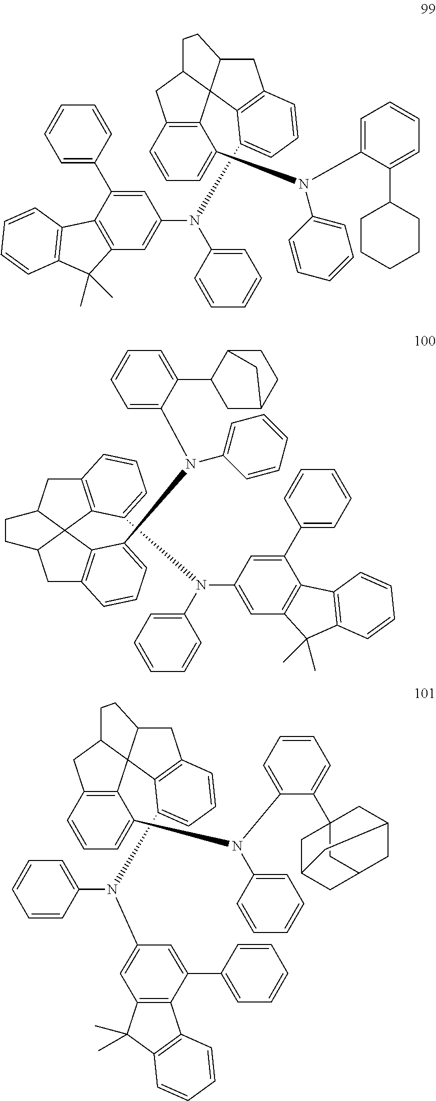

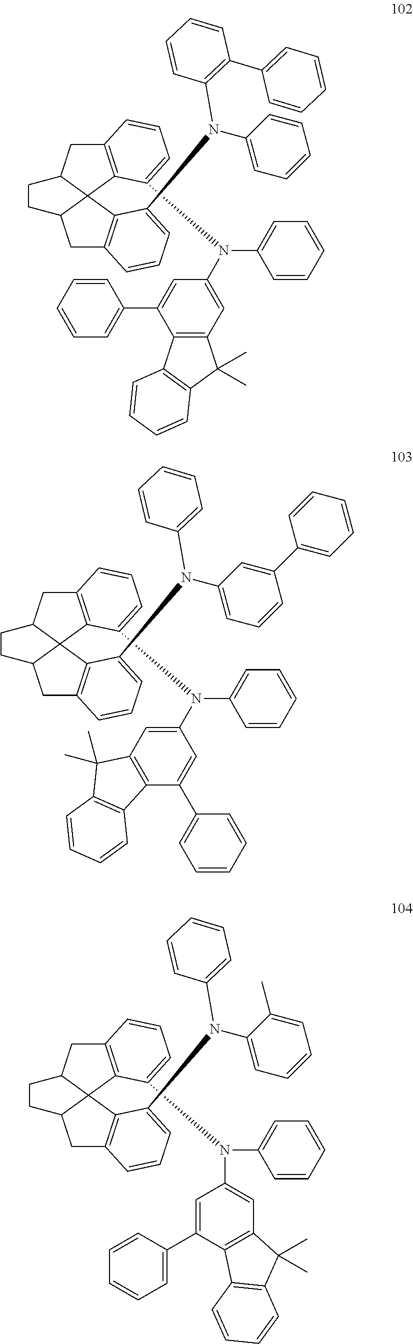

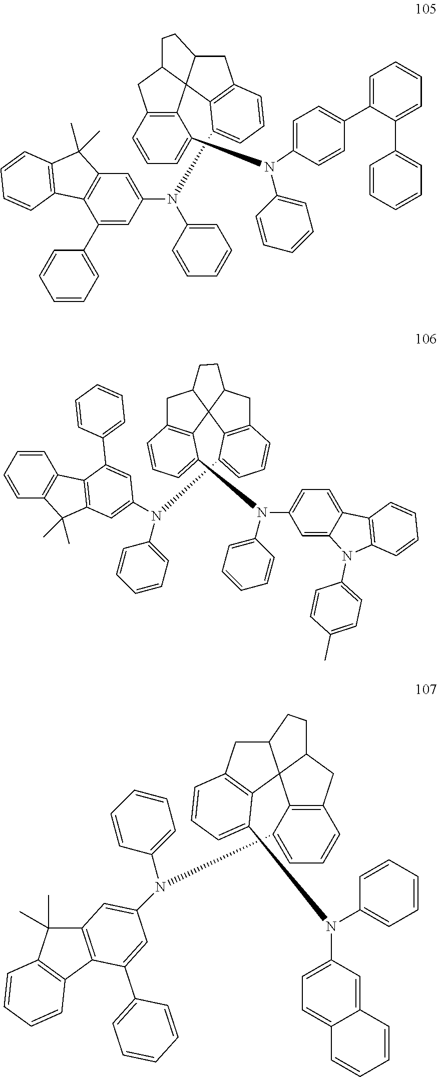

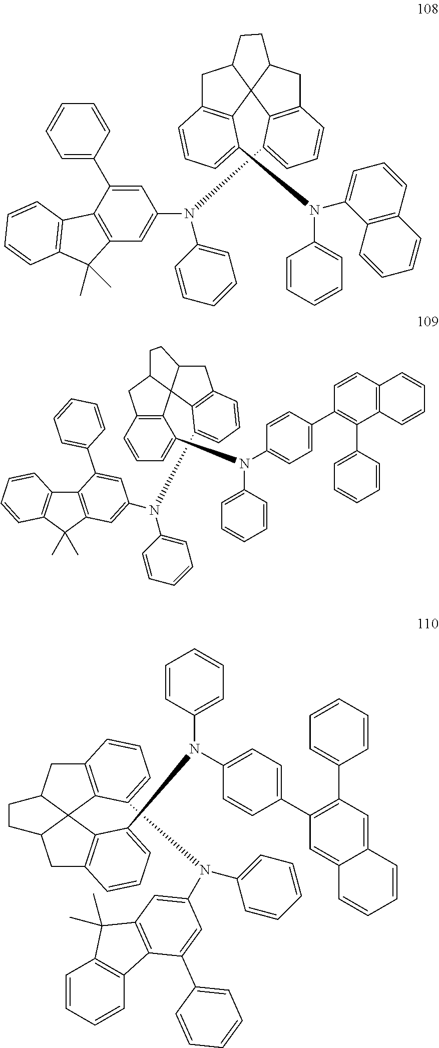

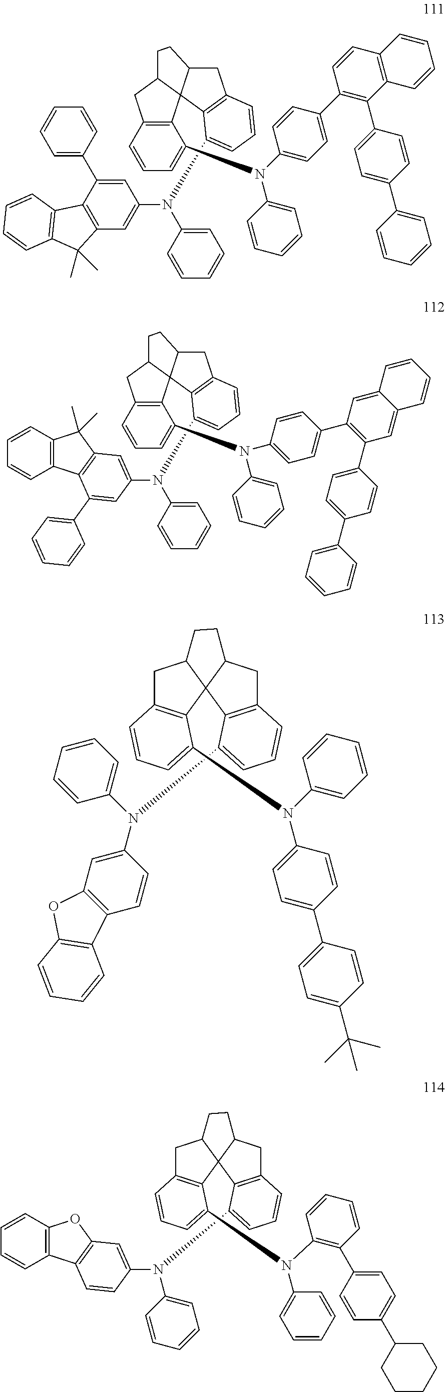

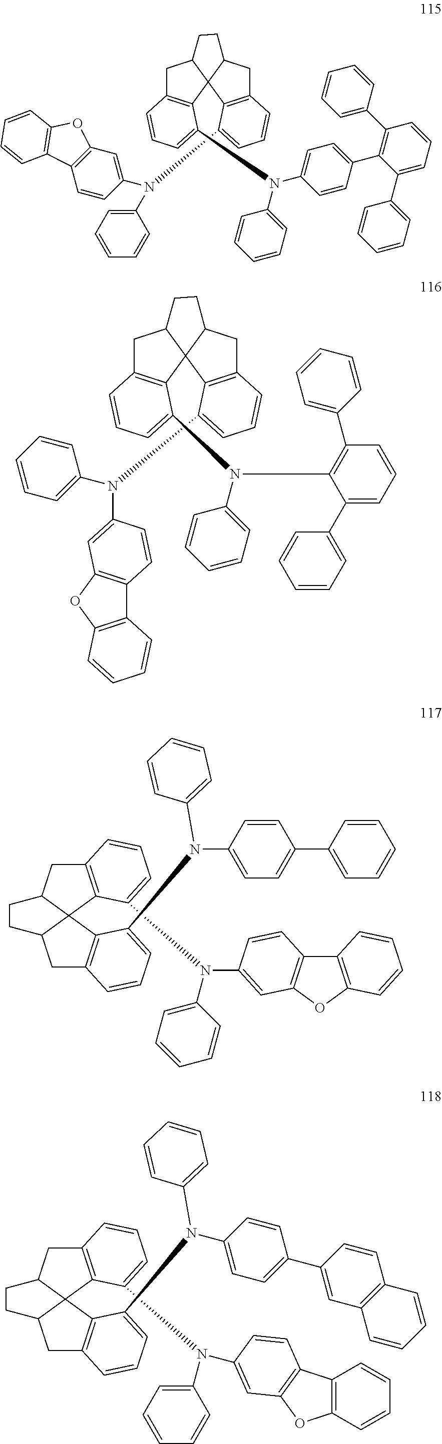

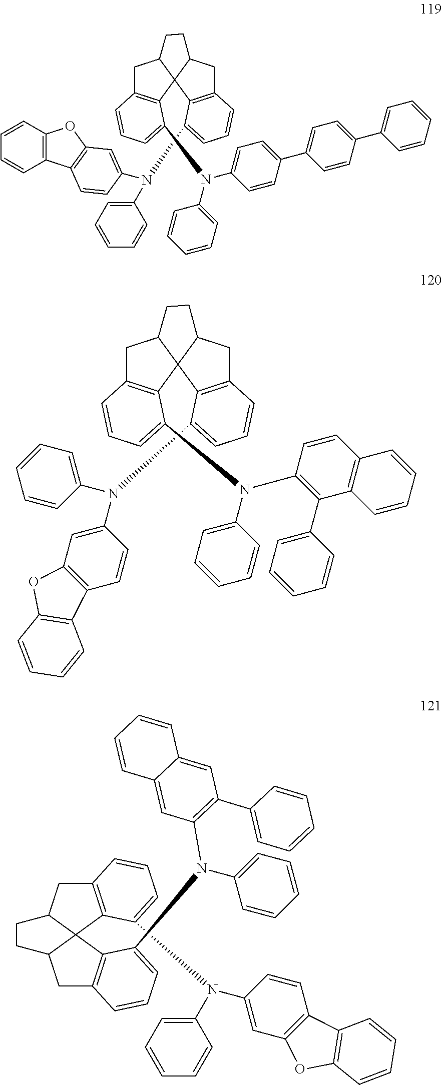

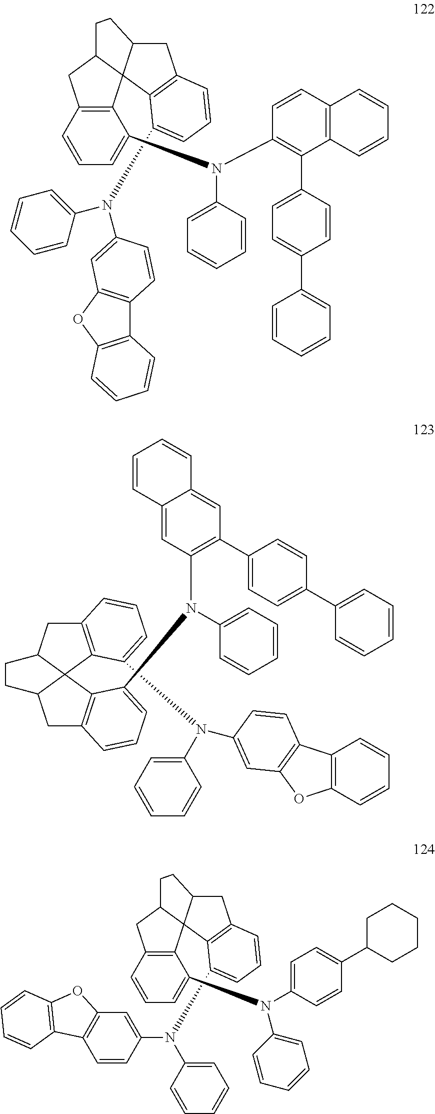

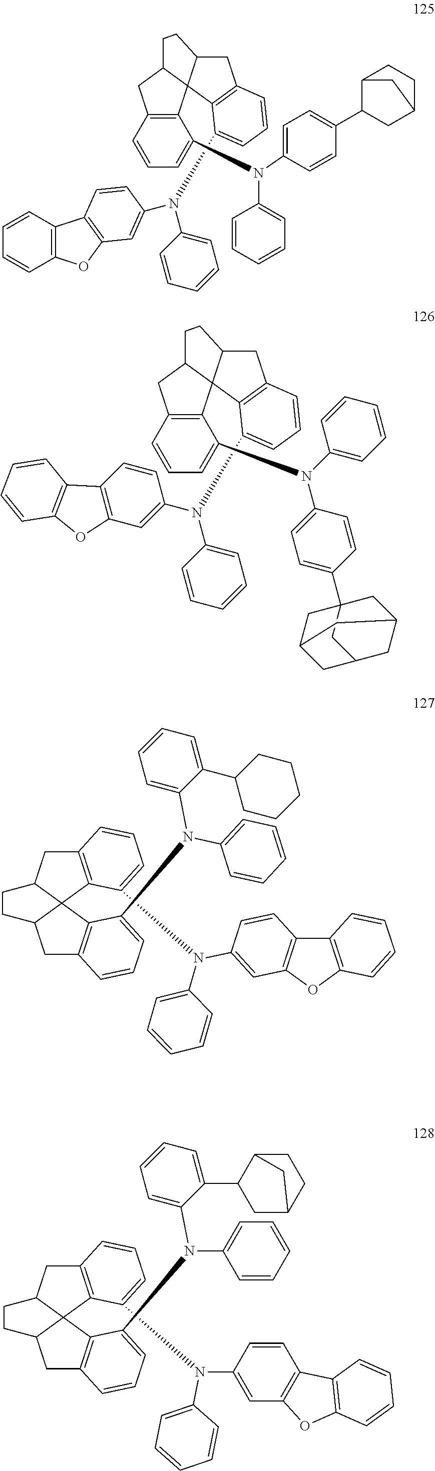

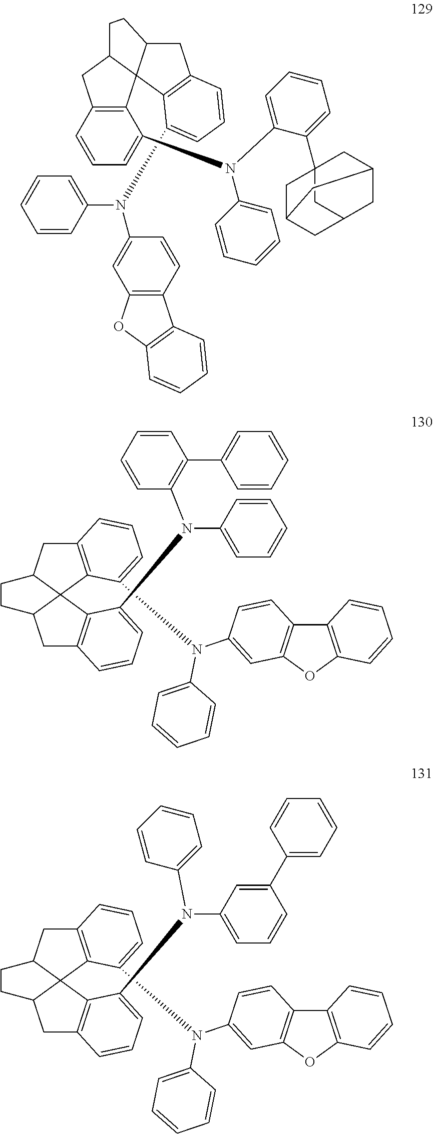

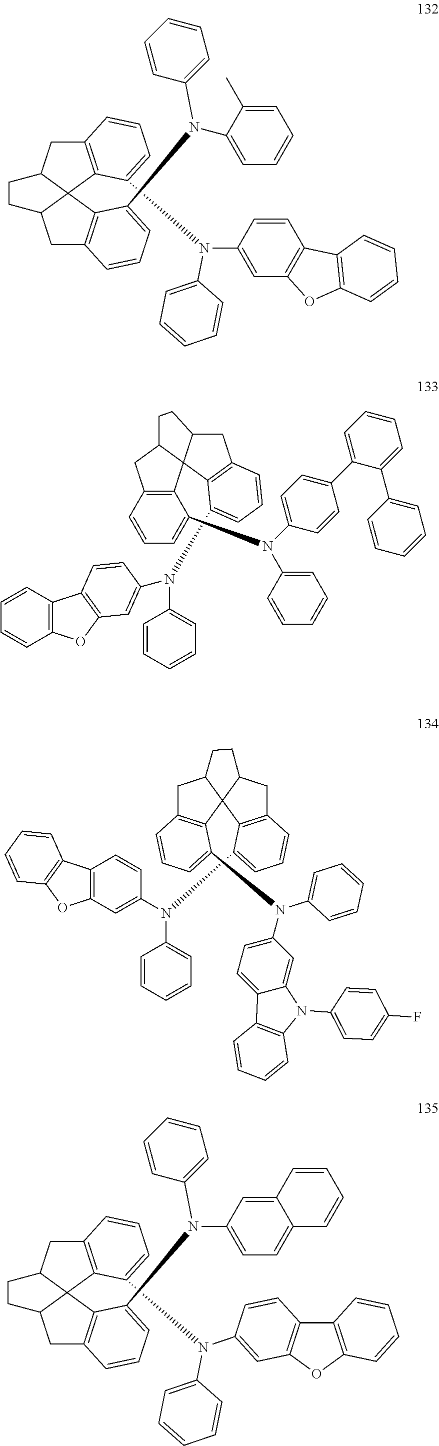

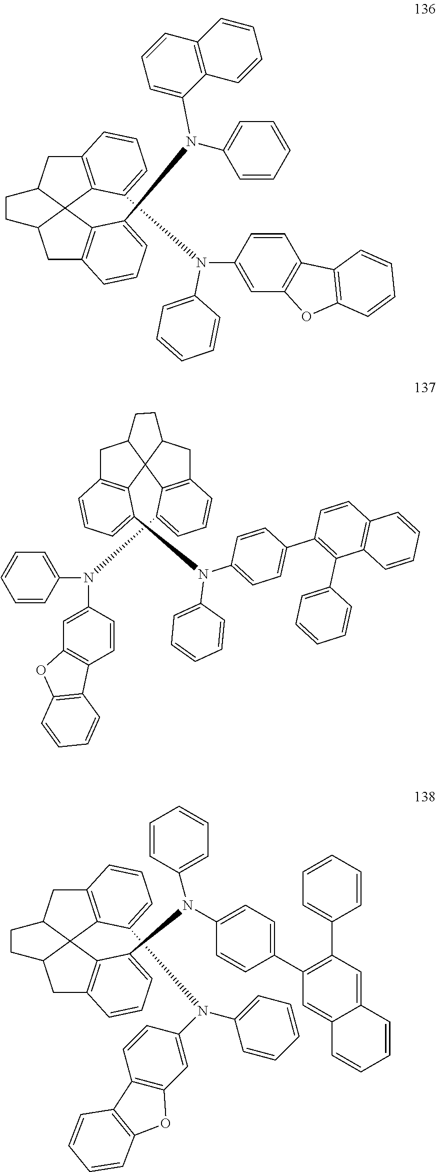

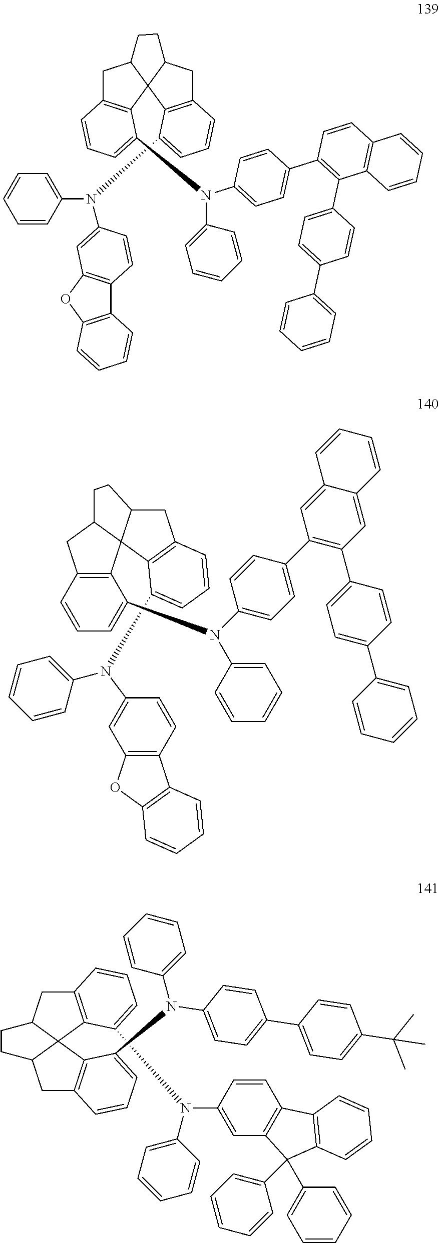

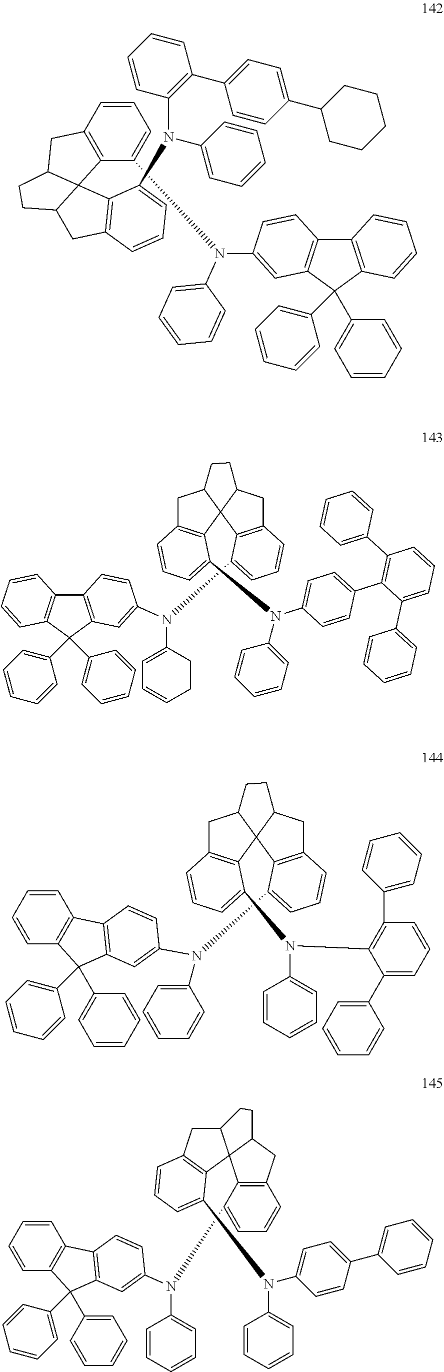

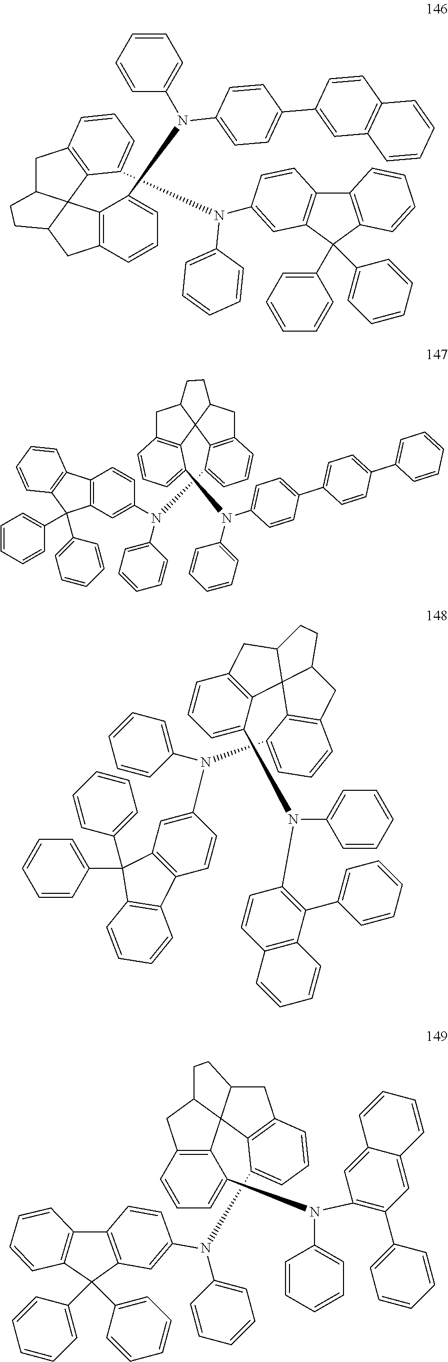

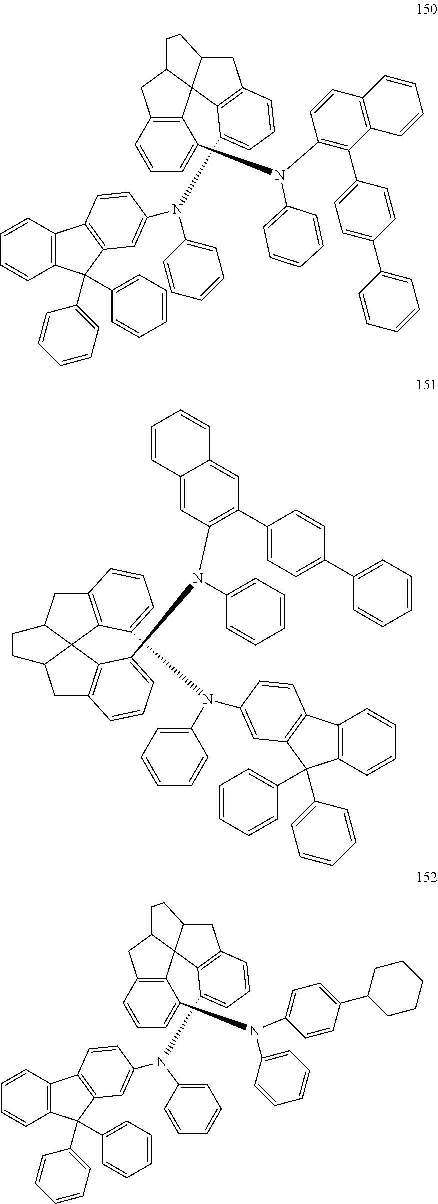

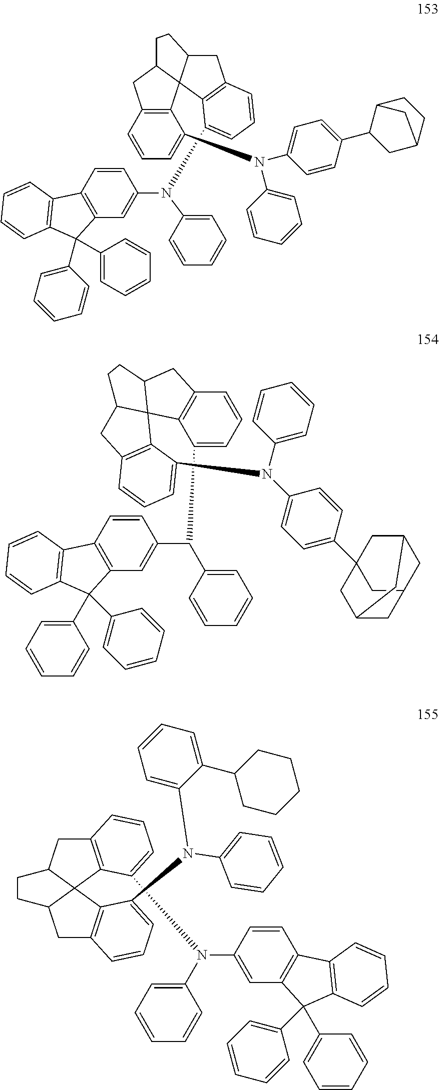

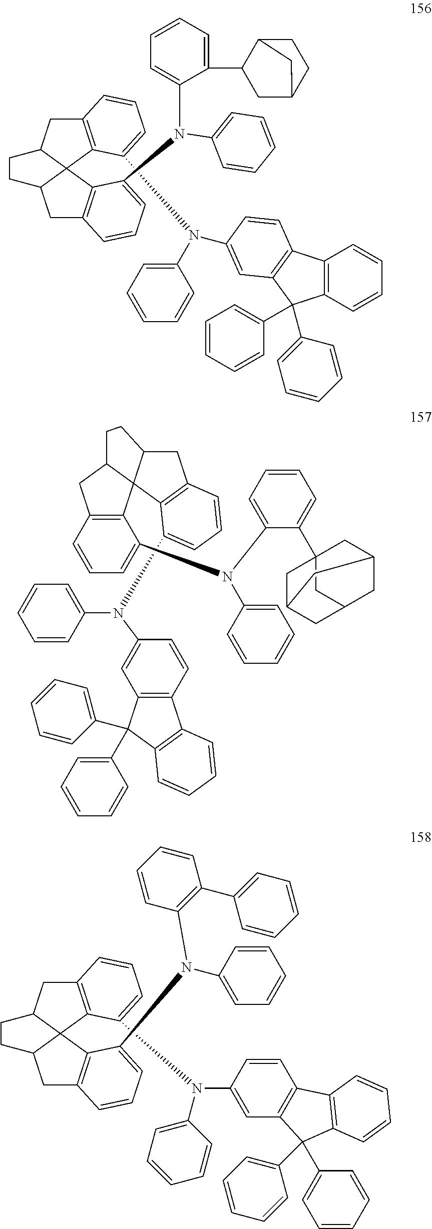

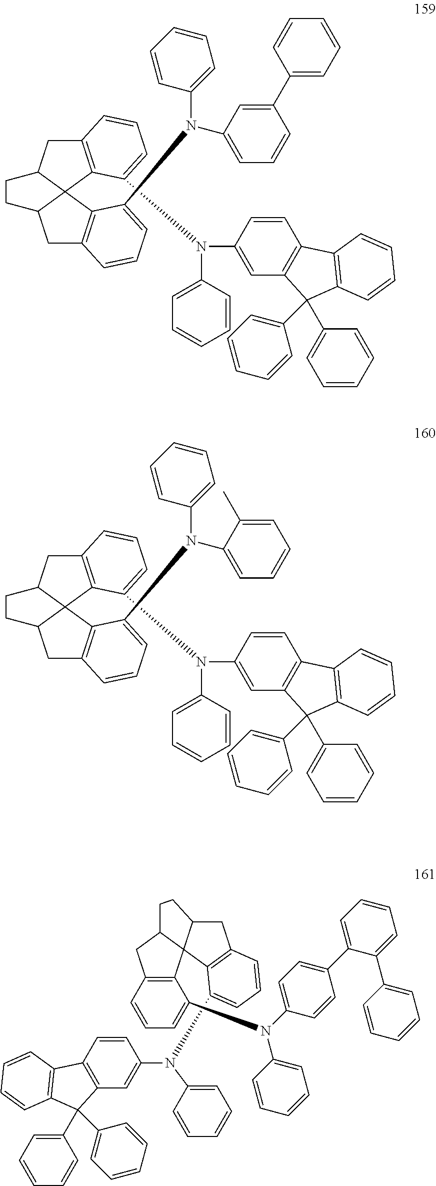

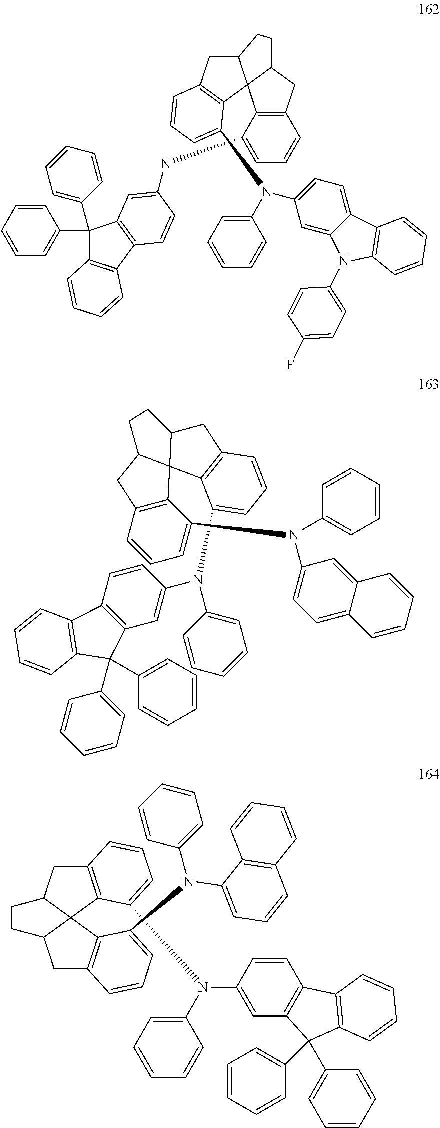

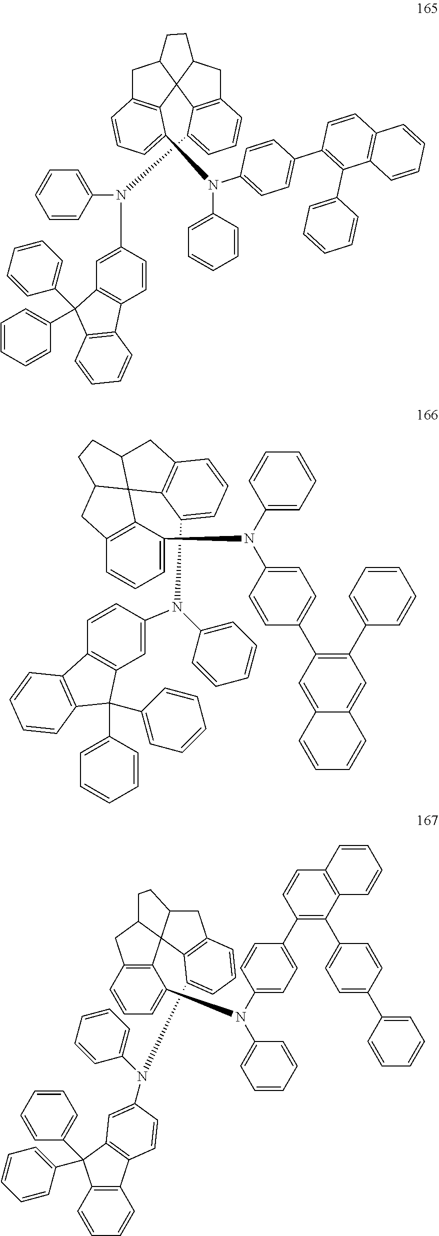

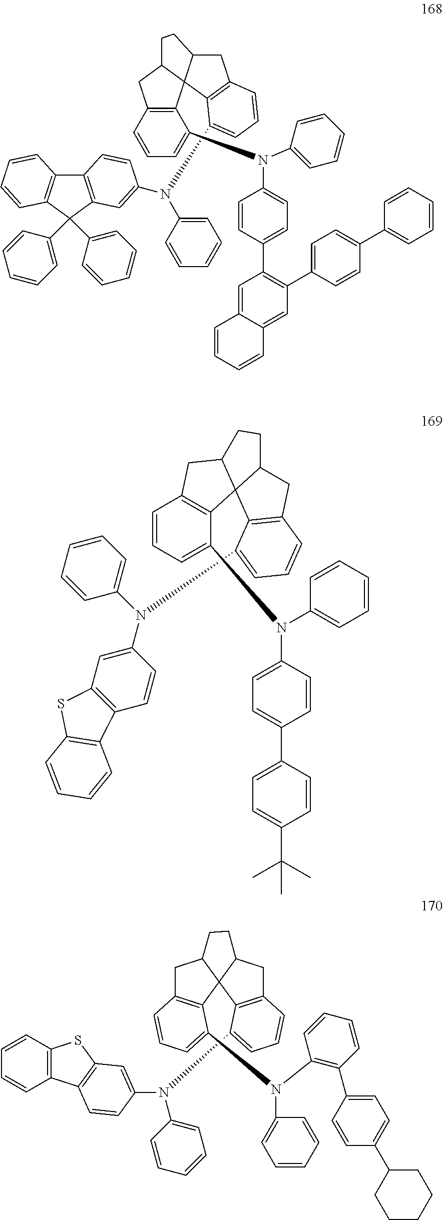

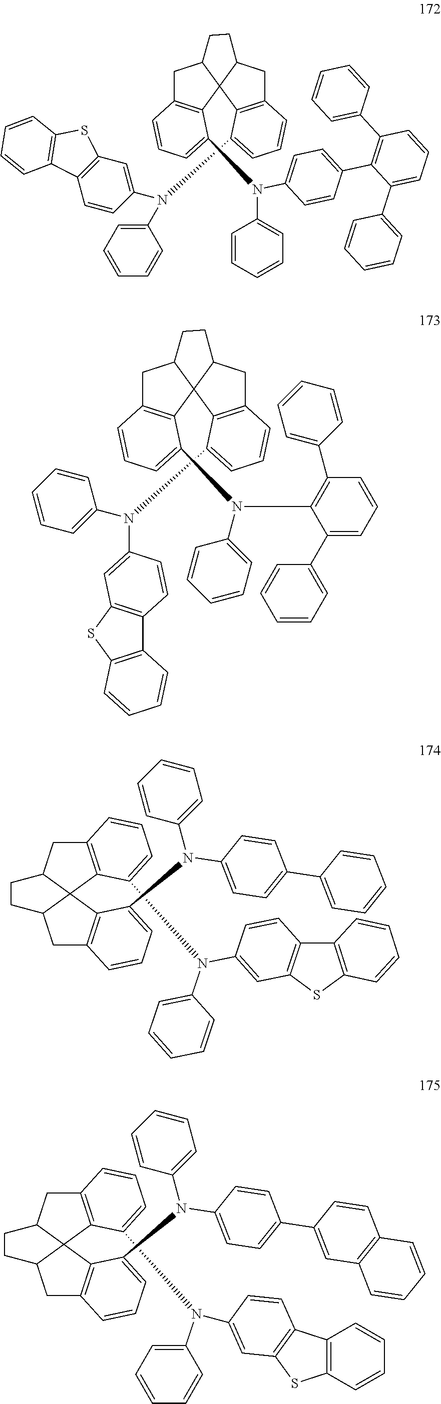

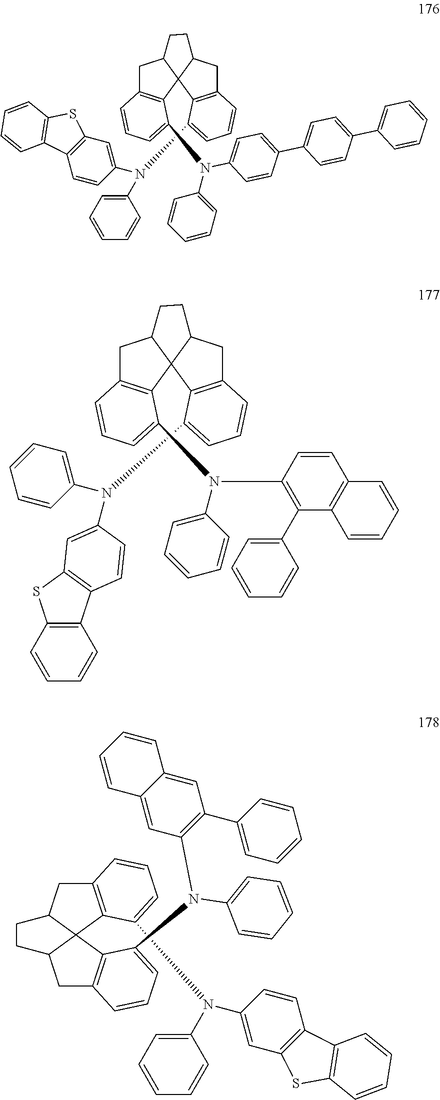

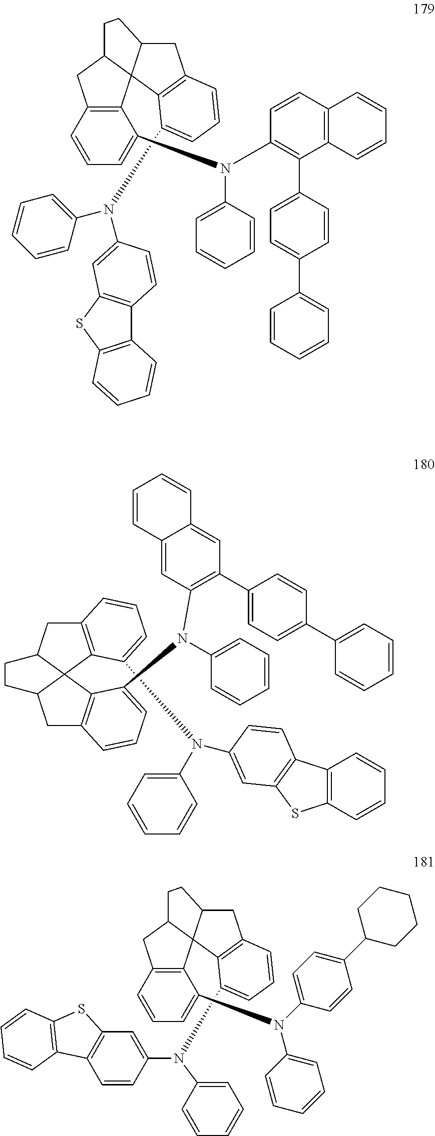

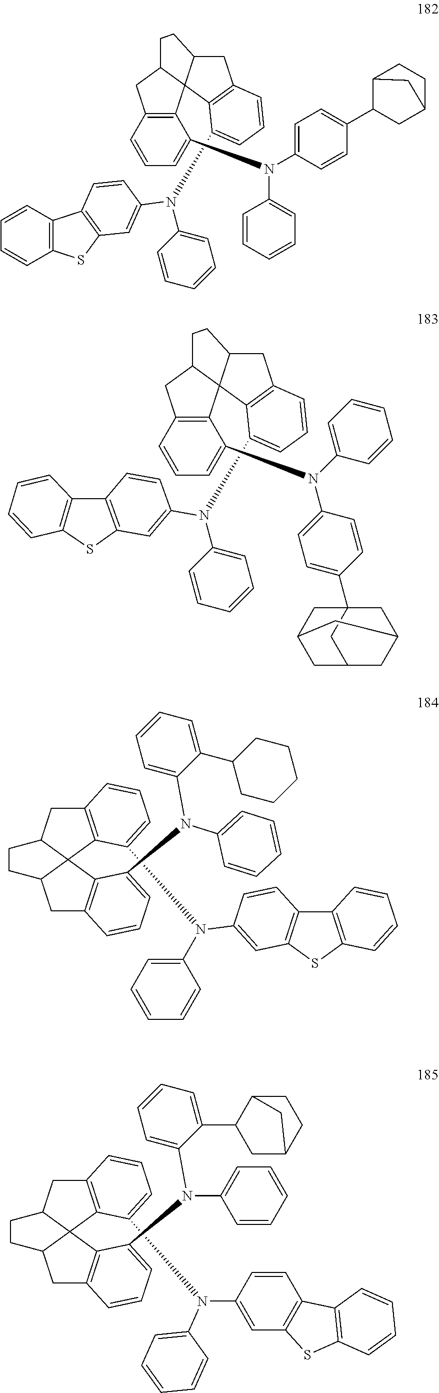

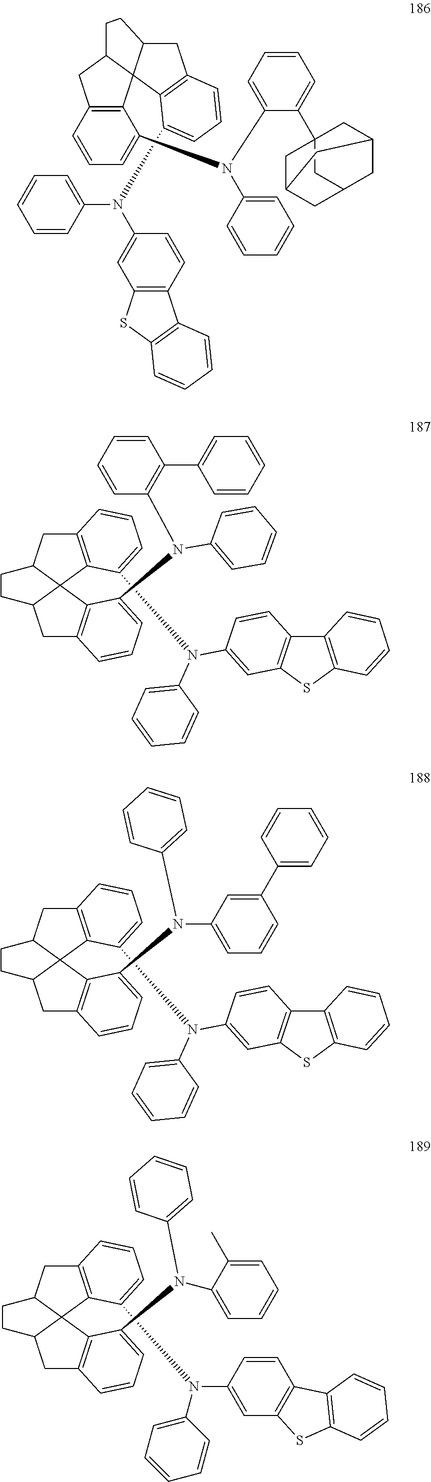

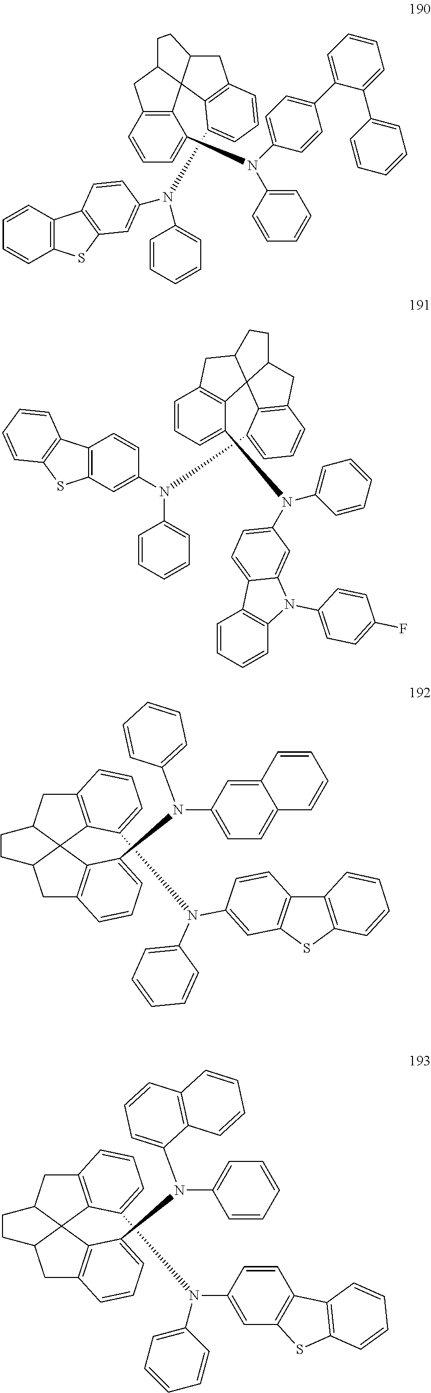

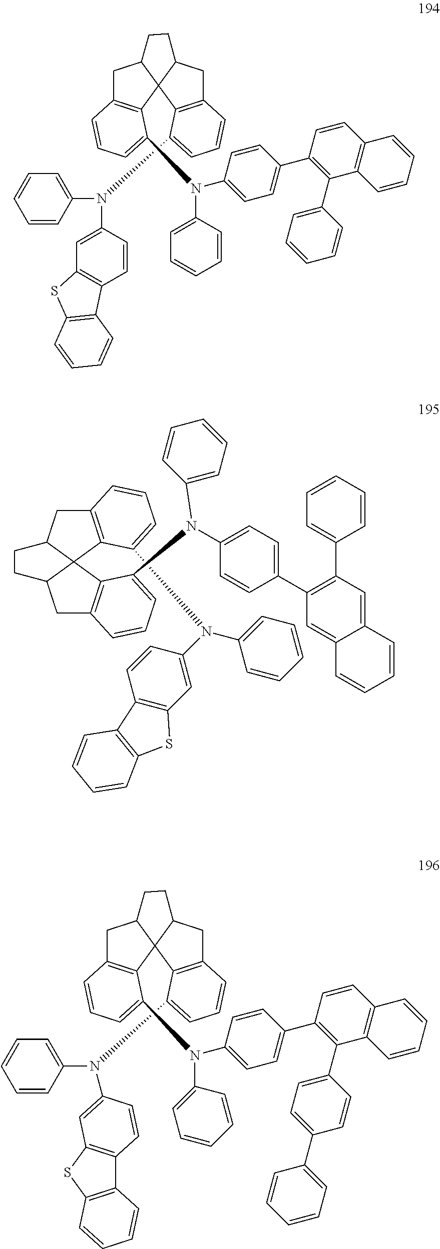

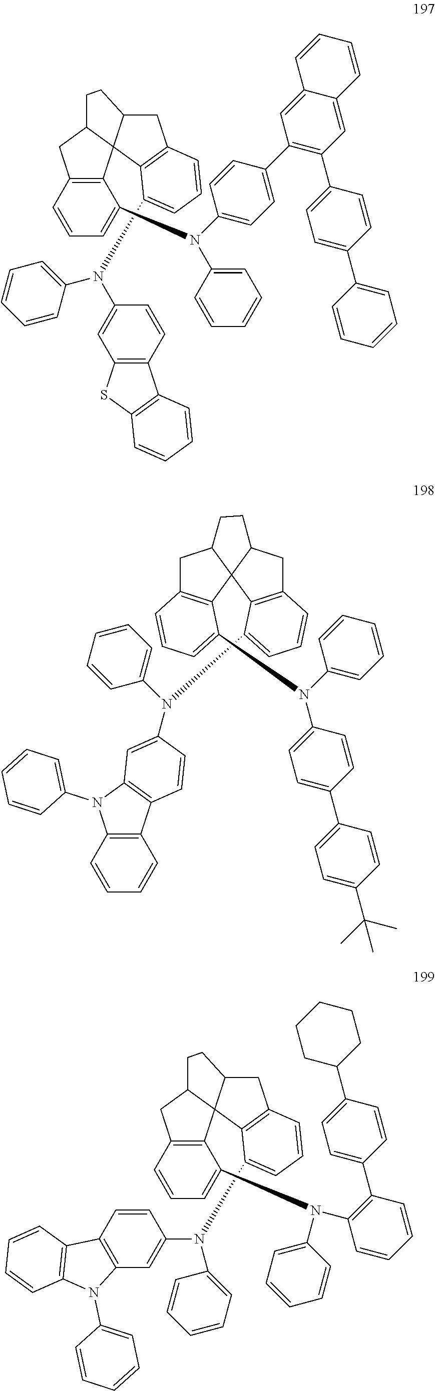

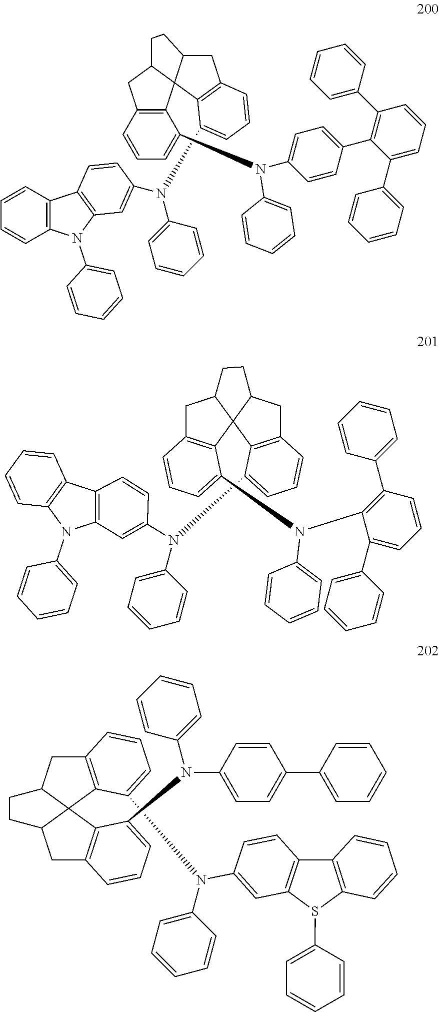

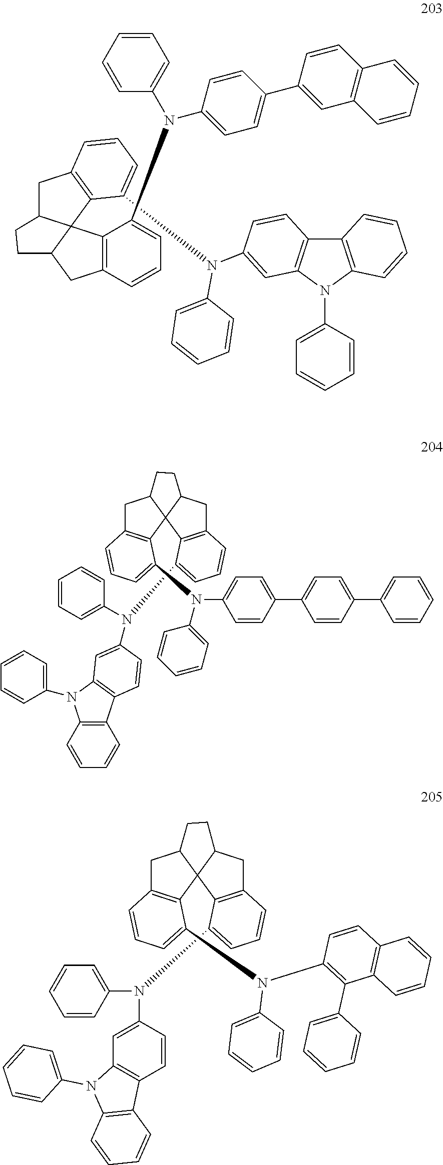

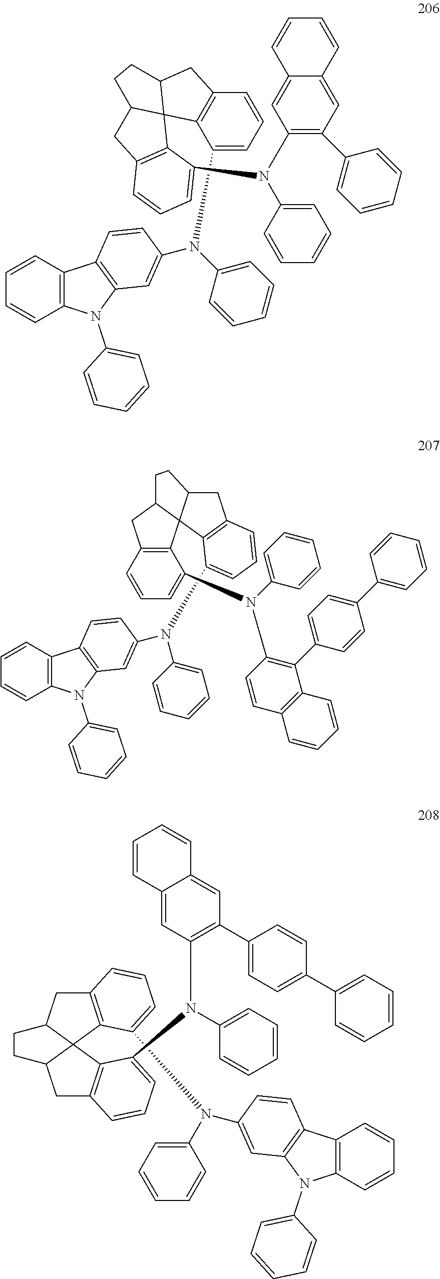

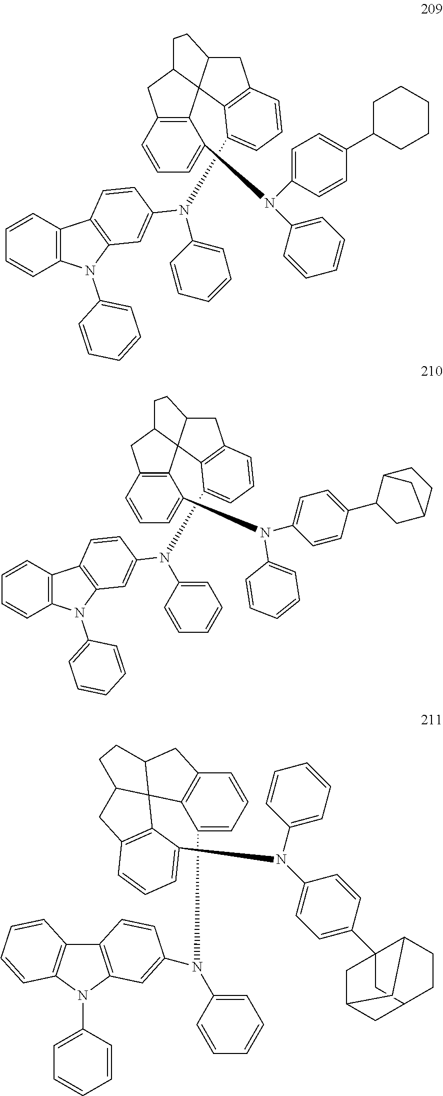

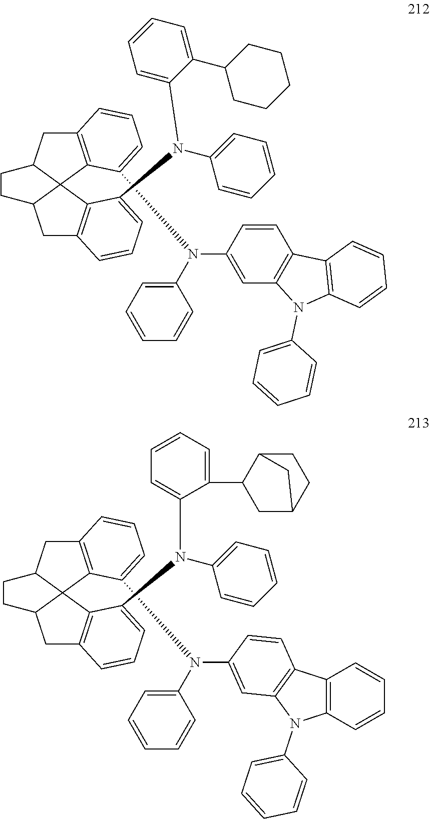

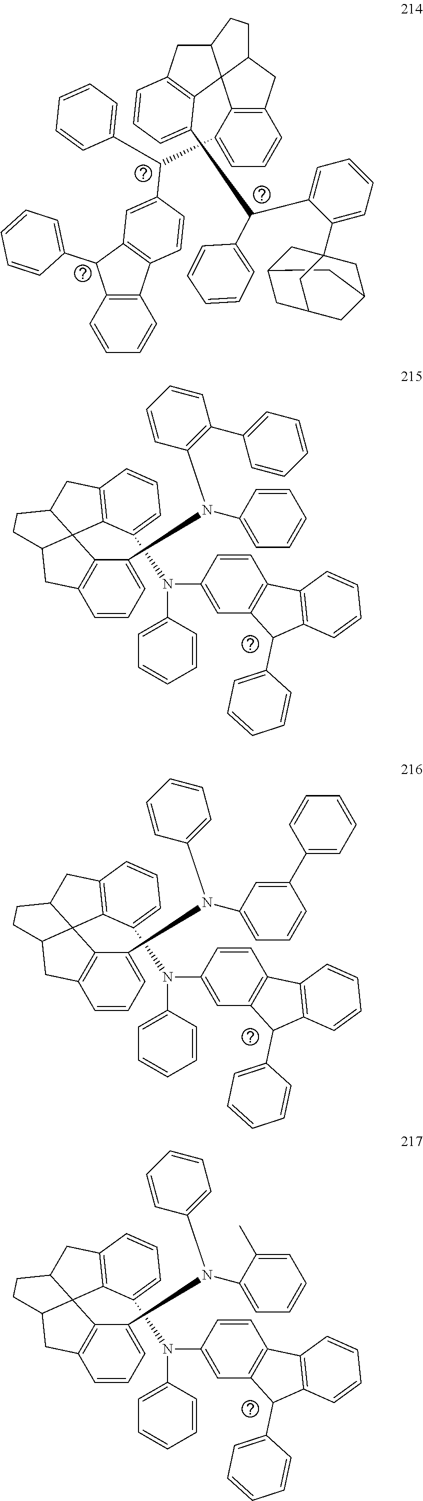

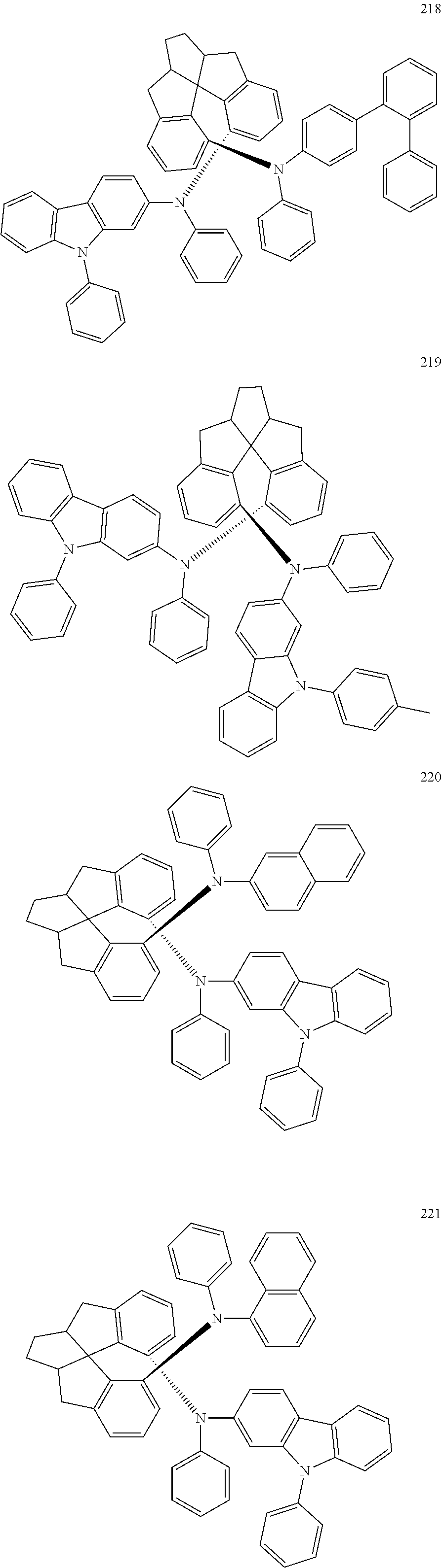

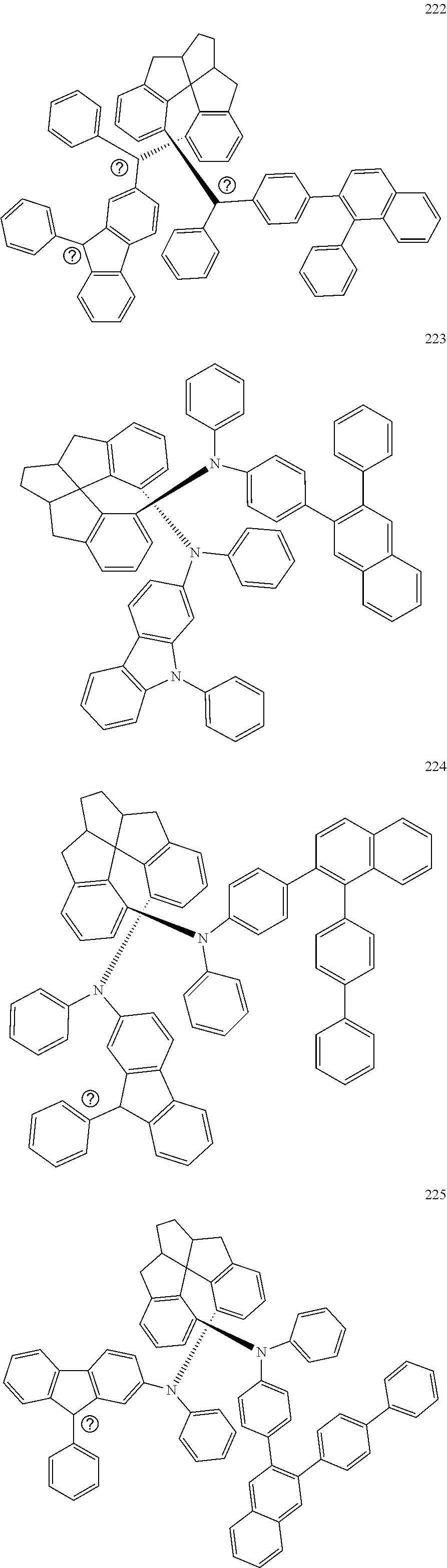

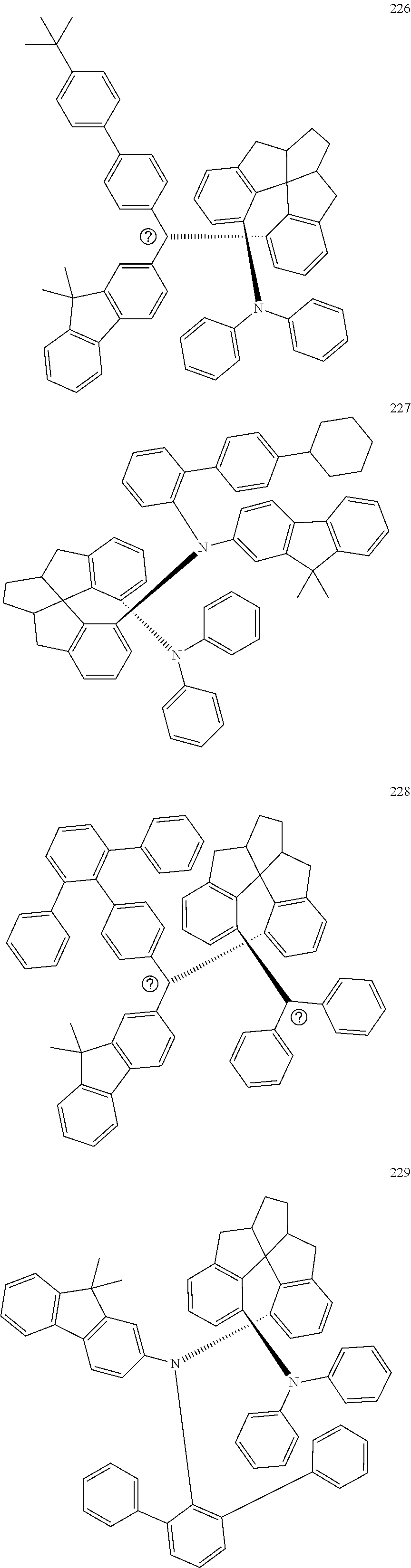

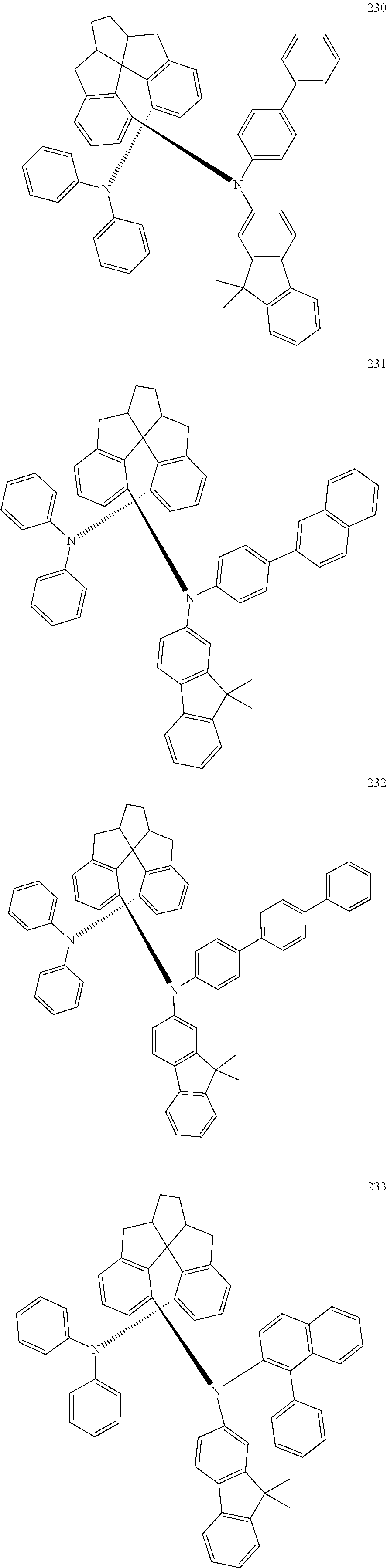

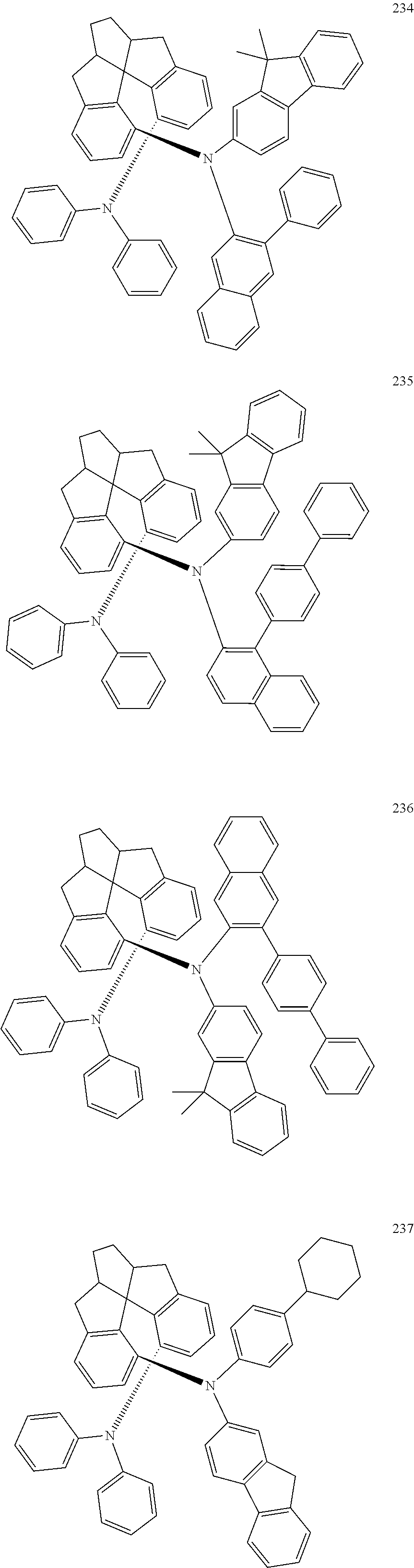

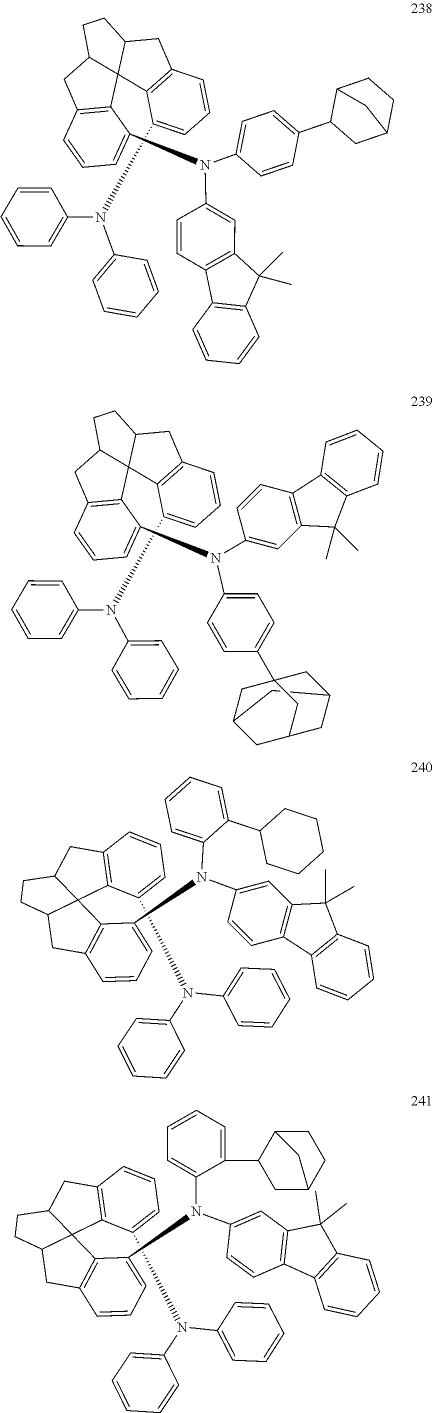

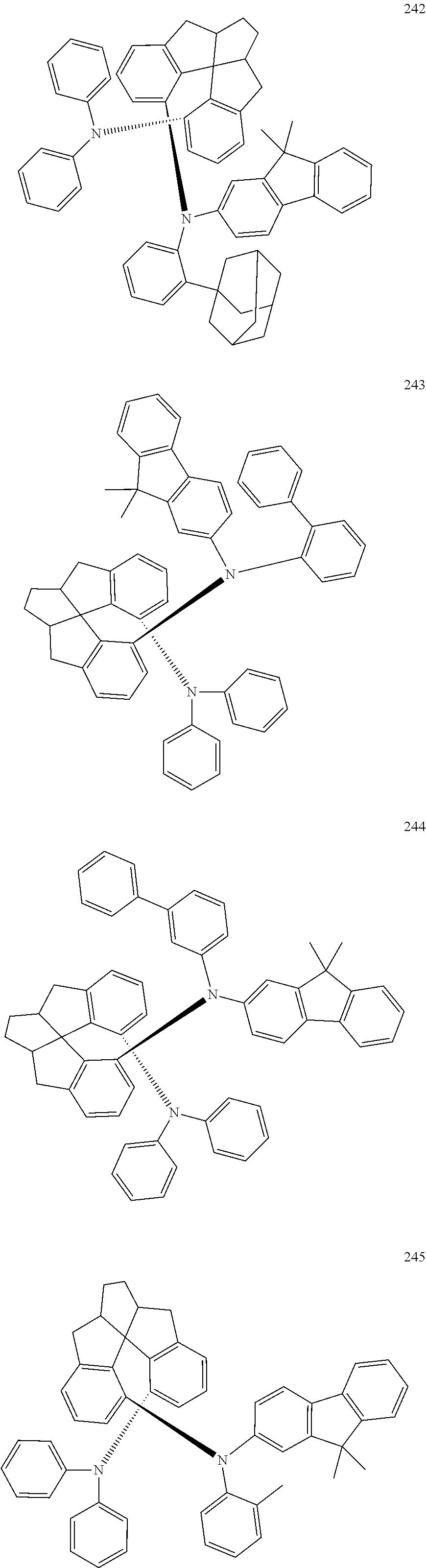

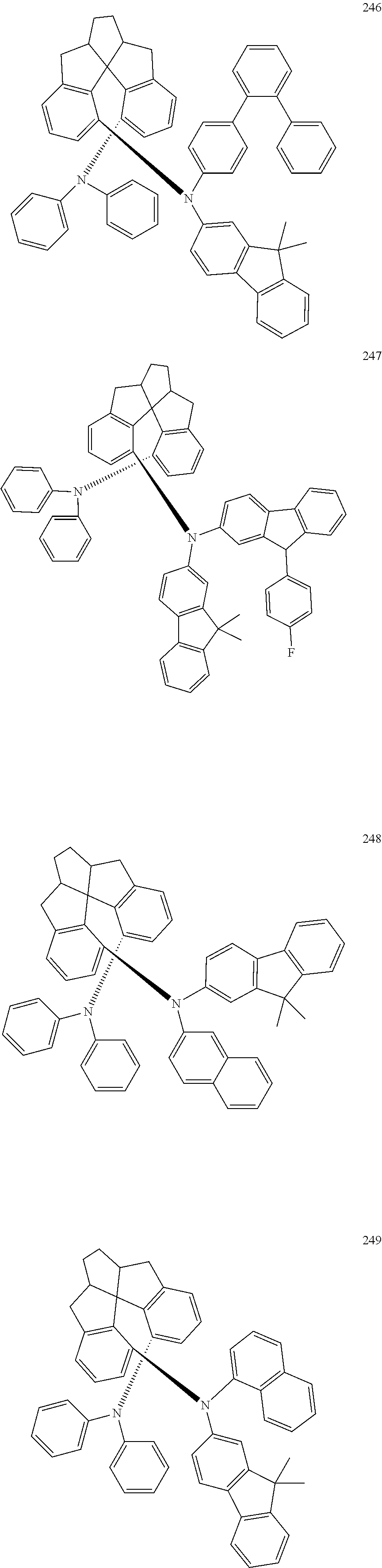

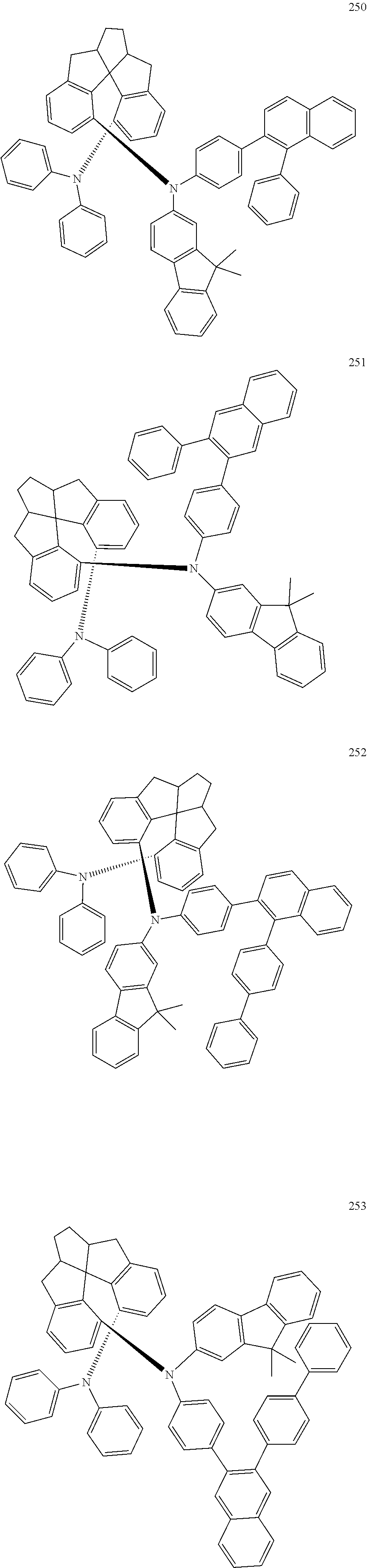

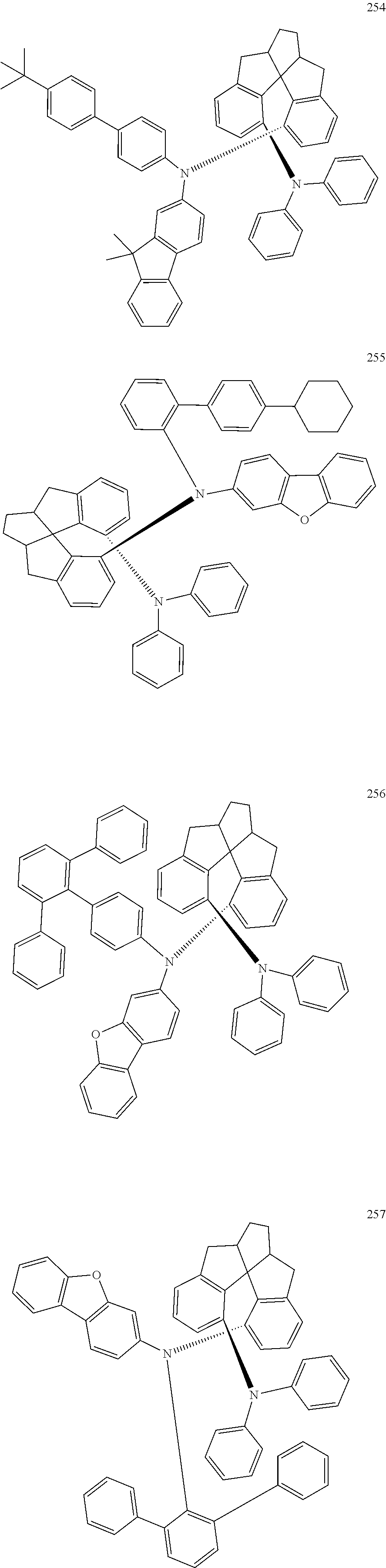

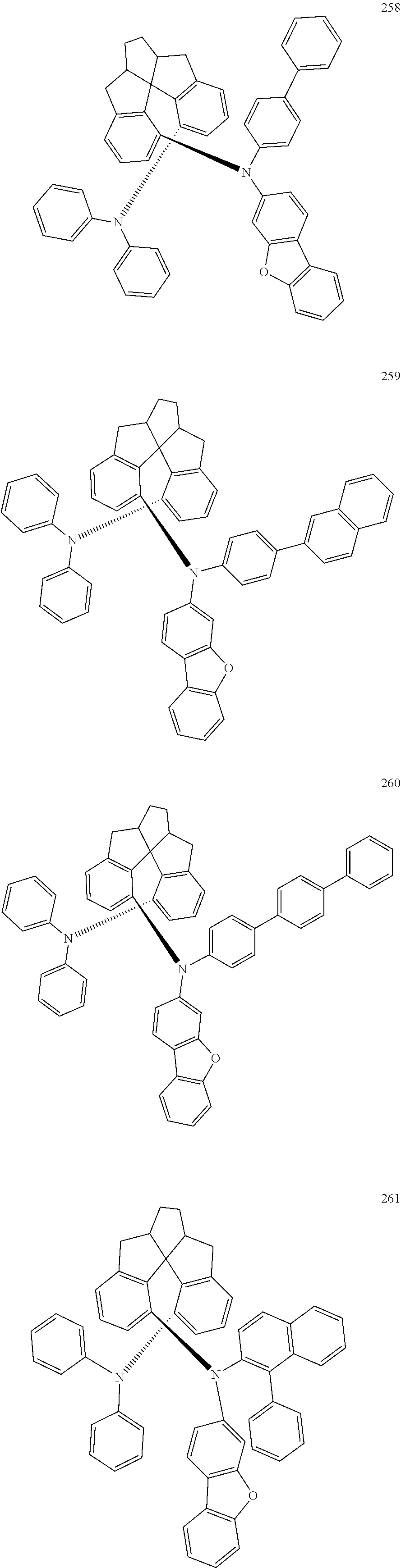

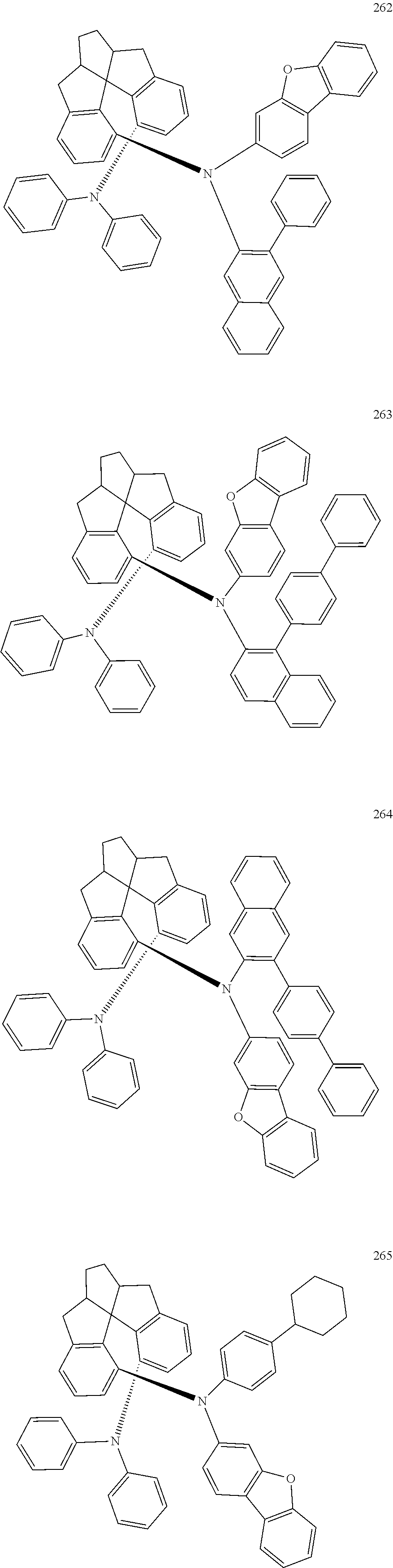

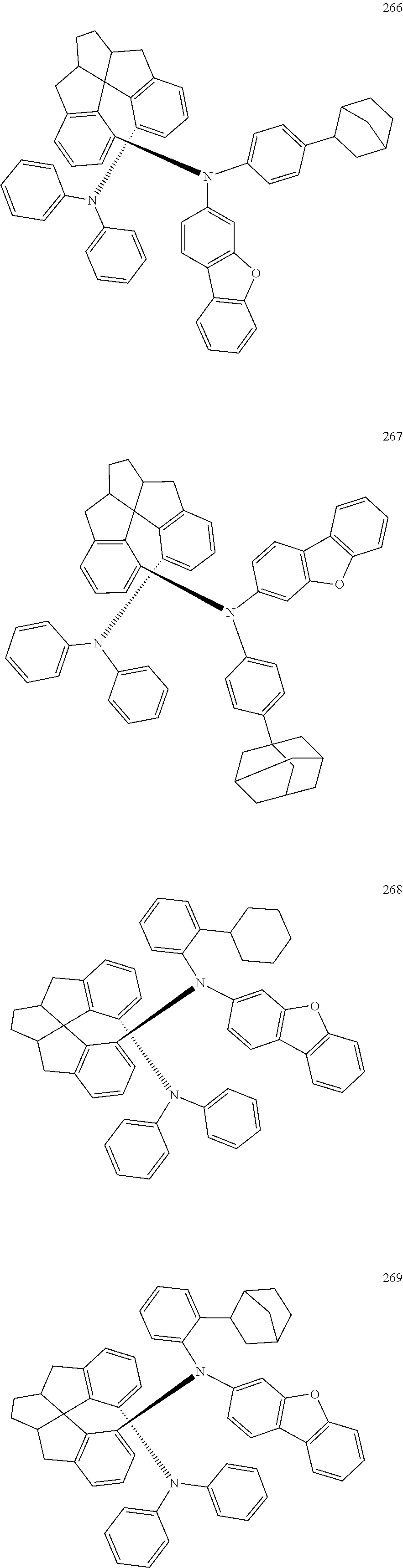

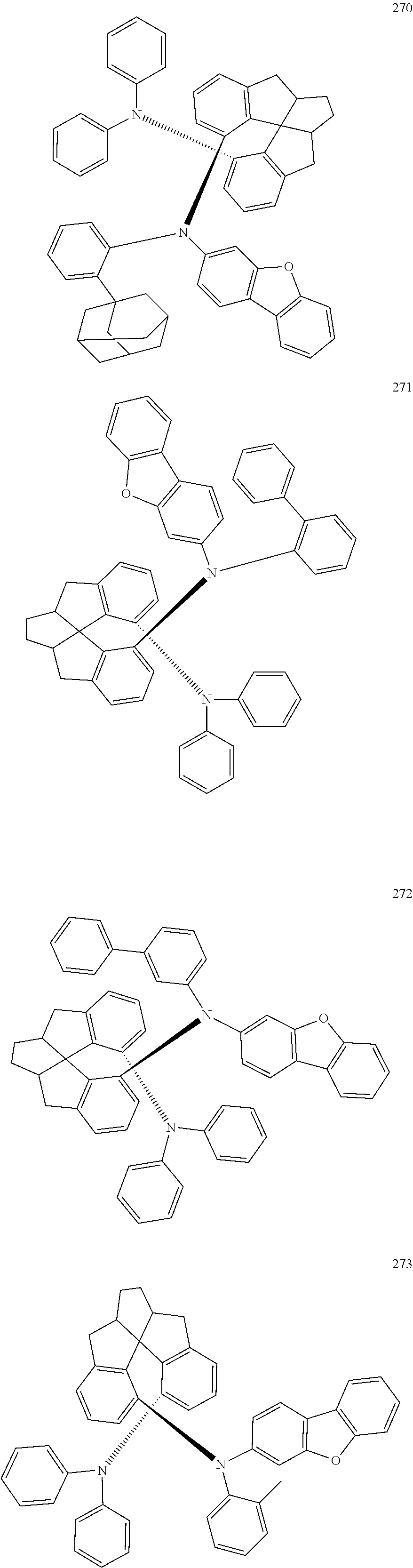

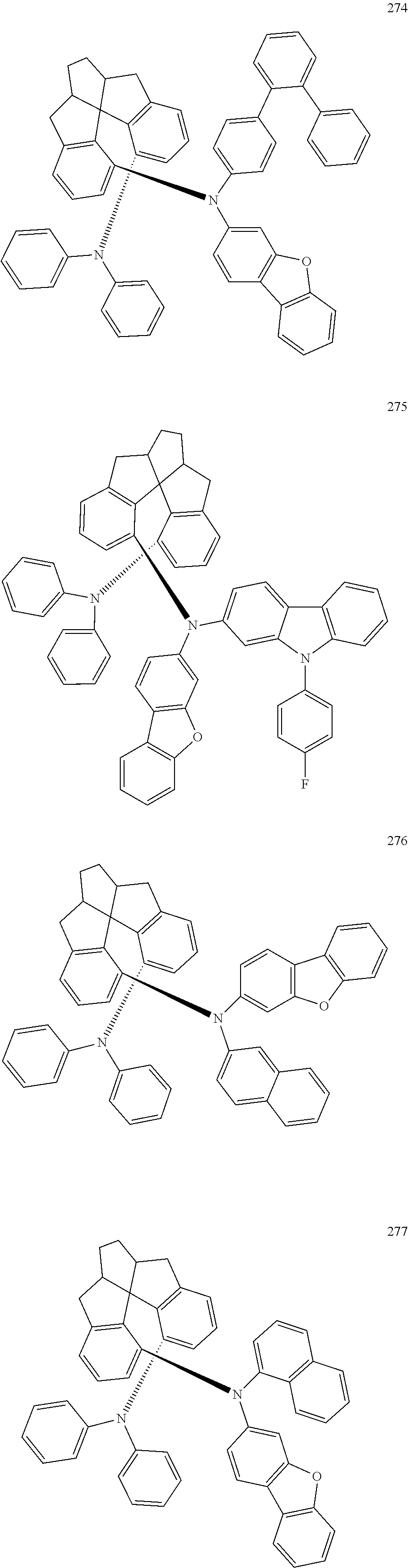

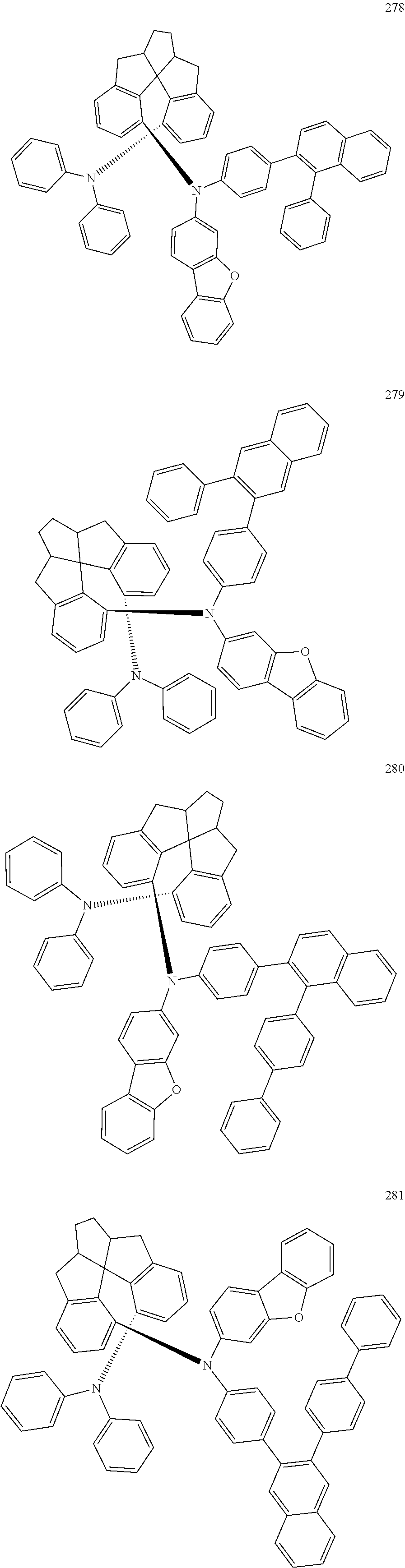

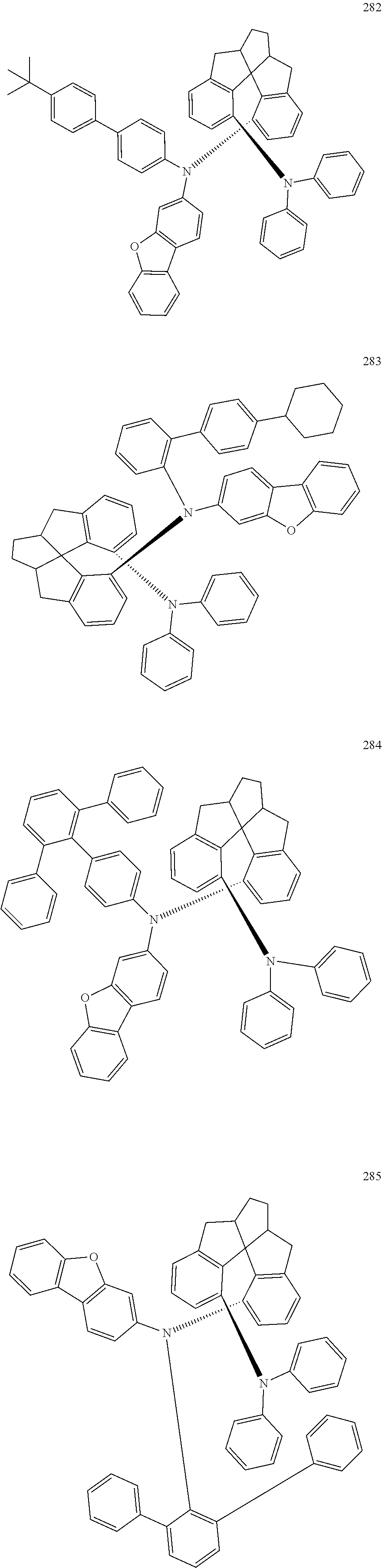

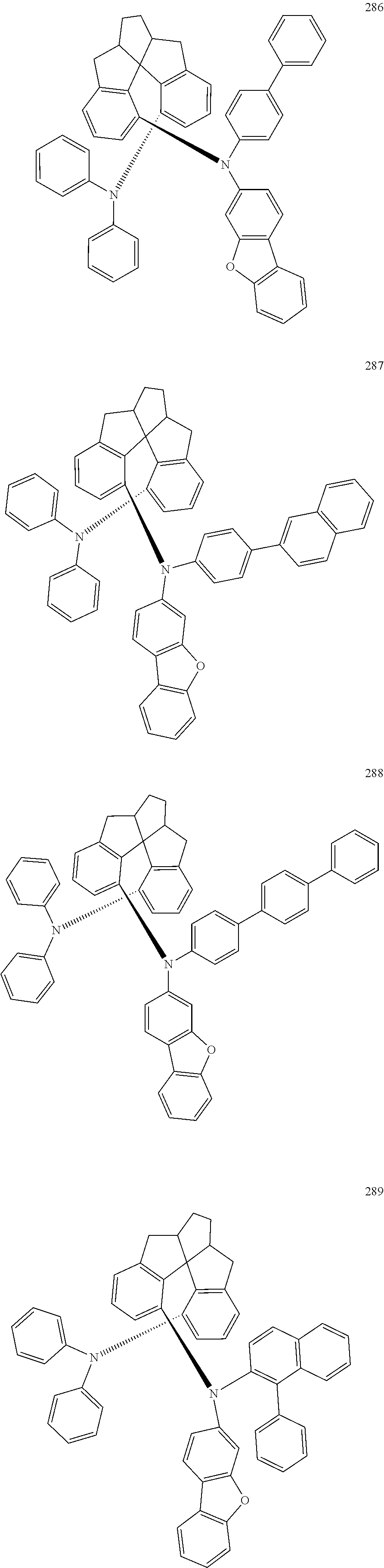

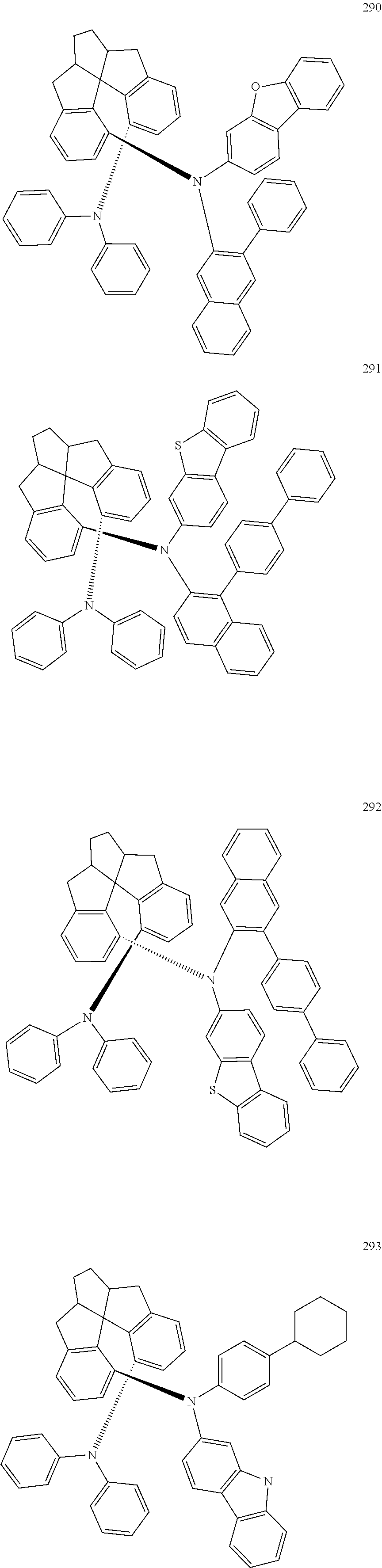

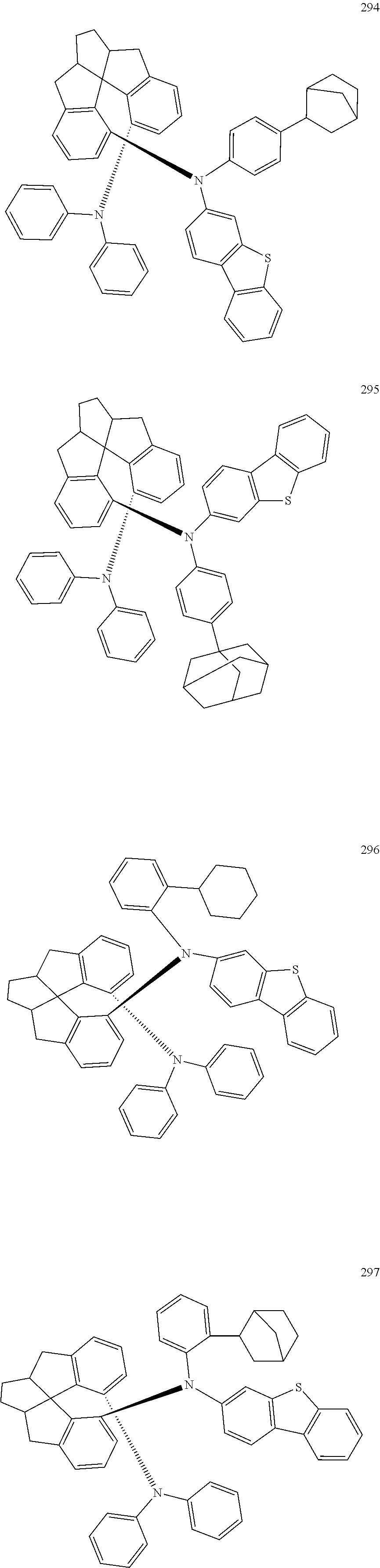

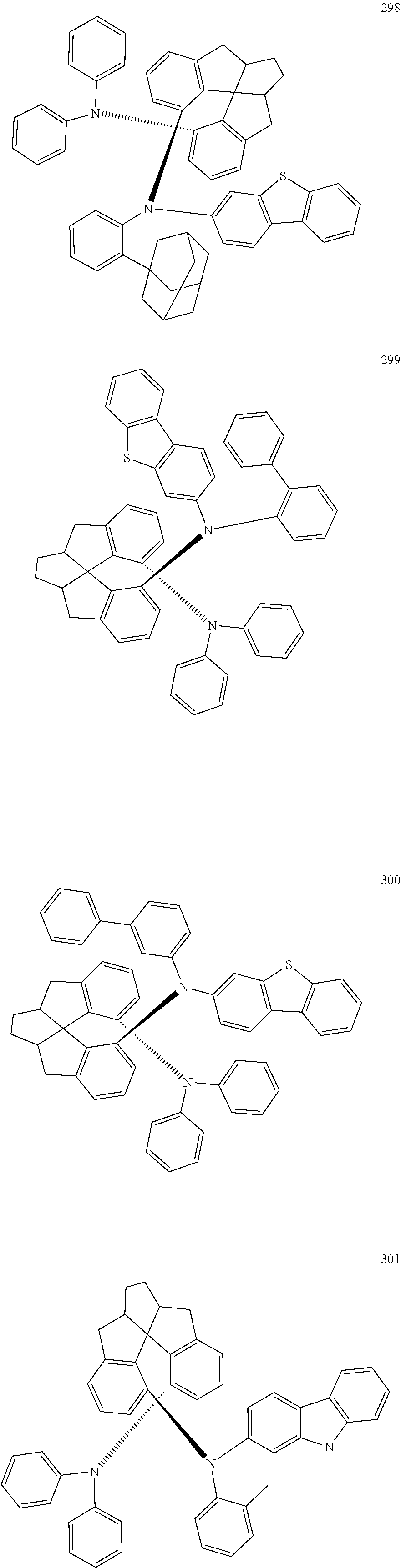

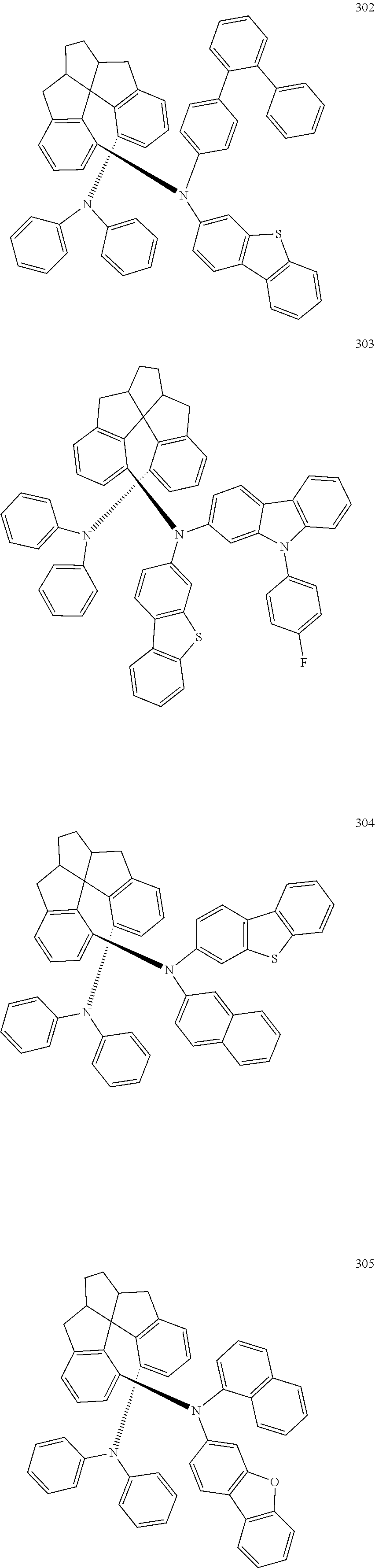

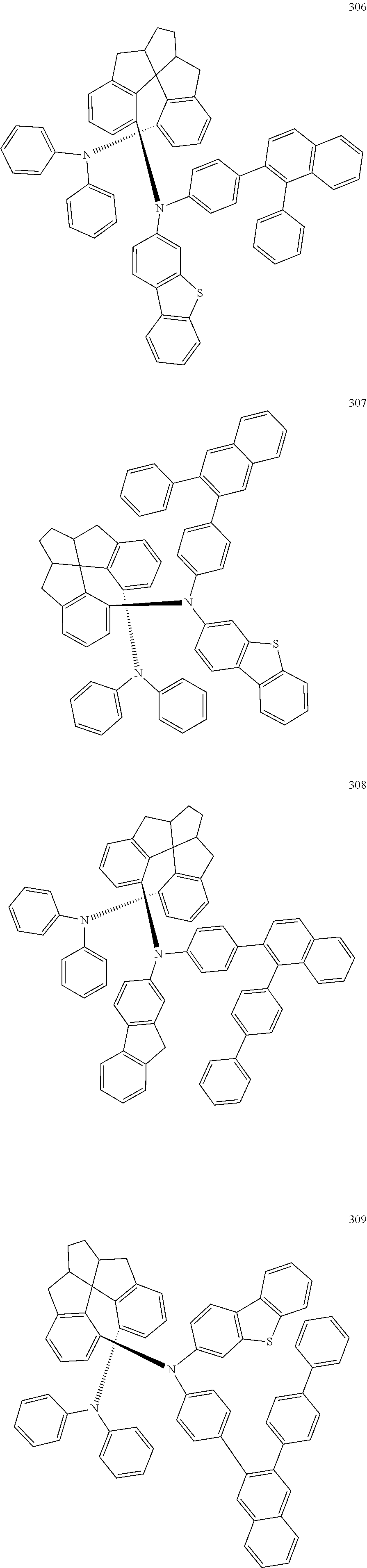

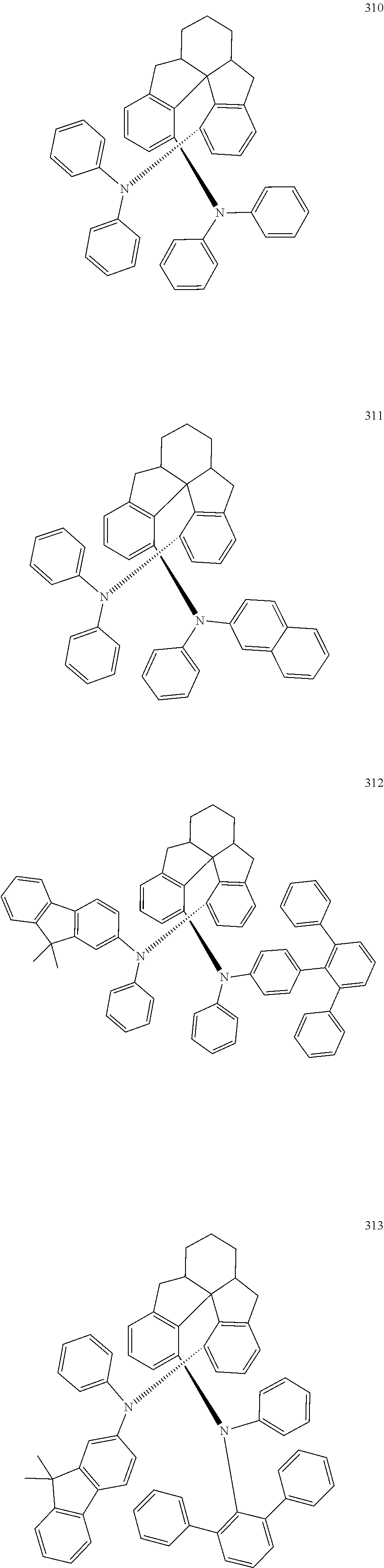

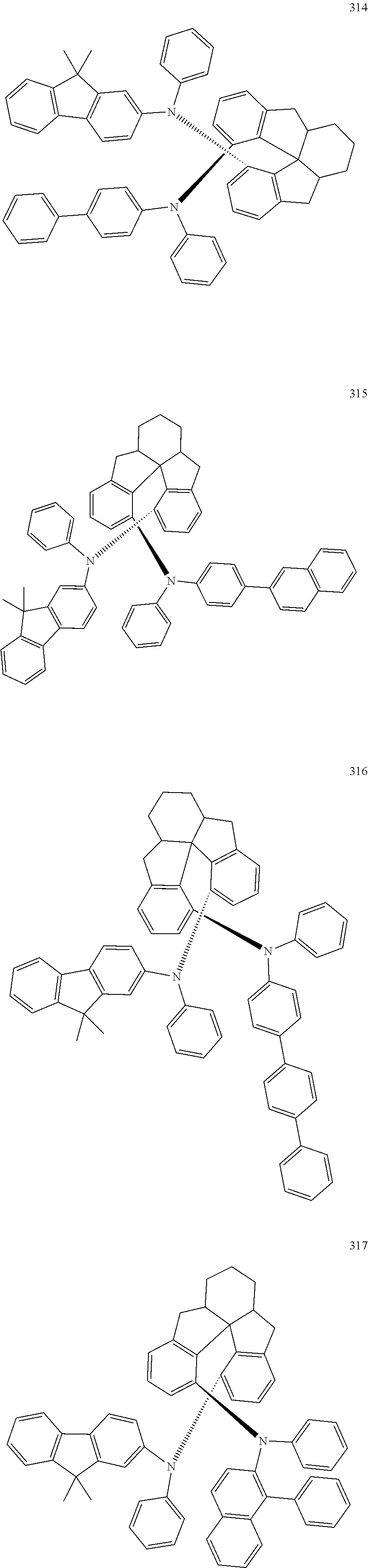

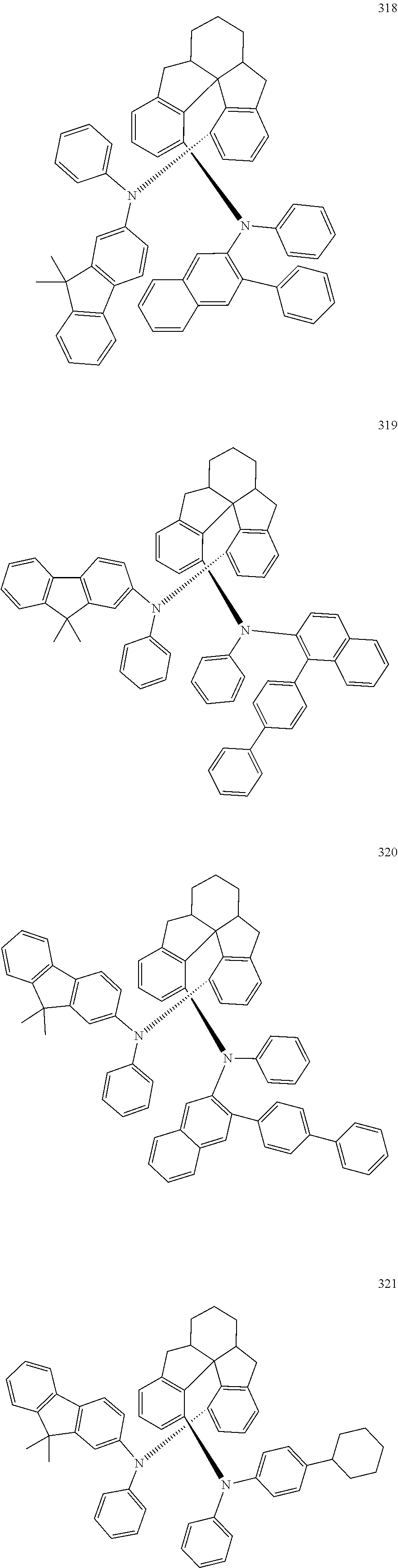

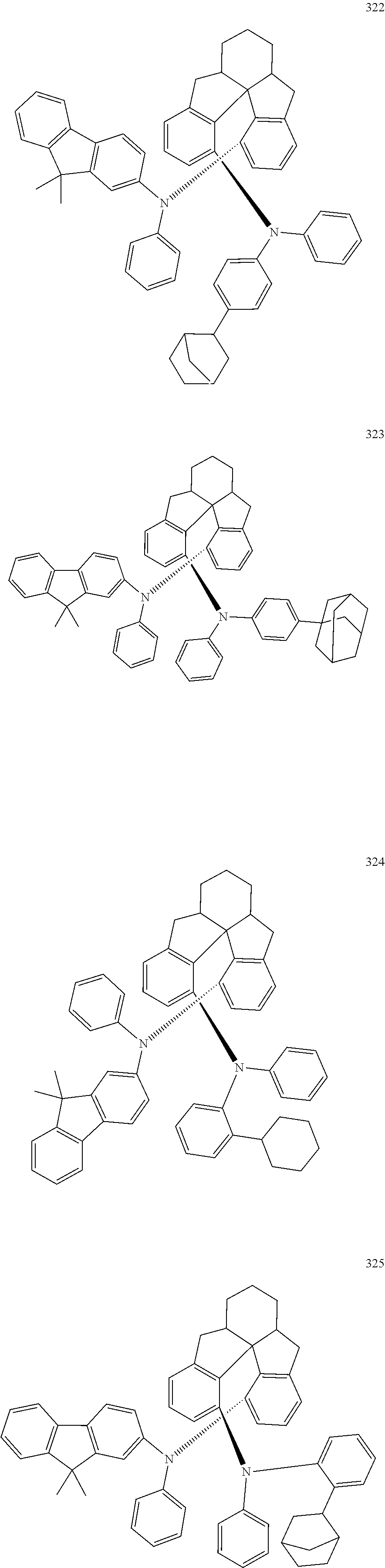

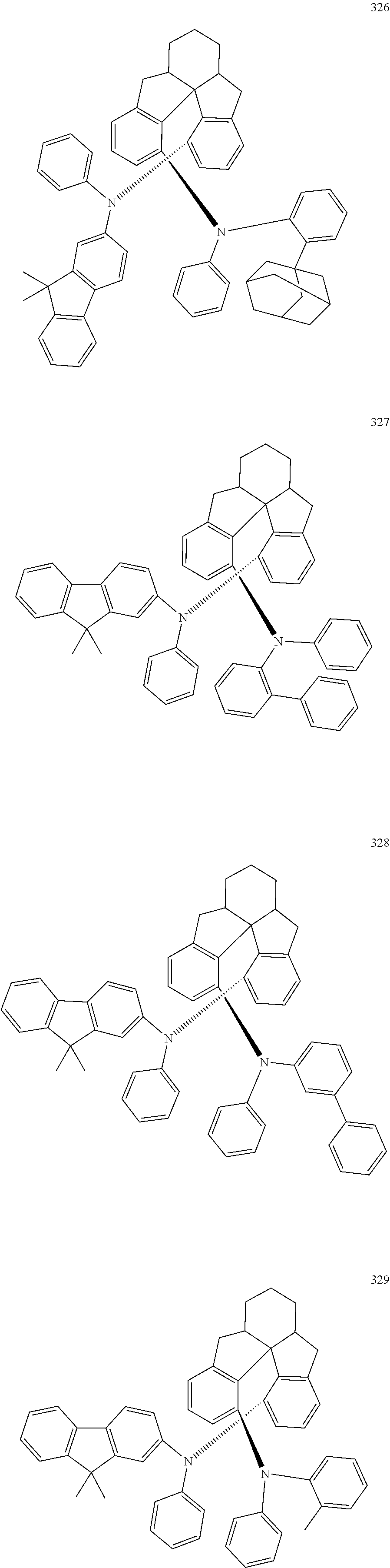

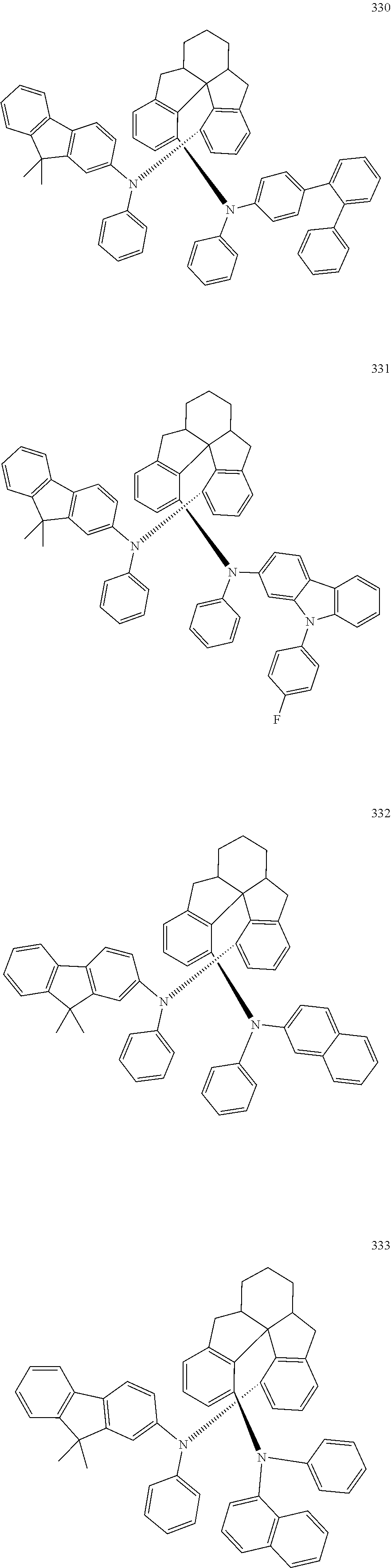

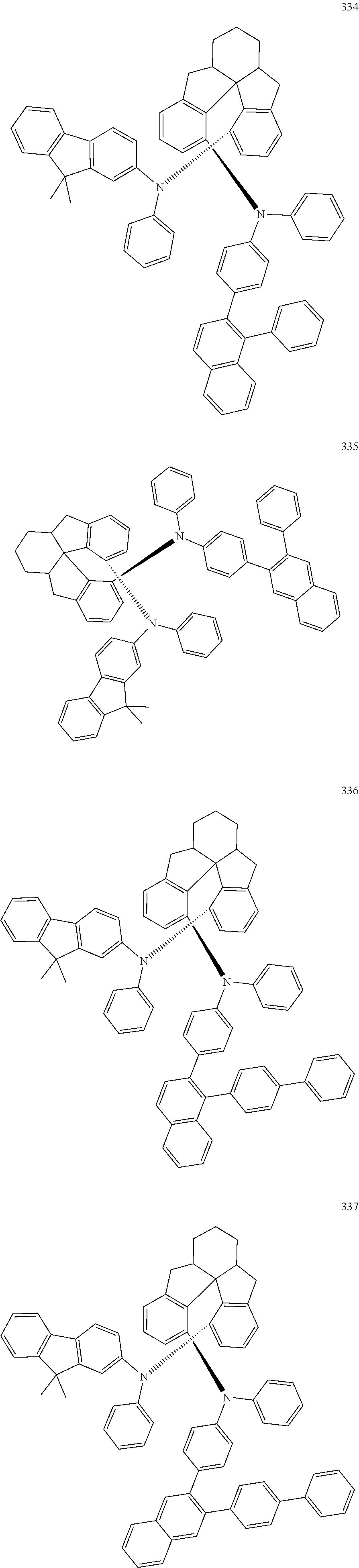

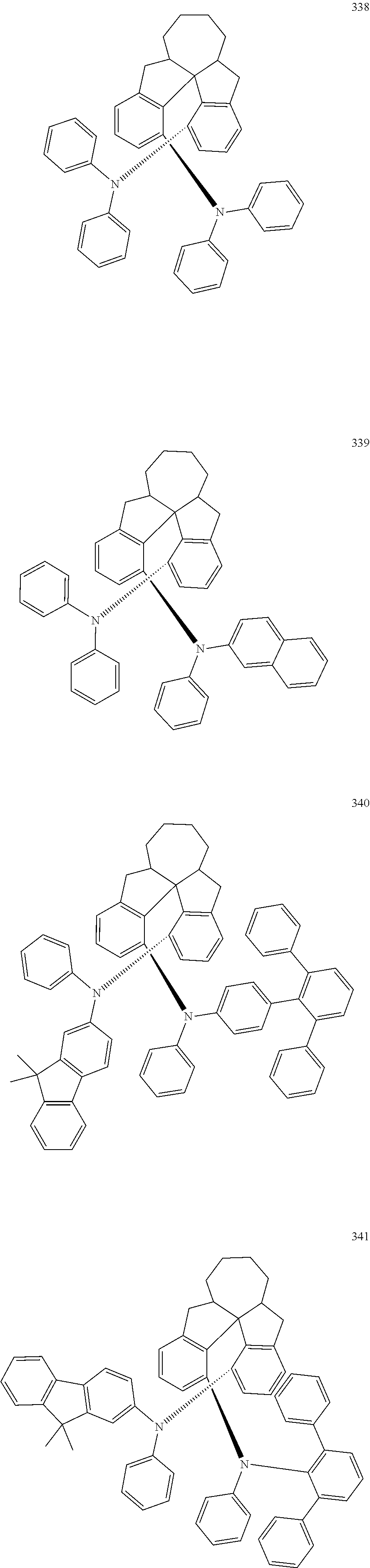

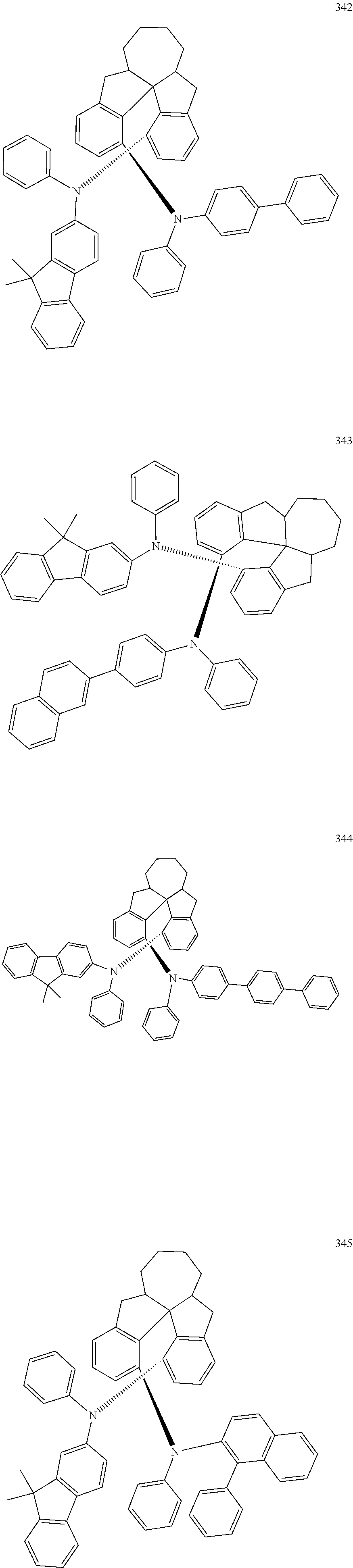

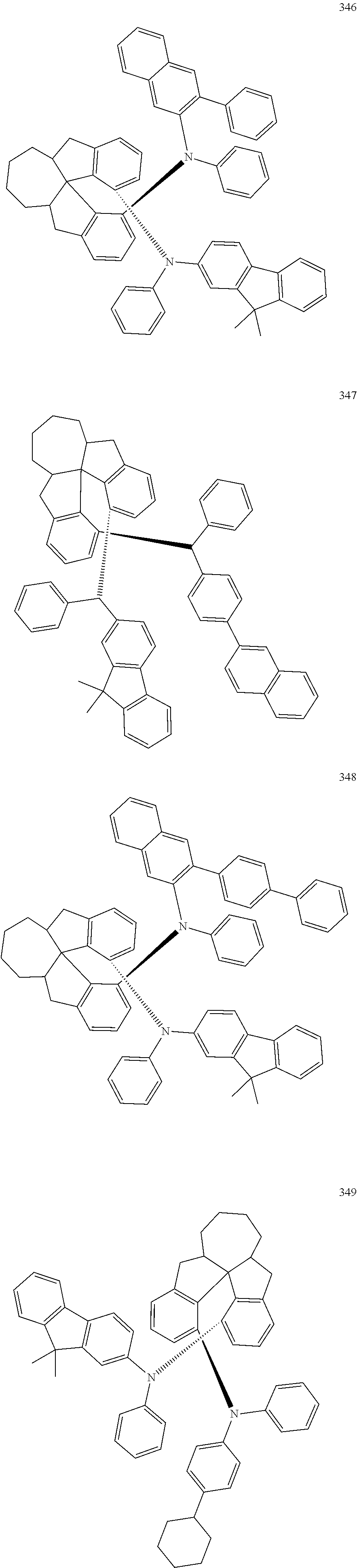

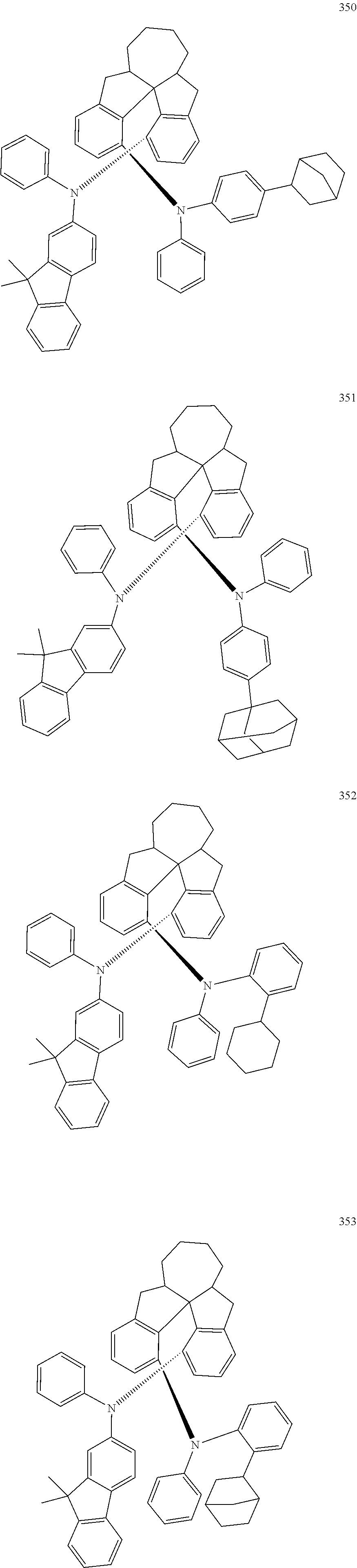

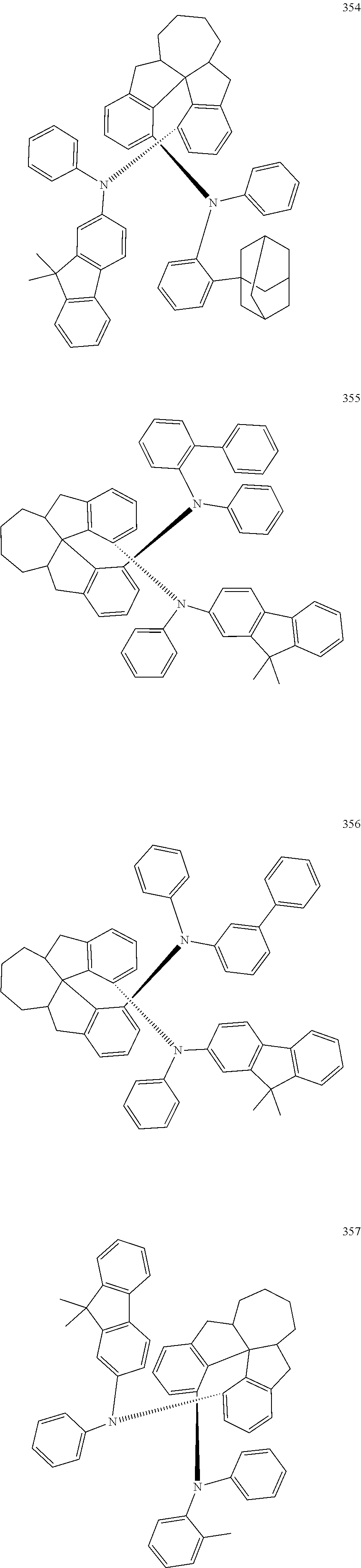

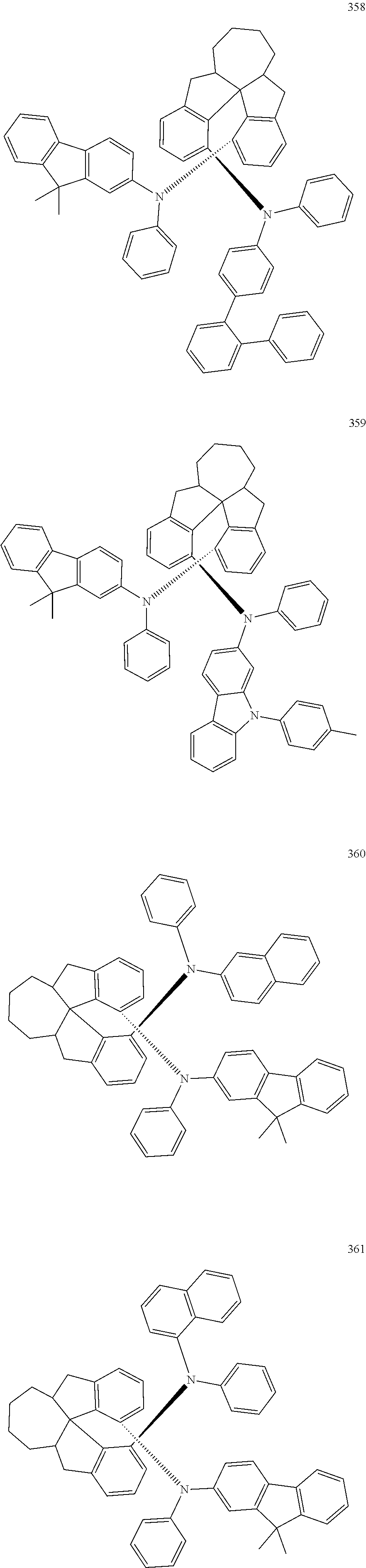

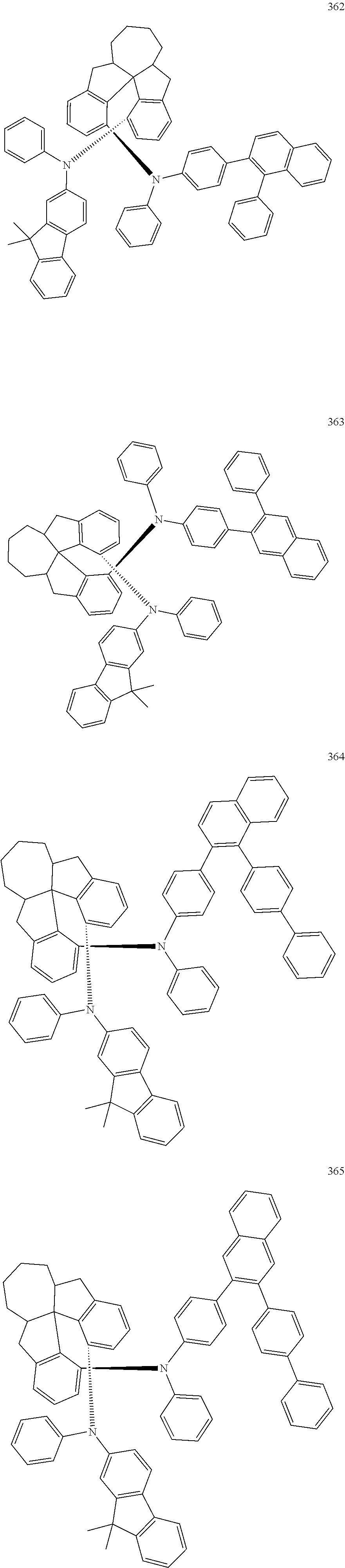

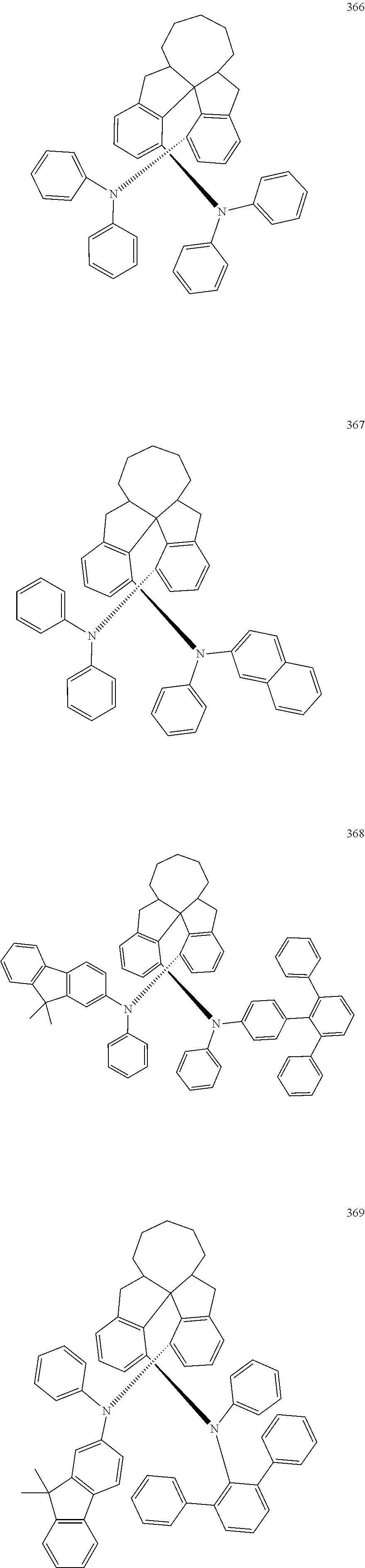

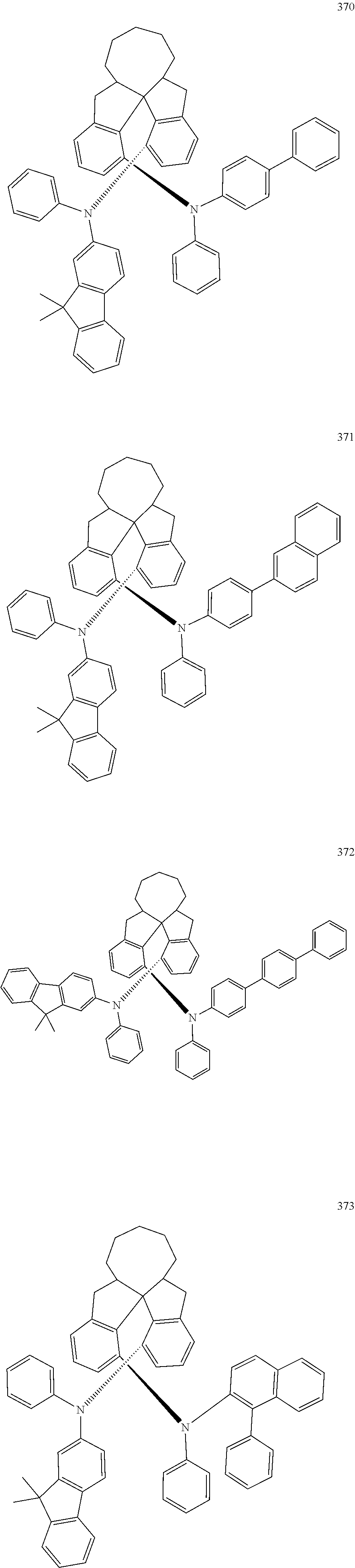

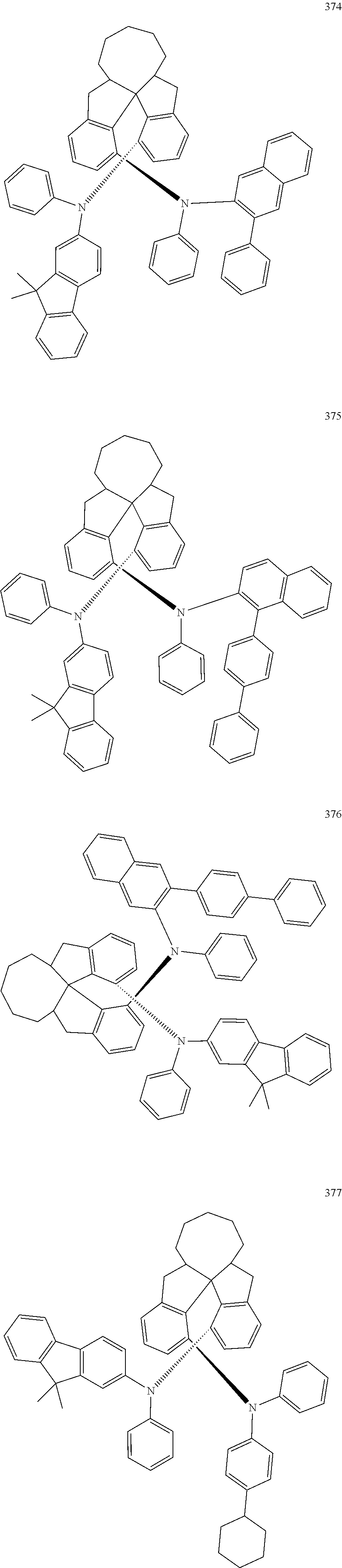

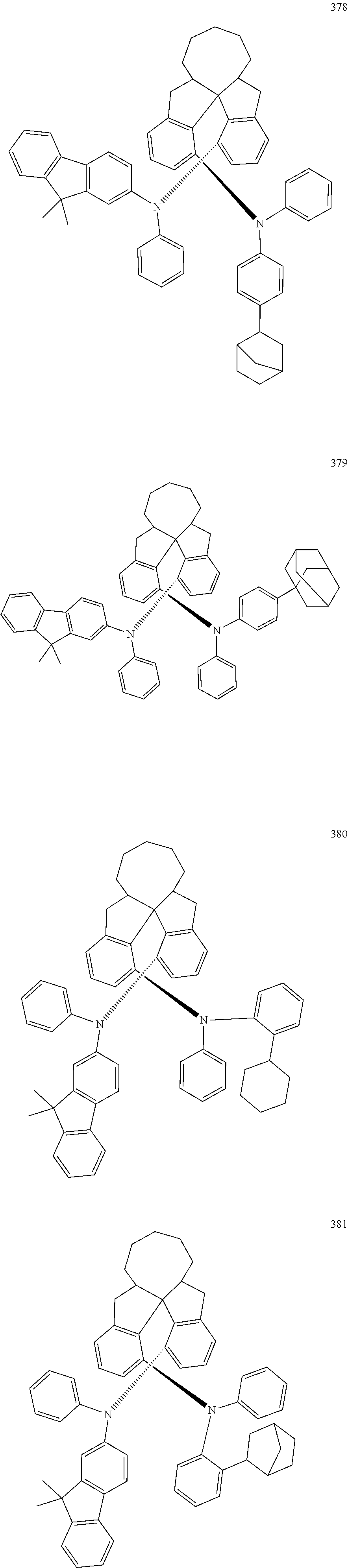

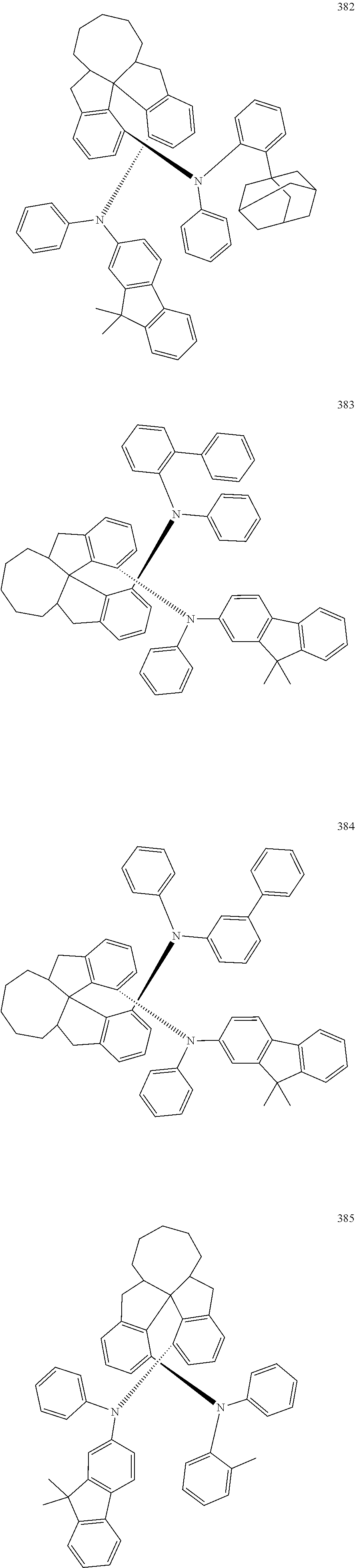

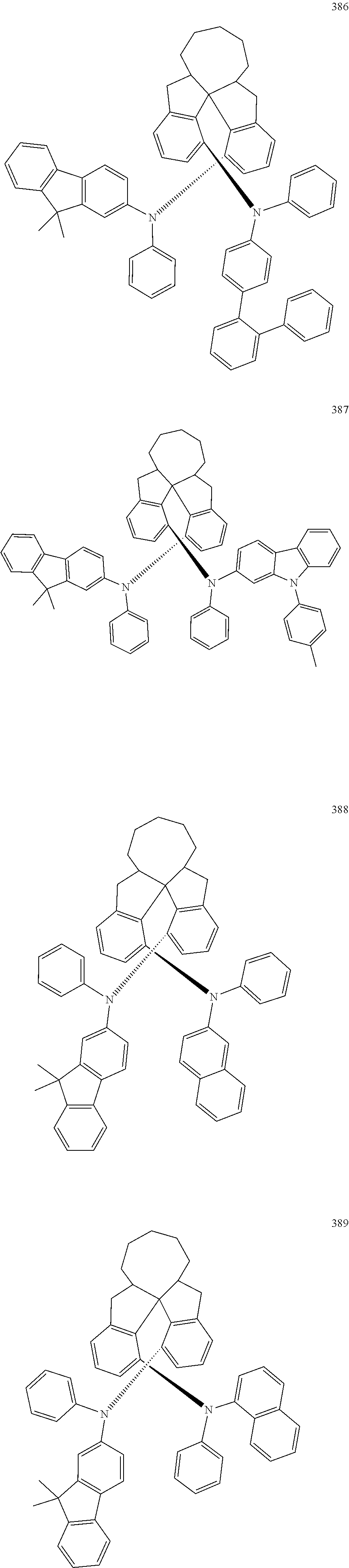

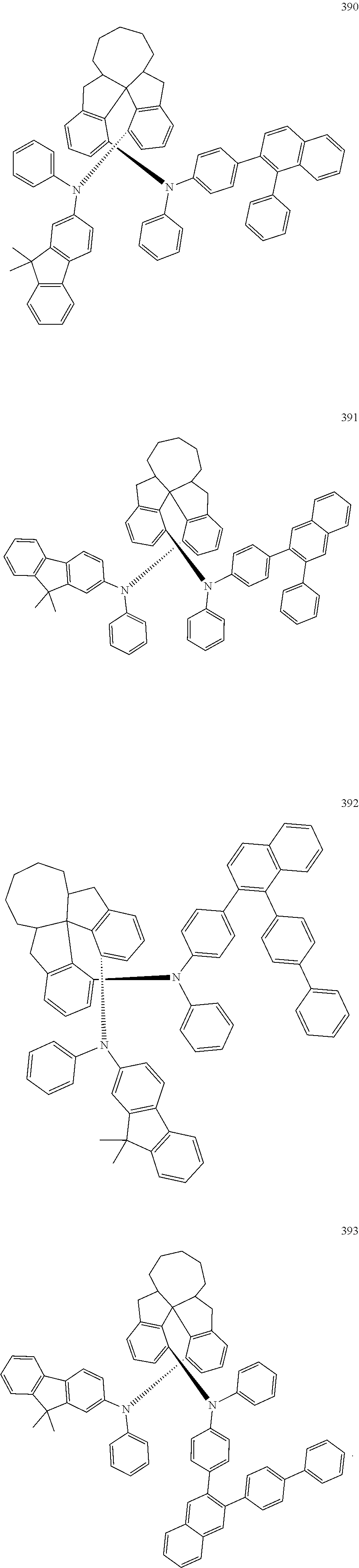

13. An amine compound represented by Formula 1: 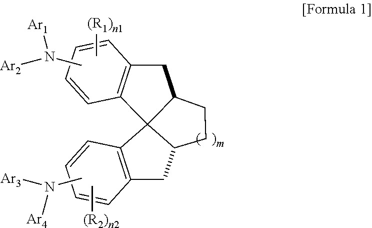
wherein in Formula 1,
Ar₁ to Ar₄ are each independently a substituted or unsubstituted aryl group having 6 to 30 ring-forming carbon atoms or a substituted or unsubstituted heteroaryl group having 2 to 30 ring-forming carbon atoms,
R₁ and R₂ are each independently a hydrogen atom, a deuterium atom, a halogen atom, a substituted or unsubstituted alkyl group having 1 to 20 carbon group, a substituted or unsubstituted aryl group having 6 to 30 ring-forming carbon atoms, or a substituted or unsubstituted heteroaryl group having 2 to 30 ring-forming carbon atoms,
n1 and n2 are each independently an integer from 0 to 3, and
m is an integer from 1 to 4.

14. The amine compound of claim 13, wherein the amine compound represented by Formula 1 is represented by Formula 2: 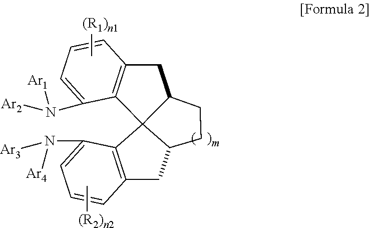
wherein in Formula 2,
Ar₁ to Ar₄, R₁, R₂, n1, n2, and m are the same as defined in Formula 1.

15. The amine compound of claim 13, wherein the amine compound represented by Formula 1 is represented by one of Formula 4-1 to Formula 4-4: 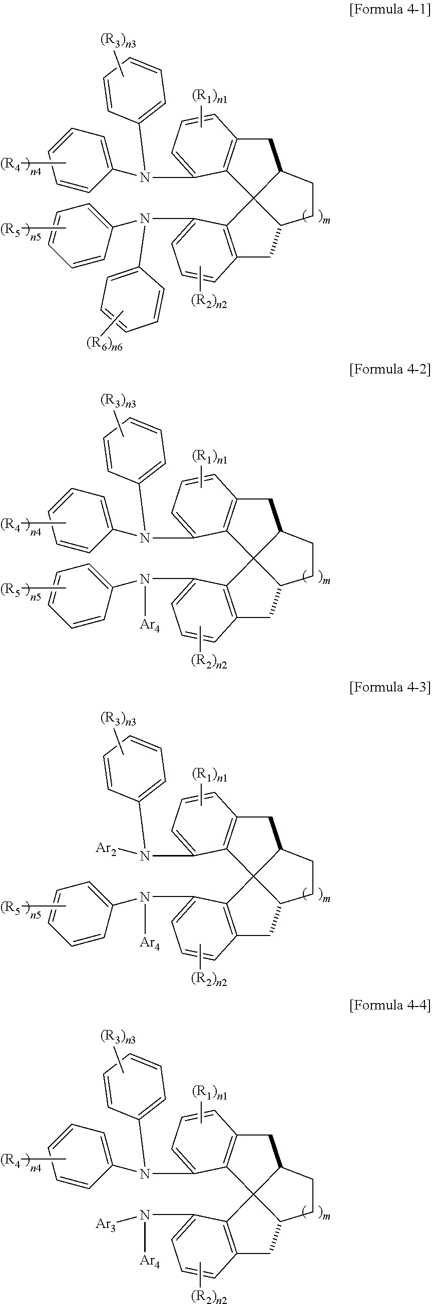
wherein in Formula 4-1 to Formula 4-4,
R₃ to R₆ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n3 to n6 are each independently an integer from 0 to 5, and
Ar₂ to Ar₄, R₁, R₂, n1, n2, and m are the same as defined in Formula 1.

16. The amine compound of claim 13, wherein the amine compound represented by Formula 1 is represented by one of Formula 5-1 to Formula 5-4: 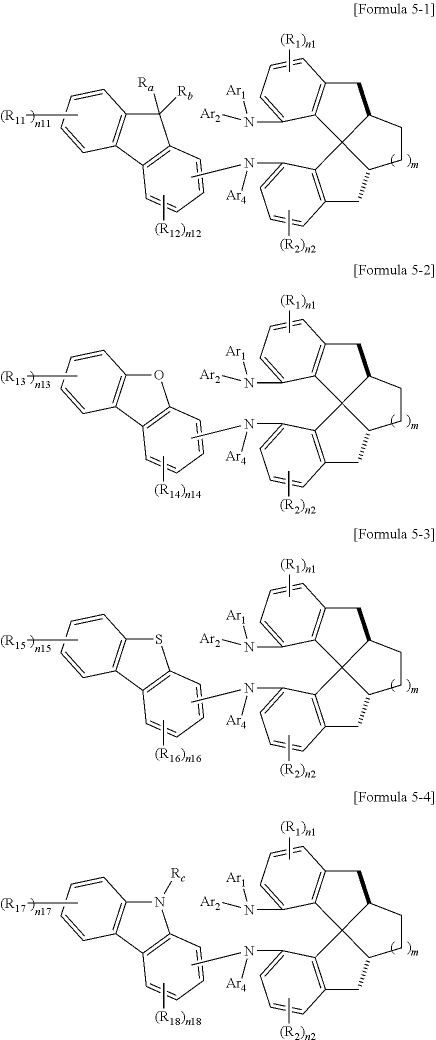
wherein in Formula 5-1 to Formula 5-4,
R₁₁ to R₁₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Rₐ to R_{c} are each independently a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring,
n11, n13, n15, and n17 are each independently an integer from 0 to 4,
n12, n14, n16, and n18 are each independently an integer from 0 to 3, and
Ar₁, Ar₂, Ar₄, R₁, R₂, n1, n2, and m are the same as defined in Formula 1.

17. The amine compound of claim 13, wherein at least one of Ar₁ to Ar₄ is each independently a group represented by one of Formula 7-1 to Formula 7-7: 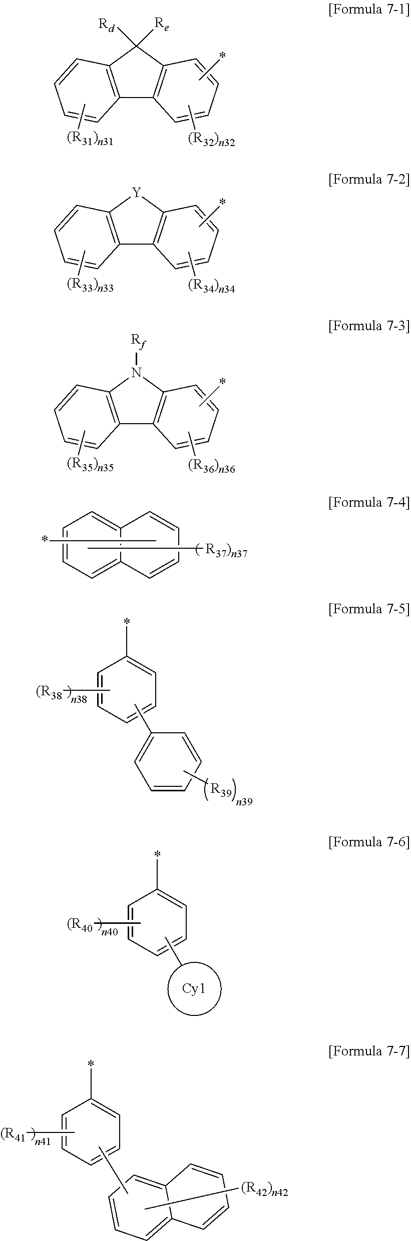
wherein in Formula 7-1 to Formula 7-7,
Y is O or S,
R₃₁ to R₄₂ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
R_{d} to R_{f} are each independently a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring,
ring Cy1 is a substituted or unsubstituted cycloalkyl group having 3 to 20 ring-forming carbon atoms,
n31, n33, n35, n38, n40, and n41 are each independently an integer from 0 to 4,
n32, n34 and n36 are each independently an integer from 0 to 3,
n37 and n42 are each independently an integer from 0 to 7,
n39 is an integer from 0 to 5, and
-* represents a bonding site to a nitrogen atom in Formula 1.

18. The amine compound of claim 13, wherein the amine compound represented by Formula 1 is represented by Formula 8-1 or Formula 8-2: 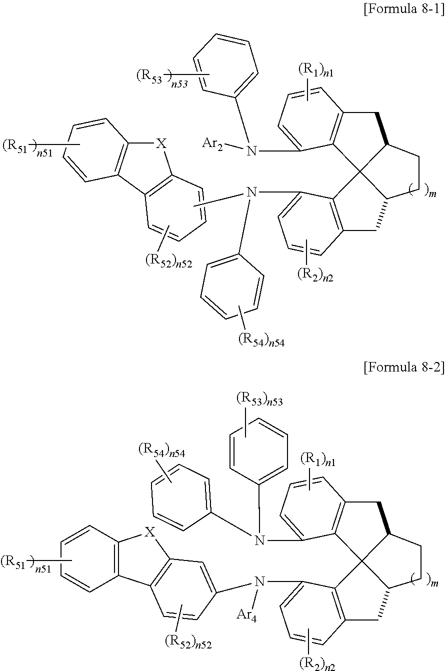
wherein in Formula 8-1 and Formula 8-2,
X is N(R₅₅), C(R₅₆)(R₅₇), O, or S,
R₅₁ to R₅₇ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and
Ar₂, Ar₄, R₁, R₂, n1, n2, and m are the same as defined in Formula 1.

19. The amine compound of claim 13, wherein the amine compound represented by Formula 1 is represented by one of Formula 9-1 to Formula 9-4: 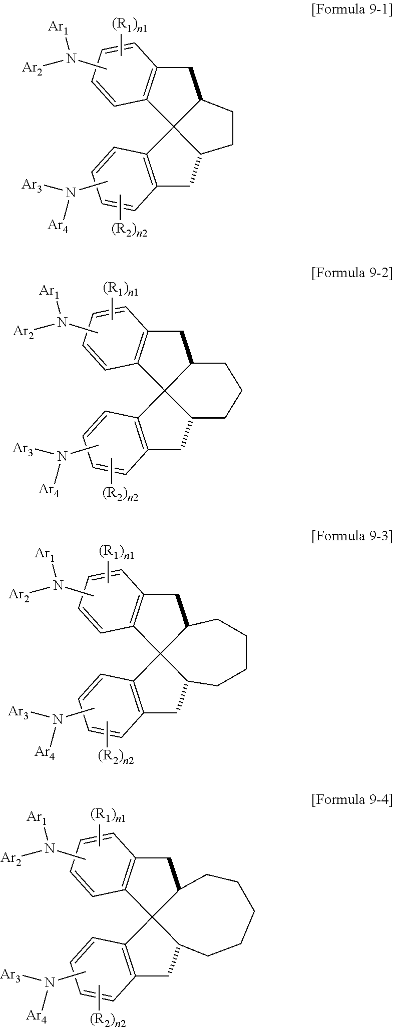
wherein in Formula 9-1 to Formula 9-4,
Ar₁ to Ar₄, R₁, R₂, n1, and n2 are the same as defined in Formula 1.

20. The amine compound of claim 13, wherein the amine compound is selected from Compound Group 1: 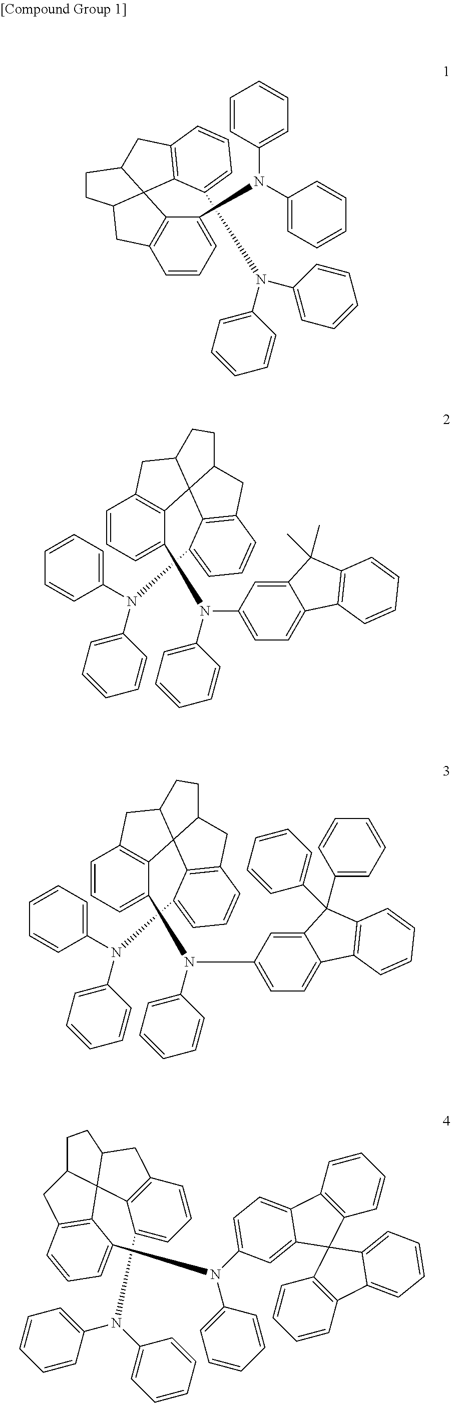 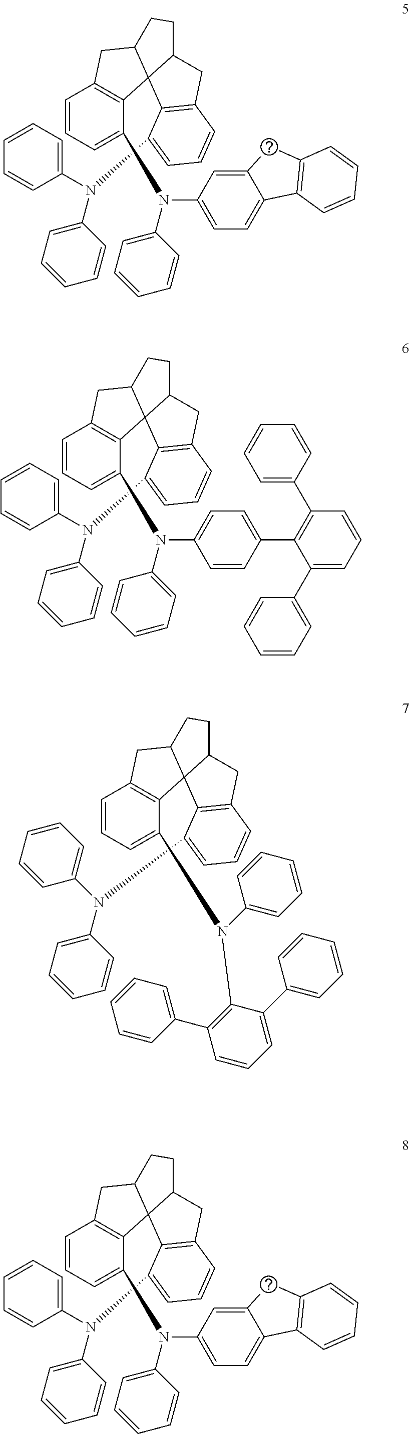 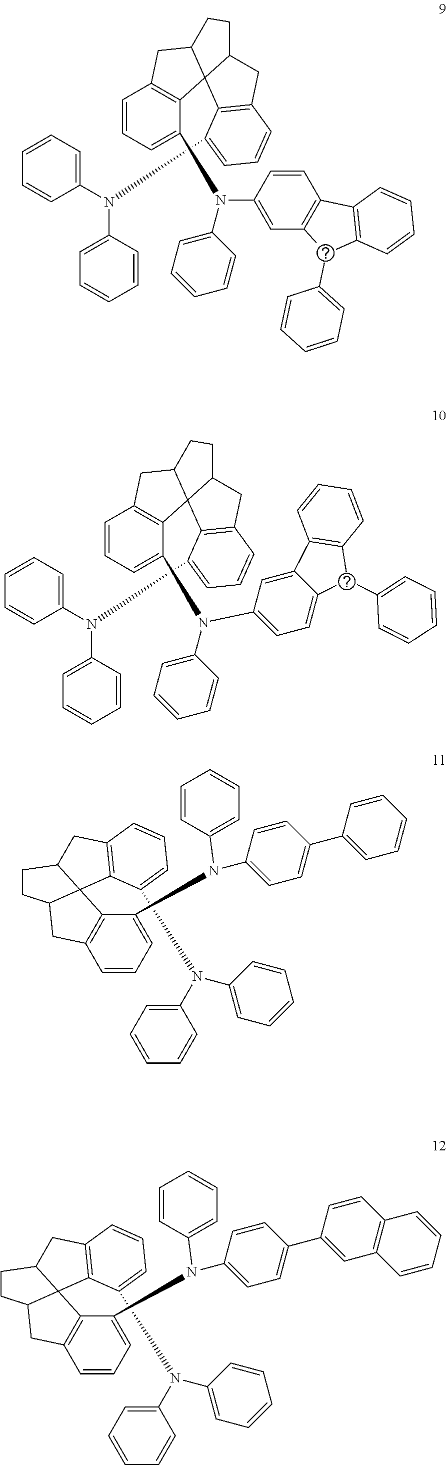 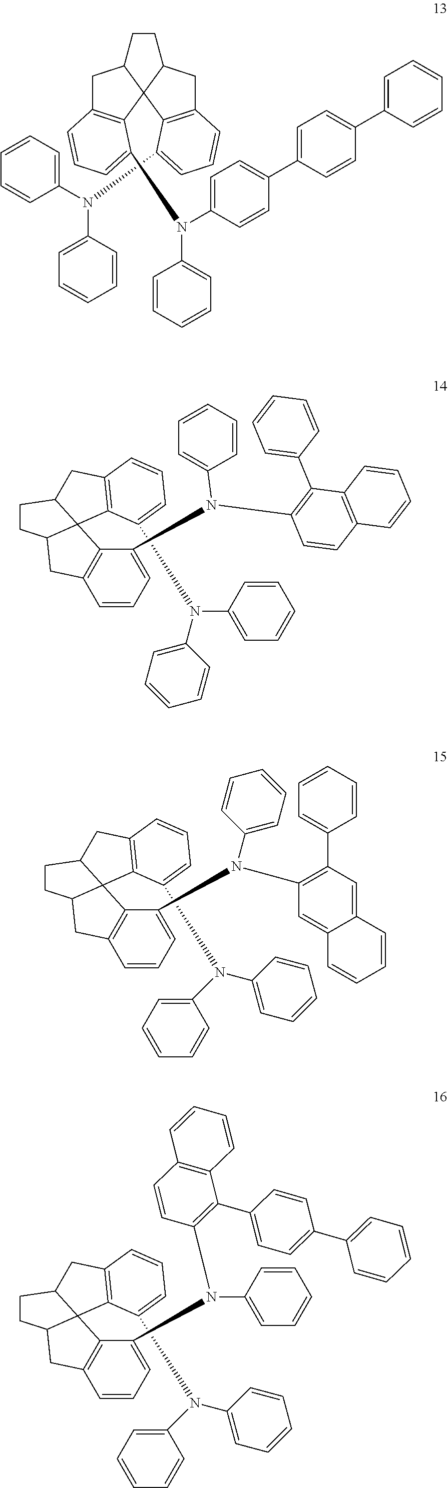 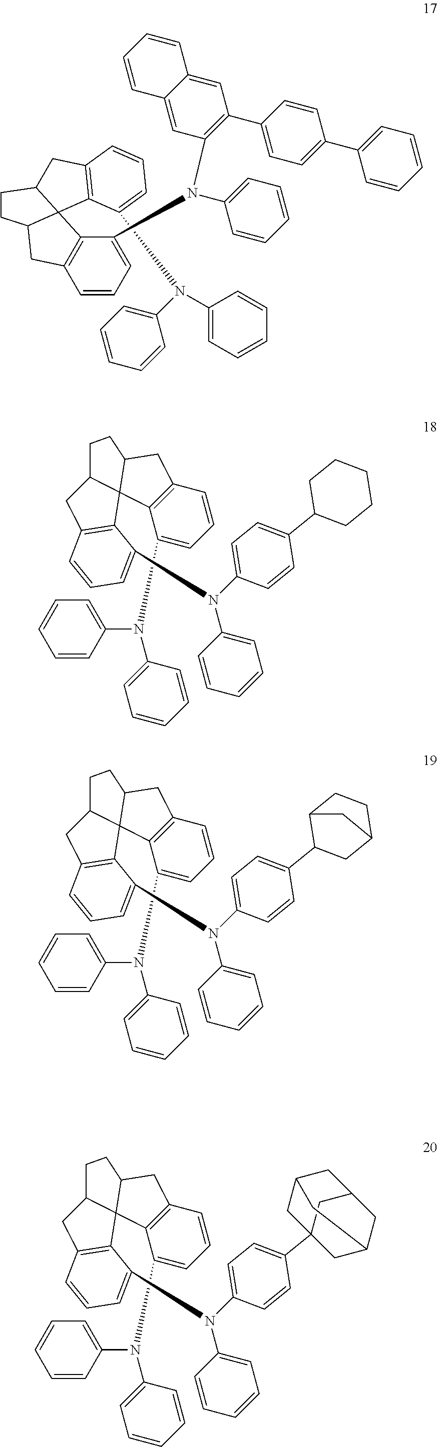 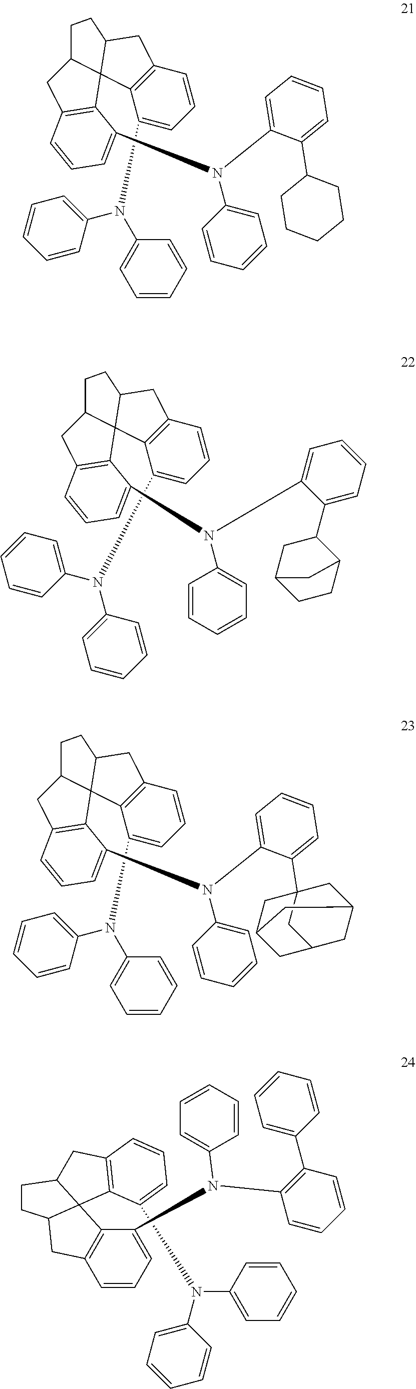 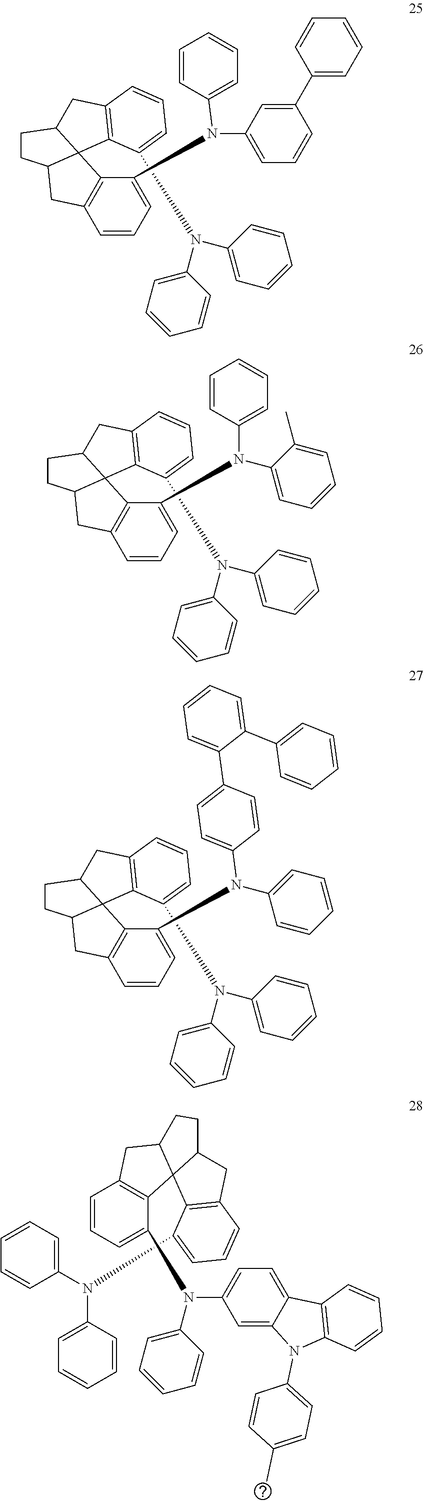 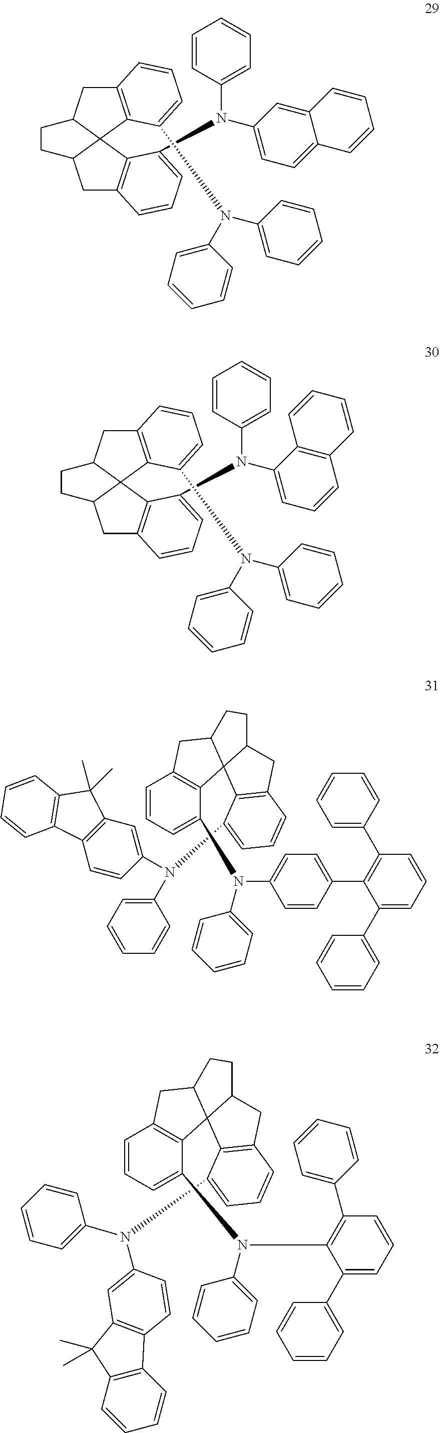 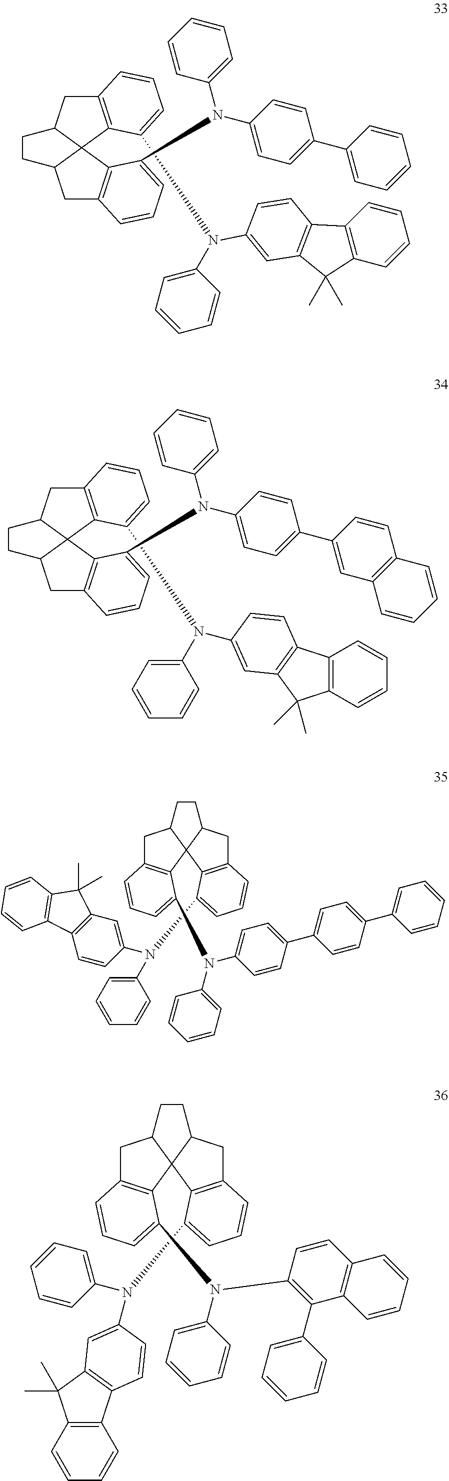 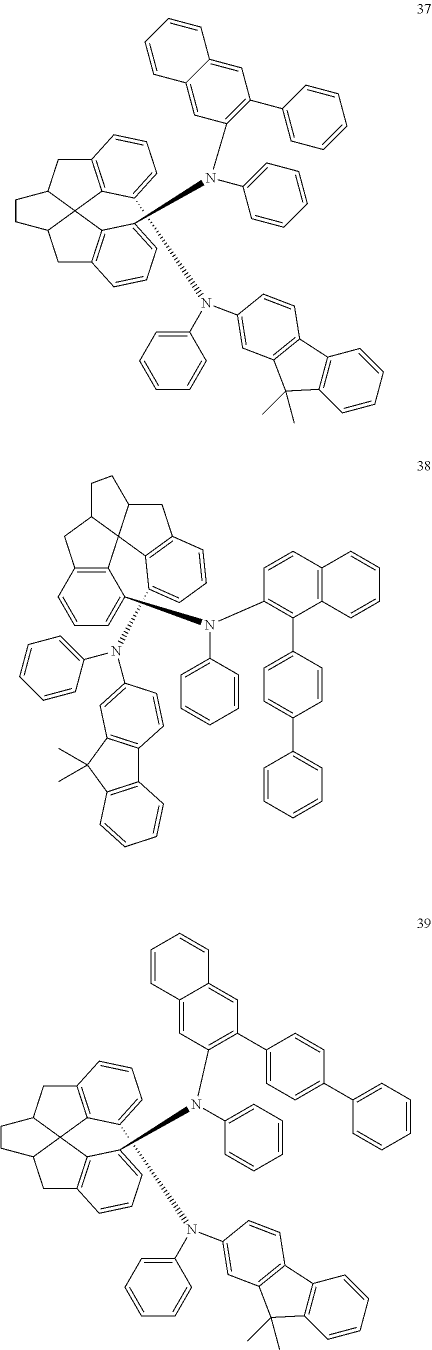 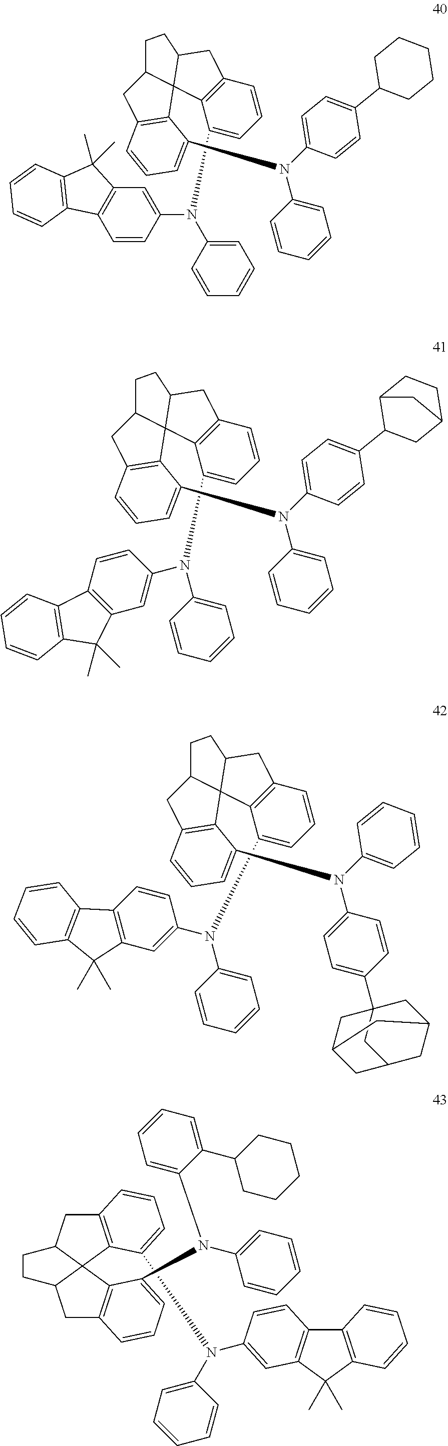 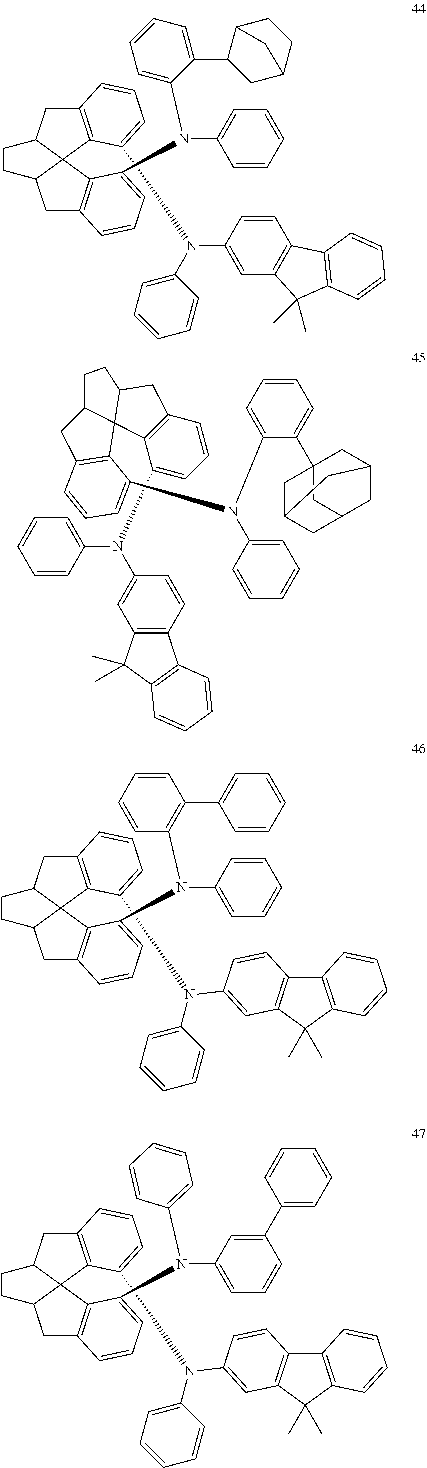 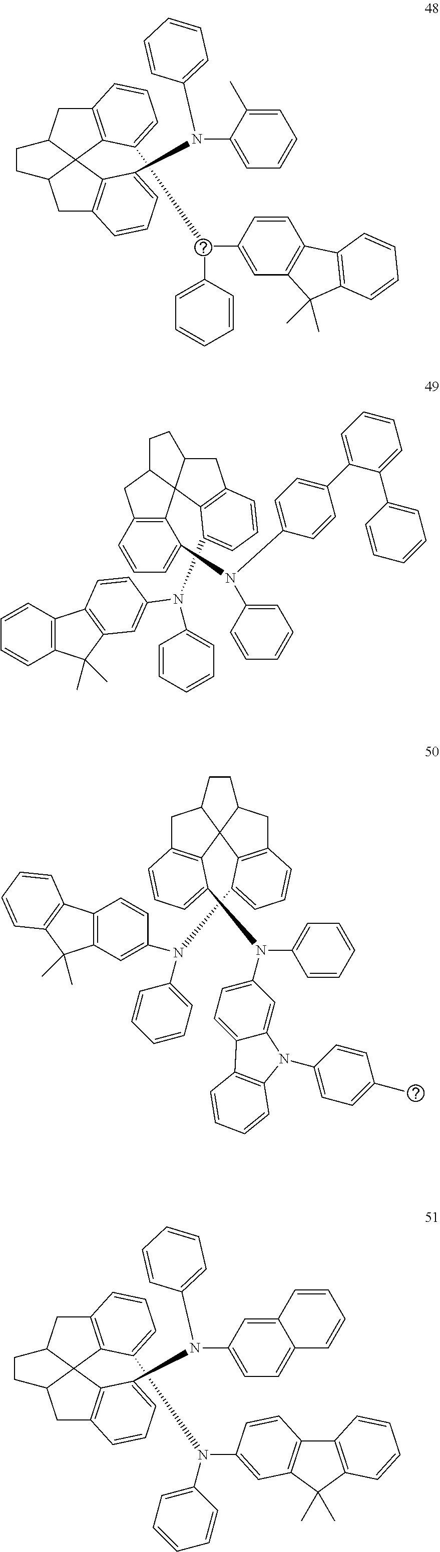 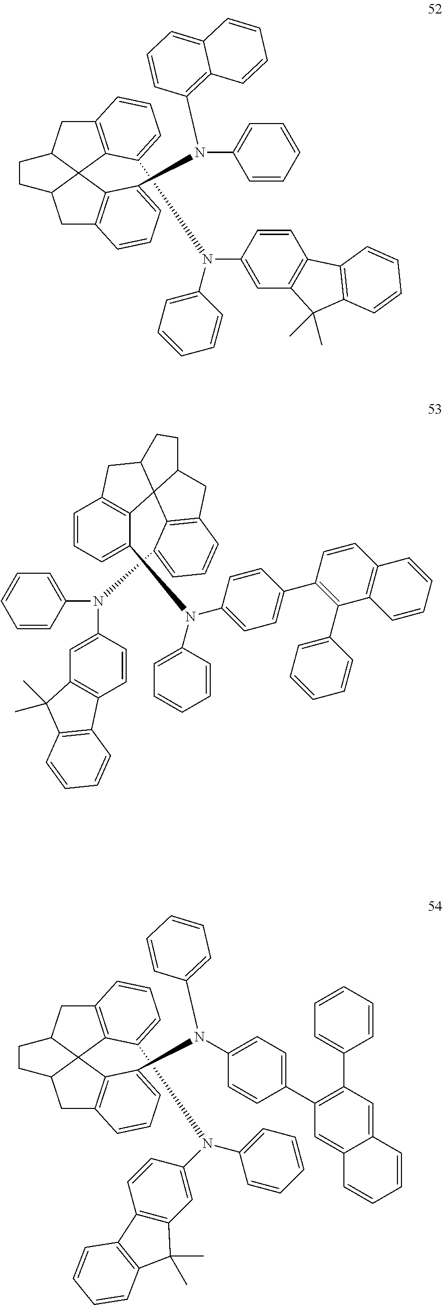 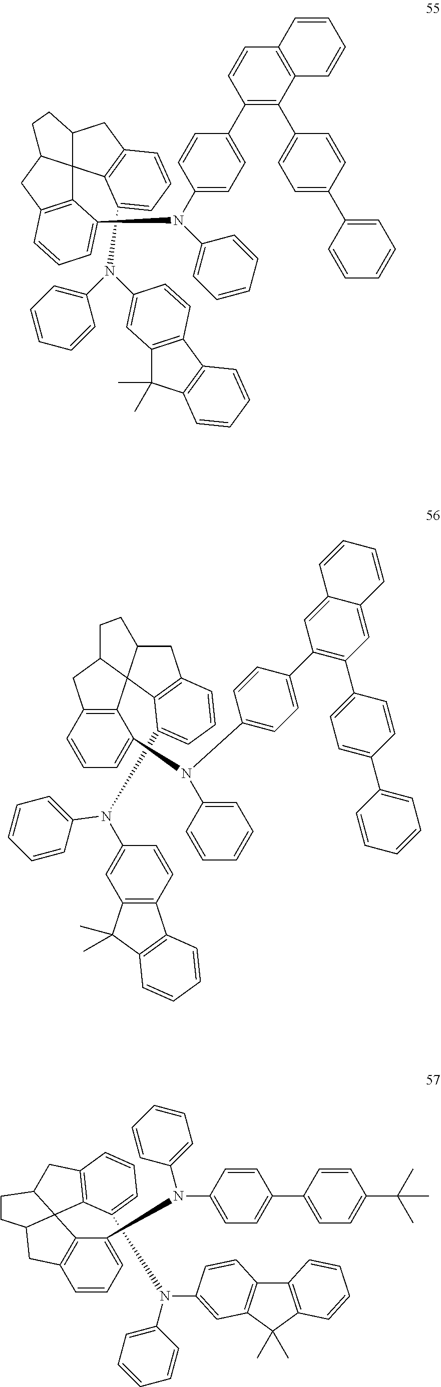 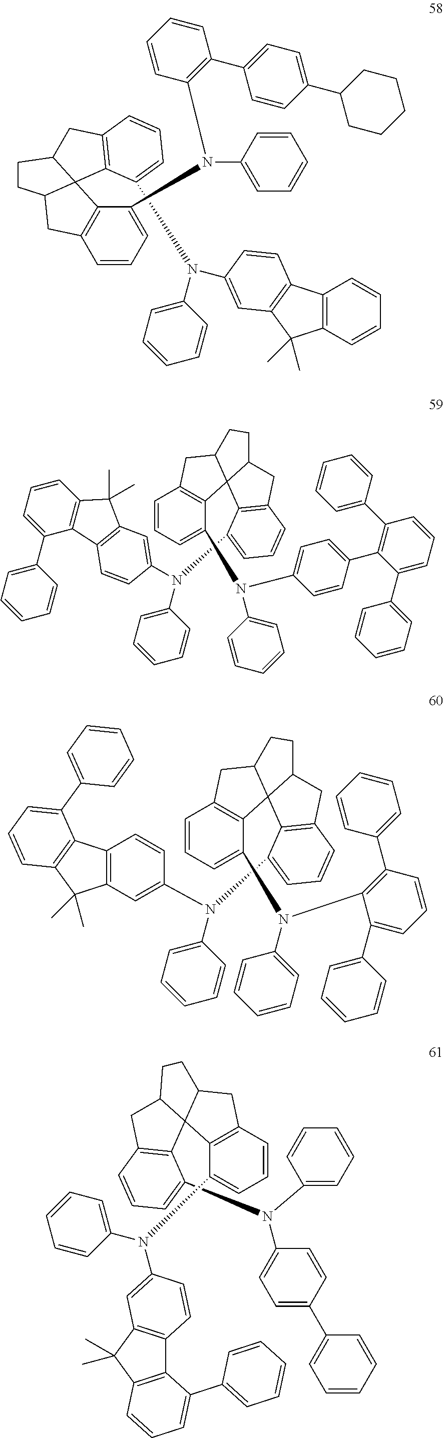 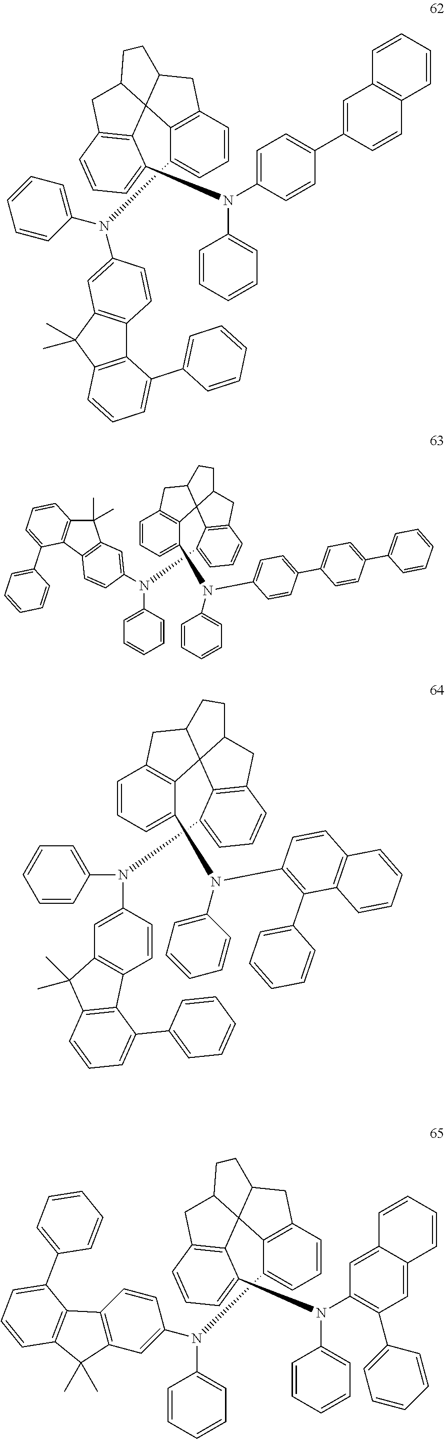 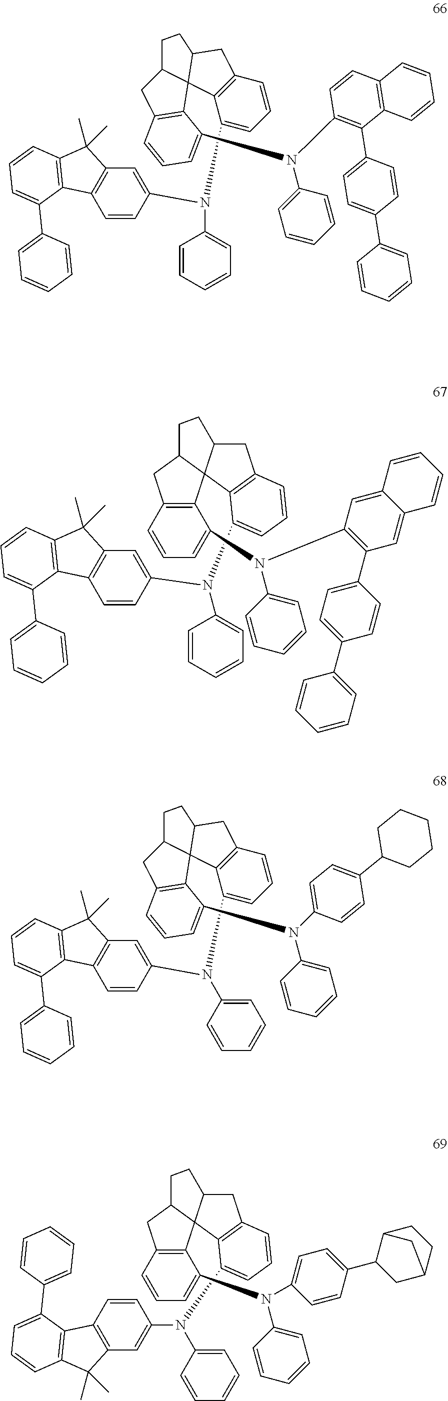 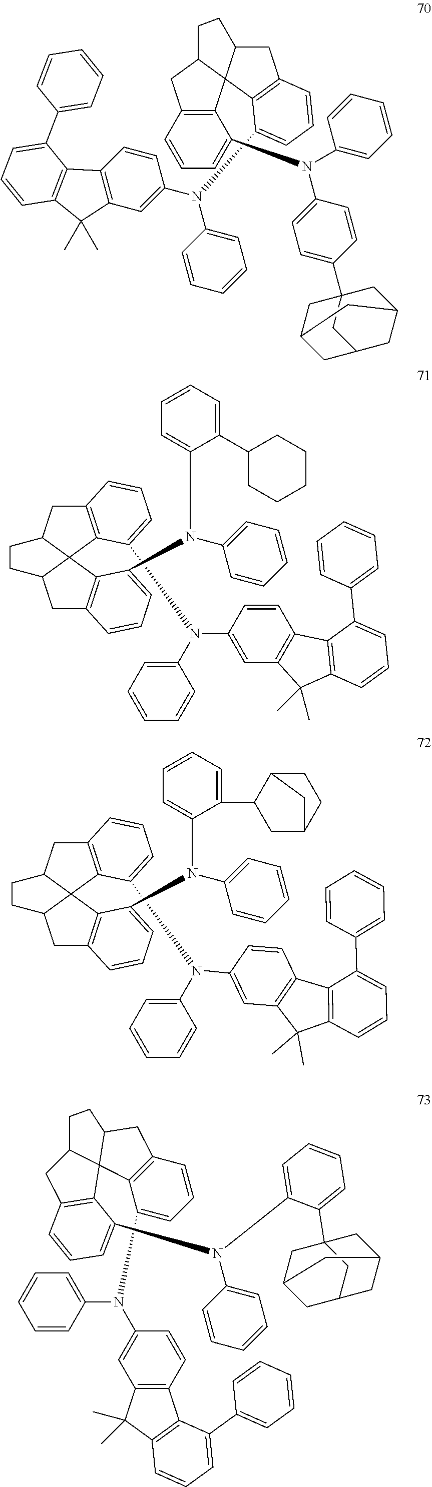 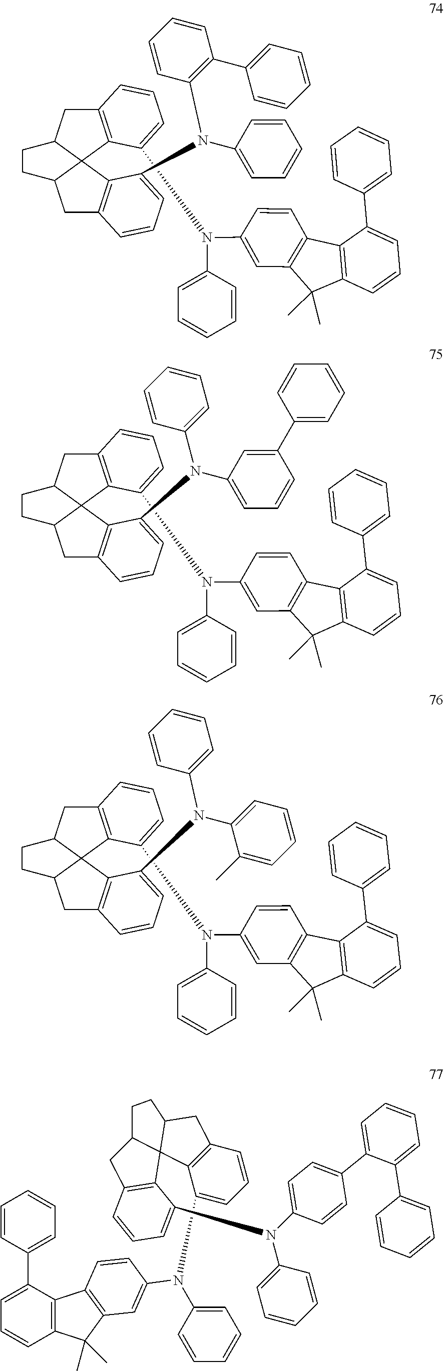 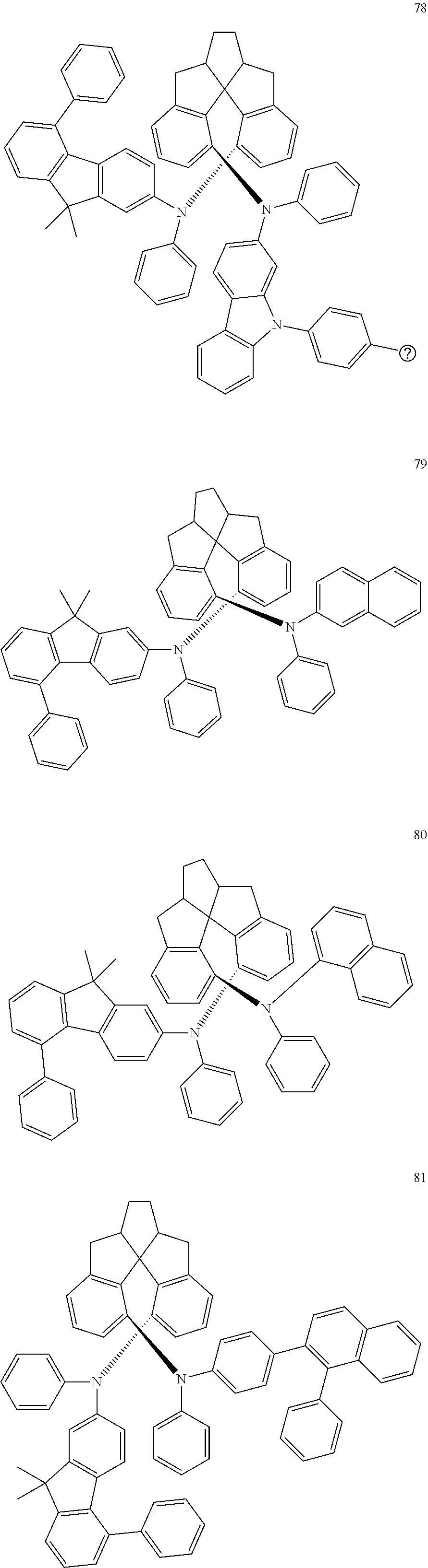 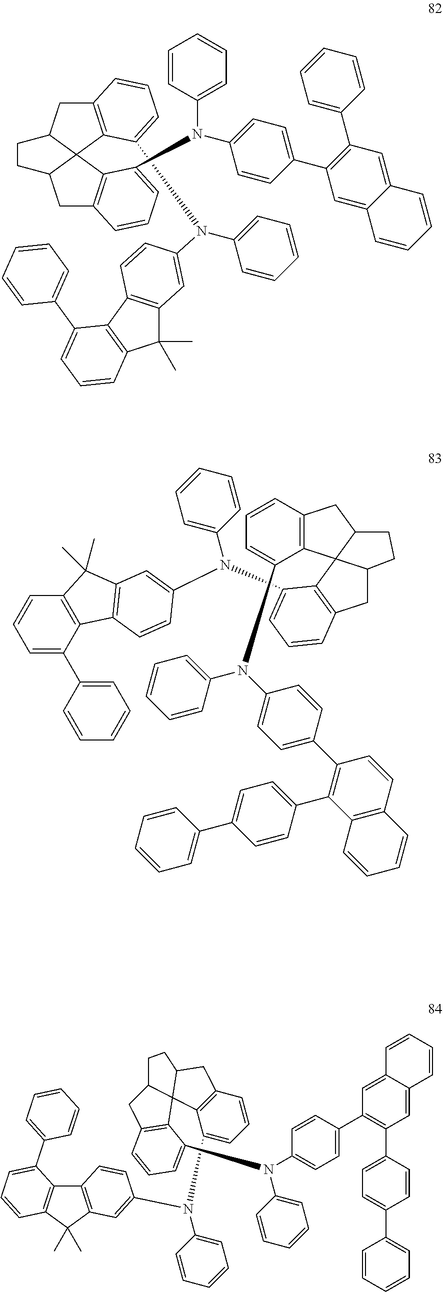 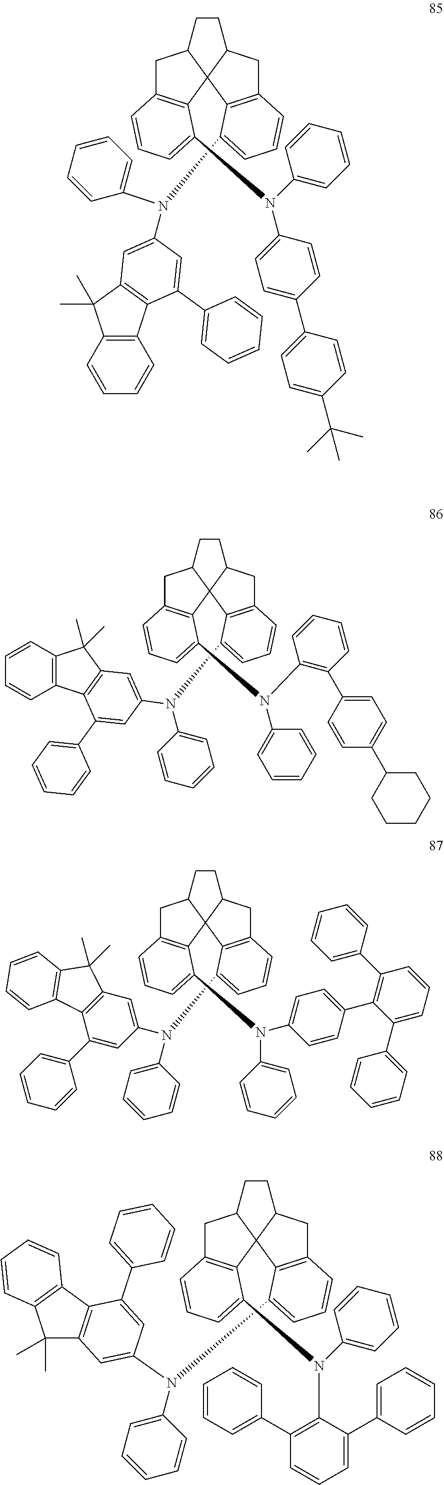 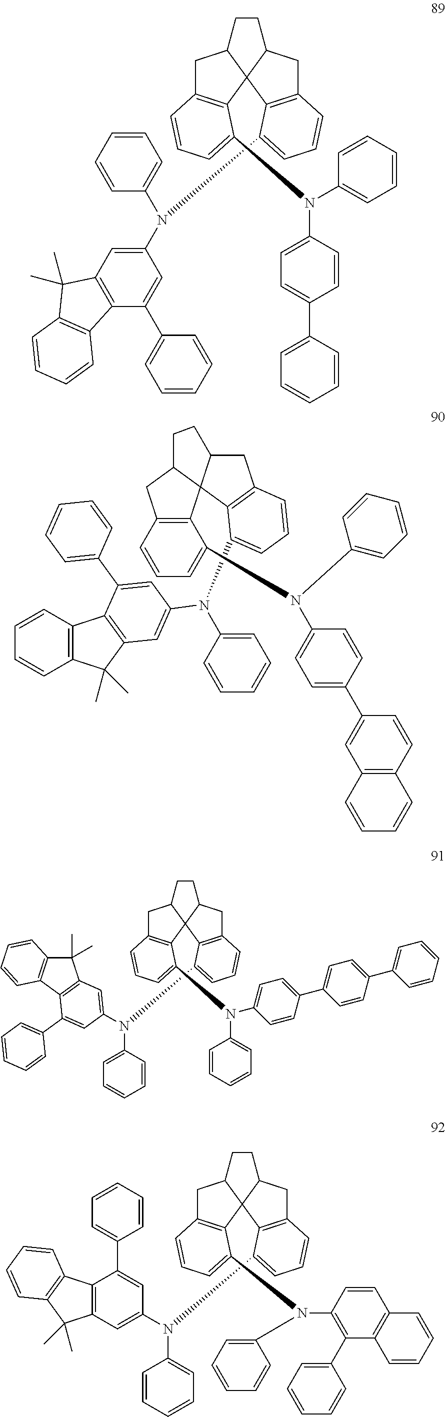 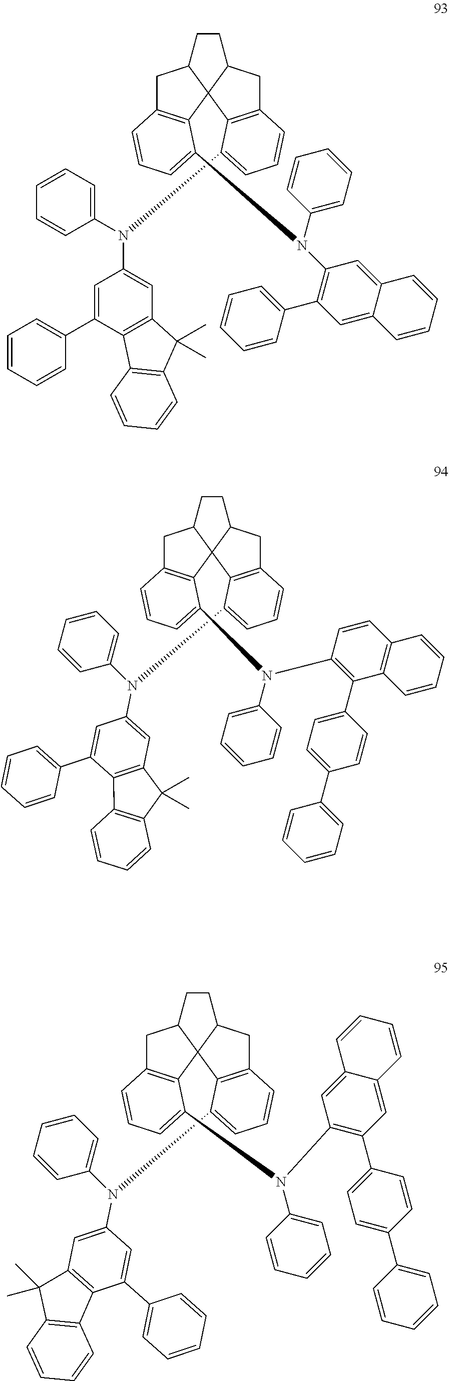 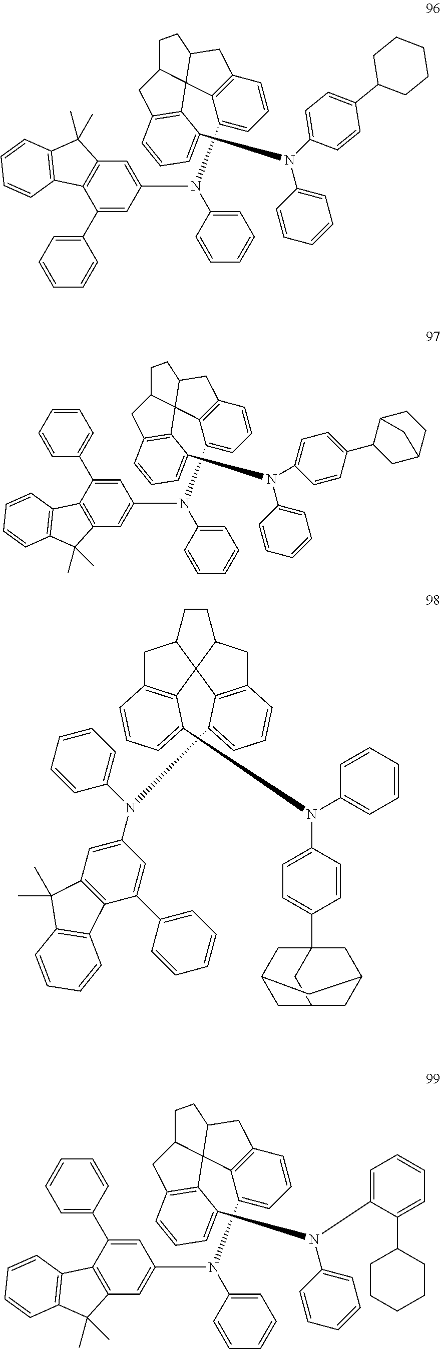 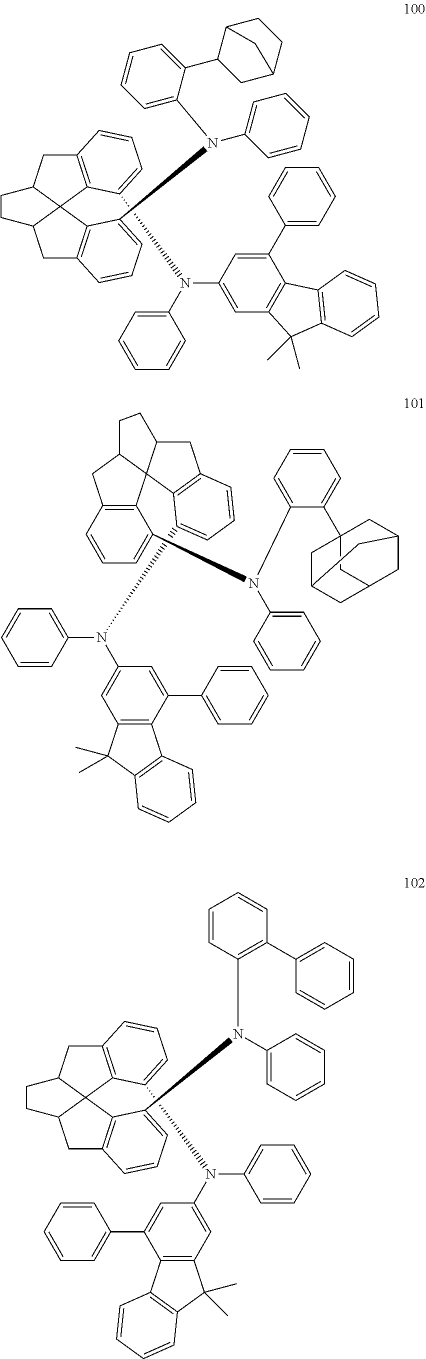 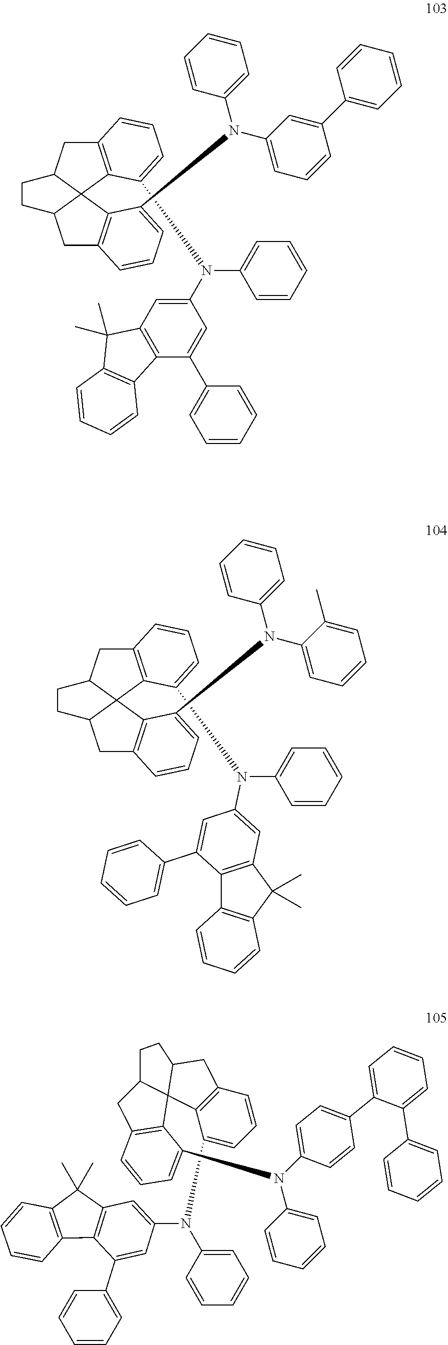 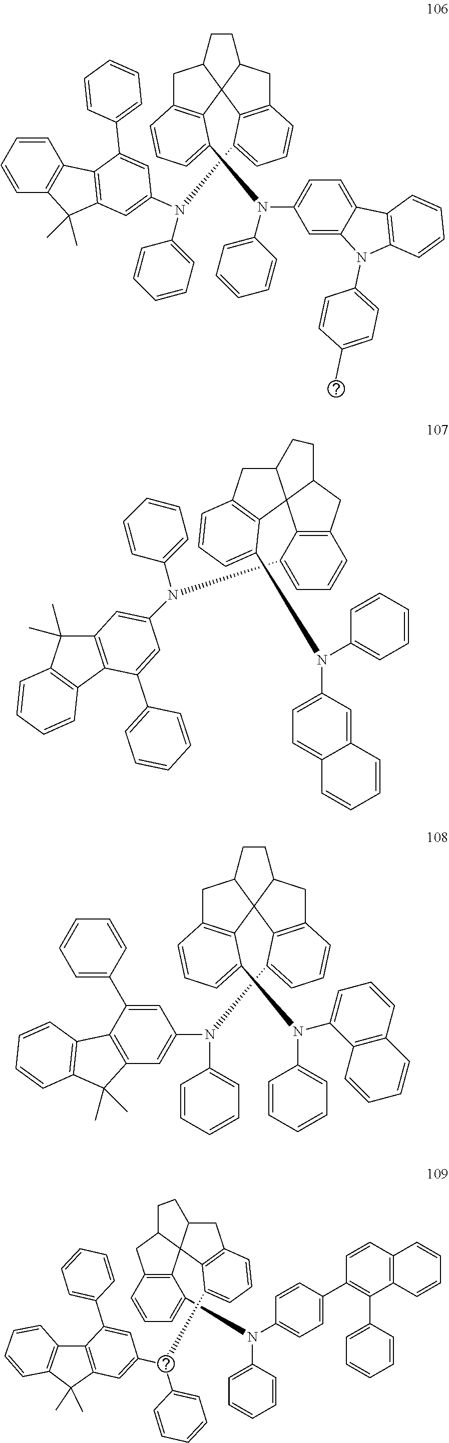 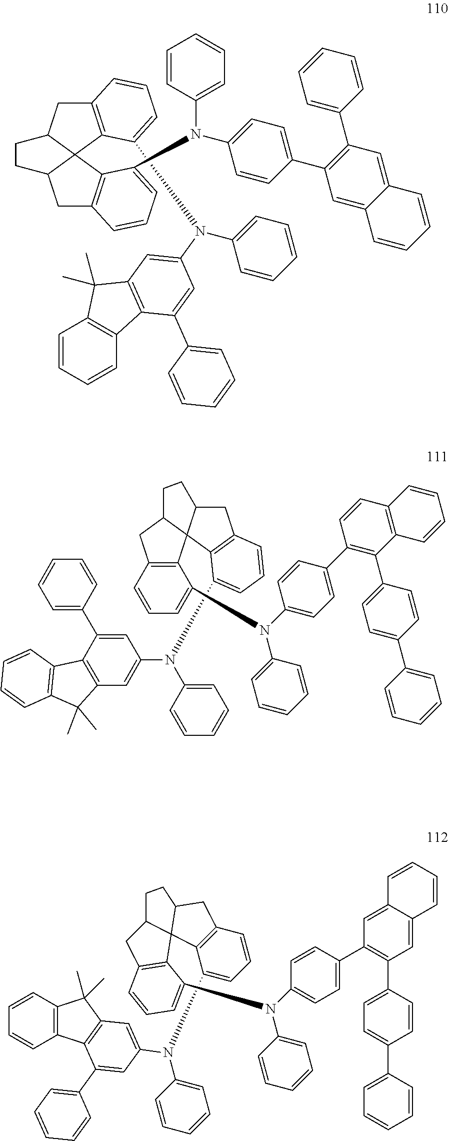 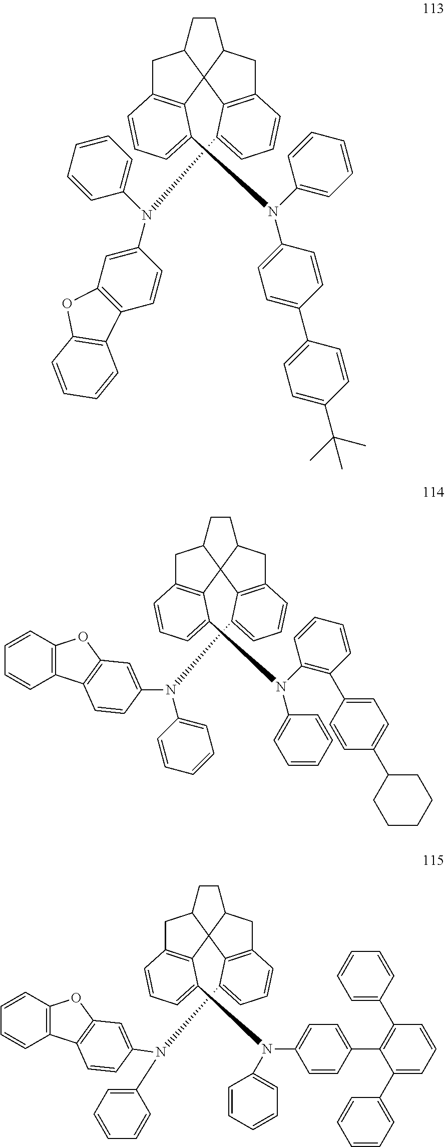 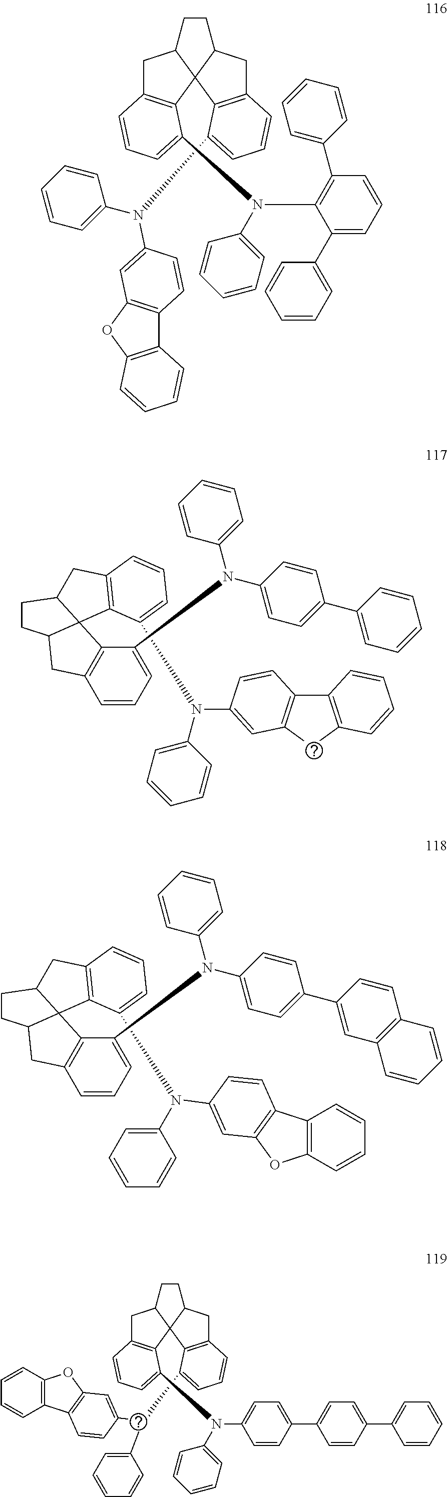 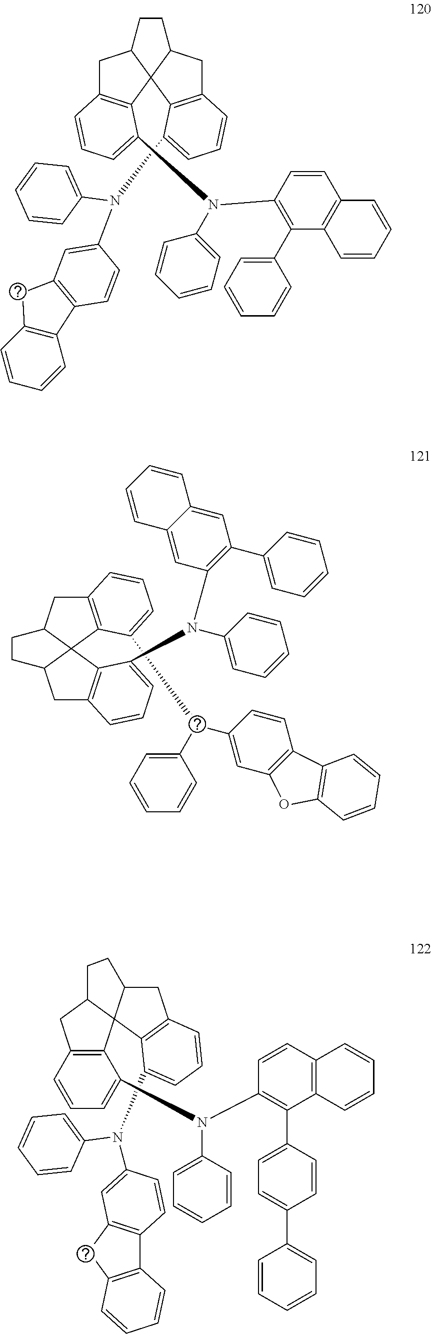 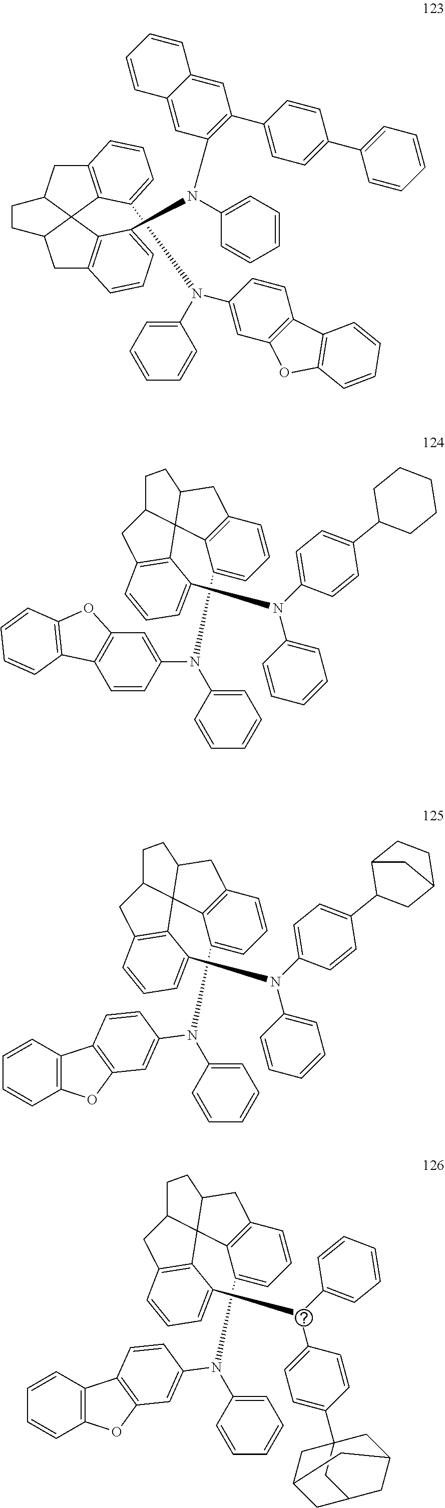 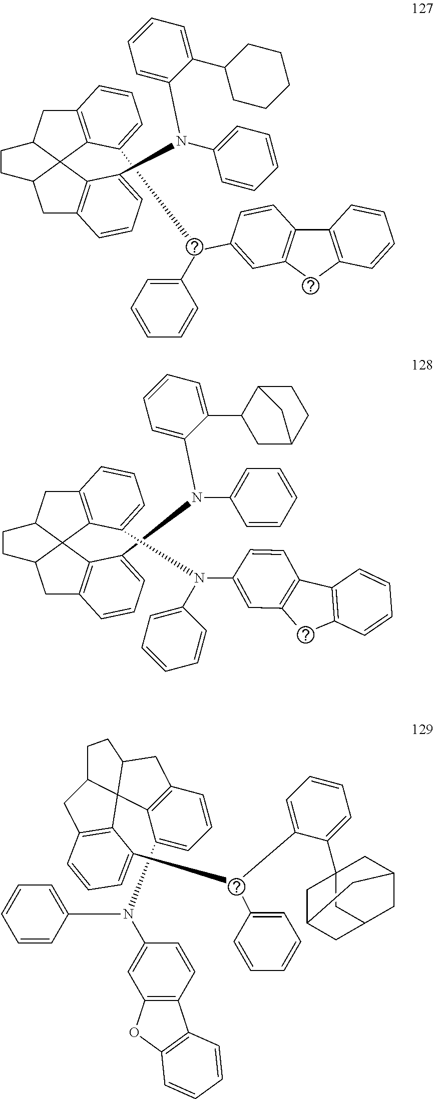 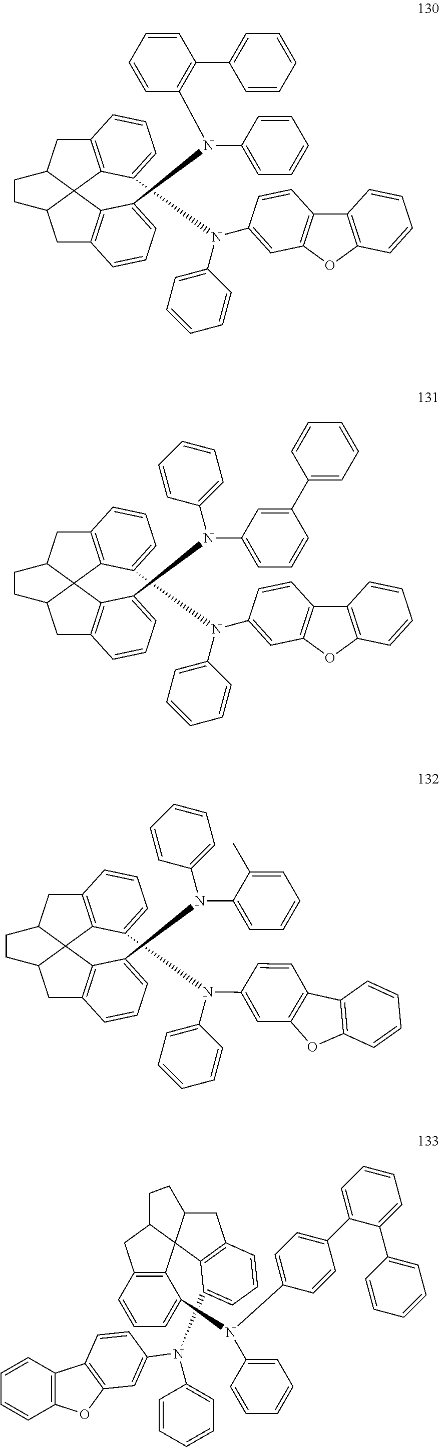 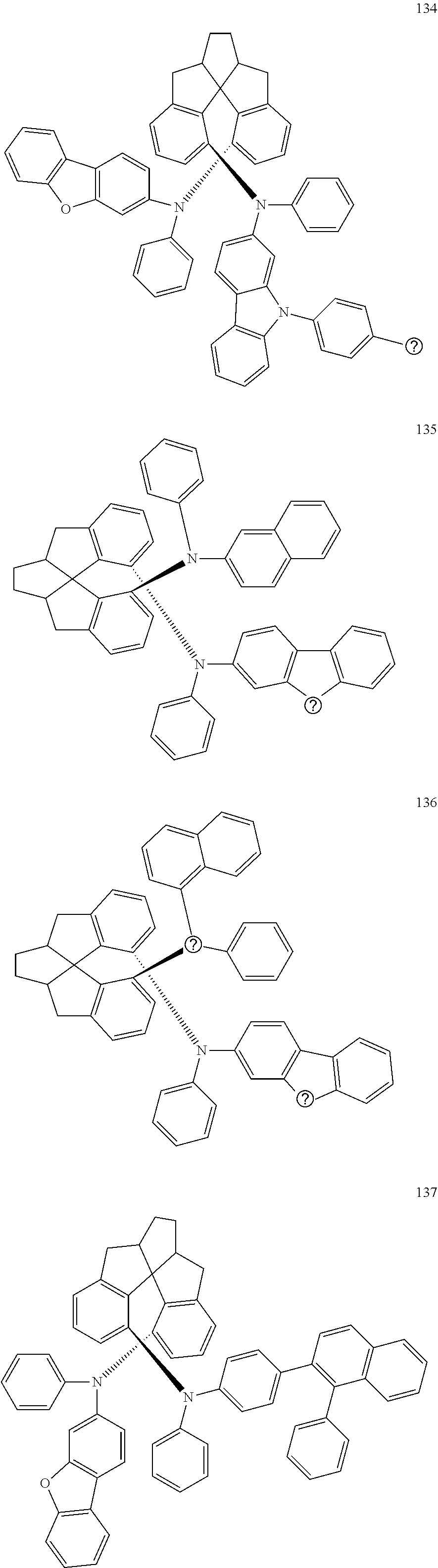 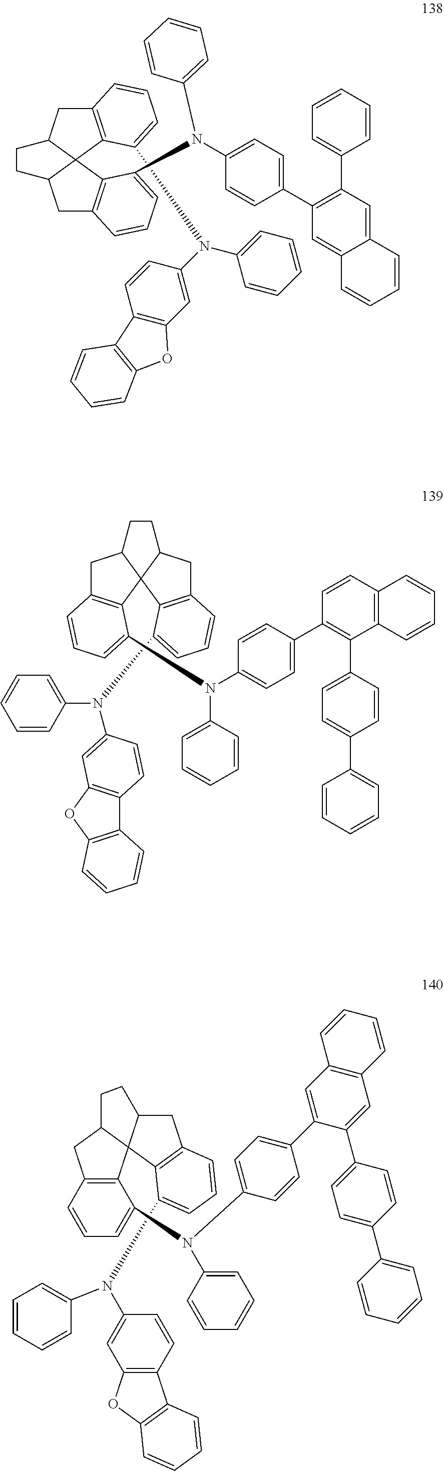 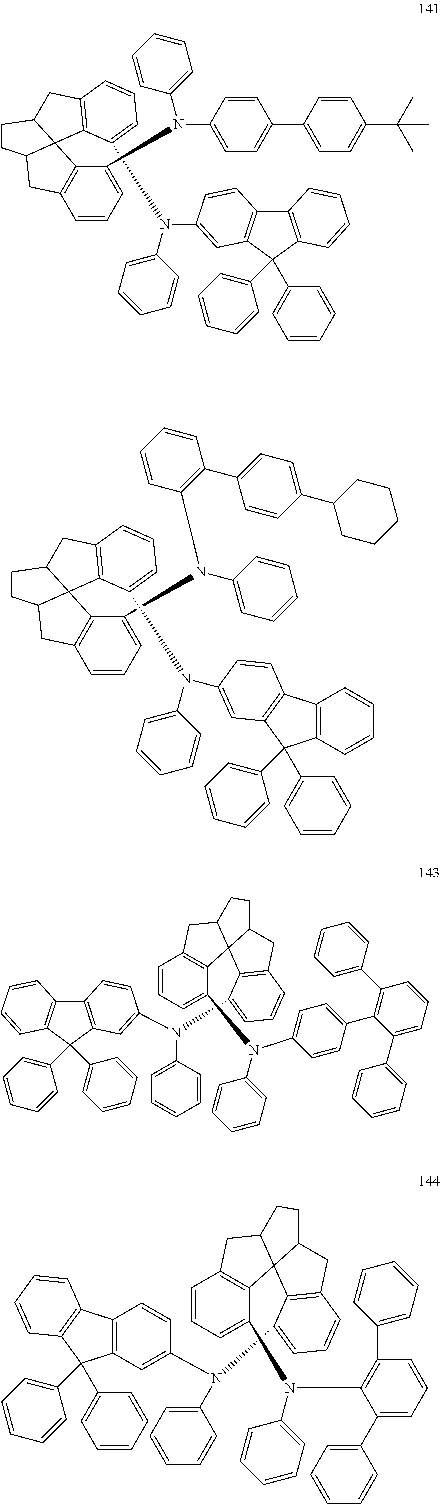 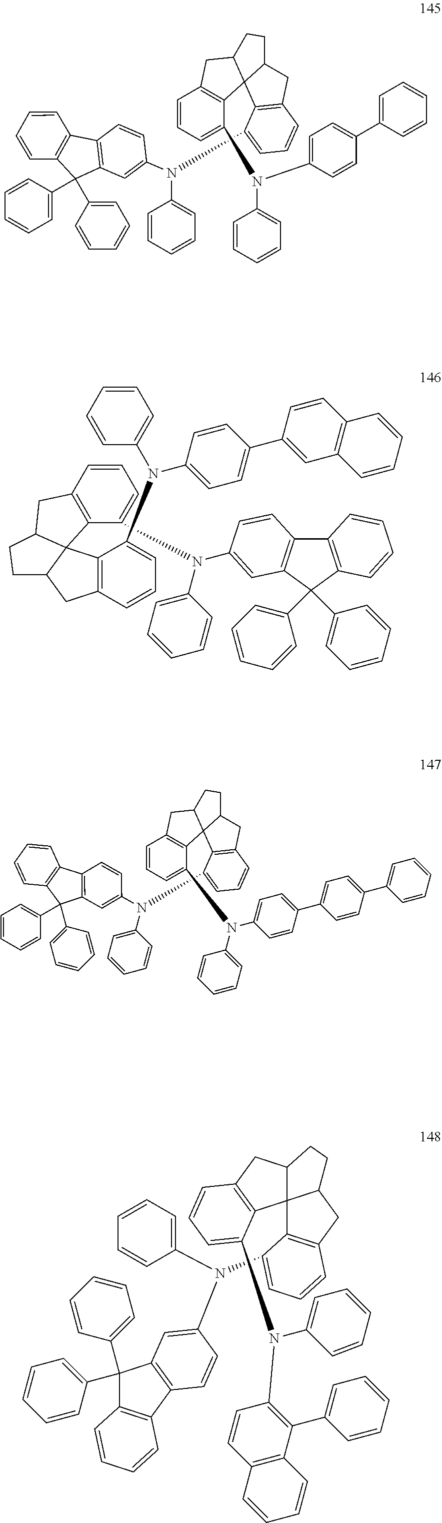 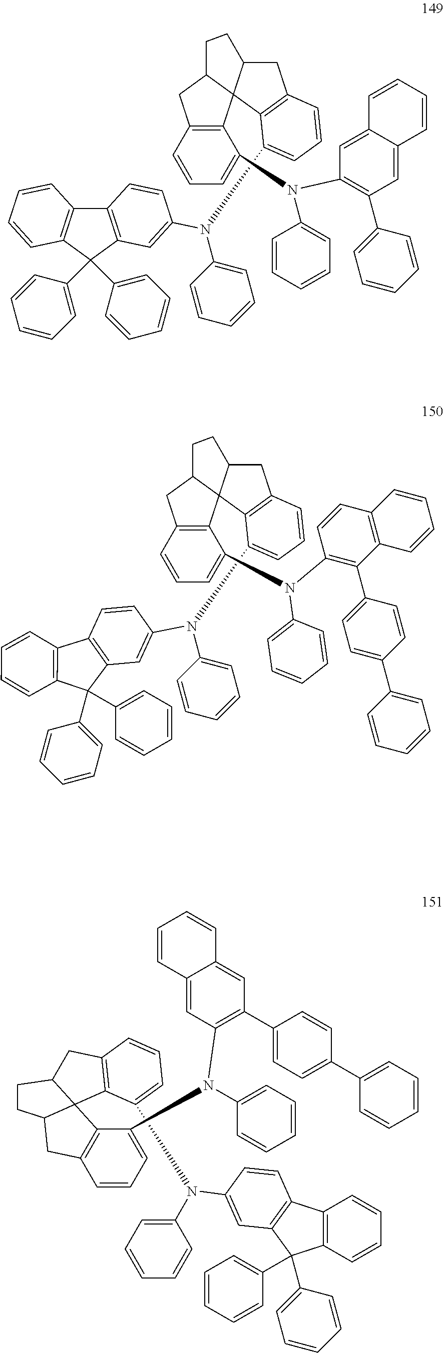 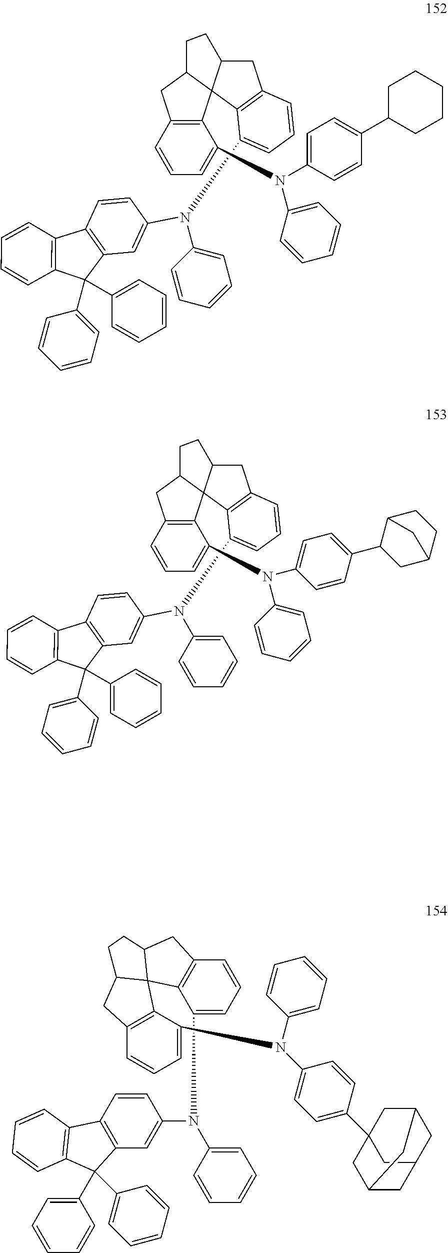 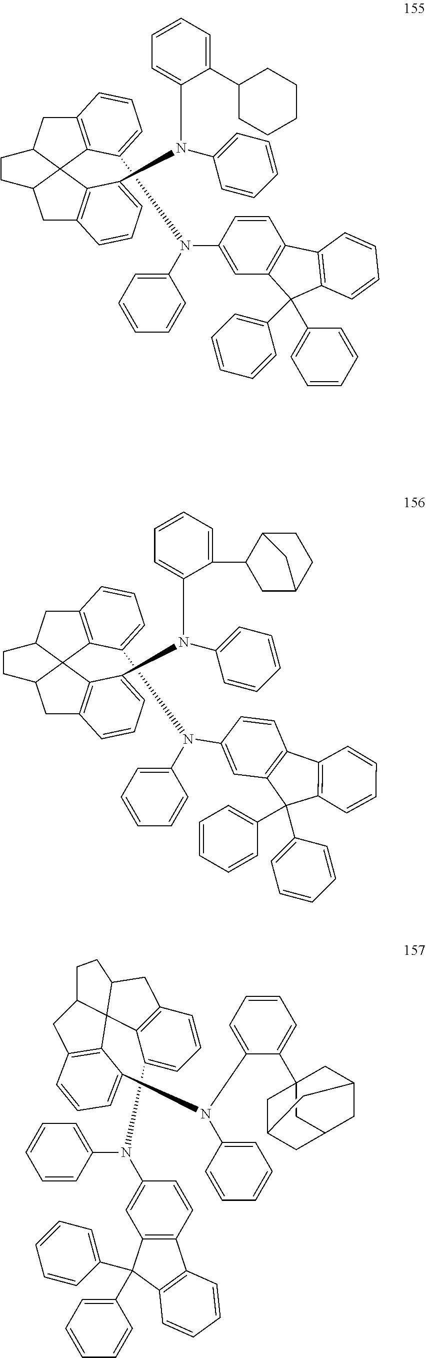 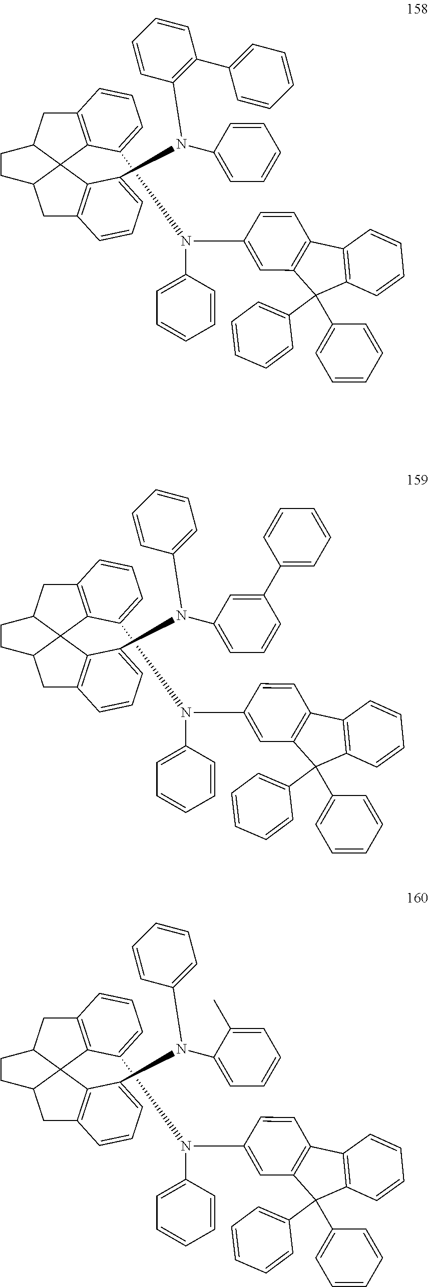 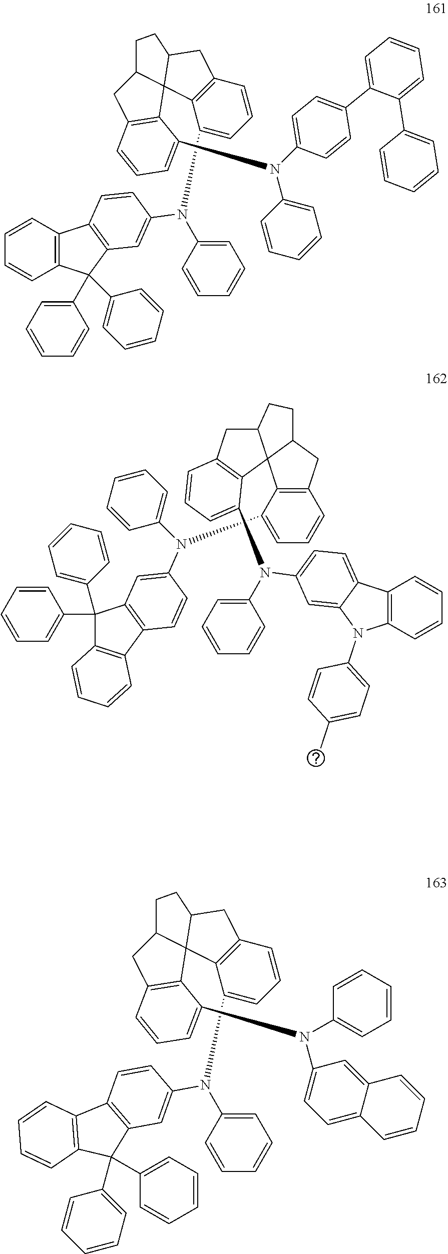 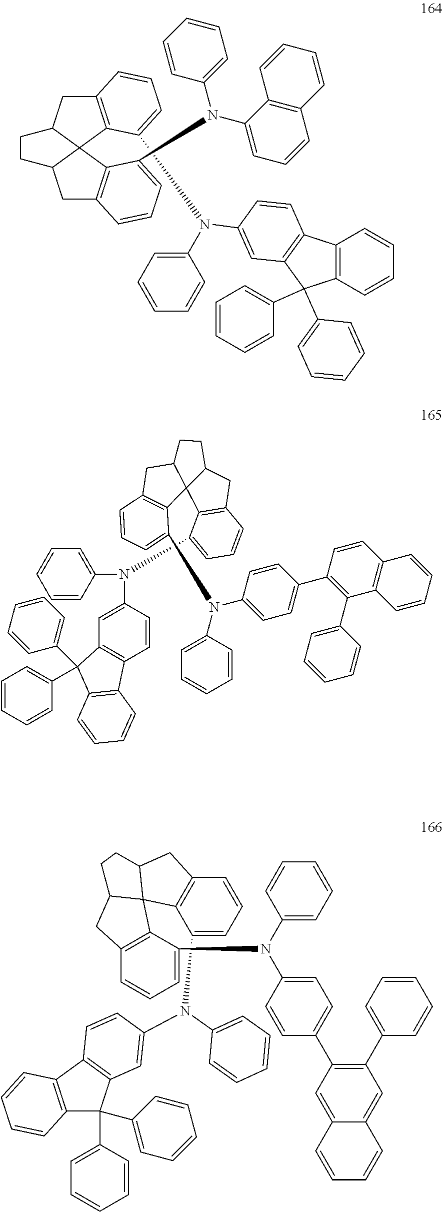 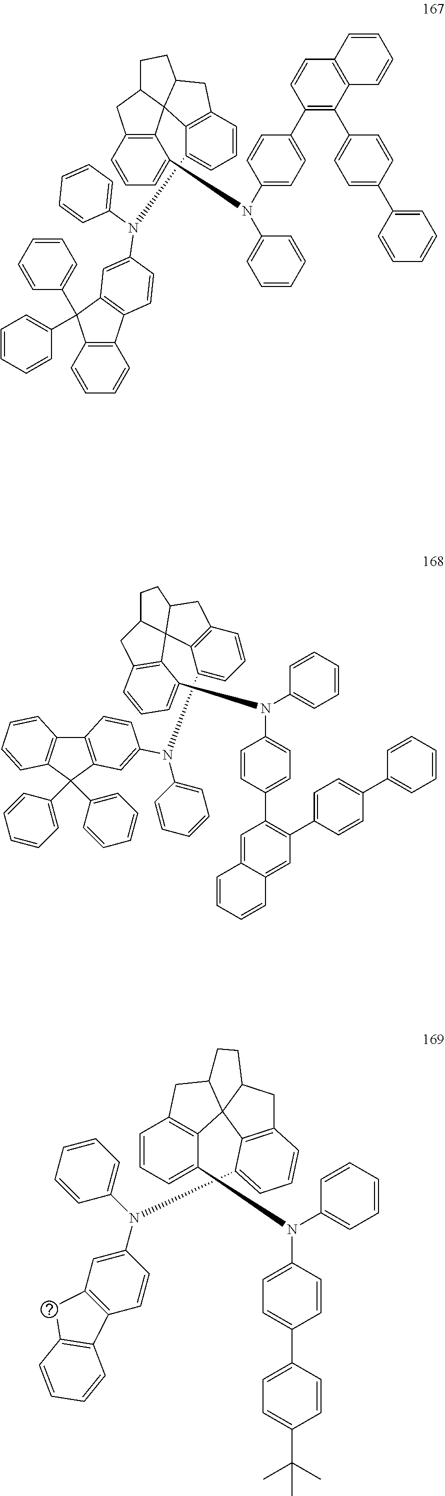 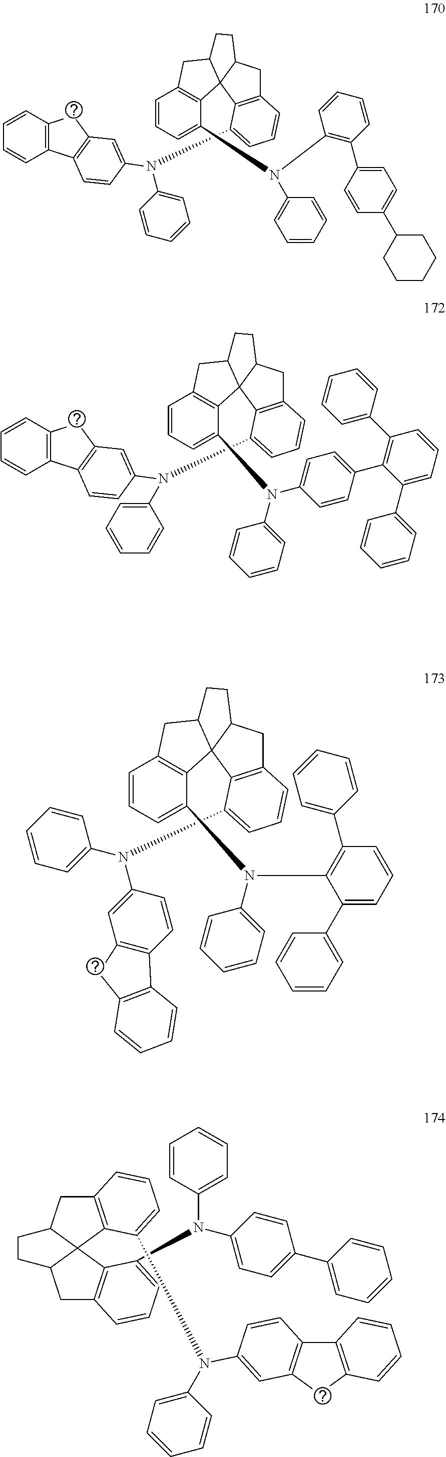 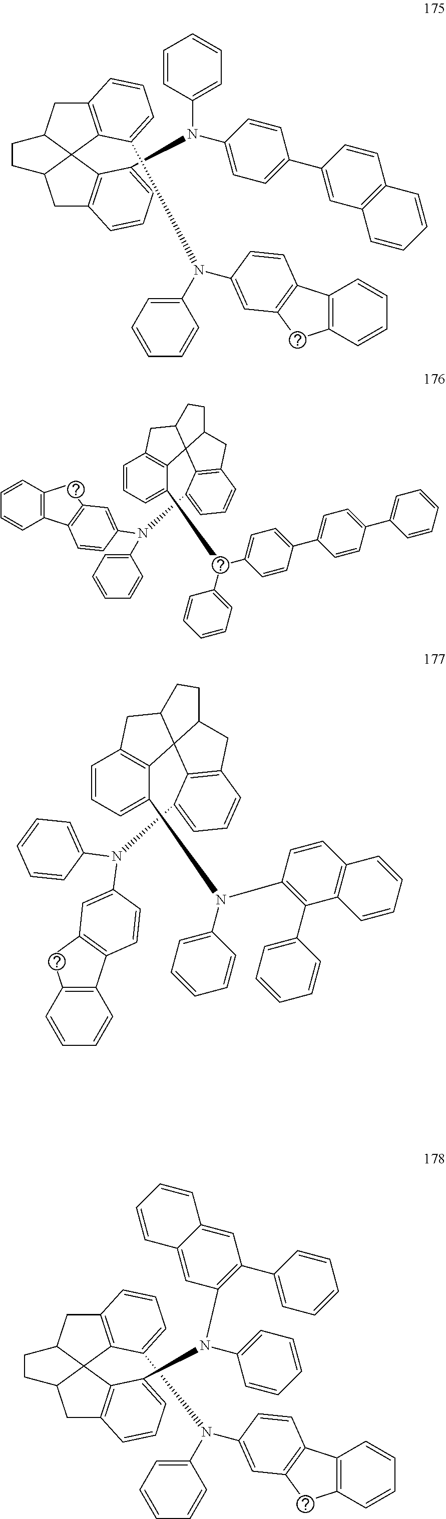 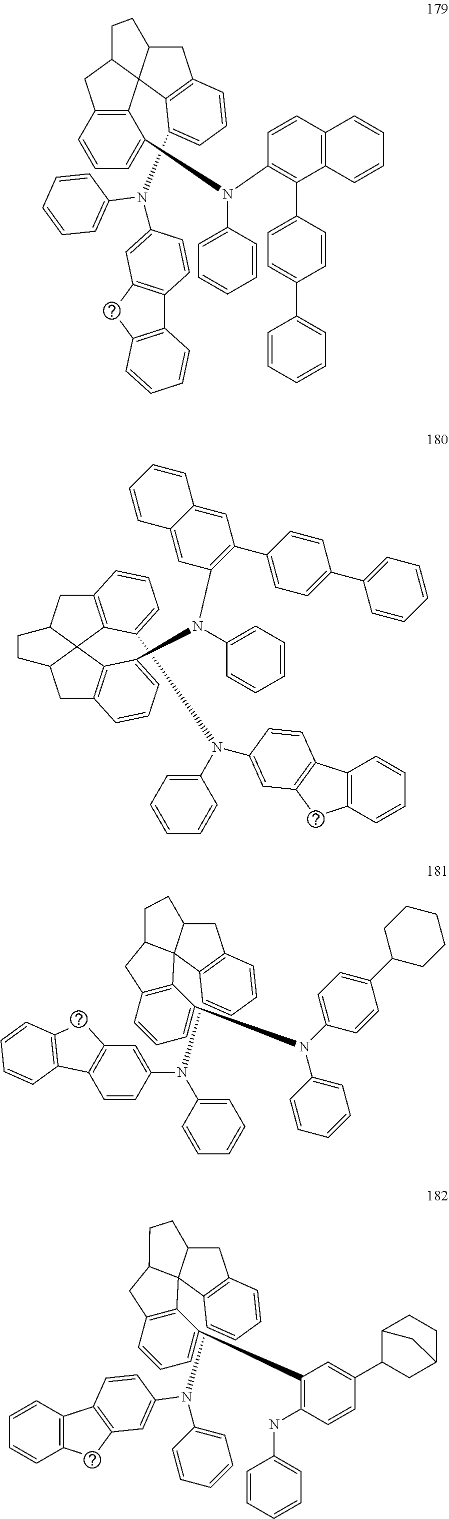 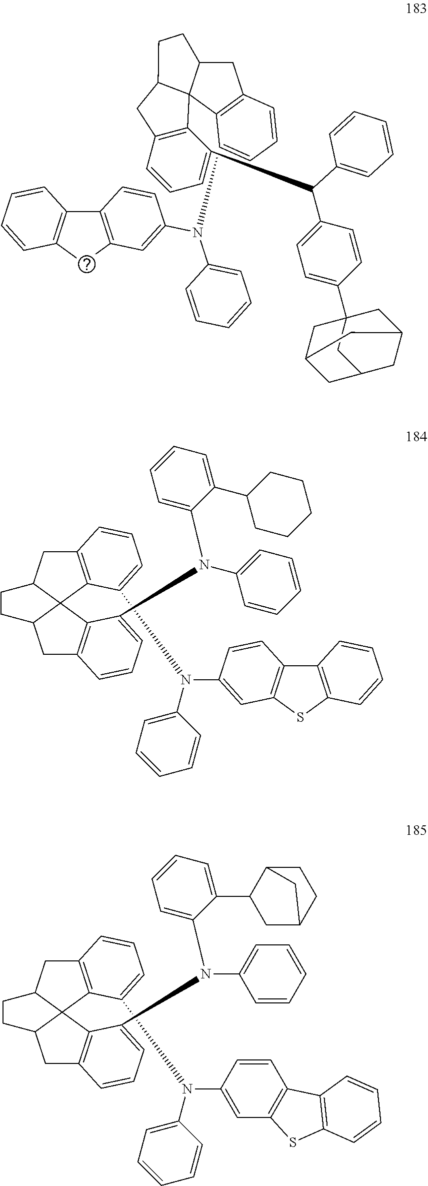 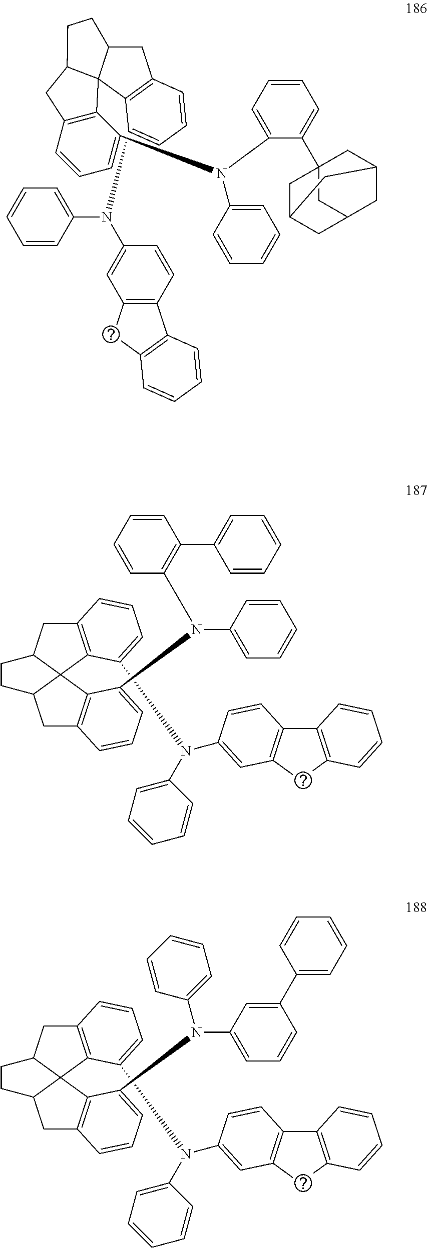 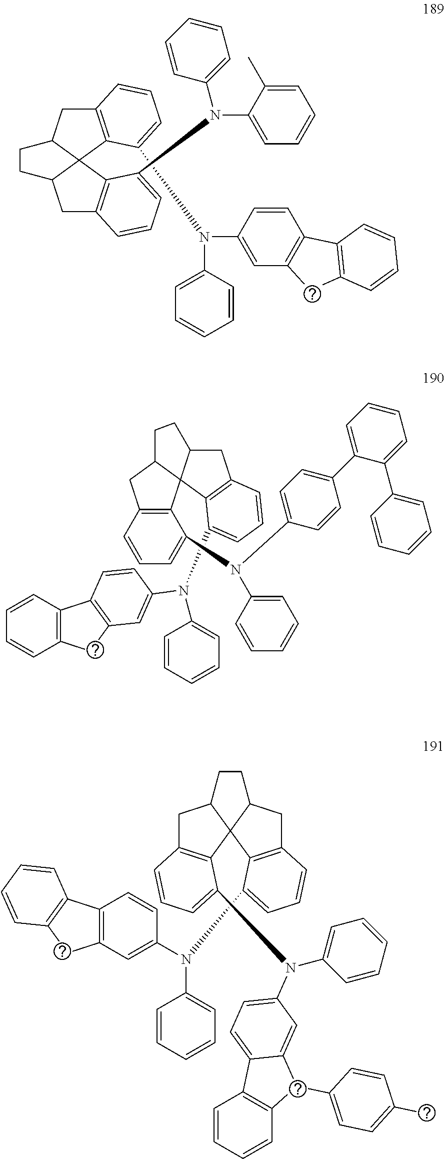 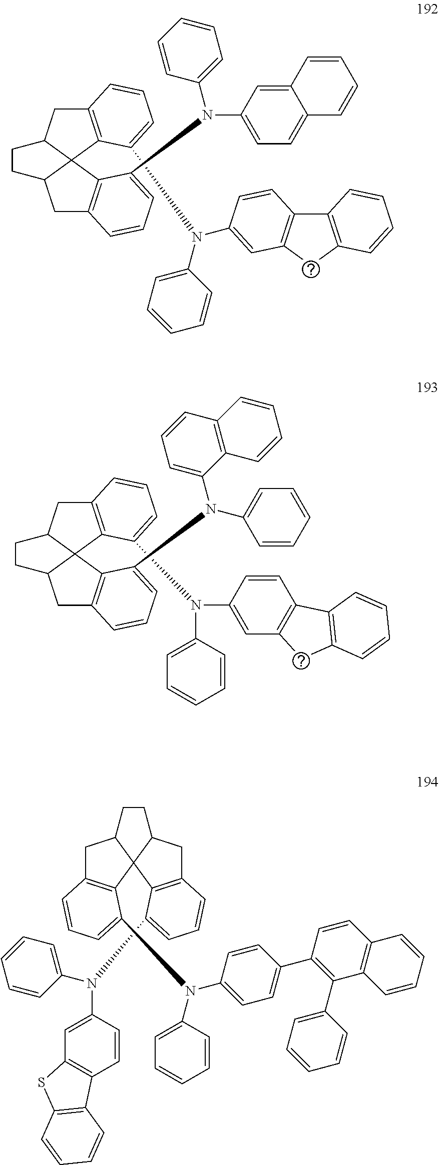 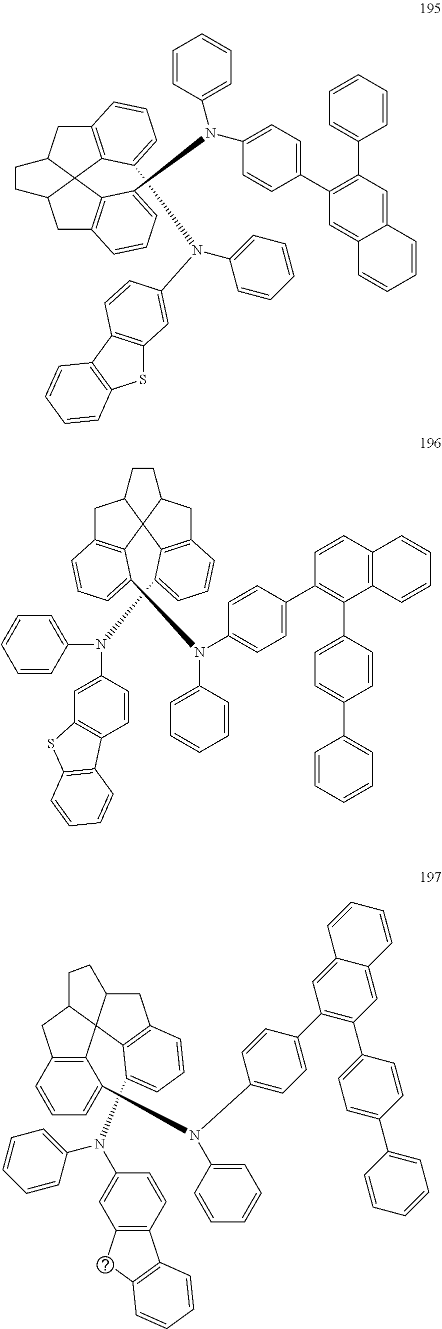 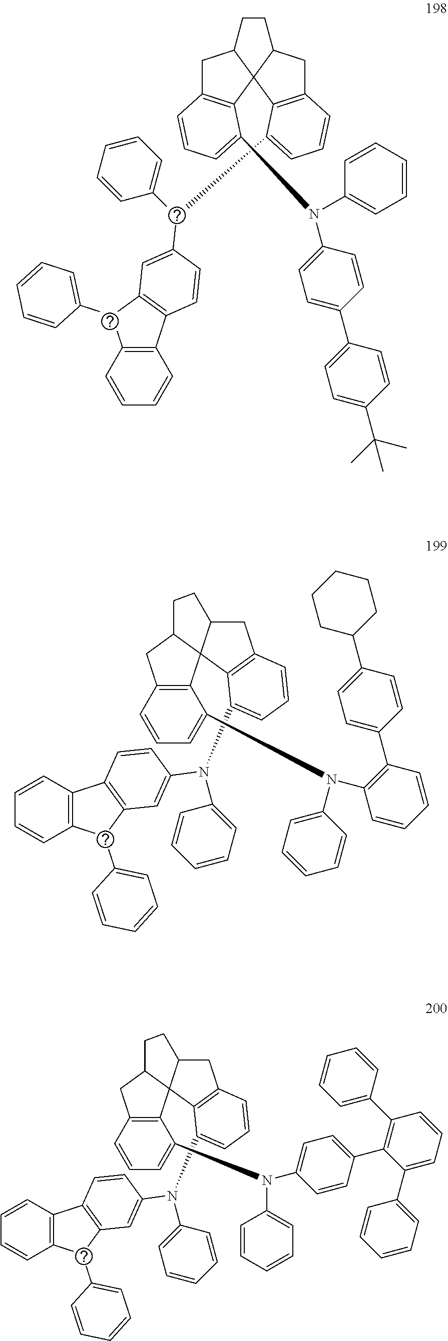 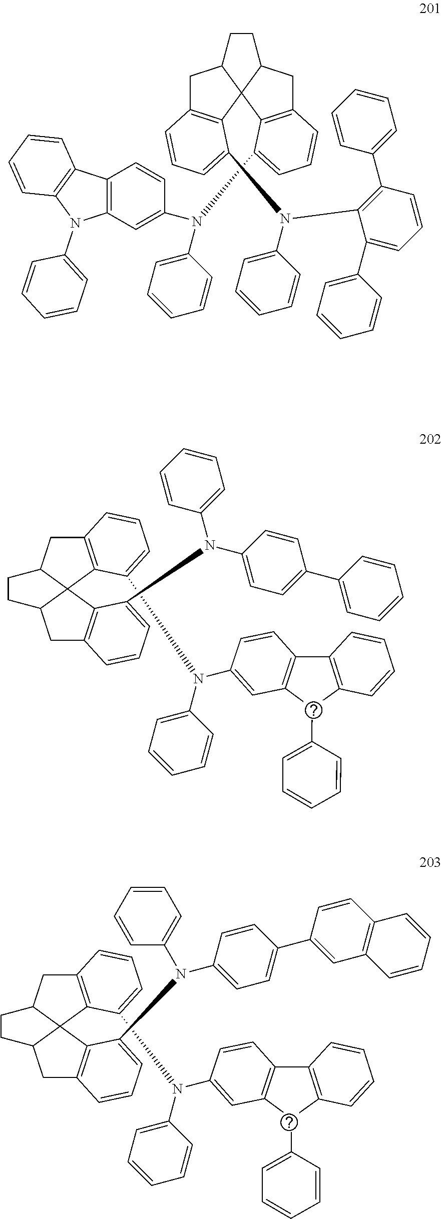 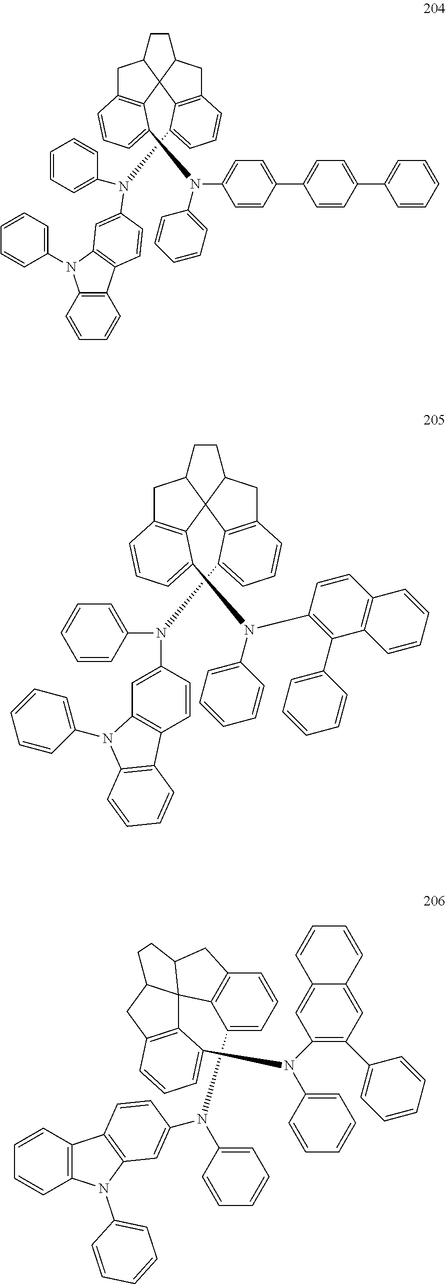 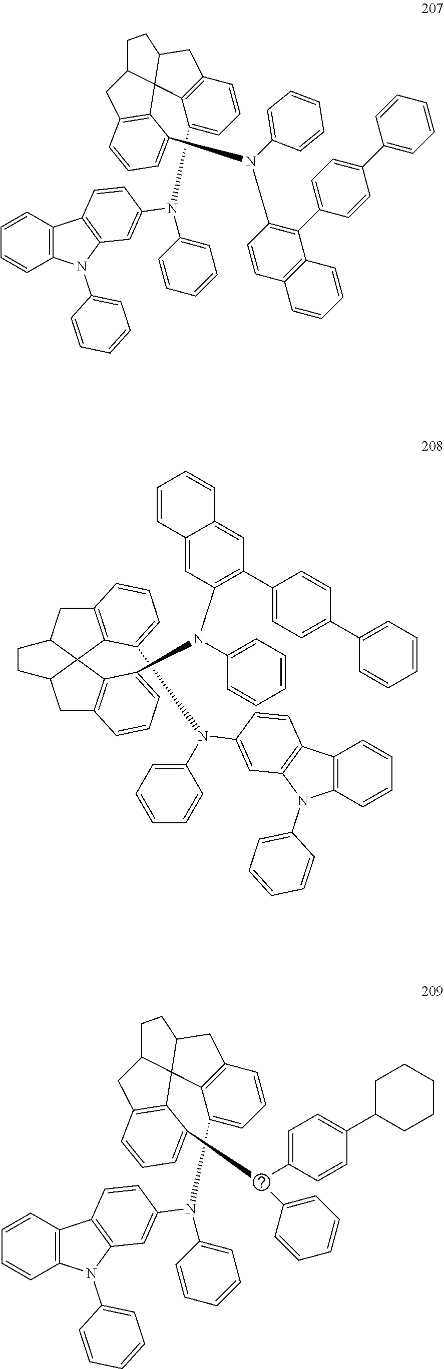 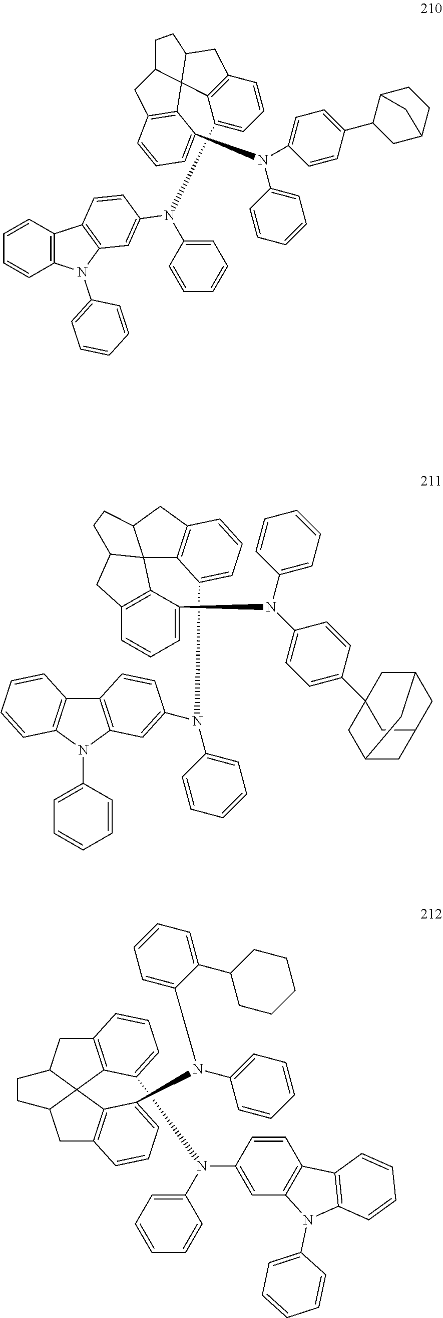 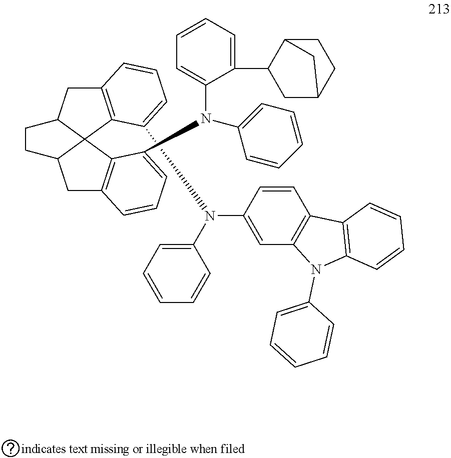 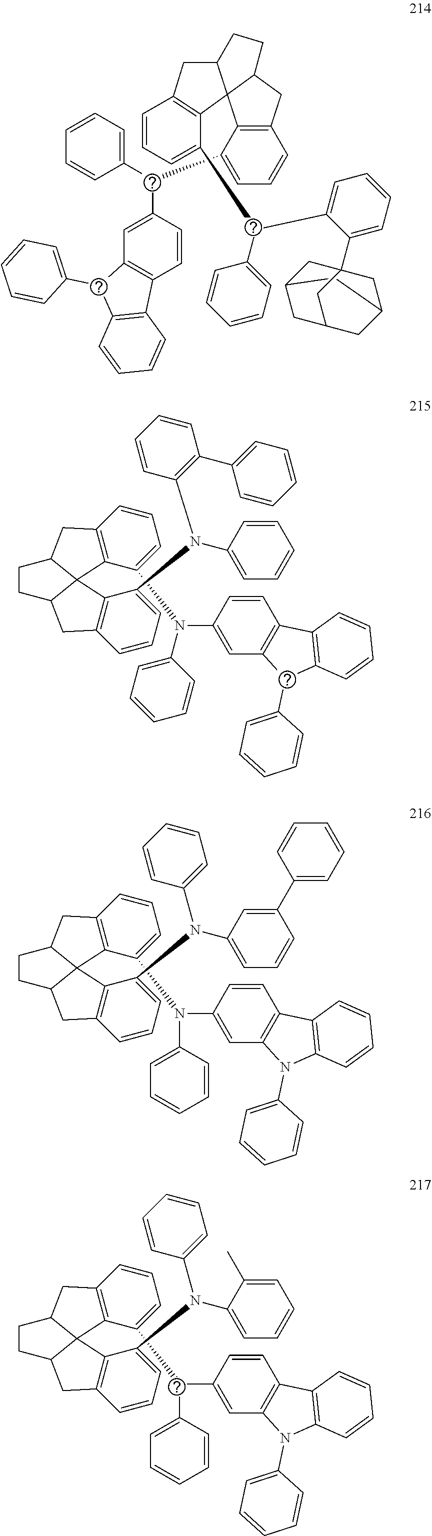 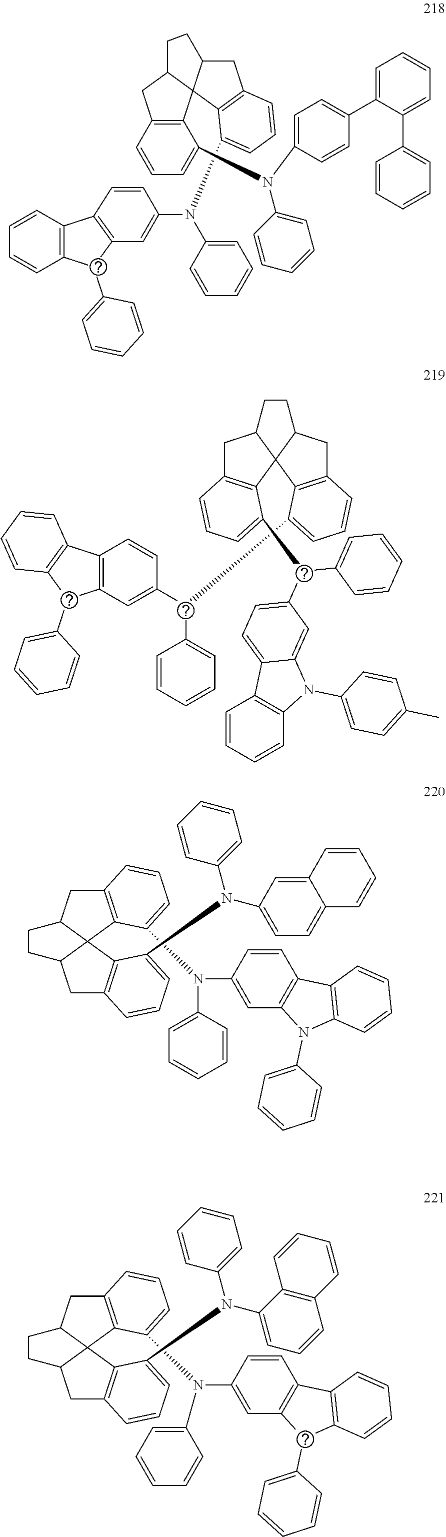 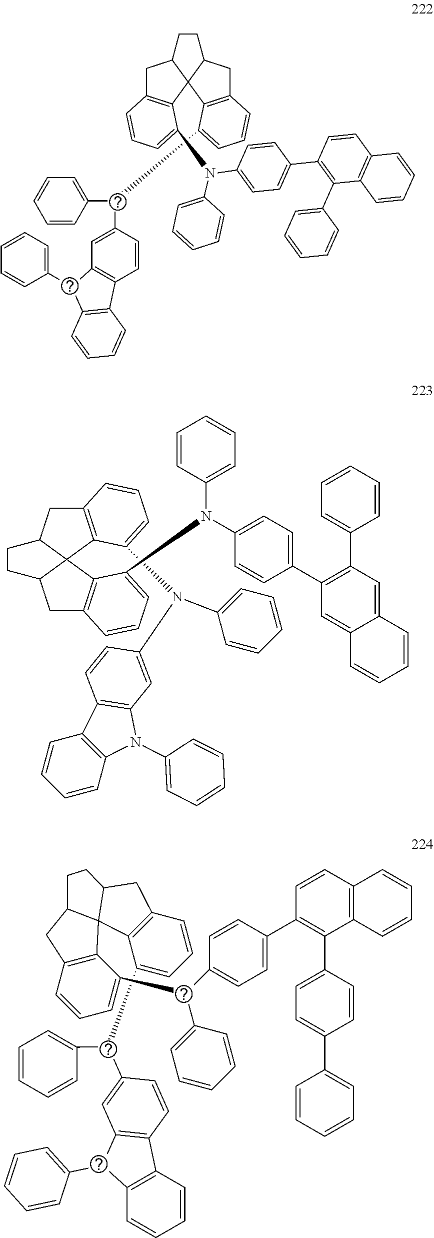 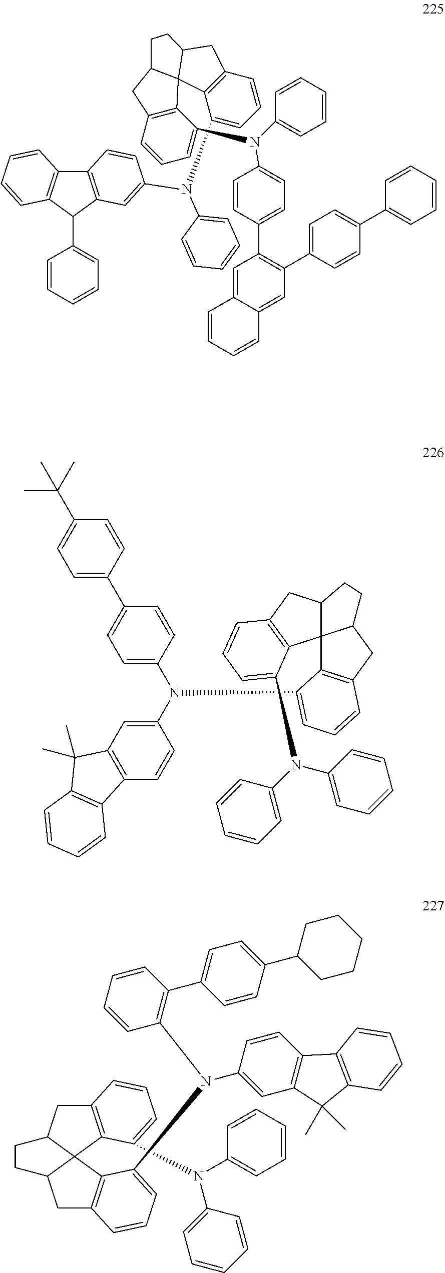 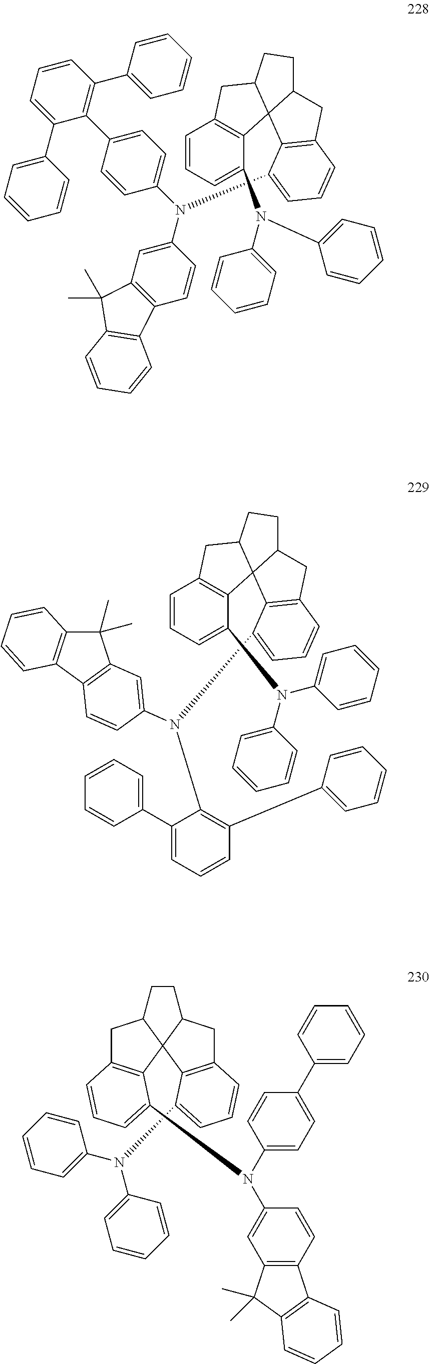 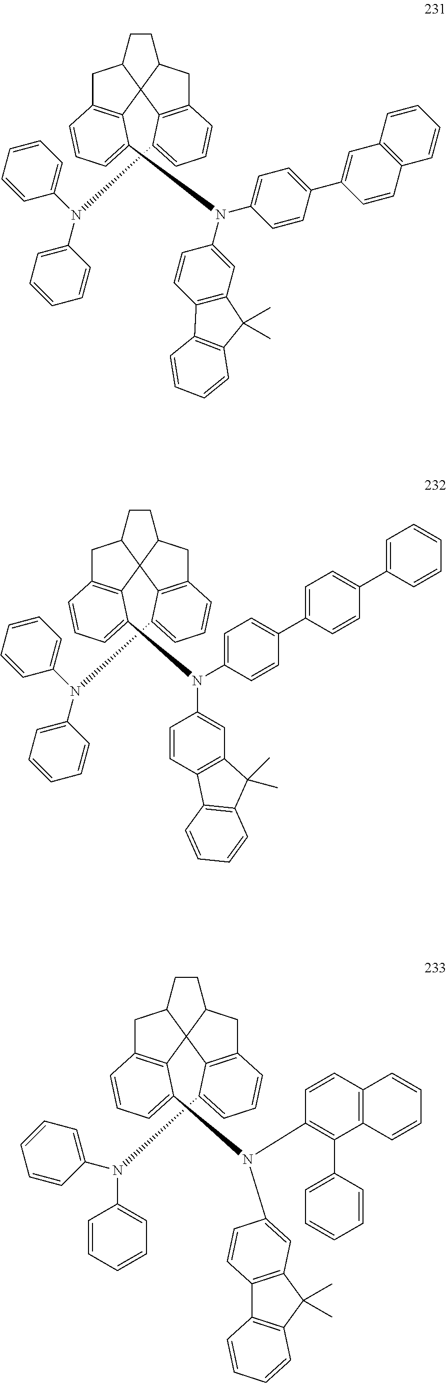 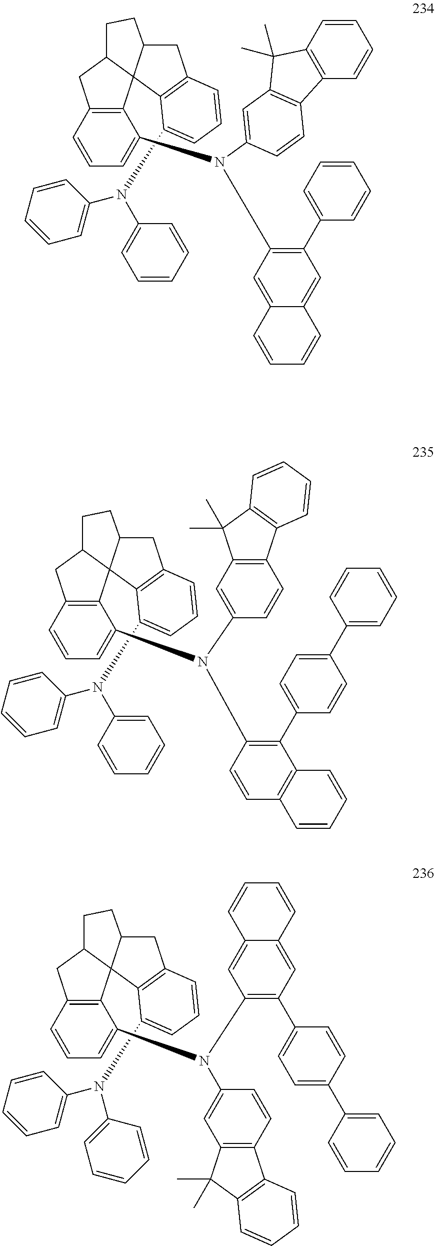 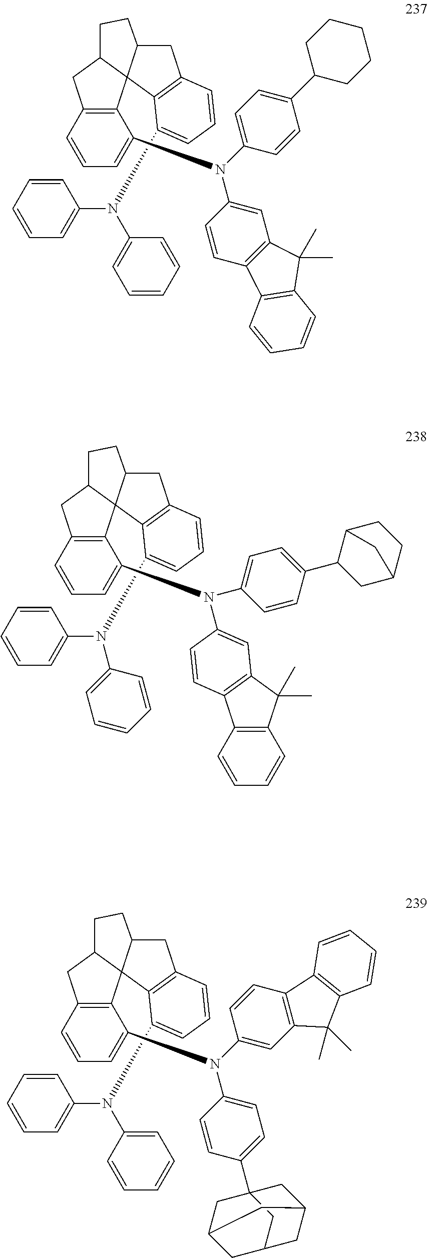 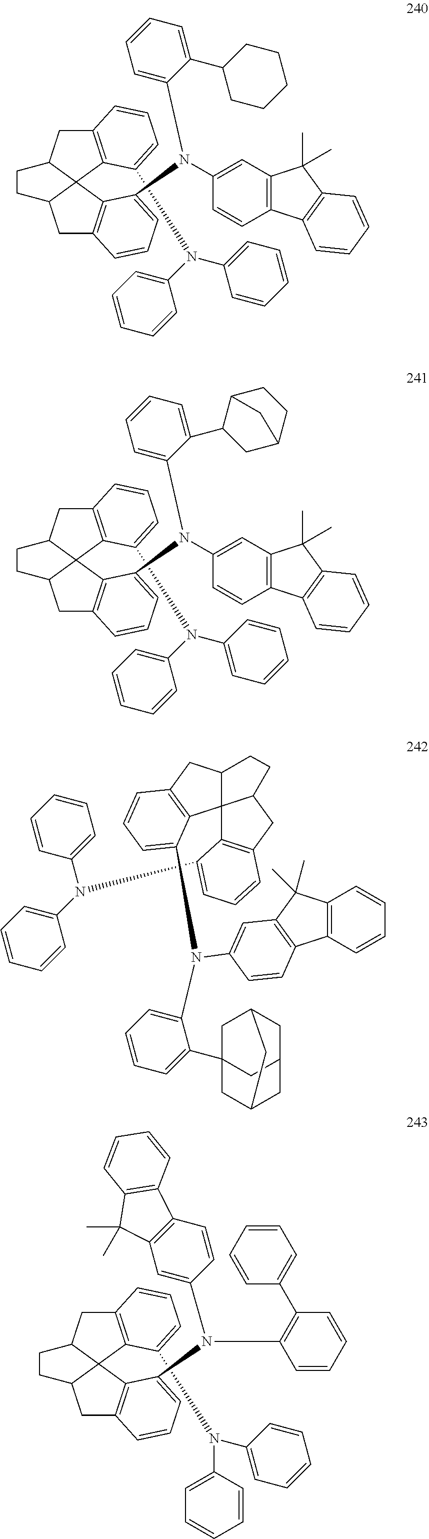 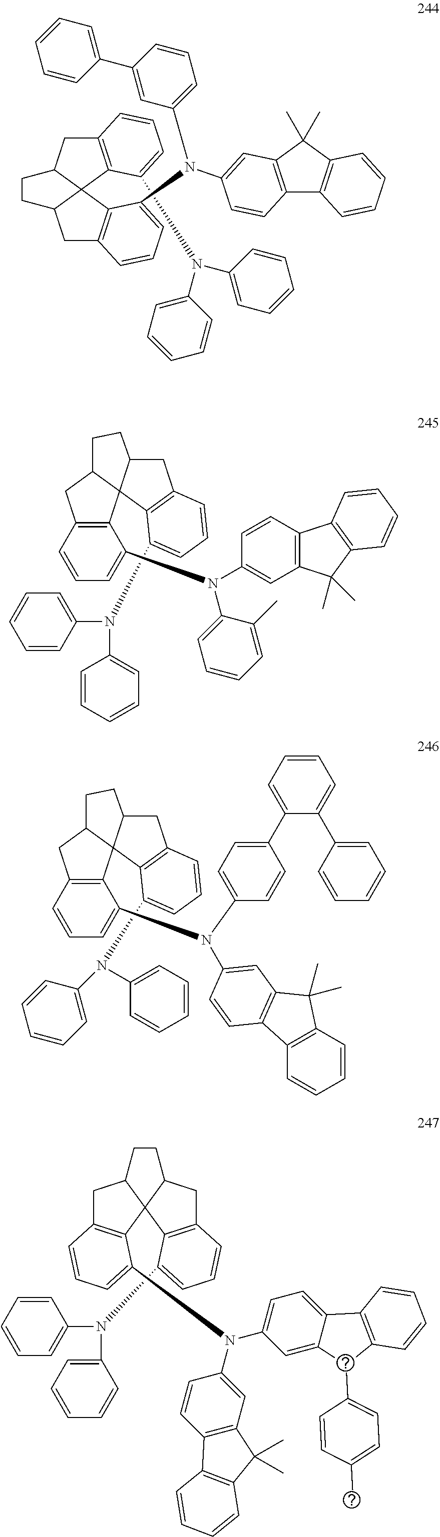 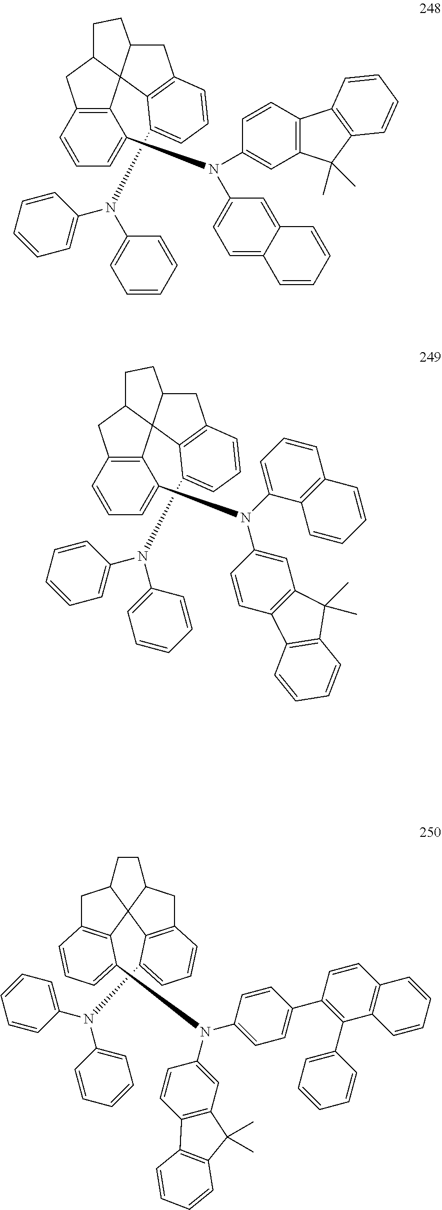 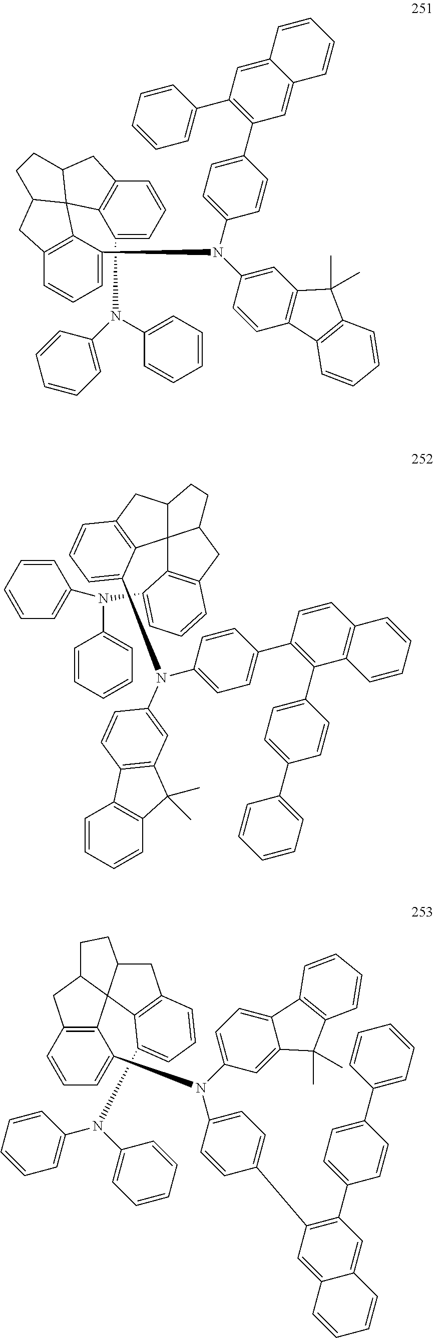 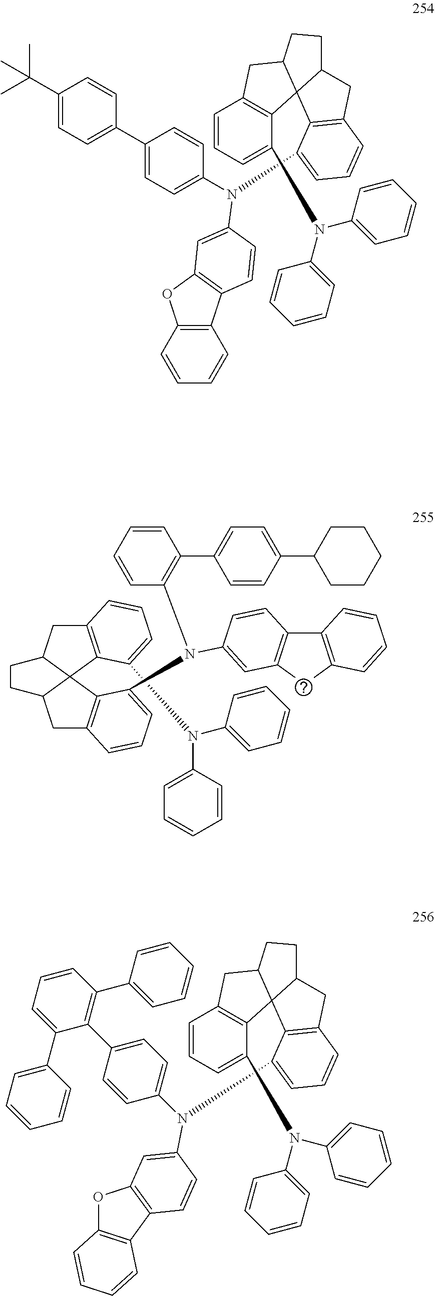 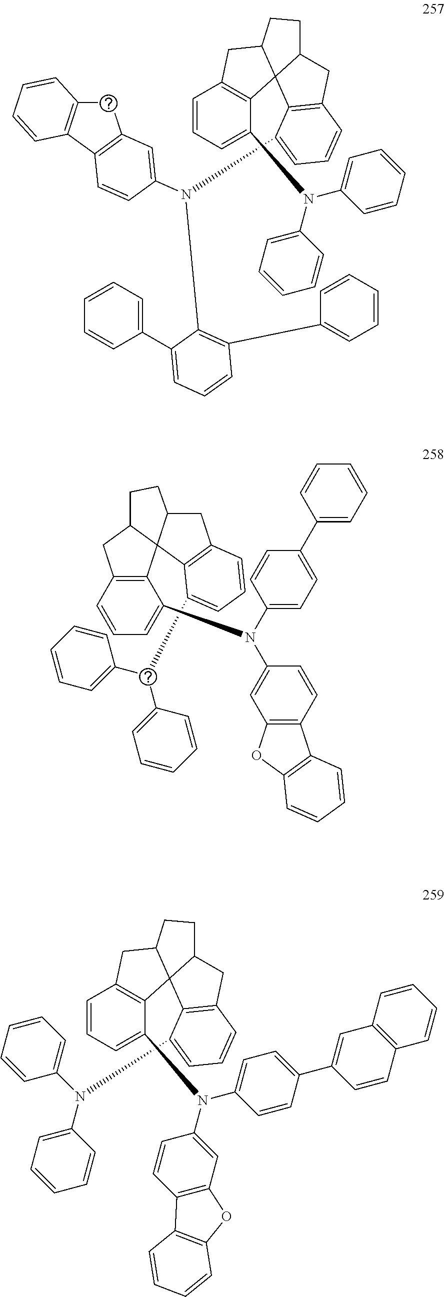 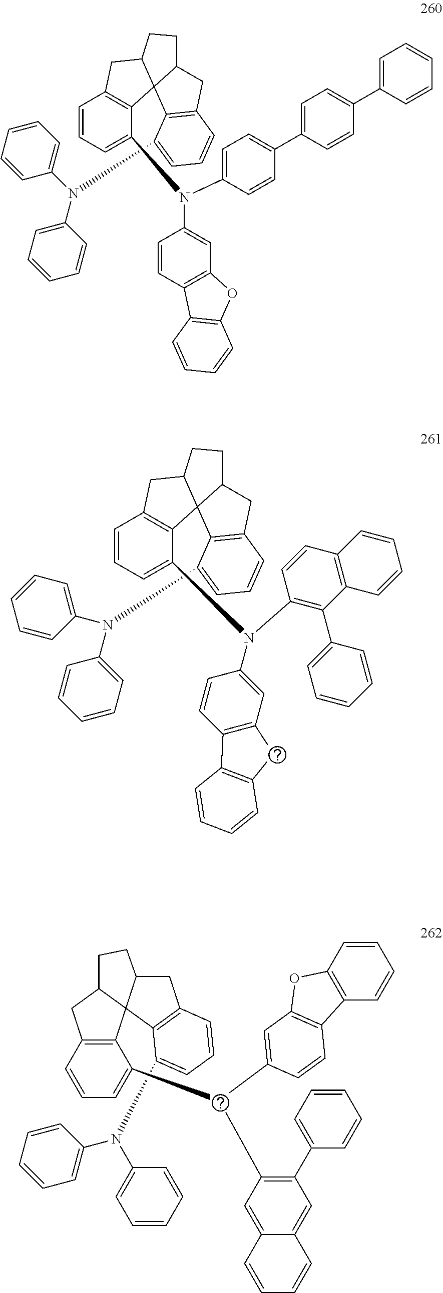 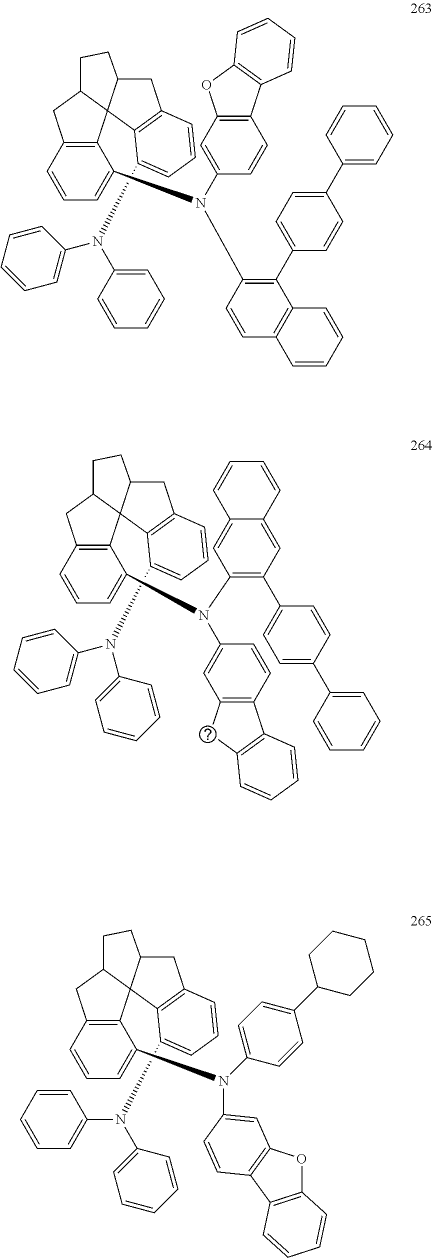 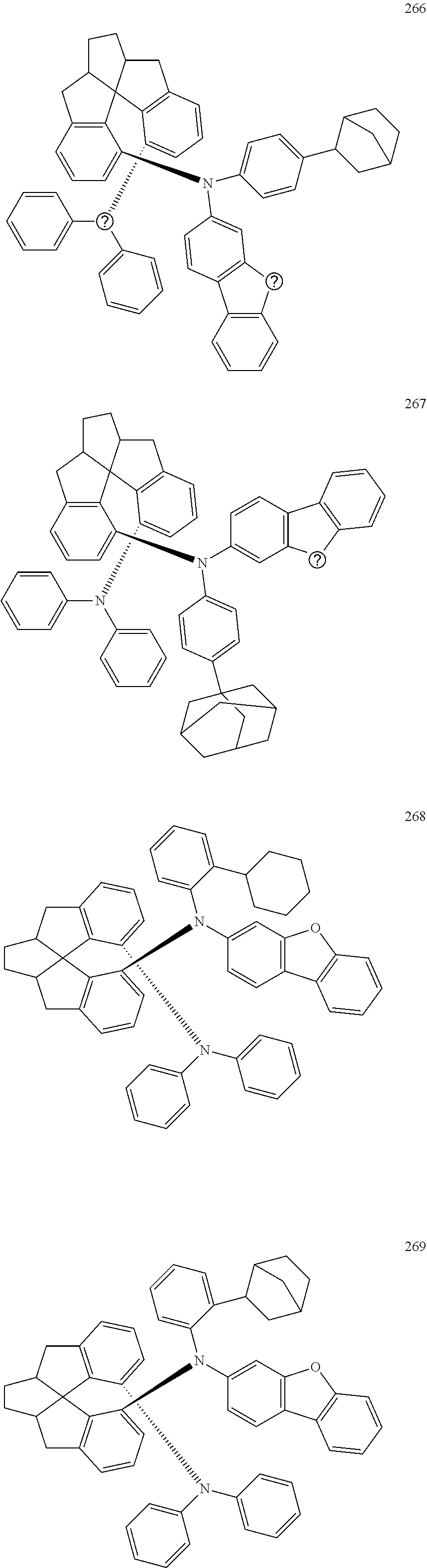 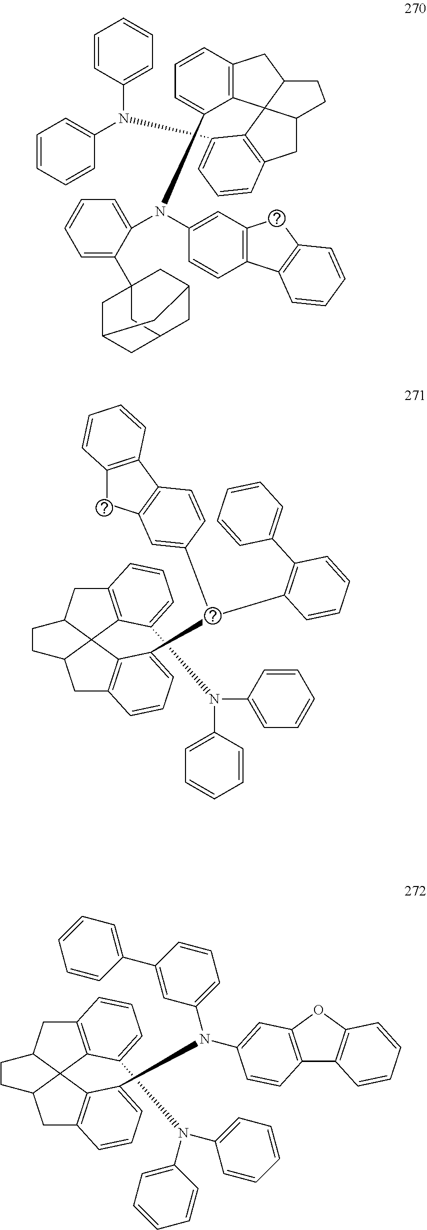 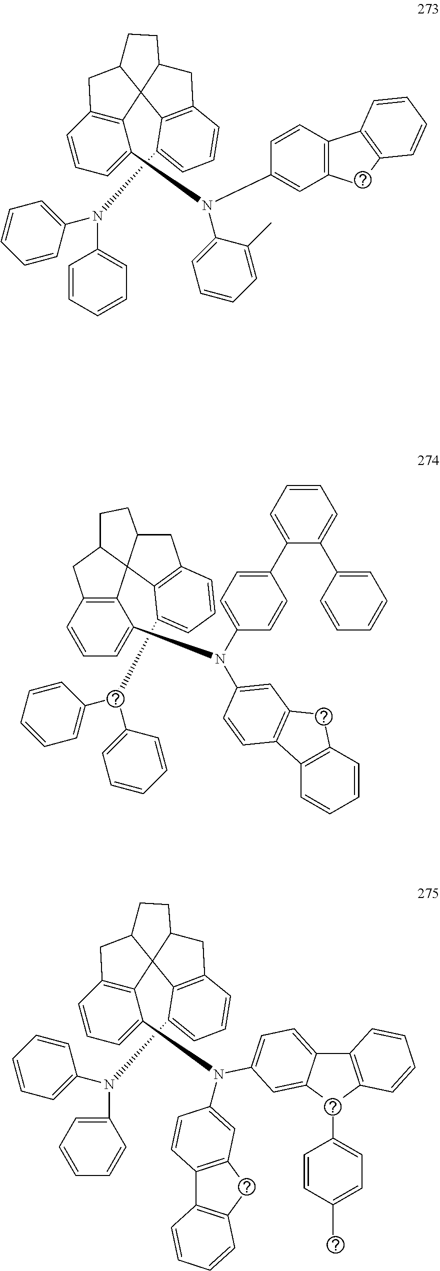 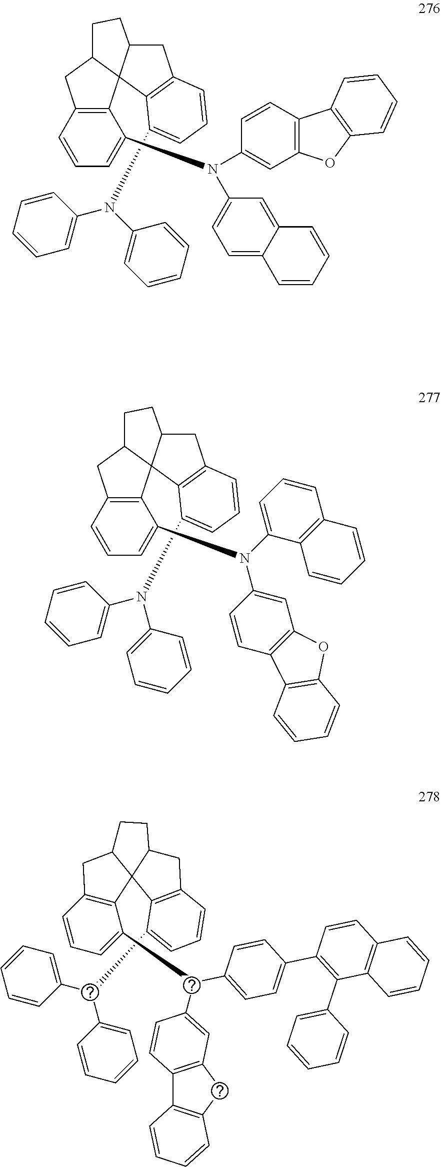 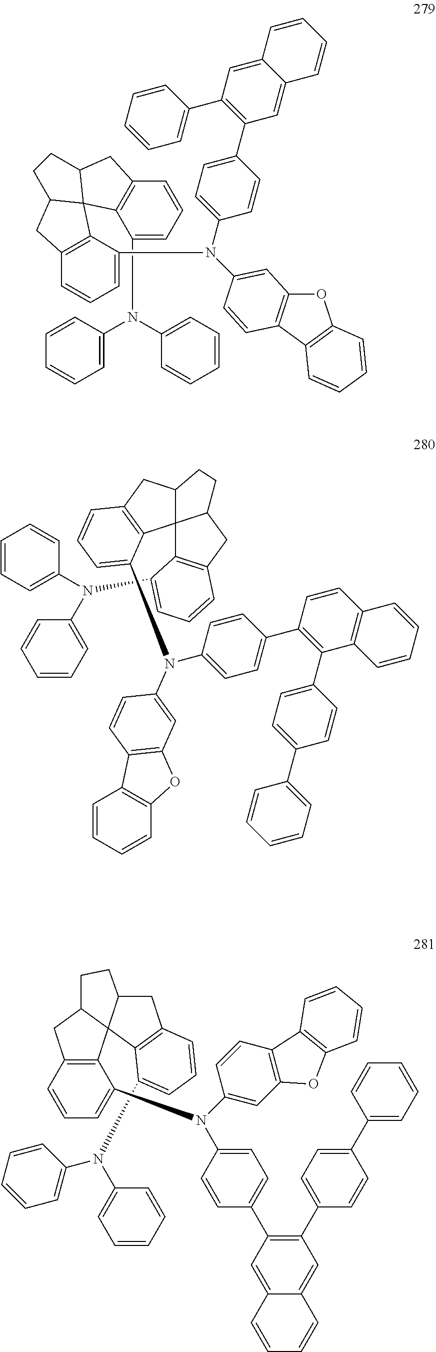 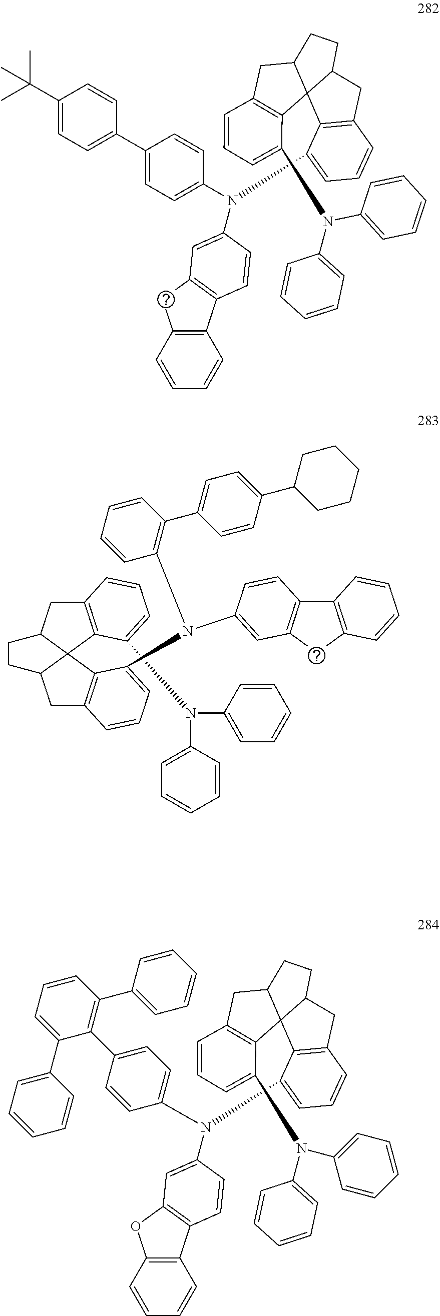 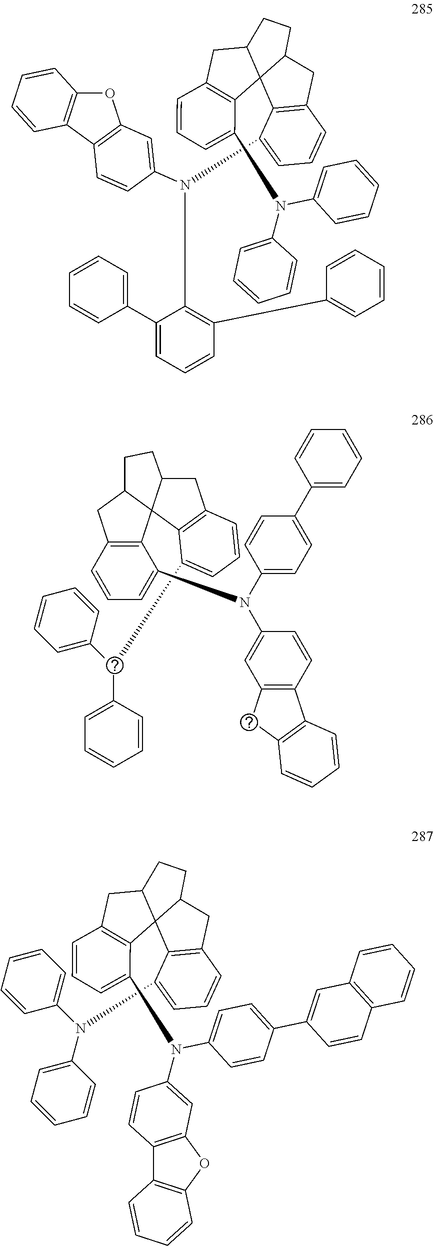 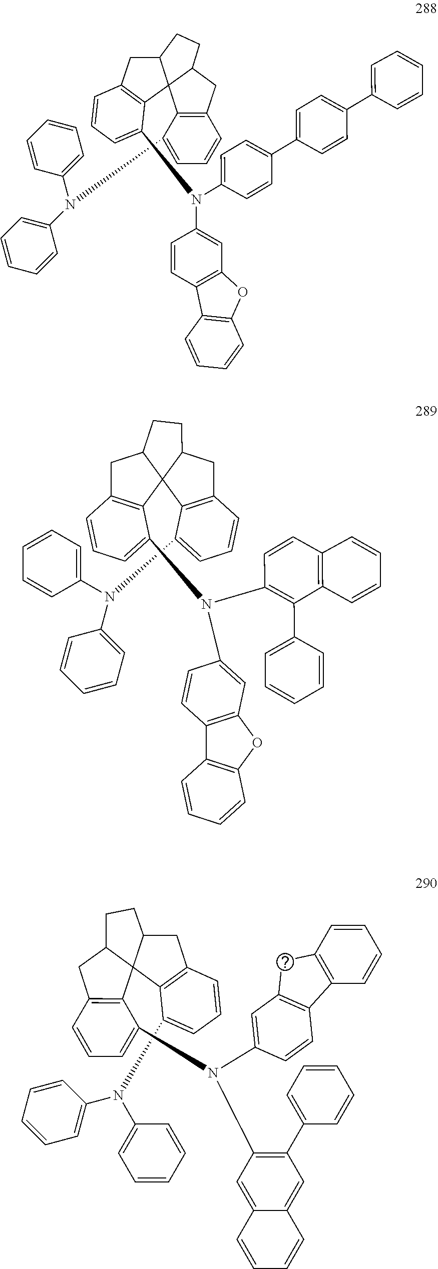 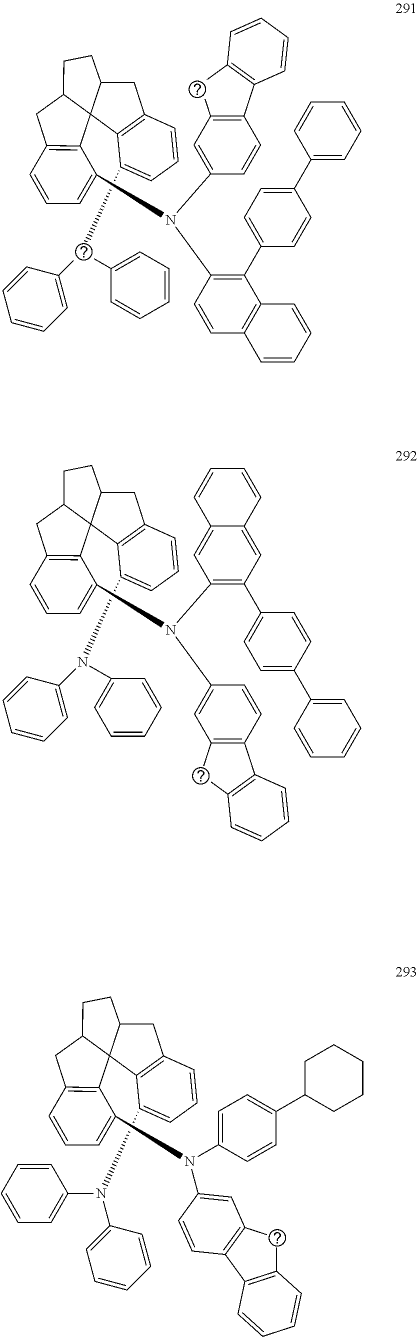 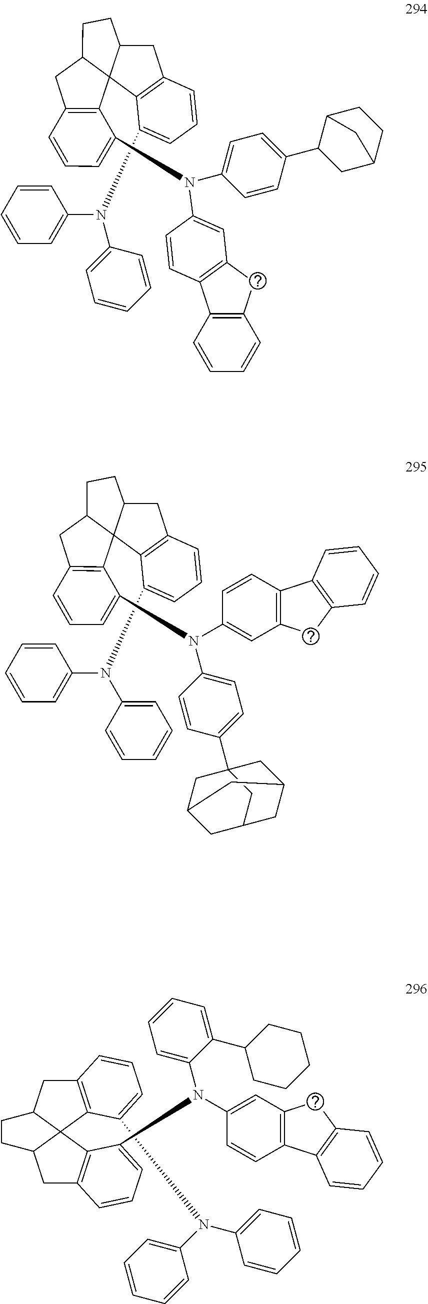 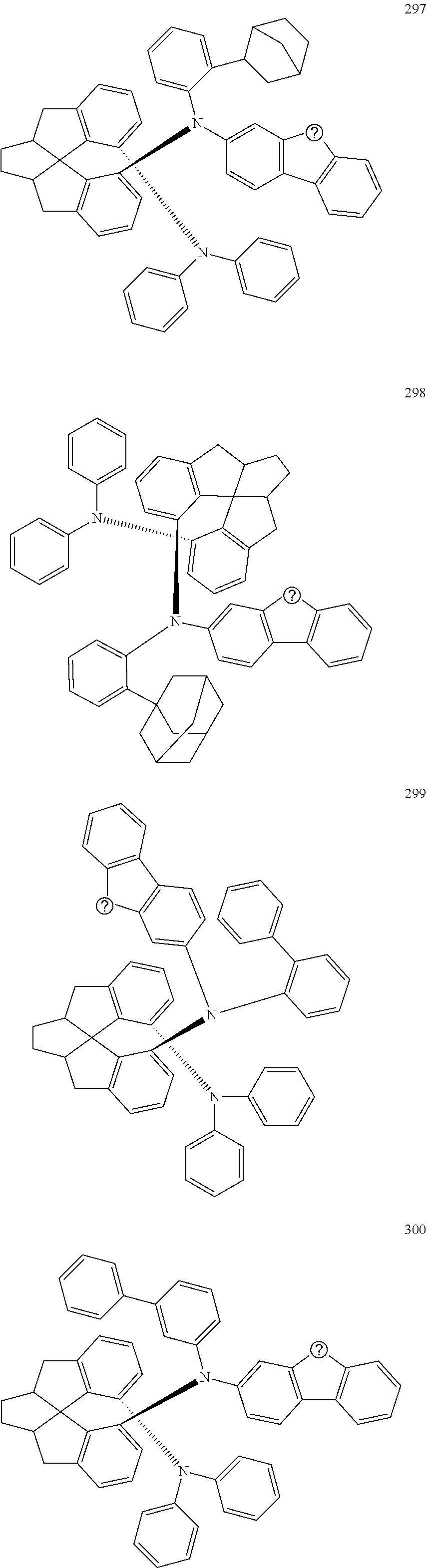 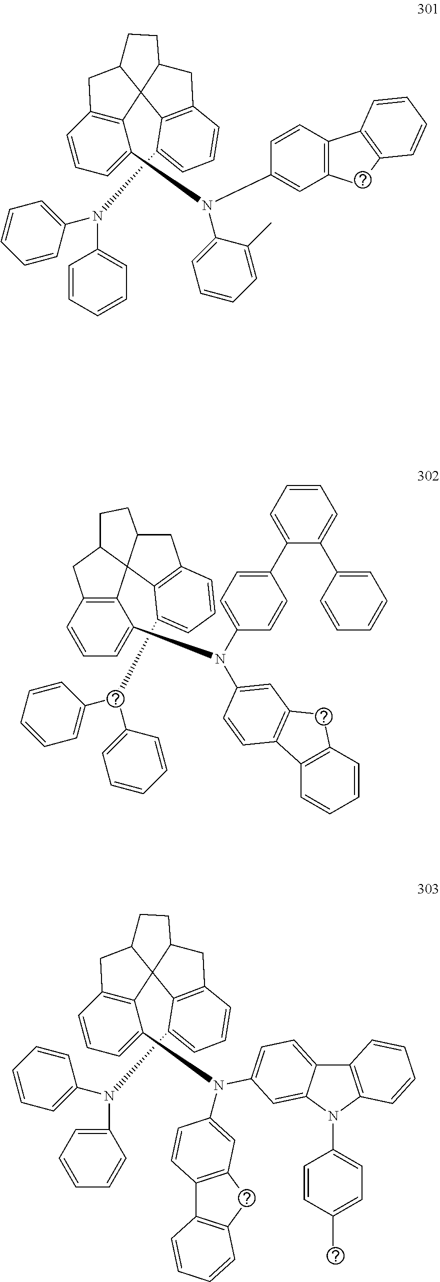 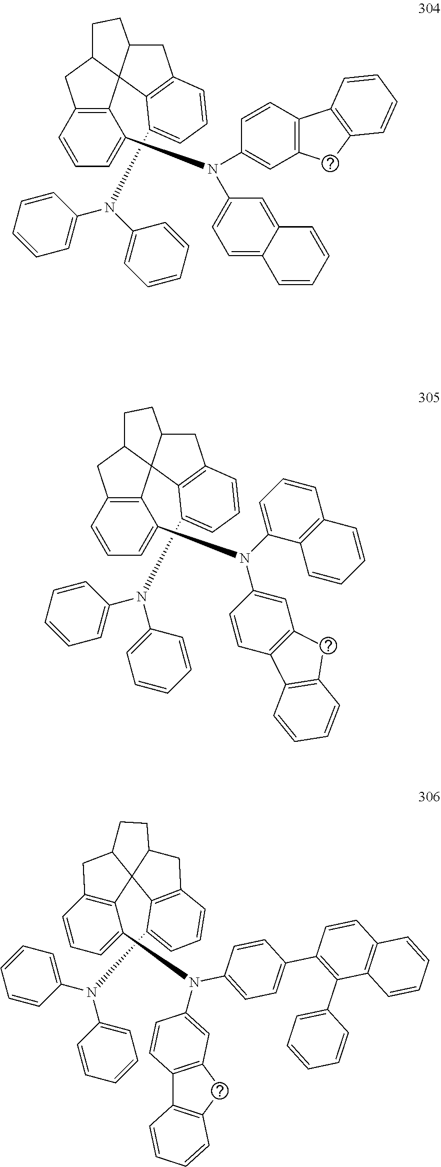 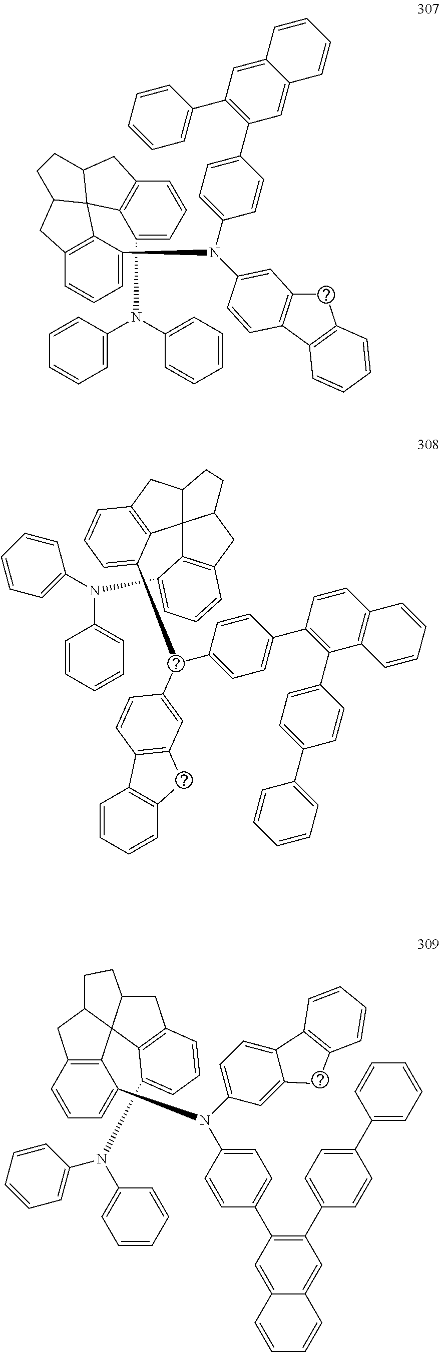 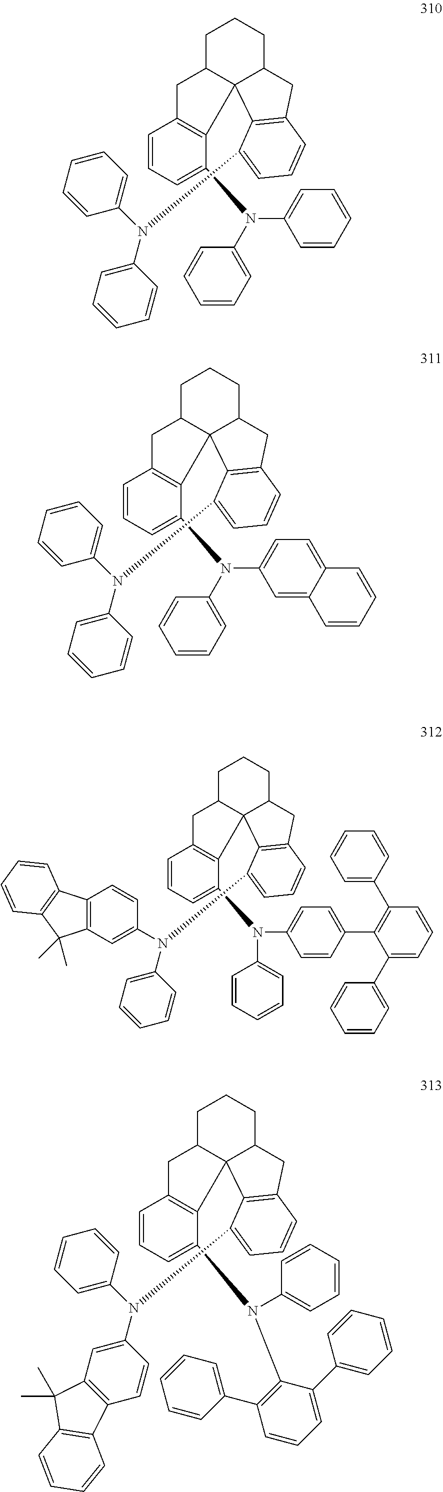 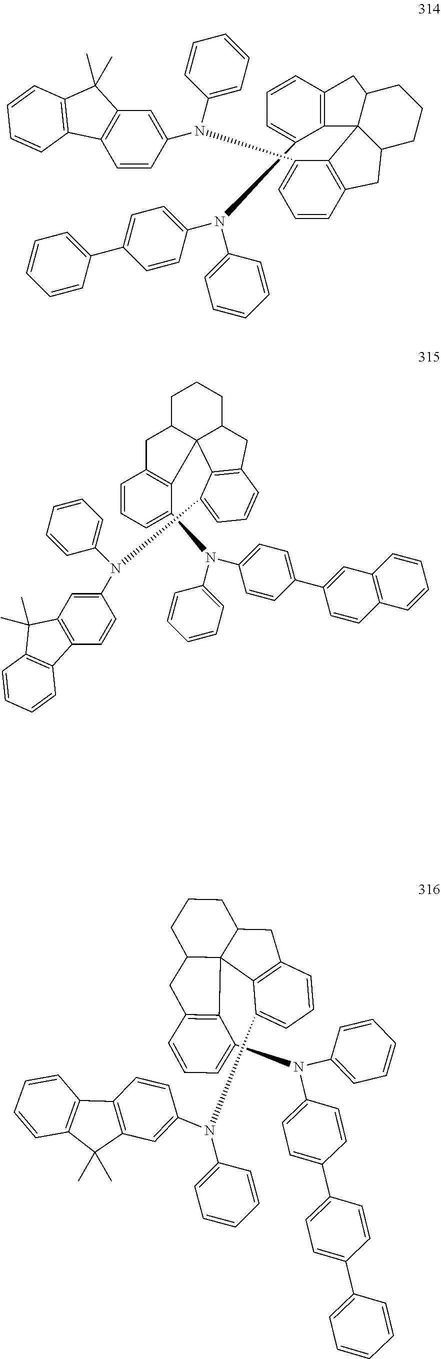 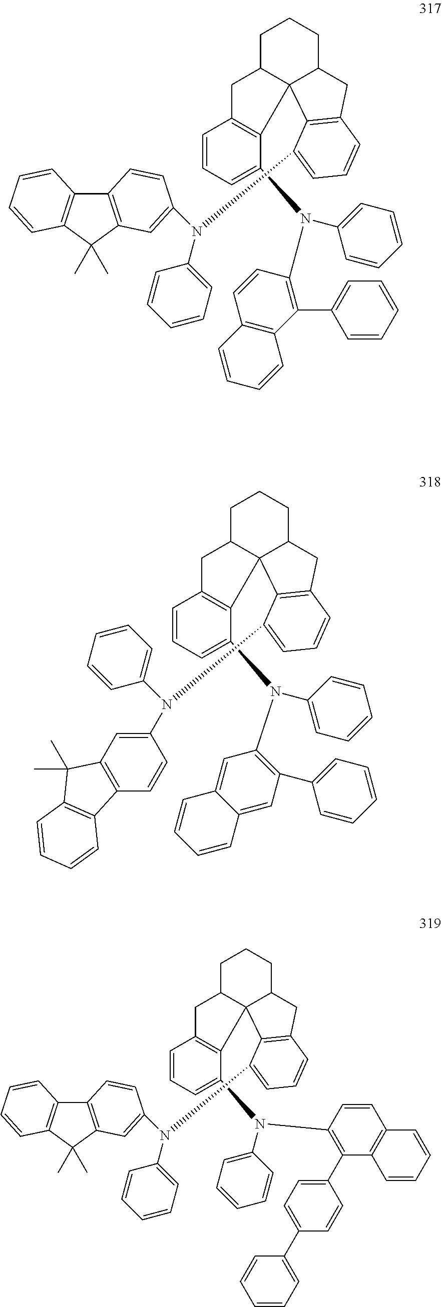 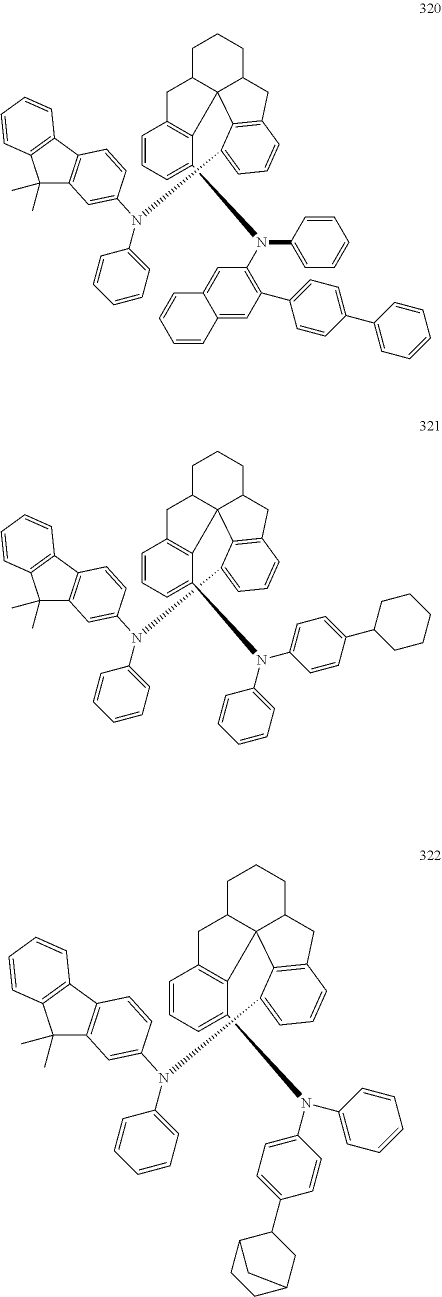 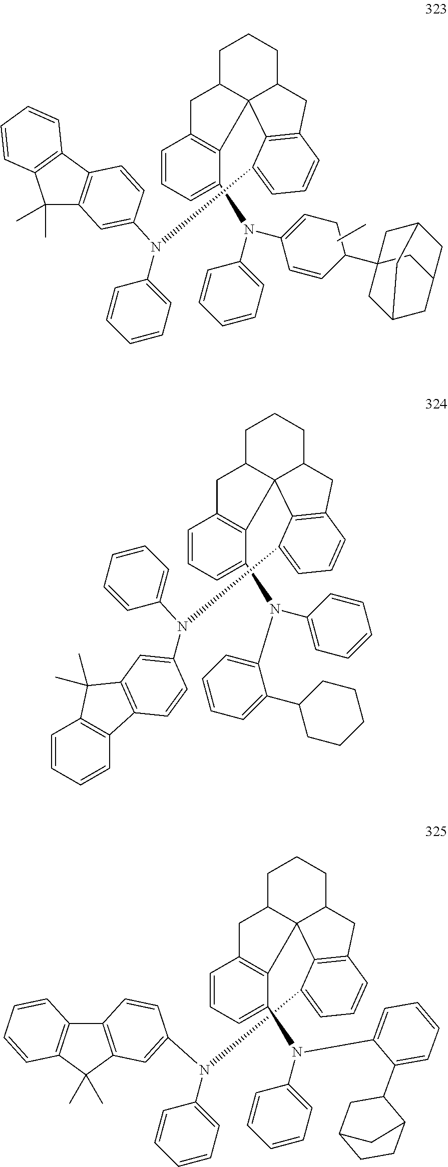 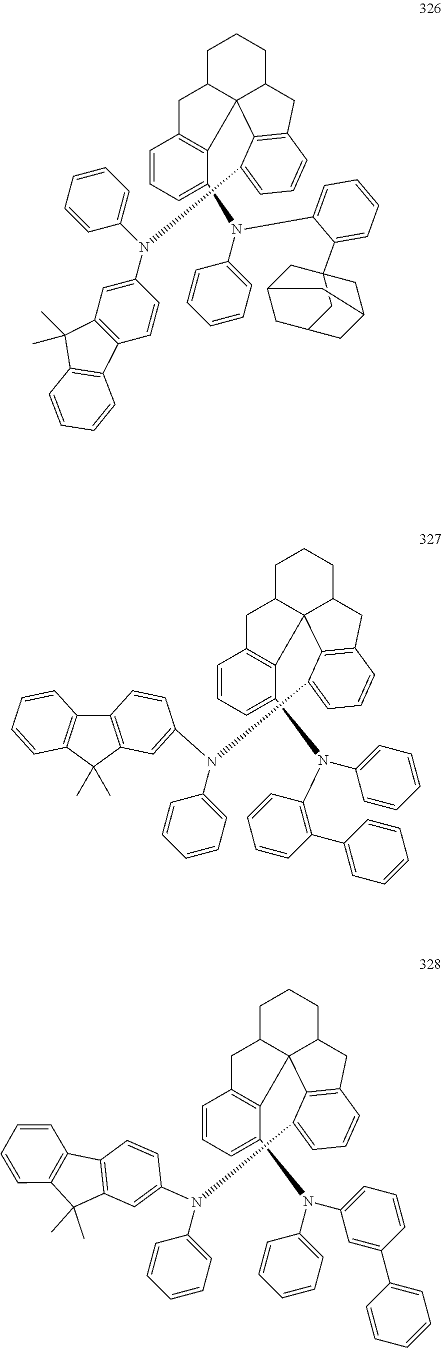 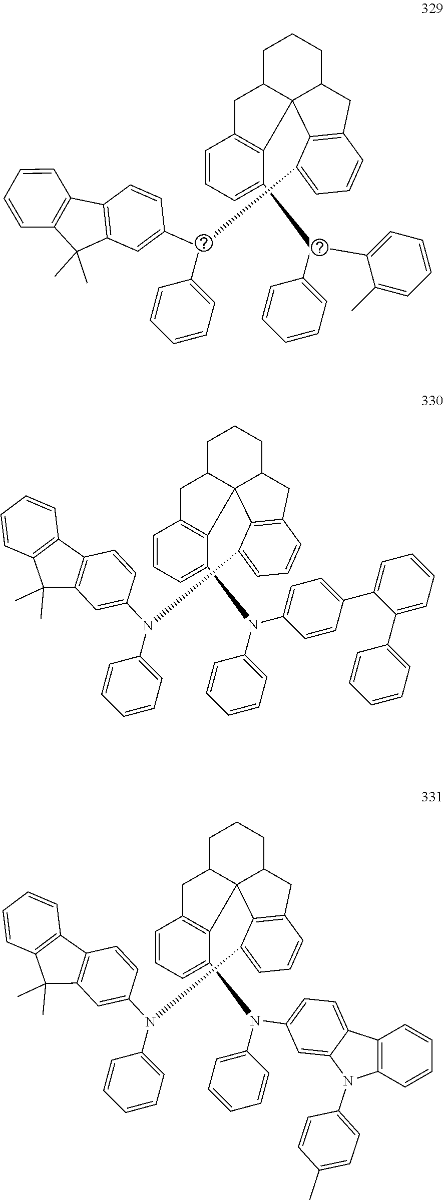 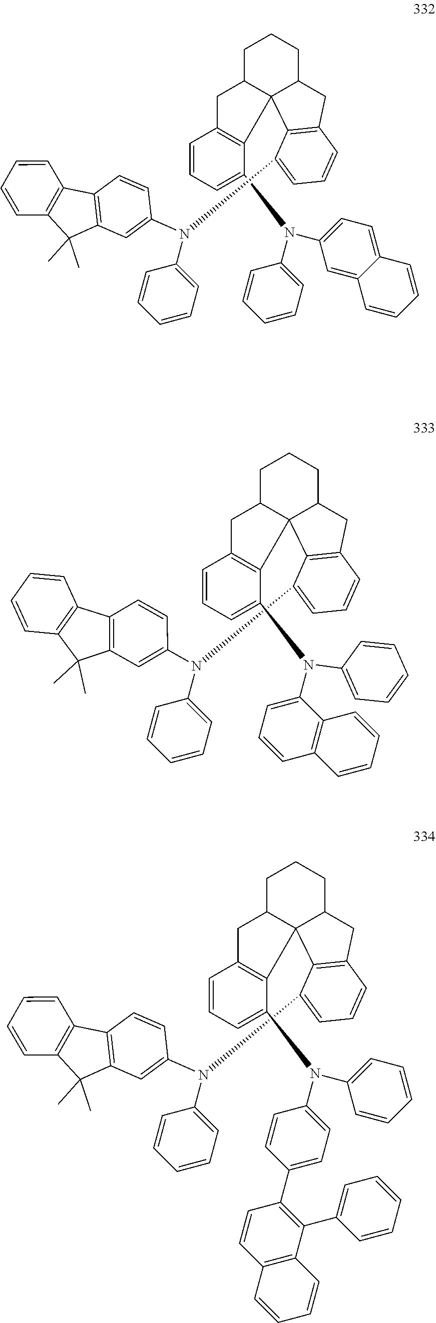 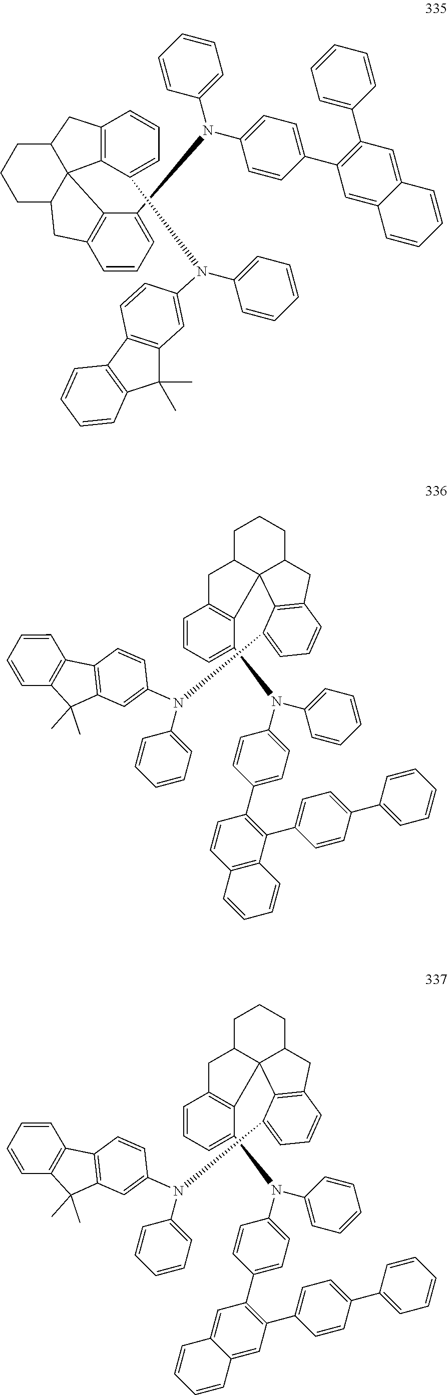 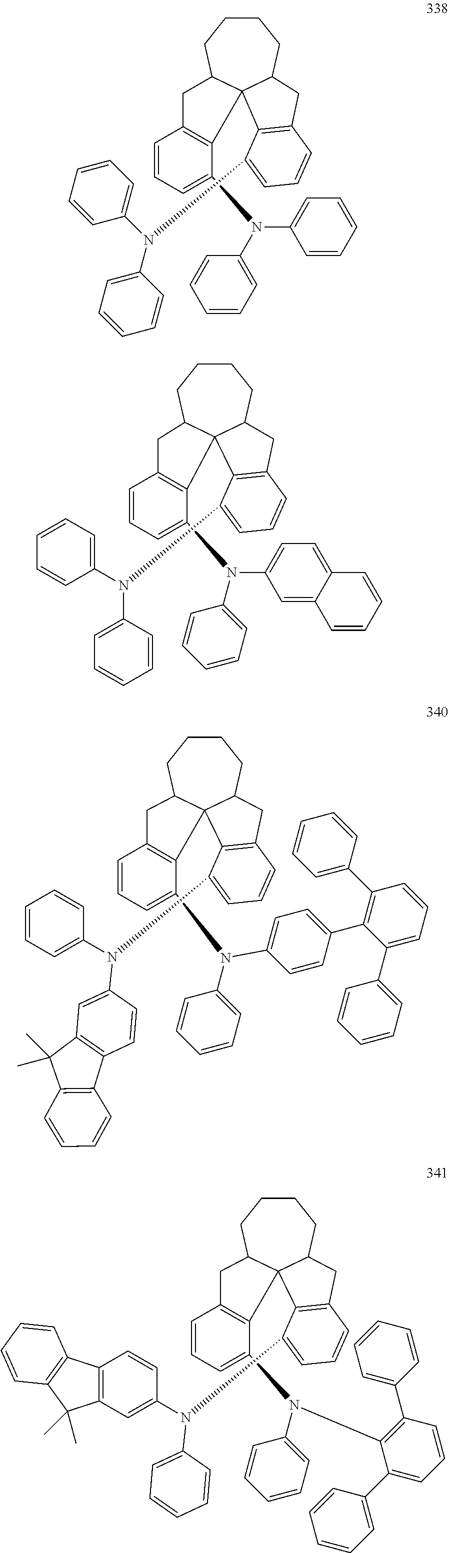 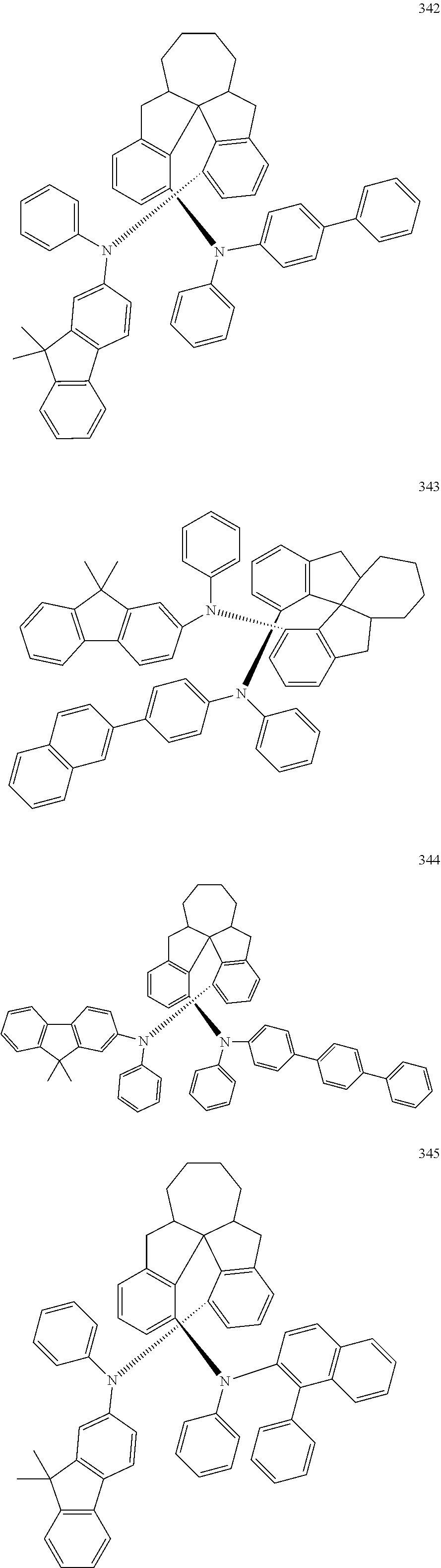 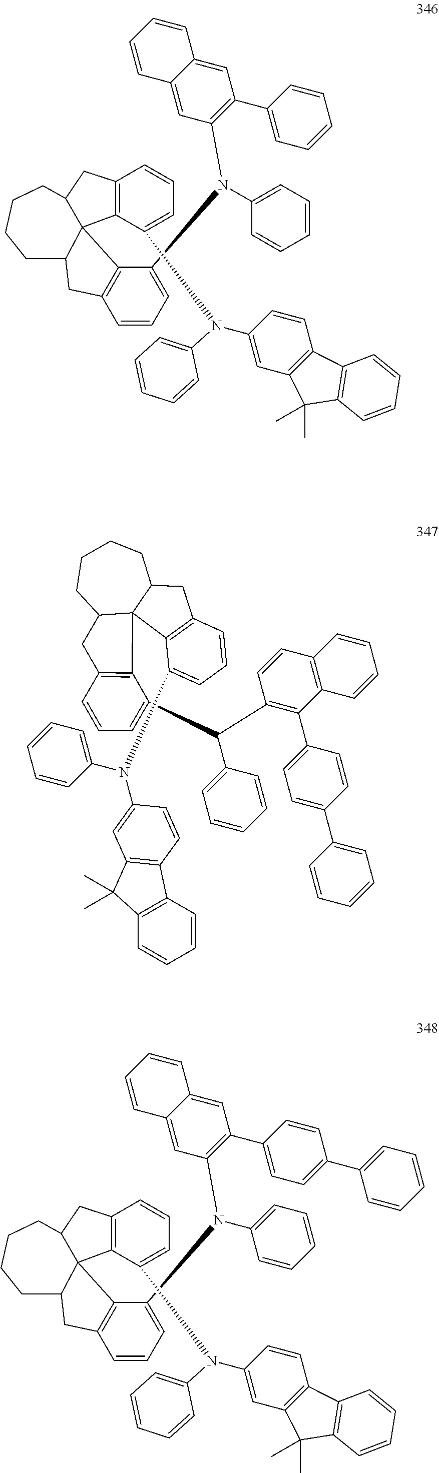 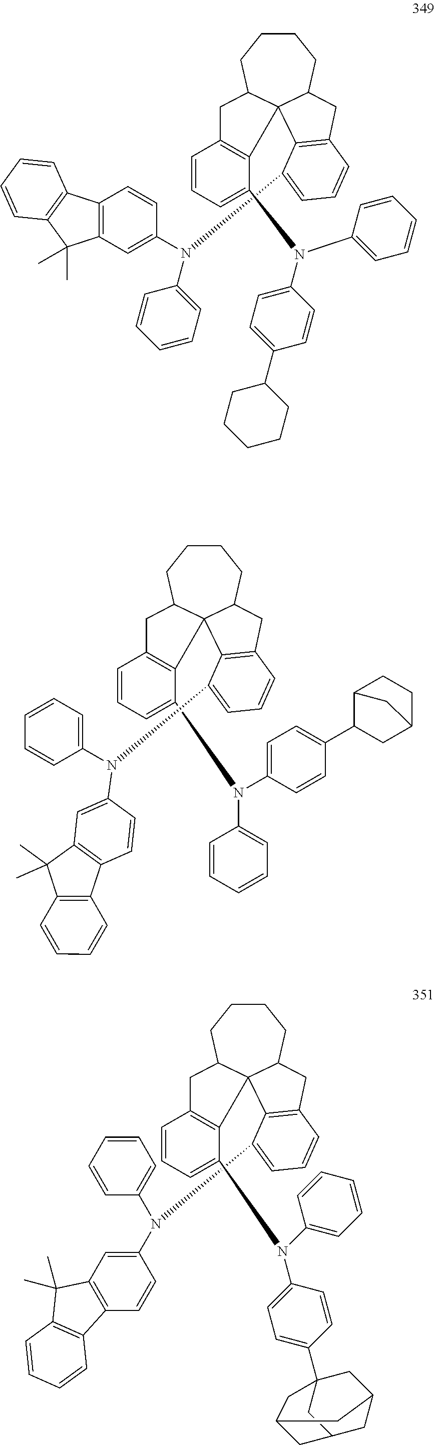 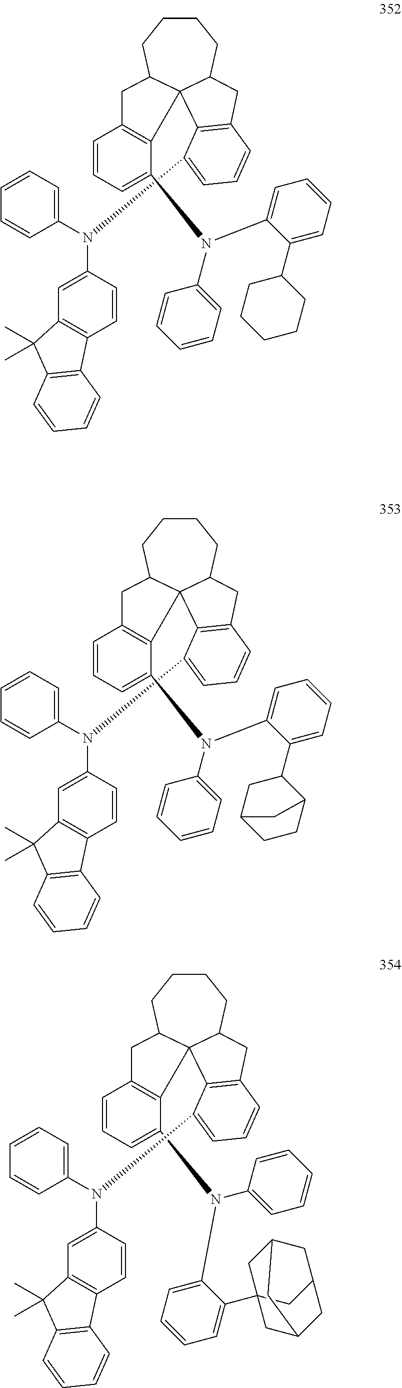 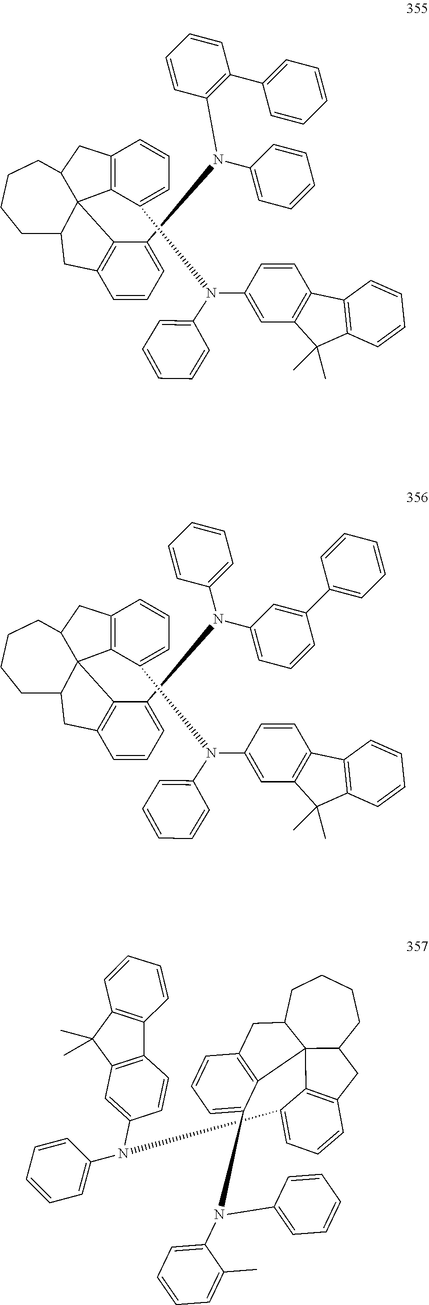 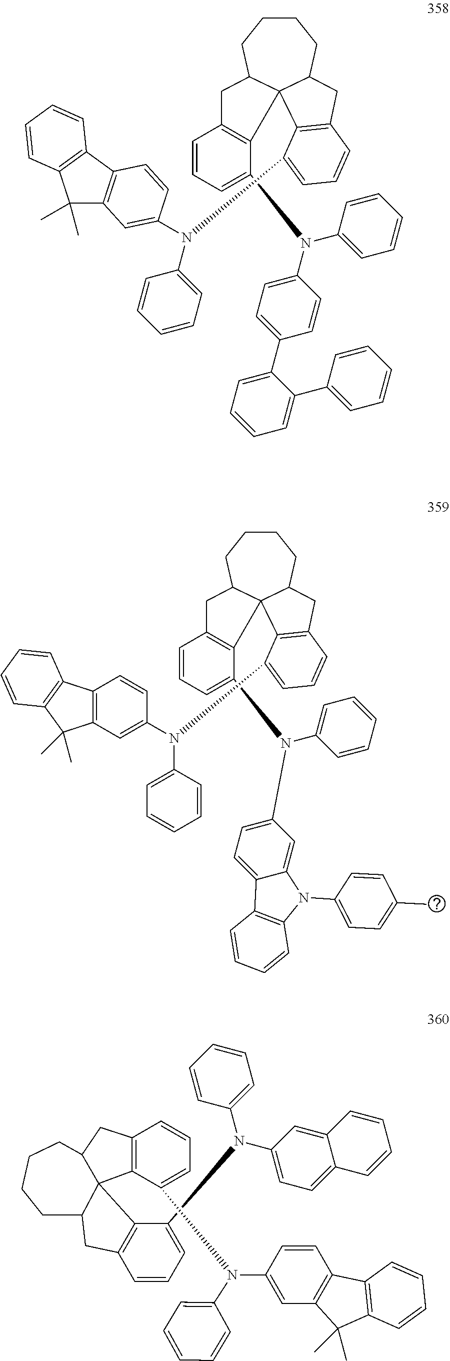 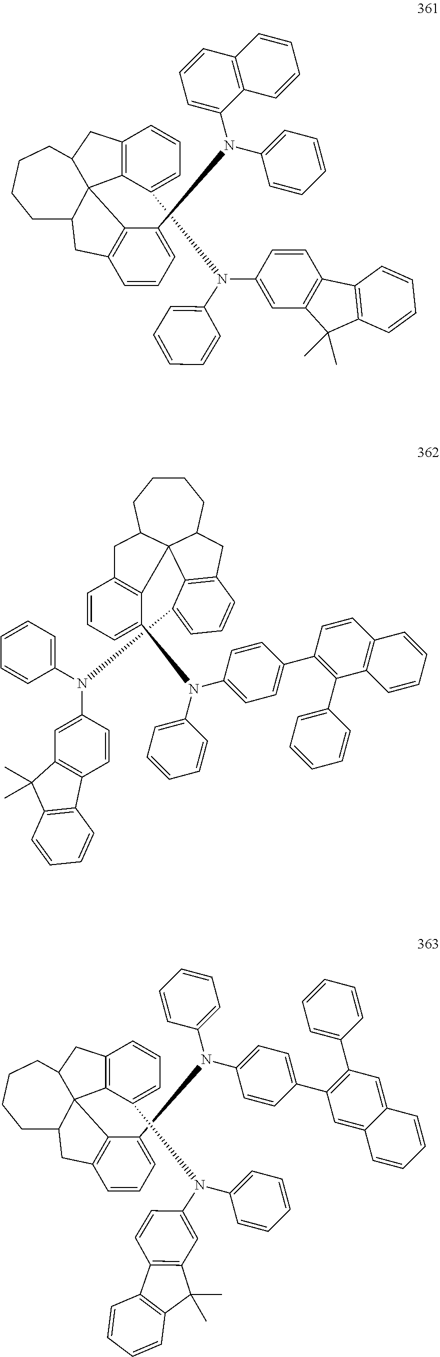 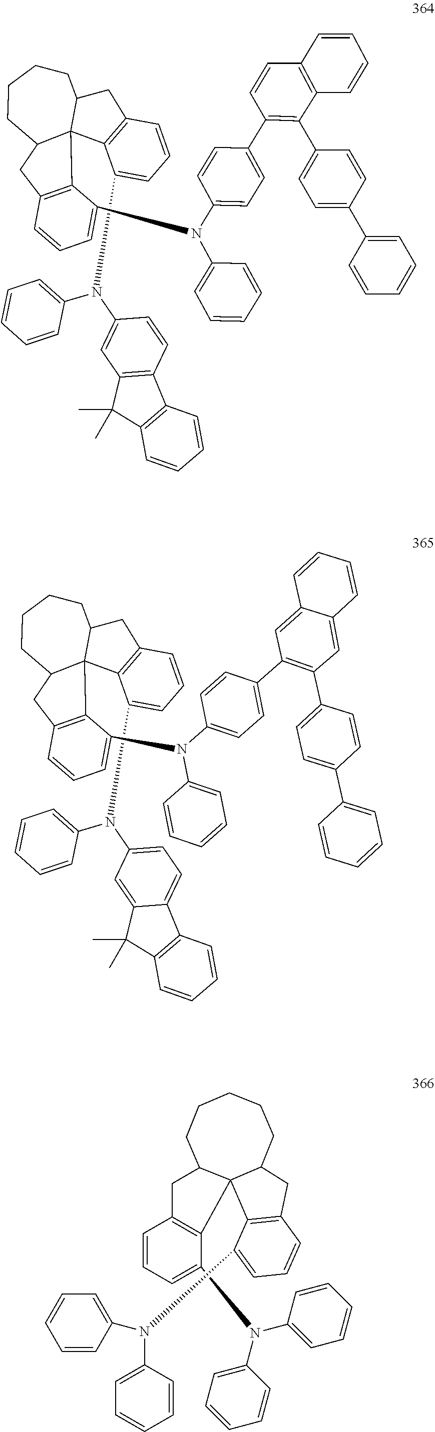 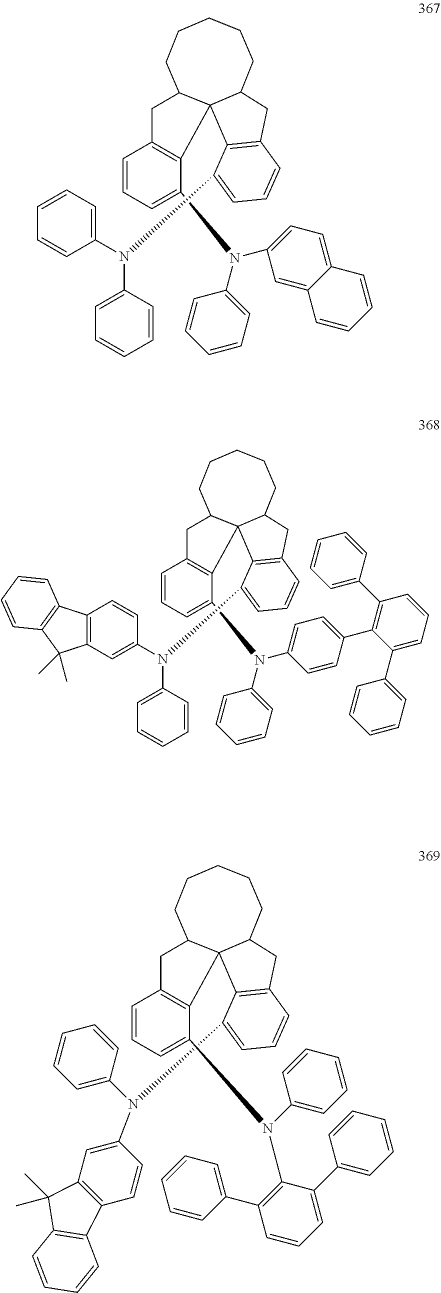 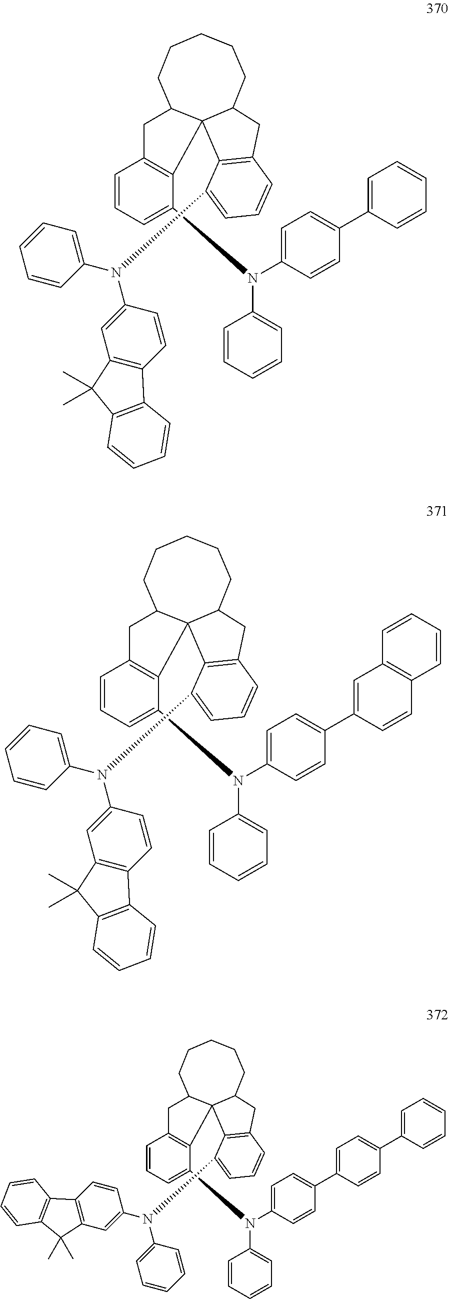 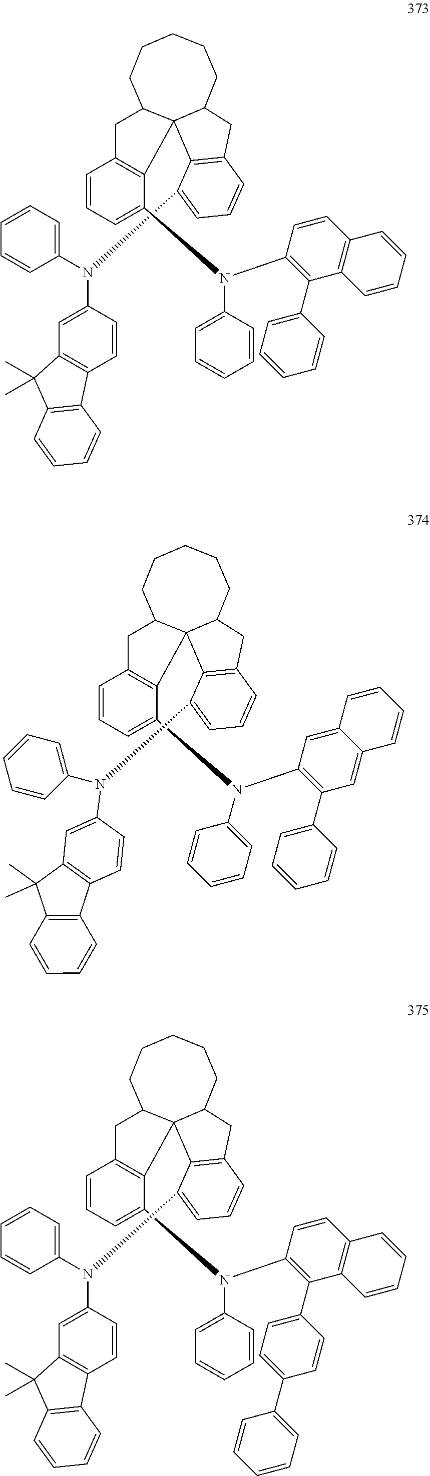 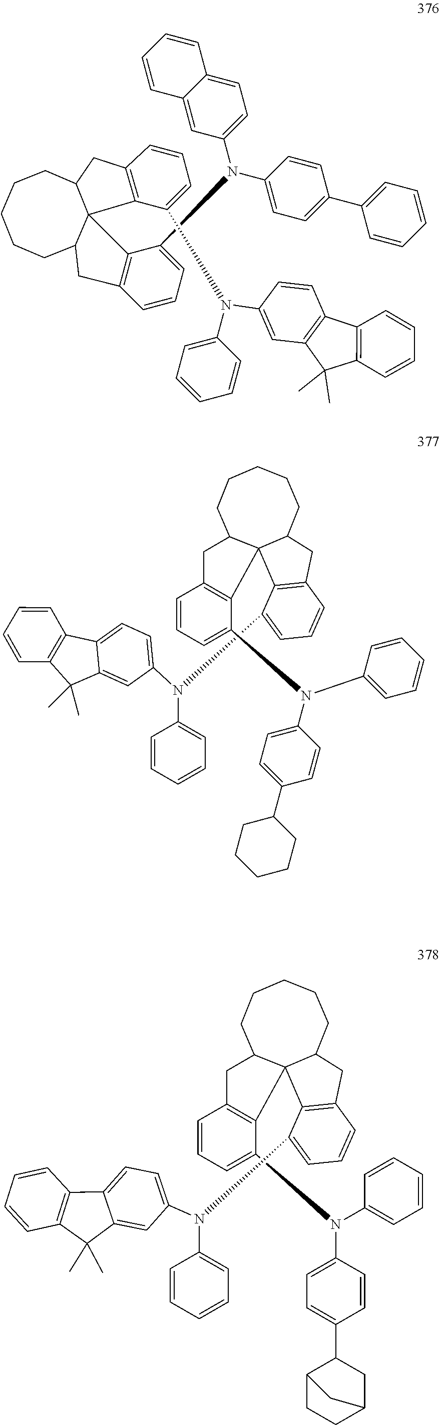 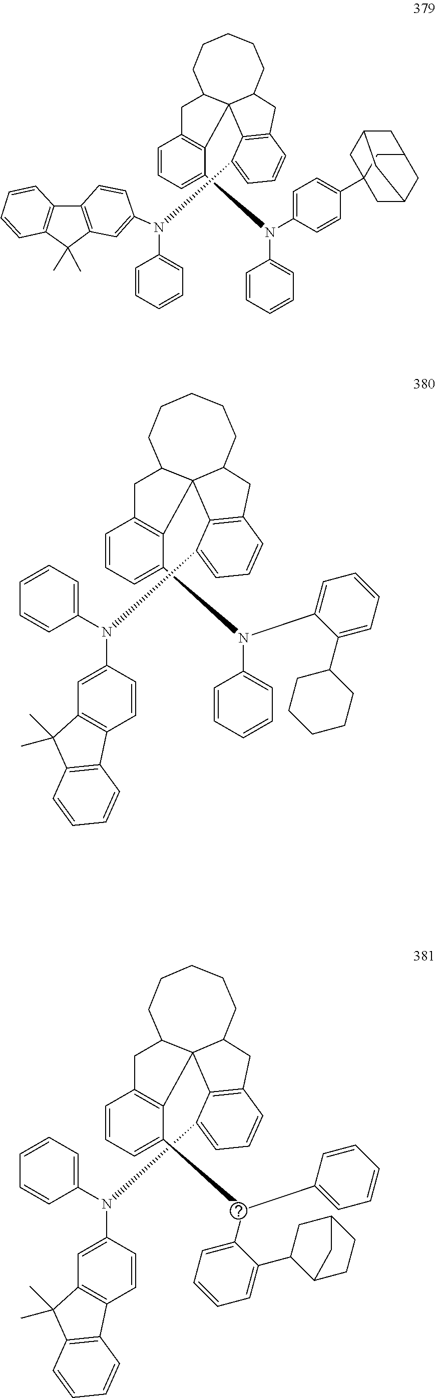 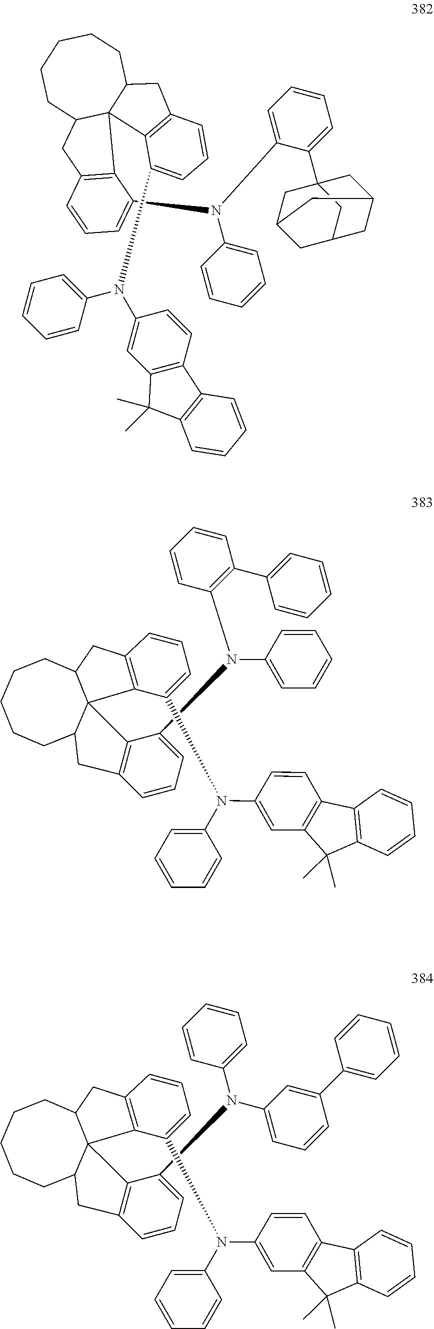 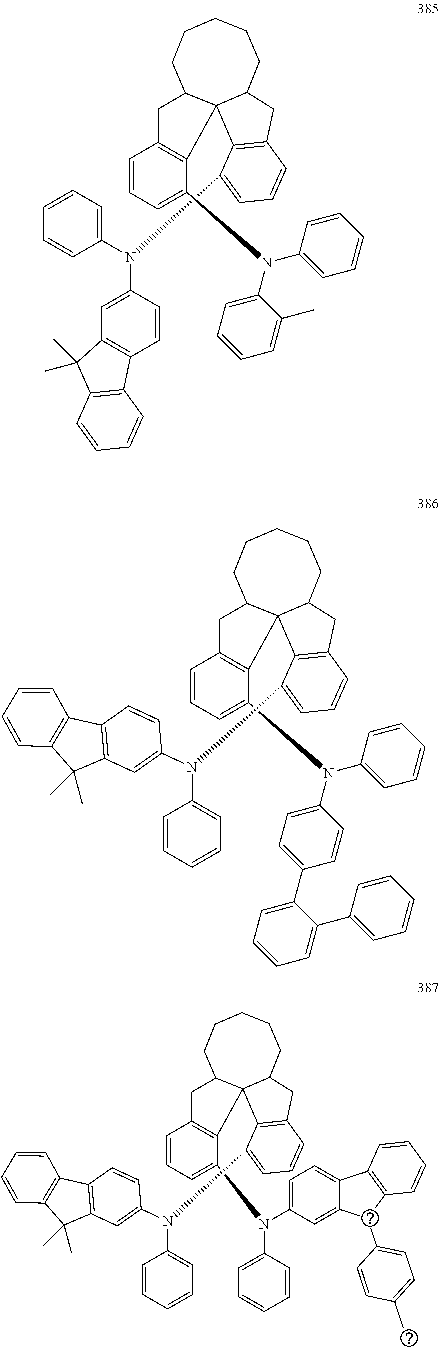 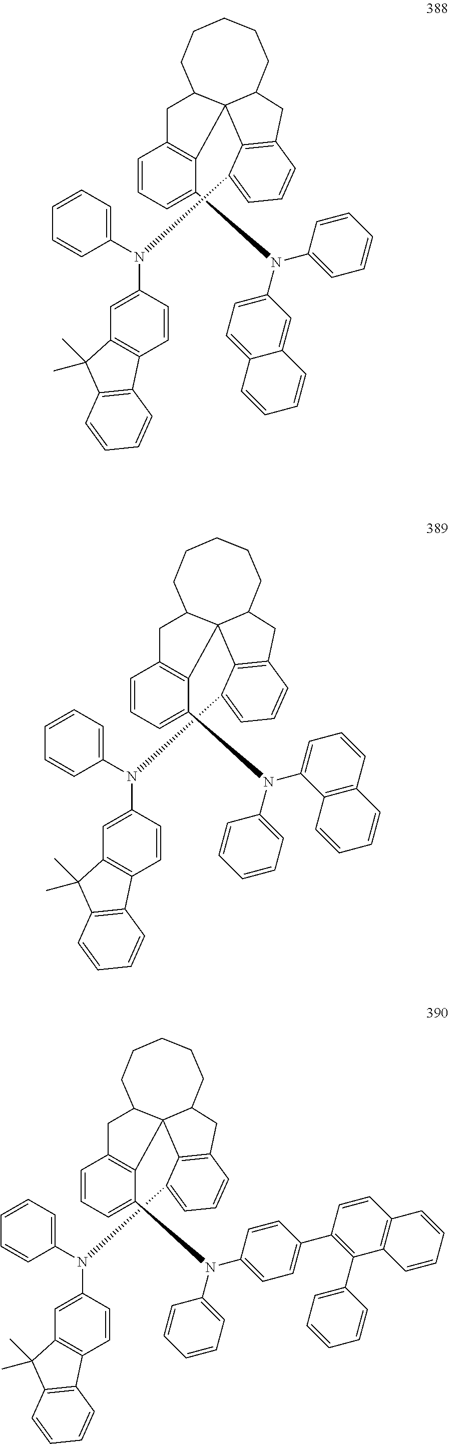 [chemical structure]
